(19) United States
(12) Patent Application Publication
(10) Pub. No.: US 2026/0152503 A1
(43) Pub. Date: Jun. 4, 2026

(54) SPIROBICYCLIC COMPOUNDS

(71) Applicant: ACERAND THERAPEUTICS (HONG KONG) LIMITED, Hong Kong (CN)

(72) Inventors: Bin LIU, Fishers, IN (US); Kuo-Long YU, Zionsville, IN (US); Weitao PAN, Old Saybrook, CT (US)

(21) Appl. No.: 18/852,541

(22) PCT Filed: Mar. 30, 2023
(86) PCT No.: PCT/US2023/016937

§ 371 Date: Sep. 30, 2024

Related U.S. Application Data
(60) Provisional application No. 63/325,721, filed on Mar. 31, 2022.
(60) Provisional application No. 63/419,438, filed on Oct. 26, 2022.

(51) Int. Cl. C07D 487/04; A61K 31/519; A61P 35/00; C07B 59/00; C07D 519/00

(52) U.S. Cl. CPC C07D 487/04; A61K 31/519; A61P 35/00; C07B 59/002; C07D 519/00

(57) ABSTRACT

Spirobicyclic compounds, particularly a compound of formula I and pharmaceutical compositions thereof are provided. The compound of formula I is effective in treating diseases associated with or modulated by FGFR2, including cancers that harbor aberrant activation of FGFR2.

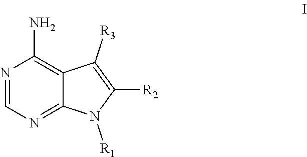

### CROSS REFERENCES TO RELATED APPLICATIONS

This application claims the benefit of priority based on U.S. Application Ser. Nos. 63/325,721 filed on Mar. 31, 2022 and 63/419,438 filed on Oct. 26, 2022. The contents and disclosures of both applications are incorporated herein by reference in their entirety.

### TECHNICAL FIELD

The present invention provides spirobicyclic compounds, pharmaceutical compositions thereof, methods of using the same, and processes for preparing the same.

### BACKGROUND

The fibroblast growth factor receptor (FGFR) family of receptor tyrosine kinases consists of four transmembrane receptors (FGFR1, FGFR2, FGFR3 and FGFR4). Fibroblast Growth Factors (FGFs) act in concert with heparin sulfate proteoglycans (HSPG) as high-affinity FGFR agonists. Binding of FGF/HSPG to FGFR induces receptor dimerization and activation of FGFR tyrosine kinases, followed by trans-autophosphorylation of tyrosine residues in the cytoplasmic kinase domain. This event triggers activation of multiple downstream signaling cascades including the RAS-MAPK-ERK, PI3K-AKT, PKC and JAK-STAT pathways, which promote cell survival, antiapoptotic, proliferation, migration, development, differentiation, and angiogenesis (Gallo, et al. Cytokine & Growth Factor Reviews 2015 26, 425; Ahmad et al. Biochimica et Biophysica Acta 2012, 1823, 850).

It has been demonstrated that aberrantly activated FGF/FGFR signaling (i.e., FGFR gene amplification, mutation, gene rearrangements or fusions) axis results in constitutive activation of downstream signaling pathways to allow cells to grow uncontrollably, leading to malignant tumors (Nakamura, npj Precis. Onc. 2021, 5, 66; Gallo, et al. Cytokine & Growth Factor Reviews 2015 26, 425; Ahmad et al. Biochimica et Biophysica Acta 2012, 1823, 850). Particularly, gene amplification of FGFR1 is identified in about 10% and 10-25% of breast cancer and squamous-cell lung cancer cases, respectively. FGFR2 gene amplification occurs in 4-10% of gastric cancer cases with a poor prognosis. FGFR2 mutations or fusions are found in approximately 10% and 15% of endometrial carcinoma and intrahepatic cholangiocarcinoma, respectively. FGFR3 fusions are common in bladder cancer, lung cancer and glioblastoma (Nakamura, npj Precis. Onc. 2021, 5, 66; Goyal et al. Cancer Treatment Reviews 2021, 95, 102170). Collectively aberrantly activated FGFRs as oncogenic drivers are promising therapeutic targets for cancers. Indeed, several pan-FGFR tyrosine kinase inhibitors (TKIs) have been shown to be a successful therapeutic strategy in numerous tumor types. However, acquired resistance to pan-FGFR-TKIs is becoming increasingly prominent due to target specific mutations such as gate keeper, molecular breaker as well as other mutations induced by FGFR-TKIs (Yue, et al. J Hematol Oncol 2021, 14, 23; Goyal, et al. Cancer Discov. 2019 9, 1064). In addition, on-target toxicity limits clinical usage of these inhibitors. One of the most common adverse events associated with pan-FGFR-TKIs is hyperphosphatemia, an on-target off-tumor effect of FGFR1 inhibition. Given those reasons of acquired resistance and adverse event of hyperphosphatemia for pan-FGFR-TKIs, there is an emerging need for the development of new generation of FGFR selective inhibitors, such as a FGFR2 selective inhibitor that can overcome resistance and reduce side effects of pan-FGFR-TKIs for those cancers with aberrant activation of FGFR2 (i.e., endometrial carcinoma, gastric cancer, intrahepatic cholangiocarcinoma, etc.).

International Publication No. WO 2020/231990 A1 describes certain compounds as FGFR inhibitors. Chinese Application Publication No. CN 115594682 also discloses certain compounds as FGFR inhibitors. Further, International Publication No. WO 2022/109551 A1 describes certain compounds as inhibitors of non-receptor tyrosine-protein kinase Src.

### SUMMARY OF THE INVENTION

The present invention provides new compounds which inhibit FGFR2. The present invention further provides new compounds which selectively inhibit FGFR2 with respect to FGFR1, FGFR3 or FGFR4. As such, compounds of the present invention are useful in treating diseases and or conditions associated with or modulated by FGFR2 including but not limited to cancers that harbor aberrant activation of FGFR2 such as endometrial carcinoma, gastric cancer, intrahepatic cholangiocarcinoma, breast cancer, urinary tract cancer and non-small cell lung cancer (NSCLC). Further, compounds of the present invention are useful in treating diseases or conditions associated with or modulated by FGFR2 with reduced or attenuated toxicity, or adverse events associated with FGFR1, FGFR3 or FGFR4.

The present invention provides new compounds which inhibit FGFR2 and, as such, are useful in treating the disorders discussed herein. The new compounds of the present invention provide an alternative for treatment of said disorders.

The present invention provides a compound of formula I.

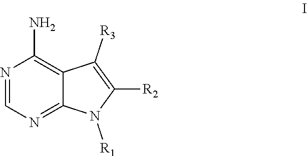

or a pharmaceutically acceptable salt thereof, wherein each of R₁, R₂ and R₃ is defined in the embodiments, classes, and subclasses herein. Further, the present invention provides a compound of formula I, pharmaceutical compositions thereof, methods of using the same, and processes for preparing the same.

The present invention provides a compound of formula I

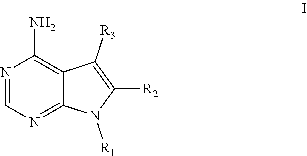

wherein
R₁ is hydrogen, C1-C6 alkyl or substituted C1-C6 alkyl;
R₂ is substituted C7-C19 spiro bicycloalkyl, substituted C7-C19 spiro bicycloalkenyl, substituted C7-C19 spiro heterobicycloalkanyl or substituted C7-C19 spiro heterobicycloalkenyl; and
R₃ is C1-C6 alkyl, substituted C1-C6 alkyl, C3-C10 cycloalkyl, substituted C3-C10 cycloalkyl, 4- to 10-membered heterocycloalkyl, substituted 4- to 10-membered heterocycloalkyl, aryl or substituted aryl, heteroaryl or substituted heteroaryl;
   or a pharmaceutically acceptable salt thereof.

Further, the present invention provides a compound of formula I

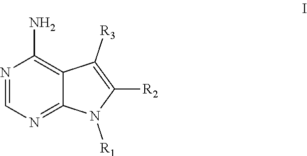

wherein
   R₁ is hydrogen, C1-C6 alkyl or substituted C1-C6 alkyl;
   R₂ is

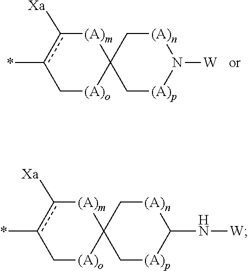

(A)ₘ is CRₐR_{b}
(A)ₙ is CRₐR_{b};
(A)ₒ is CRₐR_{b} or O;
(A)ₚ is CRₐR_{b};
Rₐ is on each occurrence independently selected from the group consisting of H and C1-C3 alkyl;
R_{b} is on each occurrence independently selected from the group consisting of H and C1-C3 alkyl;
[symbol] is a single bond or a double bond;
m, n, o and p are on each occurrence independently selected from the group consisting of 0, 1, 2 or 3;
provided when (A)ₒ is O, o is 1;
Xa is H or halogen;
W is COR₅ or SO₂R₅;
R₅ is

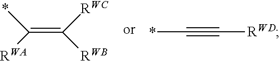

R^{WA}, R^{WB}, R^{WC} are on each occurrence independently selected from the group consisting of H, C1-C6 alkyl, substituted C1-C6 alkyl, halogen and CN;
R^{WD} is H or C1-C6 alkyl; and
R₃ is C1-C6 alkyl, substituted C1-C6 alkyl, substituted aryl, heteroaryl or substituted heteroaryl, heterocycloalkyl or substituted heterocycloalkyl, or CN;
   or a pharmaceutically acceptable salt thereof.

Further, the present invention provides a compound of formula I
wherein m, n, o and p are on each occurrence independently selected from the group consisting of 0, 1 or 2; or a pharmaceutically acceptable salt thereof.

Further, the present invention provides a compound of formula I wherein
R₅ is

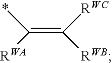

and
R^{WA}, R^{WB}, R^{WC} are on each occurrence independently selected from the group consisting of H and C1-C6 alkyl;
   or a pharmaceutically acceptable salt thereof.

Further, the present invention provides a compound of formula I wherein
R₂ is

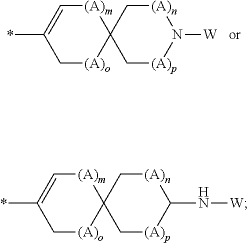

or a pharmaceutically acceptable salt thereof.

Further, the present invention provides a compound of formula I wherein
R₂ is

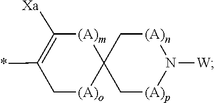

(A)ₘ is CRₐR_{b}
(A)ₙ is CRₐR_{b};
(A)ₒ is CRₐR_{b} or O;
(A)ₚ is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
provided when (A)ₒ is O, o is 1;
Xa is H or halogen;
W is COR₅ or SO₂R₅;
R₅ is

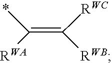

R^{WA}, R^{WB}, R^{WC} are on each occurrence independently selected from the group consisting of H, C1-C6 alkyl, substituted C1-C6 alkyl, halogen and CN;
   or a pharmaceutically acceptable salt thereof.

Further, the present invention provides a compound of formula I wherein
R₁ is CH₃;
R₂ is
R₃ is

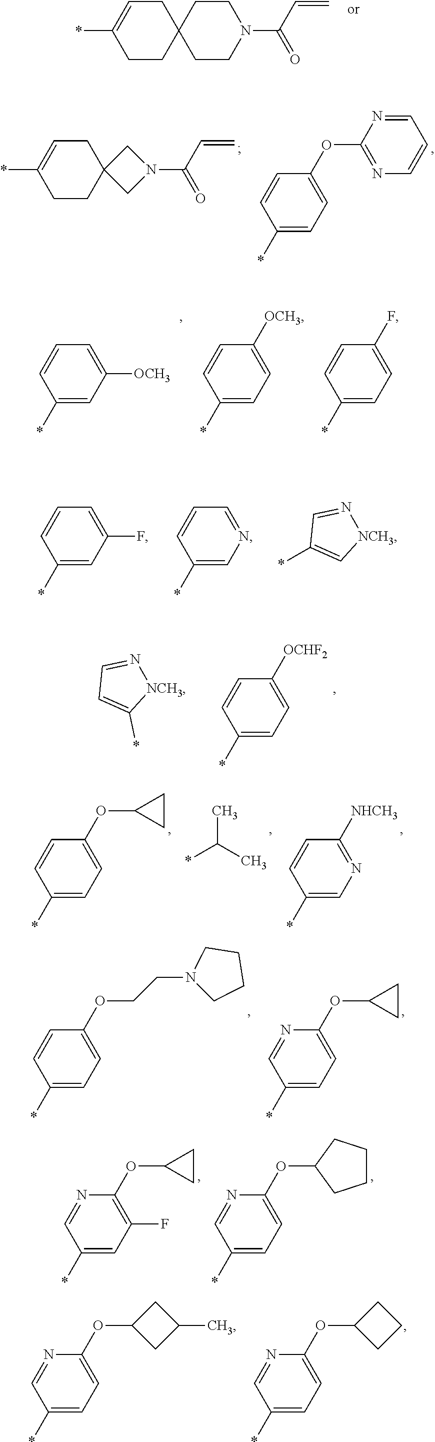

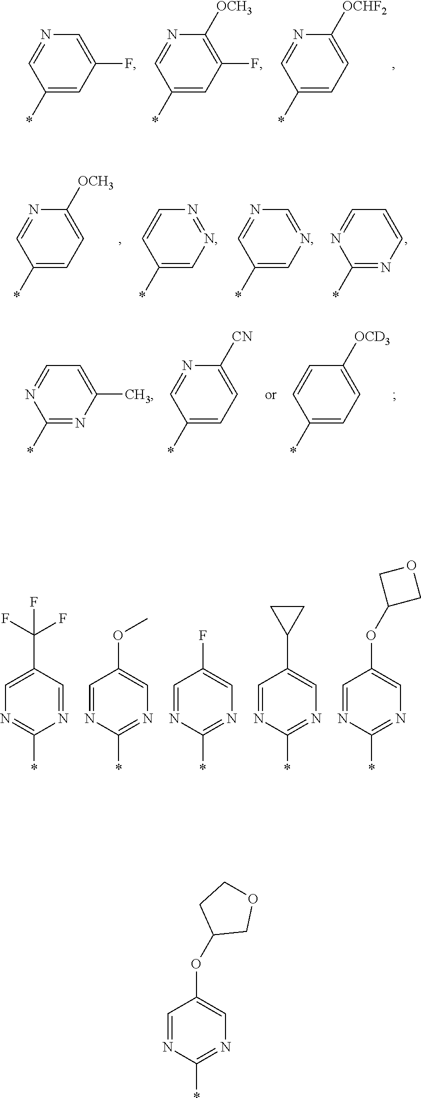

R₃ can also be

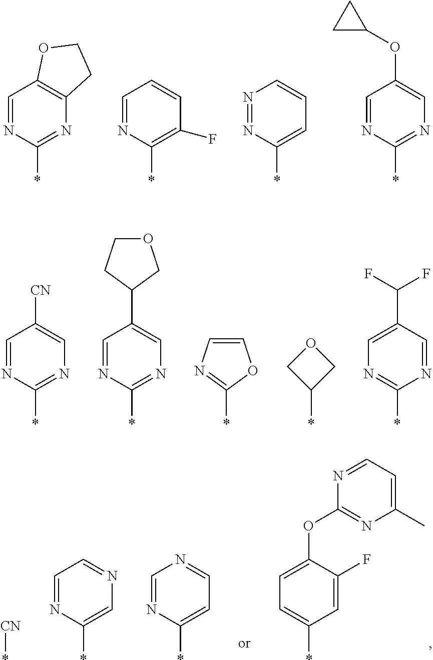

or a pharmaceutically acceptable salt thereof.

Further, the present invention provides a compound of formula I wherein
R₁ is CH₃, CHF₂, CH₂CN, CH₂CH₂OH, or CH₂POMe₂, preferably, CH₃ or CHF₂,
R₂ is

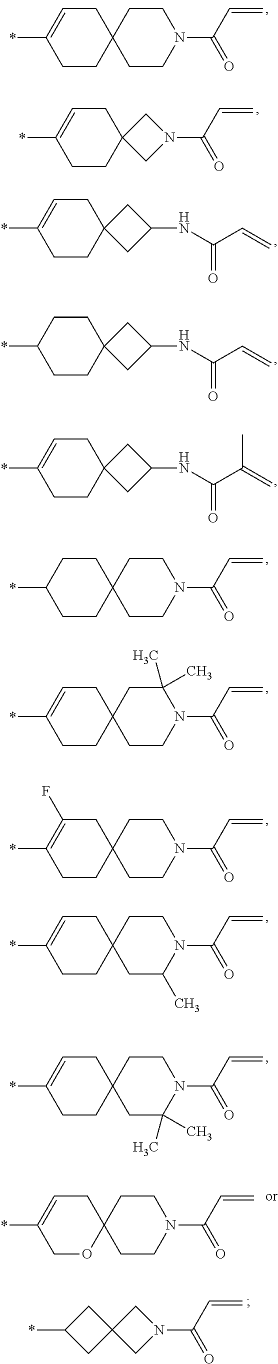

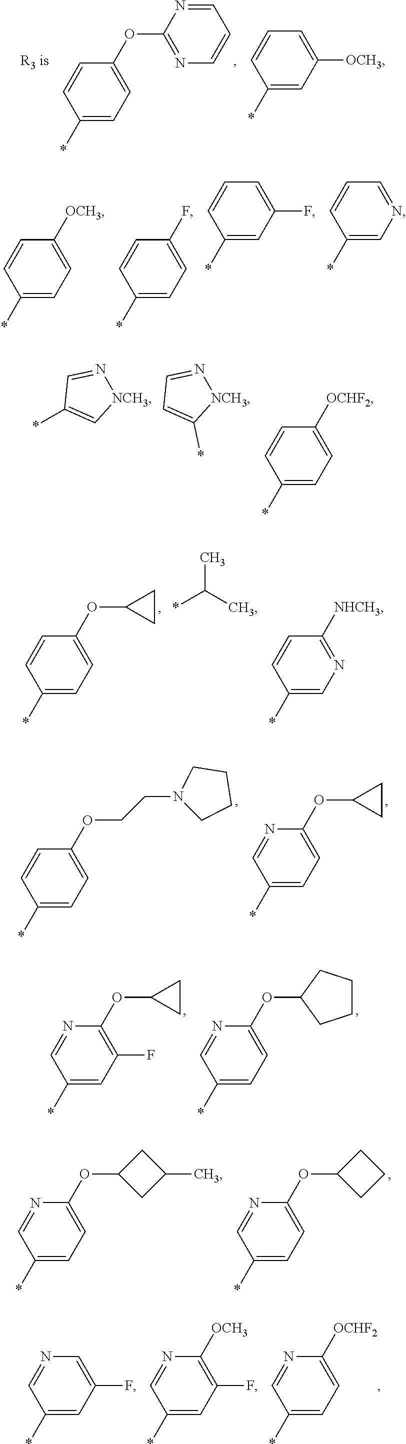

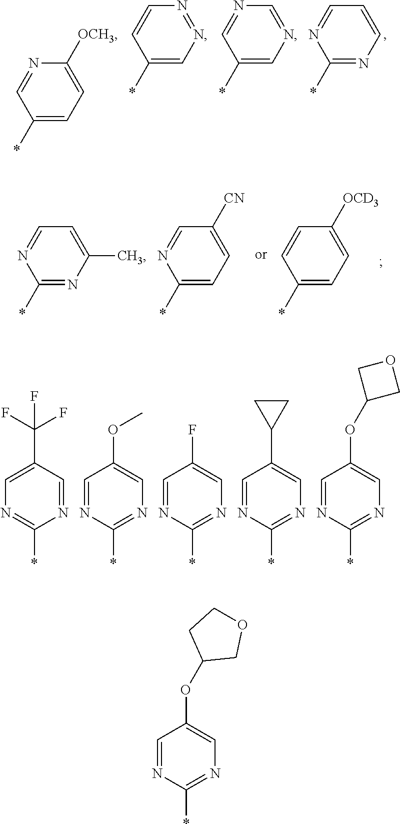

R₃ can also be

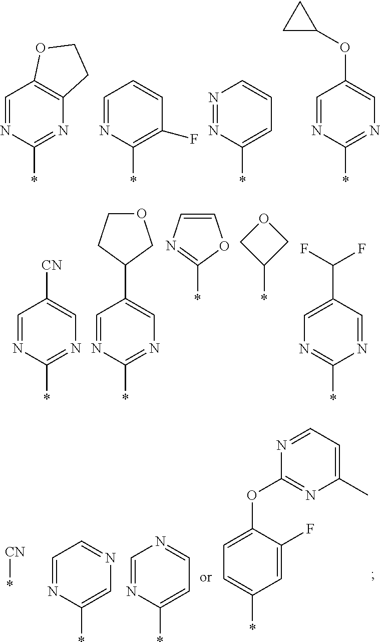

or a pharmaceutically acceptable salt thereof.

Further, the present invention provides a compound of formula I wherein R₂ is a partially unsaturated hydrocarbon having two rings sharing a common carbon atom containing from seven to nineteen carbon atoms wherein two of the ring carbon atoms are replaced with N and O; and the N is substituted with COR₅ wherein
R₅ is

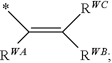

and
R^{WA}, R^{WB}, R^{WC} are on each occurrence independently selected from the group consisting of H, C1-C6 alkyl, substituted C1-C6 alkyl, halogen and CN;
   or a pharmaceutically acceptable salt thereof.

Further, the present invention provides a compound of formula I wherein R₂ is a partially unsaturated hydrocarbon having two rings sharing a common carbon atom containing from seven to nineteen carbon atoms wherein one of the ring carbon atoms is replaced with N and the N is substituted with COR₅ wherein
R₅ is

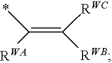

and
R^{WA}, R^{WB}, R^{WC} are on each occurrence independently selected from the group consisting of H, C1-C6 alkyl, substituted C1-C6 alkyl, halogen and CN;
   or a pharmaceutically acceptable salt thereof.

Further, the present invention provides a compound of formula I wherein COR₅ is independently selected from the group consisting of

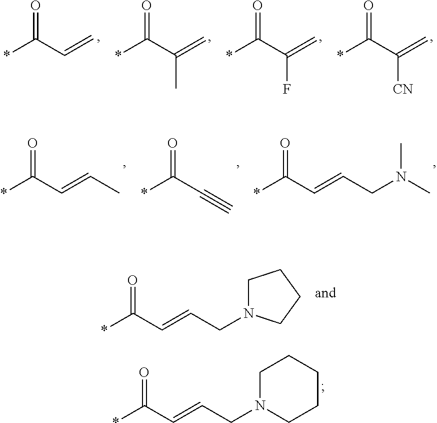

or a pharmaceutically acceptable salt thereof.

Further, the present invention provides a compound of formula I wherein COR₅ is independently selected from the group consisting of

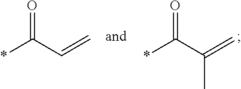

or a pharmaceutically acceptable salt thereof.

Further, the present invention provides a compound of formula I wherein R^{WA}, R^{WB}, R^{WC} are on each occurrence independently selected from the group consisting of H and C1-C6 alkyl; or a pharmaceutically acceptable salt thereof.

Further, the present invention provides a compound of formula I wherein m, n, o and p are on each occurrence independently selected from the group consisting of 0, 1 or 2; or a pharmaceutically acceptable salt thereof.

The present invention further provides a compound or a pharmaceutically acceptable salt thereof selected from the group consisting of
1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)prop-2-en-1-one;
1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)-2-methylprop-2-en-1-one;
1-(6-(4-amino-7-methyl-5-(4-(pyrimidin-2-yloxy)phenyl)-7H-pyrrolo[2,3-d]pyrimidin-6-yl)-2-azaspiro[3.3]heptan-2-yl)prop-2-en-1-one;
1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one;
1-(8-(4-amino-7-methyl-5-(4-(pyrimidin-2-yloxy)phenyl)-7H-pyrrolo[2,3-d]pyrimidin-6-yl)-2-azaspiro[4.5]dec-7-en-2-yl)prop-2-en-1-one;
1-(9-(4-amino-7-methyl-5-(4-(pyrimidin-2-yloxy)phenyl)-7H-pyrrolo[2,3-d]pyrimidin-6-yl)-3-azaspiro[5.5]undecan-3-yl)prop-2-en-1-one;
N-(7-(4-amino-5-(4-methoxyphenyl)-7-methyl-7H-pyrrolo[2,3-d]pyrimidin-6-yl)spiro[3.5]non-6-en-2-yl)acrylamide;
1-(9-(4-amino-5-(4-methoxyphenyl)-7-methyl-7H-pyrrolo[2,3-d]pyrimidin-6-yl)-3-azaspiro[5.5]undec-8-en-3-yl)prop-2-en-1-one;
N-(7-(4-amino-7-methyl-5-(4-(pyrimidin-2-yloxy)phenyl)-7H-pyrrolo[2,3-d]pyrimidin-6-yl)spiro[3.5]non-6-en-2-yl)acrylamide;
1-(9-(4-amino-5-(3-fluorophenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-fluorophenyl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyridin-3-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(1-methyl-1H-pyrazol-5-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(1-methyl-1H-pyrazol-4-yl)-7H-pyrrolo[2,3-d]pyrimidin-6-yl)-3-azaspiro[5.5]undec-8-en-3-yl)prop-2-en-1-one;
1-(9-(4-amino-5-isopropyl-7-methyl-7H-pyrrolo[2,3-d]pyrimidin-6-yl)-3-azaspiro[5.5]undec-8-en-3-yl)prop-2-en-1-one;
1-(9-(4-amino-5-(3-methoxyphenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-(difluoromethoxy)phenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-cyclopropoxyphenyl)-7-methyl-7H-pyrrolo[2,3-d]pyrimidin-6-yl)-3-azaspiro[5.5]undec-8-en-3-yl)prop-2-en-1-one;
N-(7-(4-amino-5-(4-methoxyphenyl)-7-methyl-7H-pyrrolo[2,3-d]pyrimidin-6-yl)spiro[3.5]nonan-2-yl)acrylamide;
1-(9-(4-amino-7-methyl-5-(4-(2-(pyrrolidin-1-yl)ethoxy)phenyl)-7H-pyrrolo[2,3-d]pyrimidin-6-yl)-3-azaspiro[5.5]undec-8-en-3-yl)prop-2-en-1-one;
1-(9-(4-amino-7-methyl-5-(6-(methylamino)pyridin-3-yl)-7H-pyrrolo[2,3-d]pyrimidin-6-yl)-3-azaspiro[5.5]undec-8-en-3-yl)prop-2-en-1-one;
1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-cyclopropoxy-5-fluoropyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-(cyclopentyloxy)pyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(6-(3-methylcyclobutoxy)pyridin-3-yl)-7H-pyrrolo[2,3-d]pyrimidin-6-yl)-3-azaspiro[5.5]undec-8-en-3-yl)prop-2-en-1-one;
1-(9-(4-amino-5-(6-cyclobutoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(5-((tetrahydrofuran-3-yl)oxy)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(5-cyclopropoxypyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoropyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoro-6-methoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-(difluoromethoxy)pyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyridazin-4-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-5-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-2,2-dimethyl-3-azaspiro[5.5]undec-8-en-3-yl)prop-2-en-1-one;
1-(9-(4-amino-7-methyl-5-(4-methylpyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(4-cyclopropoxyphenyl)-7-(difluoromethyl)-7H-pyrrolo[2,3-d]pyrimidin-6-yl)-3-azaspiro[5.5]undec-8-en-3-yl)prop-2-en-1-one;
5-(6-(3-acryloyl-3-azaspiro[5.5]undec-8-en-9-yl)-4-amino-7-(difluoromethyl)-7H-pyrrolo[2,3-d]pyrimidin-5-yl)picolinonitrile;
1-(9-(4-amino-5-(4-(methoxy-d3)phenyl)-7-methyl-7H-pyrrolo[2,3-d]pyrimidin-6-yl)-8-fluoro-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-2-methyl-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-2,2-dimethyl-3-azaspiro[5.5]undec-8-en-3-yl)prop-2-en-1-one;
1-(3-(4-amino-5-(4-fluorophenyl)-7-methyl-7H-pyrrolo[2,3-d]pyrimidin-6-yl)-1-oxa-9-azaspiro[5.5]undec-3-en-9-yl)prop-2-en-1-one;
1-(9-(4-amino-7-methyl-5-(5-(trifluoromethyl)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(5-methoxypyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoropyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-cyclopropylpyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(5-(oxetan-3-yloxy)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(6,7-dihydrofuro[3,2-d]pyrimidin-2-yl)-7-methyl-7H-pyrrolo[2,3-d]pyrimidin-6-yl)-3-azaspiro[5.5]undec-8-en-3-yl)prop-2-en-1-one
1-(9-(4-amino-5-(3-fluoropyridin-2-yl)-7-methyl-7H-pyrrolo[2,3-d]pyrimidin-6-yl)-3-azaspiro[5.5]undec-8-en-3-yl)prop-2-en-1-one;
2-(6-(3-acryloyl-3-azaspiro[5.5]undec-8-en-9-yl)-4-amino-7-methyl-7H-pyrrolo[2,3-d]pyrimidin-5-yl)pyrimidine-5-carbonitrile;
1-(9-(4-amino-7-methyl-5-(5-(tetrahydrofuran-3-yl)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(oxazol-2-yl)-7H-pyrrolo[2,3-d]pyrimidin-6-yl)-3-azaspiro[5.5]undec-8-en-3-yl)prop-2-en-1-one;
1-(9-(4-amino-7-(2-hydroxyethyl)-5-(pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(oxetan-3-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyridazin-3-yl)-7H-pyrrolo[2,3-d]pyrimidin-6-yl)-3-azaspiro[5.5]undec-8-en-3-yl)prop-2-en-1-one;
1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one;
6-(3-acryloyl-3-azaspiro[5.5]undec-8-en-9-yl)-4-amino-7-methyl-7H-pyrrolo[2,3-d]pyrimidine-5-carbonitrile;
1-(9-(4-amino-5-(pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrazin-2-yl)-7H-pyrrolo[2,3-d]pyrimidin-6-yl)-3-azaspiro[5.5]undec-8-en-3-yl)prop-2-en-1-one;
1-(9-(4-amino-5-(5-(difluoromethyl)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-4-yl)-7H-pyrrolo[2,3-d]pyrimidin-6-yl)-3-azaspiro[5.5]undec-8-en-3-yl)prop-2-en-1-one; and
1-(9-(4-amino-5-(3-fluoro-4-((4-methylpyrimidin-2-yl)oxy)phenyl)-7-methyl-7H-pyrrolo[2,3-d]pyrimidin-6-yl)-3-azaspiro[5.5]undec-8-en-3-yl)prop-2-en-1-one.

The present invention further provides a compound or a pharmaceutically acceptable salt thereof selected from the group consisting of
1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one;
1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one; and
1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one.

The details of one or more embodiments of the invention are set forth in the description below. Other features, objects, and advantages of the invention will be apparent from the description and from the claims.

### DETAILED DESCRIPTION OF THE INVENTION

As used herein, the term “halogen” or “halo” refers to fluoro, chloro, bromo, or iodo, unless otherwise specified herein. A particular value of halogen is fluoro.

As used herein, the term “C1-C6-alkyl” refers to a straight or branched, monovalent, saturated aliphatic chain of 1 to 6 carbon atoms and includes methyl, ethyl, n-propyl, isopropyl, n-butyl, isobutyl, t-butyl, n-pentyl, n-hexyl, and the like.

As used herein, the term “C1-C4-deuteroalkyl” refers to a straight or branched, monovalent, saturated aliphatic chain of 1 to 4 carbon atoms that is substituted by one or more deuterium atoms (D). In some embodiments, the C1-C4 deuteroalkyl is substituted with one deuterium atom. In some embodiments, the C1-C4 deuteroalkyl is substituted with one, two, or three deuterium atoms. In some embodiments, the C1-C4 deuteroalkyl is substituted with one, two, three, four, five, or six deuterium atoms. The term C1-C4 deuteroalkyl includes CD₃, CH₂D, CHD₂, CH₂CD₃, CD₂CD₃, CHDCD₃, CH₂CH₂D, CH₂CHD₂ and the like. A particular value of C1-C4 deuteroalkyl is CD₃.

As used herein, the term “substituted C1-C6 alkyl” refers to a straight or branched, monovalent, saturated aliphatic chain of 1 to 6 carbon atoms wherein one or more of the carbon atoms is substituted with one to three or preferably one or two groups independently selected from the group consisting of C3-C10 heterocycloalkyl, halogen, C2-C4 alkenyl optionally substituted with 1-3 halogens, C2-C4 alkynyl optionally substituted with 1-3 halogens, hydroxy, OR₉, cyano, phosphorous, CONR₇R₈, NR₇R₈, NR₇COR₈, NR₇SO₂R₈, NR₇COOR₈, COR₇, COOR₇, SR₇, and SONR₇R₈ wherein R₇, R₈ and R₉ are on each occurrence independently selected from the group consisting of hydrogen, C1-C6 alkyl optionally substituted with 1-3 halogens, aryl, substituted aryl, heteroaryl, substituted heteroaryl, heterocycloalkyl, substituted heterocycloalkyl, C1-C4 deuteroalkyl, C2-C4 alkenyl optionally substituted with 1-3 halogens, and C2-C4 alkynyl optionally substituted with 1-3 halogens.

As used herein, the term “C1-C4 alkyl” refers to a straight or branched, monovalent, saturated aliphatic chain of 1 to 4 carbon atoms and includes methyl, ethyl, n-propyl, isopropyl, n-butyl, isobutyl and the like.

As used herein, the term “C2-C6 alkenyl” refers to a straight or branched, monovalent, unsaturated aliphatic chain having from two to six carbon atoms and one or more carbon-carbon double bonds. Typical C2-C6 alkenyl groups include ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein, the term “C2-C4 alkenyl” refers to a straight or branched, monovalent, unsaturated aliphatic chain having from two to four carbon atoms and one or more carbon-carbon double bonds. Typical C2-C4 alkenyl groups include ethenyl (also known as vinyl), 1-methylethenyl, 1-methyl-1-propenyl, 1-butenyl, and the like.

As used herein, the term “C2-C6 alkynyl” refers to a straight or branched alkynyl chain having from two to six carbon atoms and one or more carbon-carbon triple bonds, and includes ethynyl, 2-propynyl, 2-butynyl, 3-methylbutnyl, 1-pentynyl and the like.

As used herein, the term “C2-C4 alkynyl” refers to a straight or branched alkynyl chain having from two to four carbon atoms and one carbon-carbon triple bonds, and includes ethynyl, 2-propynyl, and the like.

As used herein, the term “C3-C10 cycloalkyl” refers to a saturated hydrocarbon having one or more rings containing from three to ten carbon atoms. It is understood when multiple rings are employed, the term includes fused, bridged and spiro ring systems. Typical C3-C10 cycloalkyl groups include monocyclic, bicyclic and spiro rings such as cyclopropyl, cyclobutyl, cyclopentyl, bicyclo[1.1.1]pentyl, bicyclo[2.1.1]hexyl, cyclohexyl, cycloheptyl, cyclooctyl, decahydronaphthalene and the like.

As used herein, the term “substituted C3-C10 cycloalkyl” refers to a saturated hydrocarbon having one or more rings containing from three to ten carbon atoms wherein one or more of the ring atoms is substituted with one to three or preferably one or two groups independently selected from the group consisting of halogen, cyclopropyl, C1-C6 alkyl, substituted C1-C6 alkyl, C2-C4 alkenyl optionally substituted with 1-3 halogens, C2-C4 alkynyl optionally substituted with 1-3 halogens, aryl, substituted aryl, heteroaryl, substituted heteroaryl, heterocycloalkyl, substituted heterocycloalkyl, hydroxy, OR₉, cyano, CONR₇R₈, NR₇R₈, NR₇COR₈, NR₇SO₂R₈, NR₇COOR₈, COR₇, COOR₇, SR₇, and SONR₇R₈ wherein R₇, R₈ and R₉ are on each occurrence independently selected from the group consisting of hydrogen, C1-C6 alkyl, substituted C1-C6 alkyl, C1-C4 deuteroalkyl, C2-C4 alkenyl optionally substituted with 1-3 halogens, C2-C4 alkynyl optionally substituted with 1-3 halogens, aryl, substituted aryl, heteroaryl, substituted heteroaryl, heterocycloalkyl, and substituted heterocycloalkyl. It is understood when multiple rings are employed, the term includes fused, bridged and spiro ring systems.

As used herein, the term “substituted C7-C19 spiro bicycloalkyl” refers to a saturated hydrocarbon having two rings sharing a common carbon atom containing from seven to nineteen carbon atoms wherein one or more of the ring atoms is substituted with one to three or preferably one or two NR₆COR₅ groups wherein R₆ is H or C1-C4 alkyl, and R₅ is

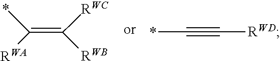

R^{WA}, R^{WB}, R^{WC} are on each occurrence independently selected from the group consisting of H, C1-C6 alkyl, substituted C1-C6 alkyl, halogen and CN; and
R^{WD} is H or C1-C6 alkyl.

In certain embodiments, substituted C7-C19 spiro bicycloalkyl groups include

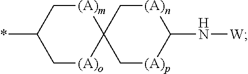

wherein
A is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
W is COR₅;
R₅ is

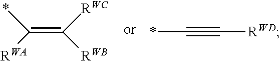

R^{WA}, R^{WB}, R^{WC} are on each occurrence independently selected from the group consisting of H, C1-C6 alkyl, substituted C1-C6 alkyl, halogen and CN; and R is H or C1-C6 alkyl.

In certain embodiments, substituted C7-C19 spiro bicycloalkyl groups include

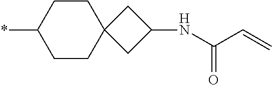 [SMILES: *C1CCC2(CC1)CC(NC(=O)C=C)C2]

and the like.

As used herein, the term “substituted C7-C19 spiro bicycloalkenyl” refers to a partially unsaturated hydrocarbon having two rings sharing a common carbon atom containing from seven to nineteen carbon atoms wherein one or more of the ring atoms is substituted with one to three or preferably one or two NR₆COR₅ groups wherein R₆ is H or C1-C4 alkyl, and R₅ is

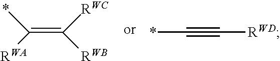

R^{WA}, R^{WB}, R^{WC} are on each occurrence independently selected from the group consisting of H, C1-C6 alkyl, substituted C1-C6 alkyl, halogen and CN; and
R^{WD} is H or C1-C6 alkyl.

In one embodiment the term “substituted C7-C19 spiro bicycloalkenyl” refers to a partially unsaturated hydrocarbon having two double bonds. In a further embodiment, the term “substituted C7-C19 spiro bicycloalkenyl” refers to a partially unsaturated hydrocarbon having one double bond.

In certain embodiments, the term “substituted C7-C19 spiro bicycloalkenyl” refers to a partially unsaturated hydrocarbon having two rings sharing a common carbon atom containing from seven to nineteen carbon atoms wherein one of the ring atoms is substituted with one or two NR₆COR₅ groups wherein R₆ is H or C1-C4 alkyl, and R₅ is

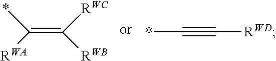

R^{WA}, R^{WB}, R^{WC} are on each occurrence independently selected from the group consisting of H, C1-C6 alkyl, substituted C1-C6 alkyl, halogen and CN; and R is H or C1-C6 alkyl.

In certain embodiments, substituted C7-C19 spiro bicycloalkenyl groups include

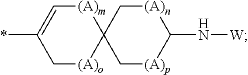

wherein
A is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
W is COR₅;
R₅ is

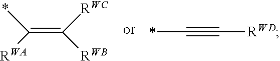

R^{WA}, R^{WB}, R^{WC} are on each occurrence independently selected from the group consisting of H, C1-C6 alkyl, substituted C1-C6 alkyl, halogen and CN; and
R^{WD} is H or C1-C6 alkyl.

In certain embodiments, substituted C7-C19 spiro bicycloalkenyl groups include

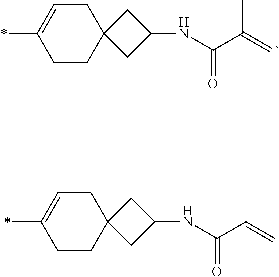 [SMILES: *C1=CCC2(CC1)CC(NC(=O)C(=C)C)C2 ; *C1=CCC2(CC1)CC(NC(=O)C=C)C2]

and the like.

As used herein, the term “substituted C7-C19 spiro heterobicycloalkanyl” refers to a saturated hydrocarbon having two rings sharing a common carbon atom containing from seven to nineteen carbon atoms wherein at least one of the ring carbon atoms is replaced with N wherein the N is substituted with COR₅ and R₅ is

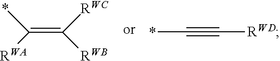

R^{WA}, R^{WB}, R^{WC} are on each occurrence independently selected from the group consisting of H, C1-C6 alkyl, substituted C1-C6 alkyl, halogen and CN; and R is H or C1-C6 alkyl.

Further to the term “substituted C7-C19 spiro heterobicycloalkanyl”, additional ring carbon atoms are optionally replaced with one or more O, S, SO or SO₂.

In certain embodiments, the term “substituted C7-C19 spiro heterobicycloalkanyl” refers to

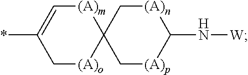

wherein
A is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
W is COR₅;
R₅ is

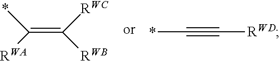

R^{WA}, R^{WB}, R^{WC} are on each occurrence independently selected from the group consisting of H, C1-C6 alkyl, substituted C1-C6 alkyl, halogen and CN; and R^{WD} is H or C1-C6 alkyl.

In certain embodiments, “substituted C7-C19 spiro heterobicycloalkanyl” groups include

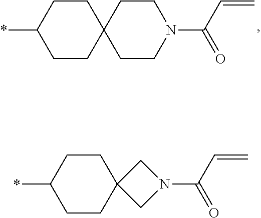 [SMILES: *C1CCC2(CC1)CCN(C(=O)C=C)CC2 ; *C1CCC2(CC1)CN(C(=O)C=C)C2]

and the like.

As used herein, the term “substituted C7-C19 spiro heterobicycloalkenyl” refers to a partially unsaturated hydrocarbon having two rings sharing a common carbon atom containing from seven to nineteen carbon atoms wherein at least one of the ring carbon atoms is replaced with N wherein the N is substituted with COR₅ and R₅ is

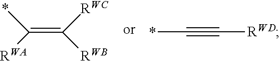

R^{WA}, R^{WB}, R^{WC} are on each occurrence independently selected from the group consisting of H, C1-C6 alkyl, substituted C1-C6 alkyl, halogen and CN; and R^{WD} is H or C1-C6 alkyl.

Further to the term “substituted C7-C19 spiro heterobicycloalkenyl”, additional ring carbon atoms are optionally replaced with one or more O, S, SO or SO₂.

In one embodiment, the term “substituted C7-C19 spiro heterobicycloalkenyl” refers to a partially unsaturated hydrocarbon having two double bonds. In a further embodiment, the term “substituted C7-C19 spiro heterobicycloalkenyl” refers to a partially unsaturated hydrocarbon having one double bond.

In certain embodiments, the term “substituted C7-C19 spiro heterobicycloalkenyl” refers to

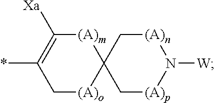

wherein
(A)ₘ is CRₐR_{b}
(A)ₙ is CRₐR_{b};
(A)ₒ is CRₐR_{b} or O;
(A)ₚ is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
provided when (A)ₒ is O, o is 1;
Xa is H or halogen;
W is COR₅;
R₅ is

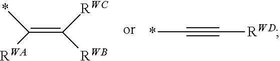

R^{WA}, R^{WB}, R^{WC} are on each occurrence independently selected from the group consisting of H, C1-C6 alkyl, substituted C1-C6 alkyl, halogen and CN; and R^{WD} is H or C1-C6 alkyl.

In certain embodiments, the term “substituted C7-C19 spiro heterobicycloalkenyl” groups include

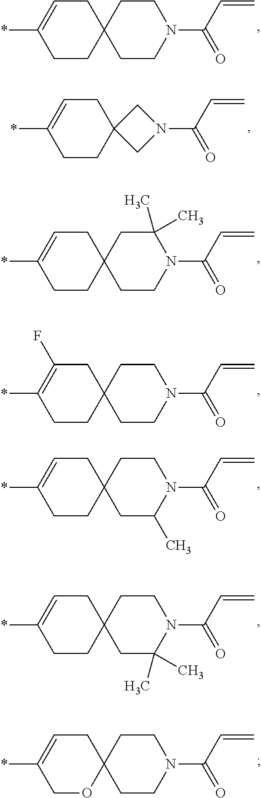

and the like.

As used herein, the term “C3-C10 heterocycloalkyl” refers to a saturated hydrocarbon having one or more rings containing from three to ten carbon atoms wherein one or more of the ring carbon atoms is replaced with N, O or S. It is understood when multiple rings are employed, the term includes fused, bridged and spiro ring systems. Typical C3-C10 heterocycloalkyl groups include aziridinyl, pyrrolidinyl, tetrahydrofuranyl, tetrahydrothiophenyl, piperidinyl, morpholinyl, tetrahydropyranyl, 2-azaspiro[3.3]heptanyl, 8-azabicyclo[3.2.1]octanyl and the like.

As used herein, the term “substituted C3-C10 heterocycloalkyl” refers to a saturated hydrocarbon having one or more rings containing from three to ten carbon atoms wherein one or more of the ring carbon atoms is replaced with N, O or S. Further to the term, one or more of the ring atoms is substituted with one to three or preferably one or two groups independently selected from the group consisting of halogen, cyclopropyl, C1-C6 alkyl, substituted C1-C6 alkyl, C2-C4 alkenyl optionally substituted with 1-3 halogens, C2-C4 alkynyl optionally substituted with 1-3 halogen, aryl, substituted aryl, heteroaryl, substituted heteroaryl, heterocycloalkyl, substituted heterocycloalkyl, hydroxy, hydroxy, OR₉, cyano, CONR₇R₈, NR₇R₈, NR₇COR₈, NR₇SO₂R₈, NR₇COOR₈, COR₇, COOR₇, SR₇, and SONR₇R₈ wherein R₇, R₈ and R₉ are on each occurrence independently selected from the group consisting of hydrogen, C3-C10 cycloalkyl, substituted C3-C10 cycloalkyl, C1-C6 alkyl, substituted C1-C6 alkyl, C1-C4 deuteroalkyl, C2-C4 alkenyl optionally substituted with 1-3 halogens, C2-C4 alkynyl optionally substituted with 1-3 halogens, aryl, substituted aryl, heteroaryl, substituted heteroaryl, heterocycloalkyl, and substituted heterocycloalkyl. It is understood when multiple rings are employed, the term includes fused, bridged and spiro ring systems.

As used herein, the term “aryl” refers to monovalent carbocyclic group containing one or more fused or non-fused phenyl rings. It is understood when multiple rings are employed, the term includes partially unsaturated ring systems. Typical aryl groups include phenyl, biphenyl, 1 or 2-naphthyl, 1,2-dihydronaphthyl, 1,2,3,4-tetrahydronaphthyl, indenyl, indanyl and the like.

As used herein, the term “substituted aryl” refers to carbocyclic group containing one or more fused or non-fused phenyl rings wherein one or more of the ring atoms is substituted with one to three or preferably one or two groups independently selected from the group consisting of halogen, C2-C4 alkenyl optionally substituted with 1-3 halogens, C2-C4 alkynyl optionally substituted with 1-3 halogens, C1-C6 alkyl, substituted C1-C6 alkyl, C3-C10 cycloalkyl, substituted C3-C10 cycloalkyl, hydroxy, OR₉, cyano, CONR₇R₈, NR₇R₈, NR₇COR₈, NR₇SO₂R₈, NR₇COOR₈, COR₇, COOR₇, SR₇, and SONR₇R₈ wherein R₇, R₈ and R₉ are on each occurrence independently selected from the group consisting of hydrogen; C3-C10 cycloalkyl; C1-C4 deuteroalkyl; C1-C6 alkyl optionally substituted with 1-3 halogens or C3-C10 heterocycloalkyl group; C2-C4 alkenyl optionally substituted with 1-3 halogens and C2-C4 alkynyl optionally substituted with 1-3 halogens. It is understood when multiple rings are employed, the term includes partially unsaturated ring systems. In certain embodiments, substituted aryl groups include

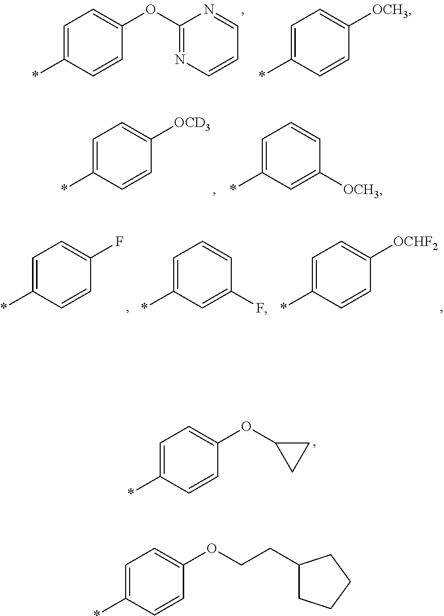

and the like.

As used herein, the term “heteroaryl” refers to an aromatic hydrocarbon having one or more rings wherein one or more of the ring carbon atoms is replaced with N, O or S. It is understood when multiple rings are employed, the term includes partially unsaturated ring systems. Typical heteroaryl groups include pyridinyl, pyrimidinyl, pyridazinyl, pyrazolyl, imidazolyl and the like.

As used herein, the term “substituted heteroaryl” refers to an aromatic hydrocarbon having one or more rings wherein one or more of the ring carbon atoms is replaced with N, O or S, and one or more of the ring atoms is substituted with one to three or preferably one or two groups independently selected from the group consisting of halogen, C1-C6 alkyl, substituted C1-C6 alkyl, C3-C10 cycloalkyl, substituted C3-C10 cycloalkyl, C2-C4 alkenyl optionally substituted with 1-3 halogens, C2-C4 alkynyl optionally substituted with 1-3 halogens, hydroxy, OR₉, cyano, CONR₇R₈, NR₇R₈, NR₇COR₈, NR₇SO₂R₈, NR₇COOR₈, COR₇, COOR₇, SR₇, and SONR₇R₈ wherein R₇, R₈ and R₉ are on each occurrence independently selected from the group consisting of hydrogen, C1-C6 alkyl optionally substituted with 1-3 halogens, C1-C4 deuteroalkyl, C2-C4 alkenyl optionally substituted with 1-3 halogens, C3-C10 heterocycloalkyl, and C2-C4 alkynyl optionally substituted with 1-3 halogens. It is understood when multiple rings are employed, the term includes partially unsaturated ring systems. Typical substituted heteroaryl groups include

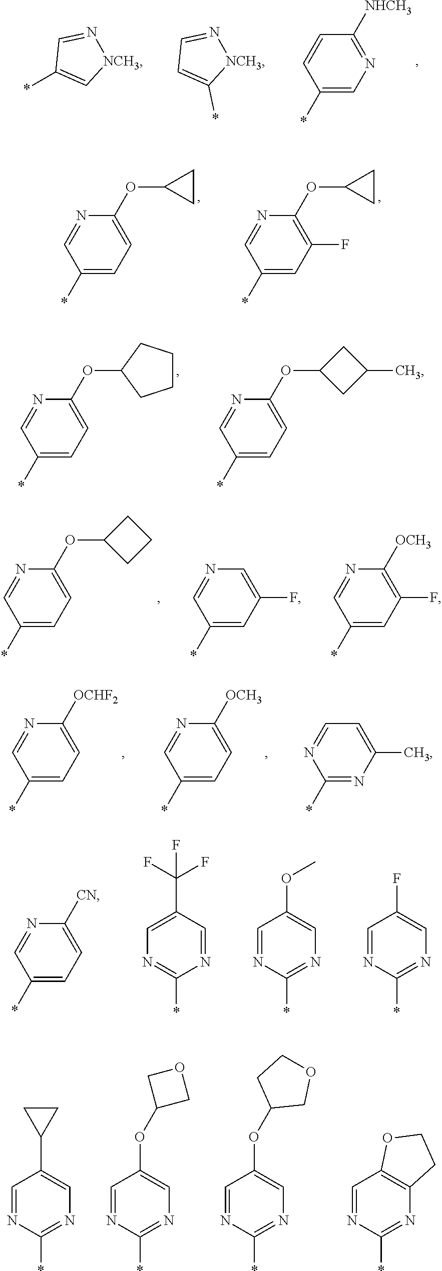

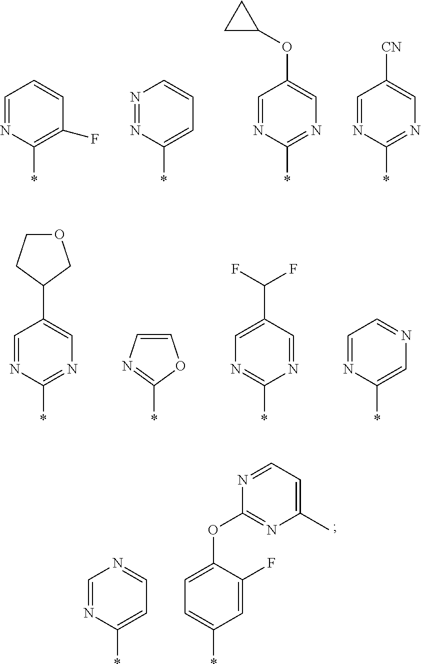

and the like.

As used herein, the term “pharmaceutically acceptable salt” includes an acid addition salt that exists in conjunction with the basic portion of a compound of formula I. Such pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011).

In addition to pharmaceutically acceptable salts, other salts are contemplated in the invention. They may serve as intermediates in the purification of compounds or in the preparation of other pharmaceutically acceptable salts, or are useful for identification, characterization, or purification of compounds of the invention.

It is understood that compounds of the present invention may contain a single or double bond. As used herein, the depiction of a solid and dashed bond in a formula such as

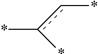

indicates a structure may contain a single bond or double bond. In illustration, a formula depicted as

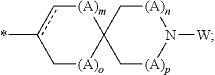

is understood to include

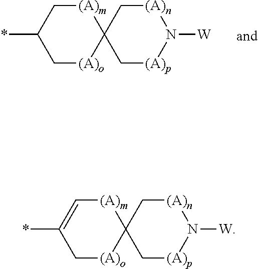

It is understood that compounds of the present invention may exist as stereoisomers. It is further understood that compounds of the present invention include all forms of stereoisomers including enantiomers, diastereomers, and mixtures thereof. Preferred stereoisomers are predominantly one diastereomer. More preferred stereoisomers are predominantly one enantiomer.

As used herein, the depiction of an asterisk (*) in a chemical formula represents the point of attachment of the group to the corresponding parent formula.

Further, the present invention provides a pharmaceutical composition comprising a compound of formula I, or a pharmaceutically acceptable salt thereof, and a pharmaceutically acceptable carrier, diluent or excipient.

Further, the present invention provides a compound of formula I, or a pharmaceutically acceptable salt thereof, for use in therapy.

Further, the present invention provides a method of treating cancer comprising administrating to a patient in need thereof an effective amount of a compound of formula I or a pharmaceutically acceptable salt thereof.

Further, the present invention provides a method of treating cancer wherein the cancer harbors aberrant activation of FGFR2 comprising administrating to a patient in need thereof an effective amount of a compound of formula I or a pharmaceutically acceptable salt thereof.

Further, the present invention provides a method of treating cancer comprising administrating to a patient in need thereof an effective amount of a compound of formula I or a pharmaceutically acceptable salt thereof wherein the cancer is selected from the group consisting of endometrial carcinoma, gastric cancer, intrahepatic cholangiocarcinoma, breast cancer, urinary tract cancer and non-small cell lung cancer (NSCLC).

Further, the present invention provides a method of treating cancer comprising administrating to a patient in need thereof an effective amount of a compound of formula I or a pharmaceutically acceptable salt thereof wherein the cancer is selected from the group consisting of endometrial carcinoma, gastric cancer, and intrahepatic cholangiocarcinoma.

Further, the present invention provides a compound of formula I, or a pharmaceutically acceptable salt thereof, for use in the treatment of cancer wherein the cancer harbors aberrant activation of FGFR2.

Further, the present invention provides a compound of formula I, or a pharmaceutically acceptable salt thereof, for use in the treatment of cancer wherein the cancer is selected from the group consisting of endometrial carcinoma, gastric cancer, intrahepatic cholangiocarcinoma, breast cancer, urinary tract cancer and non-small cell lung cancer (NSCLC).

Further, the present invention provides a compound of formula I, or a pharmaceutically acceptable salt thereof, for use in the treatment of cancer wherein the cancer is selected from the group consisting of endometrial carcinoma, gastric cancer, and intrahepatic cholangiocarcinoma.

Further, the present invention provides the use of a compound of formula I, or a pharmaceutically acceptable salt thereof, for the manufacture of a medicament for treating cancer.

As used herein, the term “patient” refers to an animal such as a mammal and includes a human. A human is a preferred patient.

It is recognized that one skilled in the art may treat cancer by administering to a patient presently displaying symptoms an effective amount of the compound of formula I. Thus, the terms “treatment” and “treating” are intended to refer to all processes wherein there may be a slowing, interrupting, arresting, controlling, or stopping of the progression of an existing disorder and/or symptoms thereof, but does not necessarily indicate a total elimination of all symptoms.

Further, it is recognized that one skilled in the art may treat cancer by administering to a patient at risk of future symptoms an effective amount of the compound of formula I and is intended to include prophylactic treatment of such.

As used herein, the term “effective amount” of a compound of formula I refers to an amount, that is a dosage, which is effective in treating a disorder, such as the diseases described herein. The attending diagnostician, as one skilled in the art, can readily determine an effective amount by the use of conventional techniques and by observing results obtained under analogous circumstances. In determining an effective amount or dose of a compound of formula I, a number of factors are considered, including, but not limited to the compound of formula I to be administered; the co-administration of other agents, if used; the species of mammal; its size, age, and general health; the degree of involvement or the severity of the disorder, such as cancer; the response of the individual patient; the mode of administration; the bioavailability characteristics of the preparation administered; the dose regimen selected; the use of other concomitant medication; and other relevant circumstances.

A compound of formula I may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition, Academic Press, 2020).

Within this invention it is to be understood that the combinations, compositions, kits, methods, uses or compounds for use according to this invention may envisage the simultaneous, concurrent, sequential, successive, alternate, or separate administration of the active ingredients or components. It will be appreciated that the FGFR2 inhibitor compound (e.g. compound of formula (I)) and the at least one other pharmacologically active substance can be administered formulated either dependently or independently, such as e.g. the FGFR2 inhibitor compound (e.g. compound of formula (I)) and the at least one other pharmacologically active substance may be administered either as part of the same pharmaceutical composition/dosage form or, preferably, in separate pharmaceutical compositions/dosage forms. In this context, “combination” or “combined” within the meaning of this invention includes, without being limited, a product that results from the mixing or combining of more than one active ingredient and includes both fixed and non-fixed (e.g., free) combinations (including kits) and uses, such as e.g., the simultaneous, concurrent, sequential, successive, alternate, or separate use of the components or ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently, or sequentially with no specific time limits, wherein such administration provides therapeutically effective levels of the two compounds in the body of the patient.

The administration of the FGFR2 inhibitor compound (e.g., compound of formula (I)) and the at least one other pharmacologically active substance may take place by co-administering the active components or ingredients, such as e.g., by administering them simultaneously or concurrently in one single or in two or more separate formulations or dosage forms. Alternatively, the administration of the FGFR2 inhibitor compound (e.g., compound of formula (I)) and the at least one other pharmacologically active substance may take place by administering the active components or ingredients sequentially or in alternation, such as e.g., in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g. by oral, enteral, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect the invention provides a method for the treatment and/or prevention of cancer comprising administering to a patient in need thereof a therapeutically effective amount of a FGFR2 inhibitor compound (e.g. a compound of formula (I)) and a therapeutically effective amount of at least one other pharmacologically active substance, wherein the FGFR2 inhibitor compound (e.g. a compound of formula (I)) is administered simultaneously, concurrently, sequentially, successively, alternately or separately with the at least one other pharmacologically active substance.

In another aspect the invention provides a FGFR2 inhibitor compound (e.g., a compound of formula (I)) for use in the treatment and/or prevention of cancer, wherein the FGFR2 inhibitor compound (e.g., a compound of formula (I)) is administered simultaneously, concurrently, sequentially, successively, alternately, or separately with the at least one other pharmacologically active substance.

In another aspect the invention provides a kit comprising a first pharmaceutical composition or dosage form comprising a FGFR2 inhibitor compound (e.g. a compound of formula (I)), and, optionally, one or more pharmaceutically acceptable carriers, excipients and/or vehicles, and at least a second pharmaceutical composition or dosage form comprising another pharmacologically active substance, and, optionally, one or more pharmaceutically acceptable carriers, excipients and/or vehicles, for use in the treatment and/or prevention of cancer, wherein the first pharmaceutical composition is to be administered simultaneously, concurrently, sequentially, successively, alternately or separately with the second and/or additional pharmaceutical composition or dosage form.

In a further embodiment of the invention the components (i.e., the combination partners) of the combinations, kits, uses, methods, and compounds for use according to the invention (including all embodiments) are administered simultaneously.

In a further embodiment of the invention the components (i.e., the combination partners) of the combinations, kits, uses, methods, and compounds for use according to the invention (including all embodiments) are administered concurrently.

In a further embodiment of the invention the components (i.e., the combination partners) of the combinations, kits, uses, methods, and compounds for use according to the invention (including all embodiments) are administered sequentially.

In a further embodiment of the invention the components (i.e., the combination partners) of the combinations, kits, uses, methods, and compounds for use according to the invention (including all embodiments) are administered successively.

In a further embodiment of the invention the components (i.e., the combination partners) of the combinations, kits, uses, methods, and compounds for use according to the invention (including all embodiments) are administered alternately.

In a further embodiment of the invention the components (i.e., the combination partners) of the combinations, kits, uses, methods, and compounds for use according to the invention (including all embodiments) are administered separately.

The “therapeutically effective amount” of the active compound(s) to be administered is the minimum amount necessary to prevent, ameliorate, or treat a disease or disorder. The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (joint) therapeutically effective amount.

In a further embodiment, the present invention provides a method of selectively inhibiting FGFR2 over one or more of FGFR1, FGFR3 and FGFR4. In some embodiments, a compound of the present invention is more than 50-fold FGFR2 selective over FGFR1, FGFR3, or FGFR4. In some embodiments, a compound of the present invention is more than 500-fold FGFR2 selective over FGFR1, FGFR3, or FGFR4. In some embodiments, a compound of the present invention is more than 1000-fold FGFR2 selective over FGFR1, FGFR3, or FGFR4. In some embodiments, a compound of the present invention is more than 2000-fold FGFR2 selective over FGFR1, FGFR3, or FGFR4.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and efficacy evaluation of compounds of this invention.

### Biological Assays

### pERK Potency Assay

The purpose of this assay is to measure the ability of test compounds to inhibit FGFR mediated downstream signaling cascades, i.e., ERK phosphorylation in cells with FGFR aberrations. FGFR aberrations become constitutively active and thus induces cascades of cellular signaling events that result in increased phosphorylation of ERK at Threonine 202 and Tyrosine 204 (pERK). The procedure called In Cell ELISA described below measures the level of cellular pERK in response to FGFR specific inhibitor compounds in FGFR aberrations of AN3CA (FGFR2/k310R, N549K), DMS114 (FGFR1 amplification), RT112 (FGFR3 overexpression/FGFR3-TACC3 fusion) and Hep3B (FGF19 amplification) cells. AN3CA, DMS114 and Hep3B cells are from ATCC. RT112 cells are from Sigma. DMS114 cells are grown and maintained using RPMI-1640 medium supplemented with 10% heat-inactivated fetal bovine serum. AN3CA, Hep3B and RT112 cells are grown and maintained using DMEM supplemented with 10% heat-inactivated fetal bovine serum. On the day before test compound addition, cells are plated in poly-D-lysine coated 96-well cell culture plates (Corning® BioCoat® Cat #356640) at 40000 cells/well/100 μL and grown overnight in a 37° C., 5% CO₂ incubator. Test compounds are prepared with 4-fold serial dilutions in DMSO, with a top concentration of 10 μM. On the day of the assay, 50 μL of test compound diluted in media was added to each well of cell culture plate with the final concentration of compounds spanning from 0.000038 μM to 10 μM. After compound addition, cells are incubated for 3 hours at 37° C., 5% CO₂ and then the culture medium is removed. Cells are fixed with 4% formaldehyde in phosphate-buffered saline (PBS) and incubated at RT for 20 min. The plate is then washed three times with PBST (PBS with 0.05% tween-20), followed by the incubation with 100 μL of pre-cooled methanol at −20° C. for 20 min. After incubation, methanol is removed, and the plate is washed once with PBST. Cells are permeabilized with 100 L/well of 0.1% Triton X-100 in PBS at RT for 20 min and then quenched with quenching buffer (PBST containing 1% H₂O₂ and 0.1% sodium azide) for 20 min at RT with gentle shaking. After washing once with PBST, blocking buffer (250 ul/well, Pierce™ Protein-Free-PBS Blocking Buffer, Cat #37572) was then added to the cells for 1-h incubation at RT, followed by anti-pERK antibody (cell signaling, 1:1000 dilution in 5% BSA in PBST) incubation overnight at 4° C. and secondary antibody-HRP (Jackson immunoresearch, 1:3000 dilution in 5% BSA in PBST) incubation at RT for 1 h. PBST wash is carried out after each step three times. Before the addition of the substrate (Advansta ELISABright), the plate is washed with PBS twice to remove detergent residues. Cellular pERK level is determined using the microplate reader (Biotek Synergy H1) to detect the chemiluminescence signal. IC₅₀ is determined by fitting a 4-parameter sigmoidal concentration-response model. FGFR wild type cells (H1975, PC-9 and AGS) are used to serve as negative control (FGFR selectivity) with the same assay procedures described above.

Results of pERK potency for FGFR1, FGFR2 and FGFR3, and selectivity for FGFR2 relative to FGFR1 and FGFR3 are presented in Table A.

In Table A, compounds having an IC50 less than or equal to 1 nM are represented as “A”; compounds having an IC50 greater than 1 nM but less than or equal to 10 nM are represented as “B”; compounds having an IC50 greater than 10 nM but less than or equal to 30 nM are represented as “C”; compounds having an IC50 greater than 30 nM but less than or equal to 100 nM are represented as “D”; and compounds having an IC50 greater than 100 nM are represented as “E”. The value “ND” refers to not determined.

Further in Table A, compounds having a selectivity ratio greater than 50 but less than or equal to 150 are represented as “F”; compounds having a selectivity ratio greater than 150 but less than or equal to 500 are represented as “G”; compounds having a selectivity ratio greater than 500 but less than or equal to 1000 are represented as “H”; compounds having a selectivity ratio greater than 1000 but less than or equal to 2000 are represented as “I”; and compounds having a selectivity ratio greater than 2000 are represented as “J”. The value “ND” refers to not determined.

These data demonstrate that Example compounds of the invention for which results are obtained listed in Table A are potent and selective inhibitors of FGFR2 with respect to FGFR1 or FGFR3.

TABLE A

| | | | | FGFR1 | FGFR3 |
|---|---|---|---|---|---|
| Example | FGFR2 | FGFR1 | FGFR3 | IC50/FGFR2 | IC50/FGFR2 |
| No. | IC50 | IC50 | IC50 | IC50 | IC50 |
| 1 | B | E | E | I | F |
| 2 | ND | ND | ND | ND | ND |
| 3 | E | E | E | ND | ND |
| 4 | B | E | E | F | G |
| 5 | ND | ND | ND | ND | ND |
| 6 | E | E | E | ND | ND |
| 7 | C | E | E | | |
| 8 | A | D | E | G | H |
| 9 | C | E | E | F | <50 |
| 10 | B | E | E | G | G |
| 11 | B | E | E | I | J |
| 12 | C | E | E | G | H |
| 13 | E | E | E | | |
| 14 | ND | ND | ND | ND | ND |
| 15 | C | E | E | | F |
| 16 | B | E | E | F | G |
| 17 | A | D | E | G | G |
| 18 | A | E | E | G | G |
| 19 | ND | ND | ND | ND | ND |
| 20 | D | E | E | | F |
| 21 | ND | ND | ND | ND | ND |
| 22 | B | E | E | G | G |
| 23 | B | E | E | G | H |
| 24 | B | E | E | F | F |
| 25 | B | D | E | | F |
| 26 | B | E | E | F | G |
| 27 | B | E | E | F | H |
| 28 | B | E | E | F | G |
| 29 | A | E | E | H | J |
| 30 | A | E | E | G | H |
| 31 | E | E | E | | |
| 32 | D | E | E | F | G |
| 33 | B | E | E | G | I |
| 34 | B | E | E | H | J |
| 35 | C | E | E | F | G |
| 36 | B | D | E | F | G |
| 37 | C | E | E | G | G |
| 38 | A | D | D | F | F |
| 39 | B | E | E | G | I |
| 40 | B | E | E | G | I |
| 41 | B | E | E | H | H |
| 42 | A | E | E | J | J |
| 43 | A | E | E | H | J |
| 44 | B | E | E | H | J |
| 45 | A | E | E | G | H |
| 46 | B | E | E | H | J |
| 47 | A | E | E | G | I |
| 48 | B | E | E | H | J |
| 49 | D | E | E | G | G |
| 50 | A | E | E | G | I |
| 51 | A | E | E | J | J |
| 52 | B | E | E | H | I |
| 53 | E | E | E | | |
| 54 | B | E | E | F | G |
| 55 | A | C | D | | F |
| 56 | A | E | E | G | I |
| 57 | B | E | E | G | H |
| 58 | B | E | E | G | H |
| 59 | B | E | E | F | H |
| 60 | C | E | E | | F |
| 61 | C | E | E | G | G |
| 62 | B | E | E | F | G |

### 2D) Cell Proliferation Assay

Cell proliferation assays are used to examine the potency with which compounds inhibit in vitro cell proliferation of cancer cell lines carrying FGFR aberrations or FGFR wild type. This demonstrates the molecular mode of action of compounds. Low IC₅₀ values are indicative of the high potency of the FGFR inhibitor compounds in this assay selling. It is observed that FGFR inhibitor compounds demonstrate potent inhibitory effect on the proliferation of human cancer cell lines carrying FGFR aberrations. Cell proliferation assays are performed in two-dimensional (2D)) anchorage-dependent conditions in 96 well plates (Corning® Black/Clear flat Bottom, Cat #3603) with the following cell lines:
Ba/F3 FGFR2-BICC1 (human FGFR2/V546F): Mouse Ba/F3 cell line stably expressed exogenous human FGFR2-BICC1 fusion gene bearing V546F mutation in FGFR2 Ba/F3;
FGFR2-BICC1 (human FGFR2/N546K): Mouse Ba/F3 cell line stably expressed exogenous human FGFR2-BICC1 fusion gene bearing N549K mutation in FGFR2;
Snu-16: human gastric cancer cell with FGFR2 amplification;
KATO III: human gastric cancer cell with FGFR2 amplification;
AN3CA: Human endometrial cancer with FGFR2 mutation (k310R, N549K);
MFM-223: Human breast cancer with FGFR1/2 amplification;
MFE-296: Human endometrial cancer with FGFR2 mutation (N549K);
MFE-280: Human endometrial cancer with FGFR2 mutation (B252W);
NCI-H1581: human non-small cell lung cancer with FGFR1 amplification;
KG-1a: Acute myelogenous leukemia with FGFR1OP2-FGFR1 translocation;
DMS114: human small cell carcinoma of the lung with FGFR1 amplification;
RT112: human bladder cancer with FGFR3 overexpression/FGFR3-TACC3 fusion;
SW780: human bladder cancer with FGFR3 overexpression/FGFR3-BALAP2L1 fusion;
Hep3B: Liver cancer with FGF19 amplification (FGFR4 ligand);
AGS: Human gastric adenocarcinoma with Kras G12D mutation and FGFR wild type;
PC-9: human non-small cell lung cancer (NSCLC) with wild-type FGFR and an EGFR del19 mutation; or
H1975: human non-small cell lung cancer (NSCLC) with wild-type FGFR and EGFR L858R/T790M mutation.

Cell lines are purchased from the American Type Culture Collection (ATCC) or Sigma. All cell lines are maintained in RPMI-1640 or DMEM supplemented with 10% heat-inactivated fetal bovine serum.

### 2D Cell Proliferation Assay Conditions

Cells growing in the log phase are detached with Gibco™ TrypLE™ Express Enzyme and plated in 96 well plate at 3000 to 5000 cells/well in 100 μl of media. After overnight incubation at 37° C., 5% CO₂ incubator, cells are treated with compounds (50 uL/well) at the final concentrations spanning from 0.000038 uM to 10 μM (4-fold serial dilution for total of 10 points). Cells are incubated at 37° C., 5% CO₂, and 95% humidity incubator for 96 hours. At the end of incubation 2D CTG reagent from Promega was added to each well according to vendors recommendation and mixed for 10 min in dark. The luminescence signals are determined with a Biotek™ plate reader. Data is fitted using a sigmoidal curve analysis program (GraphPad Prism®) with variable hill slope and IC50 is determined.

In Table B, compounds having an IC50 less than or equal to 1 nM are represented as “A”; compounds having an IC50 greater than 1 nM but less than or equal to 10 nM are represented as “B”; compounds having an IC50 greater than 10 nM but less than or equal to 30 nM are represented as “C”; compounds having an IC50 greater than 30 nM but less than or equal to 100 nM are represented as “D”; and compounds having an IC50 greater than 100 nM are represented as “E”. The value “ND” refers to not determined.

These data demonstrate that the tested compounds each possess potent inhibitory effects on the proliferation of cancer cell lines carrying FGFR2 aberrations including FGFR2 amplification (KATO III, SNU-16) or FGFR2 mutations (AN3CA, Ba/F3 FGFR2-N549K, Ba/F3 FGFR2-V564F).

TABLE B

Cell Line, IC50

| | | Ba/F3 | Ba/F3 | | |
|---|---|---|---|---|---|
| Example | | FGFR2- | FGFR2- | KATO | SNU- |
| No. | AN3CA | N549K | V564F | III | 16 |
| 4 | D | ND | ND | C | B |
| 8 | B | ND | ND | B | B |
| 10 | D | ND | ND | C | C |
| 11 | D | B | D | D | C |
| 16 | D | ND | ND | C | C |
| 17 | C | ND | ND | B | B |
| 18 | C | B | B | B | B |
| 22 | C | B | B | B | B |
| 23 | C | B | C | B | B |
| 24 | C | B | D | C | C |
| 26 | C | B | B | B | B |
| 28 | C | B | C | B | B |
| 29 | ND | B | D | B | B |
| 33 | C | B | A | B | B |
| 39 | C | B | C | C | C |
| 42 | C | B | B | C | C |
| 43 | ND | B | A | ND | ND |
| 44 | ND | B | A | ND | ND |
| 45 | ND | B | B | ND | ND |
| 47 | ND | E | E | ND | ND |
| 50 | ND | B | A | ND | ND |
| 51 | D | B | B | C | C |
| 52 | ND | B | A | ND | ND |
| 56 | A | B | A | B | B |
| 57 | B | C | E | B | B |
| 58 | B | C | B | C | C |
| 59 | D | B | A | D | D |
| 62 | D | C | A | C | C |

The abbreviations as used herein are accustomed to the art, some of which are also provided with the corresponding definitions below.

| Abbreviation | Name | Abbreviation | Name |
|---|---|---|---|
| MS | mass spectrometry | NMR | nuclear magnetic resonance |
| TLC | thin layer | HPLC | High-Performance Liquid |
| | chromatography | | Chromatography |
| LCMS | Liquid | DMF | dimethylformamide |
| | Chromatography-Mass | | |
| | Spectrometry | | |
| DCE | 1,2-dichloroethane | PE | petroleum ether |
| DCM | dichloromethane | DMSO | dimethyl sulfoxide |
| IPA | Isopropyl alcohol | DEA | diethylamine |
| MTBE | methyl tert-butyl ether | THF | tetrahydrofuran |
| Pd(dppf)Cl2 | [1,1′-bis(diphenylpho- | HATU | (1-[Bis(dimethylamino)- |
| | sphino)ferrocene]di- | | methylene]-1H-1,2,3-triazolo[4,5- |
| | chloropalladium (II) | | b]pyridinium 3-oxide |
| | | | hexafluorophosphate |
| TFA | trifluoroacetic acid | TEA | triethylamine |
| LiHMDS | lithium hexamethyl- | DIPEA | N,N-diisopropylethylamine |
| | disilylamide | | |
| Dmap | 4-(dimethylamino)- | TsOH | p-toluenesulfonic acid |
| | pyridine | | |
| HCCP | phosphonitrilic chloride | DMF-DMA | N,N-Dimethylformamide |
| | trimer | | dimethyl acetal |
| TMSA | (trimethylsilyl)acetylene | LDA | lithium diisopropylamide |
| X-PHOS | 2-dicyclohexyl- | Pd₂(dba)₃ | tris(dibenzylideneacetone) |
| | phosphino-2′,4′,6′- | | dipalladium (0) |
| | triisopropylbiphenyl | | |
| GSD | geometric standard | Pg | Protecting group |
| | deviation | | |
| NIS | N-iodosuccinimide | B₂Pin₂ | Bis(pinacolato)diboron |
| Pd(DtBPF)Cl₂ | [1,1′-Bis(di-tert-butyl- | NBS | N-bromosuccinimide |
| | phosphino)ferrocene]- | | |
| | dichloropalladium(II) | | |
| SEMCI | Chloromethyl 2- | | |
| | trimethylsilylethyl ether | | |

### General Synthetic Procedure

A compound of formula I may be prepared by a procedure known in the chemical arts or by a novel procedure described herein. A process for the preparation of a compound of formula I and novel intermediate compounds useful for the manufacture of a compound of formula I provide further features of the invention and are illustrated in the following procedures.

Generally, a compound of formula I may be synthesized using the procedure shown in Scheme 1. More specifically, a compound of formula IVa where X is Iodo, bromo, chloro (preferably iodo) is reacted with a compound of formula Va where Pg is a suitable amine protecting group such as BOC in the presence of a Pd coupling catalyst and a base such as potassium carbonate in a solvent such as dioxane and water to provide a compound of formula IIIa. A compound of formula IIIa is reacted with a suitable deprotecting reagent such as trifluoroacetic acid in a solvent such as dichloromethane to provide a compound of formula IIa. A compound of formula IIa is reacted with acryloyl chloride under acylation conditions to provide a compound of formula Ia.

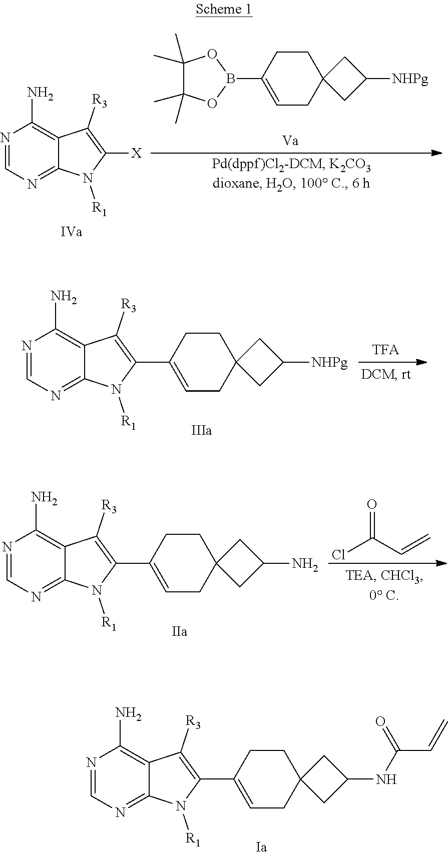

Generally, a compound of formula IVa may be synthesized using the procedure shown in Scheme 2. More specifically, a compound of formula IIXa where Y is bromo is reacted with a R₃ halide such as methyl iodide and a base such as cesium carbonate in a solvent such as DMF to provide a compound of formula VIIa where Y is bromo and R₁ is methyl. A compound of formula VIIa is reacted with a boronic acid derivative of R₃ in the presence of a Pd coupling catalyst and a base such as potassium carbonate in a solvent such as dioxane and water to provide a compound of formula VIa. A compound of VIa is reacted with N-iodosuccinimide and trifluoroacetic acid in a solvent such as dichloromethane to provide a compound of formula IVa where X is iodo. Alternatively, NBS (where X is Br) or NCS (where X is Cl) is used instead of N-iodosuccinimide.

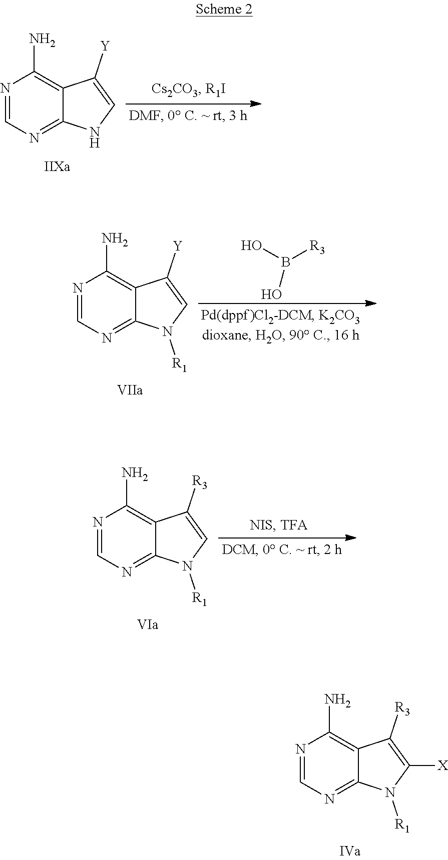

Generally, a compound of formula I may be further synthesized using the procedure shown in Scheme 3. More specifically, a compound of formula IXb where Y is bromo (alternatively, iodo or chloro) is reacted with a boronic acid derivative of R₃ in the presence of a Pd coupling catalyst and a base such as potassium carbonate in a solvent such as dioxane and water to provide a compound of formula IIIb. A compound of formula IIIb is reacted with a suitable deprotecting reagent such as trifluoroacetic acid in a solvent such as dichloromethane to provide a compound of formula IIb. A compound of formula IIb is reacted with acryloyl chloride under acylation conditions to provide a compound of formula Ib.

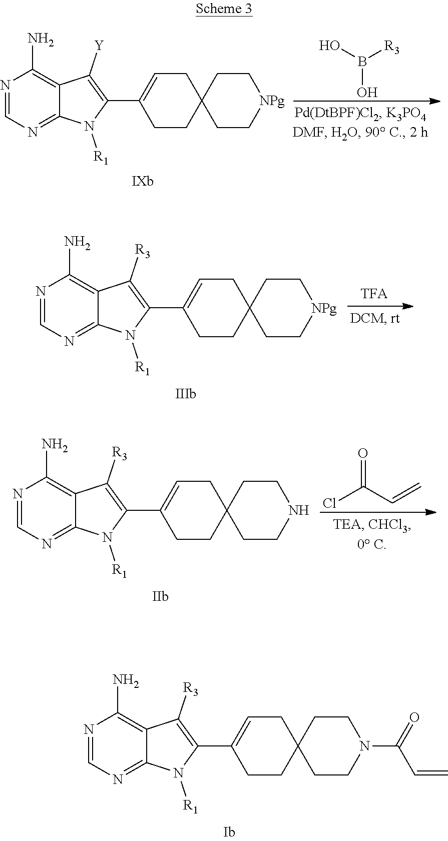

Generally, a compound of formula IXb may be synthesized using the procedure shown in Scheme 4. More specifically, a compound of formula XIb where Y is bromo (alternatively, iodo or chloro) is reacted with N-iodosuccinimide and trifluoroacetic acid in a solvent such as dichloromethane to provide a compound of formula Xb where X is iodo. NBS (where X is BR) or NCS (where X is Cl) can be used instead of N-iodosuccinimide. A compound of formula Xb is reacted with a compound of formula Vb where Pg is a suitable amine protecting group such as BOC in the presence of a Pd coupling catalyst and a base such as potassium carbonate in a solvent such as dioxane and water to provide a compound of formula IXb. A compound of formula XIb may be synthesized by a procedure known in the chemical arts or by a procedure described in the preparations and examples.

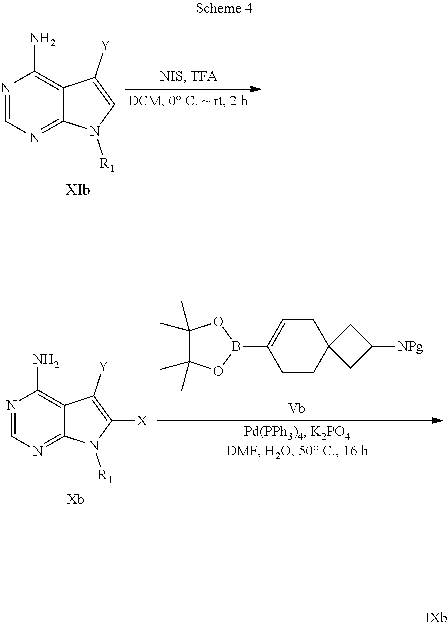

In the description of synthetic procedures herein, the phrase “dried and concentrated” generally refers to drying of a solution in an organic solvent over either sodium sulfate or magnesium sulfate, followed by filtration and removal of the solvent from the filtrate (generally under reduced pressure and at a temperature suitable to the stability of the material being prepared). Column chromatography is performed with regular gravity or flash chromatography, or pre-packed silica gel cartridges using a medium pressure chromatography apparatus (e.g., Biotage Isolera One®) eluting with the solvent or solvent mixture indicated. In some instances, the final products are purified by preparative thin layer chromatography using 20 cm×20 cm×0.5 mm or 20 cm×20 cm×1 mm silica gel plates developed in a suitable solvent system. Preparative high performance liquid chromatography (HPLC) is performed using a reverse phase column (e.g., Waters® Sunfire™ C18, Waters® Xbridge™ C18) of a size appropriate to the quantity of material being separated, generally eluting with a gradient of increasing concentration of methanol or acetonitrile in water, also containing 0.05% or 0.1% formic acid (or trifluoroacetic acid) or 10 mM ammonium acetate, at a rate of elution suitable to the column size and separation to be achieved. Chemical names are generated using ChemDraw® Professional version 19.1. The selection and use of suitable protecting groups is known in the chemical arts. See for example Greene's Protective Groups in Organic Synthesis 4th ed. P. Wuts and T. Greene, John Wiley & Sons, 2006. The mass to charge ratio of intermediates and products are recorded according to the most abundant isotope. Other isotope mass to charge ratio might or might be recorded.

### EXAMPLES

### Preparation 1

### Synthesis of 7-methyl-5-(4-(pyrimidin-2-yloxy)phenyl)-6-(2-azaspiro[3.5]non-6-en-7-yl)-7H-pyrrolo[2,3-d]pyrimidin-4-amine

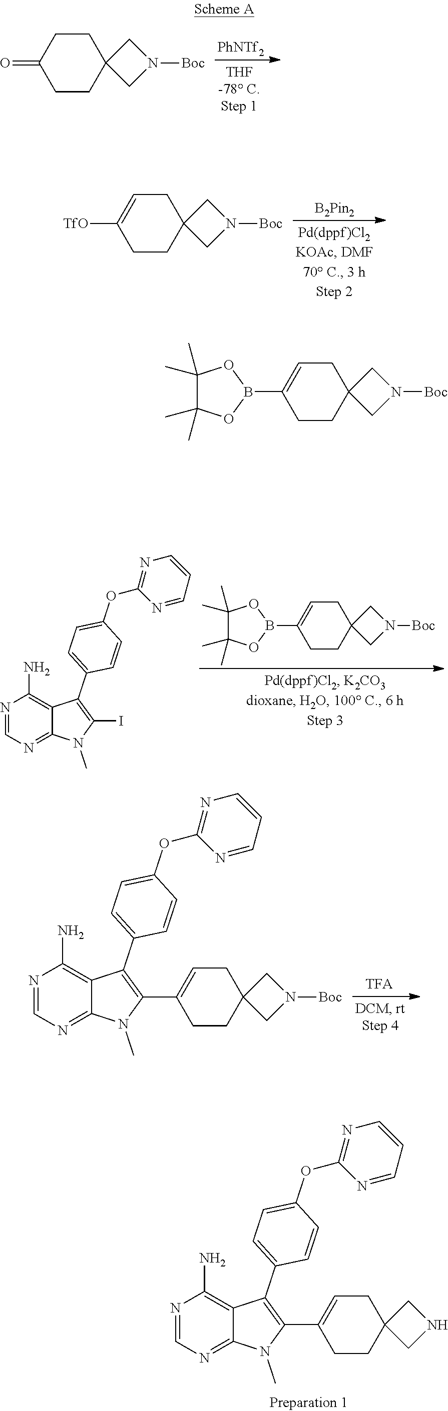

### Scheme A, Step 1. Synthesis of tert-butyl 7-(((trifluoromethyl)sulfonyl)oxy)-2-azaspiro[3.5]non-6-ene-2-carboxylate

To a stirred solution of tert-butyl 7-oxo-2-azaspiro[3.5]nonane-2-carboxylate (1 g, 4.18 mmol, 1 eq) in dry THF (20 mL) was added dropwise a solution of LiHMDS (5 mL, 5.02 mmol, 1.2 eq, 1M in THF) at −78° C. under N₂ and the mixture was stirred at −78° C. for 1 hr, then PhNTf₂ (1.65 g, 4.59 mmol, 1.1 eq) was added, and the mixture was warmed to room temperature slowly and stirred for 16 hrs. The reaction mixture was quenched with sat. NH₄Cl aqueous solution and extracted with EtOAc (3×15 mL). The combined organic layers were dried over anhydrous Na₂SO₄ and filtered. The filtrate was concentrated under reduced pressure. The residue was purified by silica gel column chromatography, eluting with hexanes/EtOAc (5:1) to afford the title compound (890 mg, 57% yield) as a colorless oil.

### Scheme A, Step 2. Synthesis of tert-butyl 7-(4,4,5,5-tetramethyl-1,3,2-dioxaborolan-2-yl)-2-azaspiro[3.5]non-6-ene-2-carboxylate

To a stirred solution of compound tert-butyl 7-(((trifluoromethyl)sulfonyl)oxy)-2-azaspiro[3.5]non-6-ene-2-carboxylate (0.89 g, 2.4 mmol, 1 eq) in DMF (15 mL) was added B₂Pin₂ (0.67 g, 2.6 mmol, 1.1 eq), KOAc (0.71 g, 7.2 mmol, 3 eq) and Pd(dppf)Cl₂ (196 mg, 0.24 mmol, 0.1 eq), and then stirred at 70° C. for 3 hrs under N₂. The reaction mixture was concentrated in vacuo, diluted with EtOAc (20 mL), filtered through a pad of filter Celite®, and concentrated in vacuo. The residue was used for next step.

### Scheme A, Step 3. Synthesis of tert-butyl 7-(4-amino-7-methyl-5-(4-(pyrimidin-2-yloxy)phenyl)-7H-pyrrolo[2,3-d]pyrimidin-6-yl)-2-azaspiro[3.5]non-6-ene-2-carboxylate

A mixture of 6-iodo-7-methyl-5-(4-(pyrimidin-2-yloxy)phenyl)-7H-pyrrolo[2,3-d]pyrimidin-4-amine (533 mg, 1.2 mmol, 1 eq), tert-butyl 7-(4,4,5,5-tetramethyl-1,3,2-dioxaborolan-2-yl)-2-azaspiro[3.5]non-6-ene-2-carboxylate (838 mg, 2.4 mmol, 2 eq), Pd(dppf)Cl₂ (98 mg, 0.12 mmol, 0.1 eq) and K₂CO₃ (497 mg, 3.6 mmol, 3 eq) in dioxane (15 mL) and H₂O (1.5 mL) was stirred at 100° C. for 6 h under N₂. The resulting mixture was diluted with H₂O (20 mL) and extracted with EtOAc (3×15 mL). The combined organic layers were dried over anhydrous Na₂SO₄ and filtered. The filtrate was concentrated under reduced pressure. The residue was purified by silica gel column chromatography, eluting with DCM/MeOH (30:1) to afford the title compound (330 mg, 51% yield) as a yellow solid.

LCMS: m/z 540.15 [M+1]⁺.

### Scheme A, Step 4. Synthesis of 7-methyl-5-(4-(pyrimidin-2-yloxy)phenyl)-6-(2-azaspiro[3.5]non-6-en-7-yl)-7H-pyrrolo[2,3-d]pyrimidin-4-amine

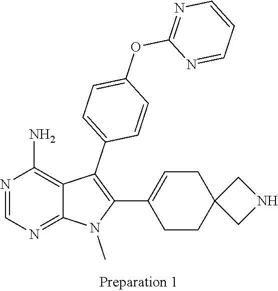 [SMILES: Cn1c(C2=CCC3(CC2)CNC3)c(-c2ccc(Oc3ncccn3)cc2)c2c(N)ncnc21]

To a stirred solution of tert-butyl 7-(4-amino-7-methyl-5-(4-(pyrimidin-2-yloxy)-phenyl)-7H-pyrrolo[2,3-d]pyrimidin-6-yl)-2-azaspiro[3.5]non-6-ene-2-carboxylate (330 mg, 0.61 mmol, 1 eq) in DCM (5 mL) was added TFA (5 mL) and the mixture was stirred at room temperature for 2 hrs. The solution was concentrated. The crude 7-methyl-5-(4-(pyrimidin-2-yloxy)phenyl)-6-(2-azaspiro[3.5]non-6-en-7-yl)-7H-pyrrolo[2,3-d]pyrimidin-4-amine, was treated with NH₄OH aqueous solution and extracted with DCM (3×15 mL). The combined organic layer was washed with brine (15 mL) and dried over anhydrous Na₂SO₄ and filtered. The filtrate was concentrated under reduced pressure and purified by Prep-TLC (DCM/MeOH=10/1) to afford the title compound (160 mg, 60% yield) as a yellow solid.

LCMS: m/z 440.20 [M+1]⁺.

### Example 1

### Synthesis of 1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)prop-2-en-1-one

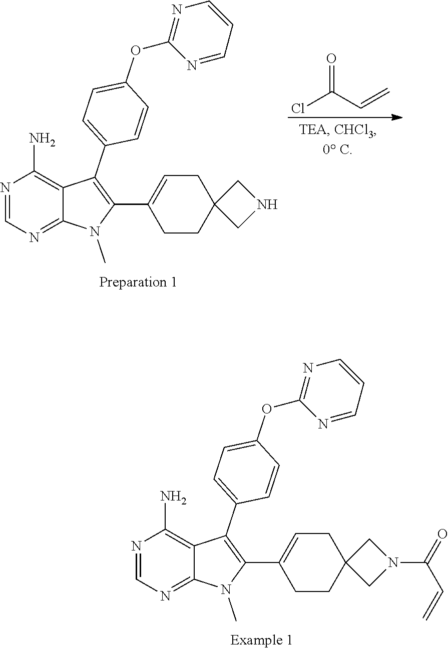

To a solution of 7-methyl-5-(4-(pyrimidin-2-yloxy)phenyl)-6-(2-azaspiro-[3.5]non-6-en-7-yl)-7H-pyrrolo[2,3-d]pyrimidin-4-amine (62 mg, 0.141 mmol, 1 eq) in dry CHCl₃ (5 mL) was added TEA (43 mg, 0.423 mmol, 3 eq). A solution of acryloyl chloride (15.3 mg, 0.169 mmol, 1.2 eq) in dry CHCl₃ (0.5 mL) was added dropwise at 0° C. and the mixture was stirred at 0° C. for 0.5 hr under N₂. The reaction mixture was quenched with MeOH. The solvent was removed under reduced pressure. The residue was purified by Prep-HPLC (0.1% TFA in ACN/H₂O) to afford the title compound (30.7 mg, 44% yield) as a white solid.

LCMS: m/z 494.35 [M+1]⁺.

¹H NMR (400 MHz, CD₃OD) δ 8.63 (d, J=4.0 Hz, 2H), 8.30 (s, 1H), 7.49 (d, J=8.0 Hz, 2H), 7.33 (d, J=8.0 Hz, 2H), 7.26 (t, J=4.0 Hz, 1H), 6.35-6.18 (m, 2H), 5.94 (brs, 1H), 5.71 (d, J=12.0 Hz, 1H), 4.01-3.92 (m, 2H), 3.80 (s, 3H), 3.78-3.70 (m, 2H), 2.49-2.46 (m, 2H), 2.23-2.16 (m, 2H), 1.90 (t, J=4.0 Hz, 2H).

### Example 2

### Synthesis of 1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)-2-methylprop-2-en-1-one

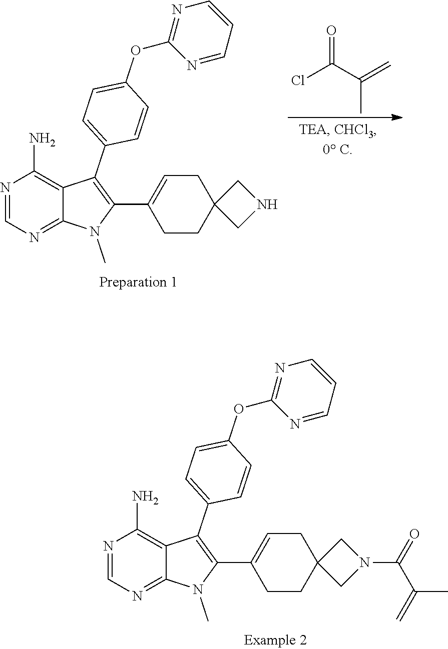

To a solution of 7-methyl-5-(4-(pyrimidin-2-yloxy)phenyl)-6-(2-azaspiro[3.5]-non-6-en-7-yl)-7H-pyrrolo[2,3-d]pyrimidin-4-amine (65 mg, 0.15 mmol, 1 eq) in dry CHCl₃ (5 mL) was added TEA (43 mg, 0.423 mmol, 3 eq). The solution of methacryloyl chloride (18.6 mg, 0.178 mmol, 1.2 eq) in dry CHCl₃ (0.5 mL) was added dropwise at 0° C. and the mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (23.9 mg, 32% yield) as a white solid.

LCMS: m/z 508.45 [M+1]⁺.

¹H NMR (400 MHz, CD₃OD) δ 8.63 (d, J=4.0 Hz, 2H), 8.30 (s, 1H), 7.48 (d, J=8.0 Hz, 2H), 7.30 (d, J=8.0 Hz, 2H), 7.26 (t, J=4.0 Hz, 1H), 5.93 (brs, 1H), 5.43 (s, 1H), 5.33 (s, 1H), 3.97-3.89 (m, 2H), 3.79 (s, 3H), 3.76-3.68 (m, 2H), 2.47-2.42 (m, 2H), 2.22-2.12 (m, 2H), 1.90-1.85 (m, 5H).

### Preparation 2

### Synthesis of 6-(4-amino-7-methyl-5-(4-(pyrimidin-2-yloxy)phenyl)-7H-pyrrolo[2,3-d]pyrimidin-6-yl)-2-azaspiro[3.3]heptane

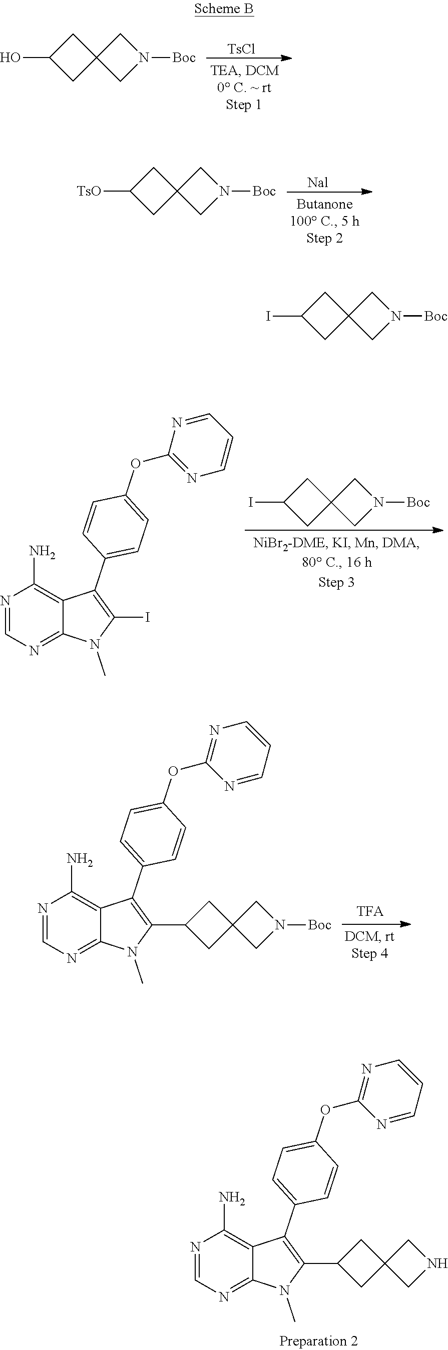

### Scheme B, Step 1. Synthesis of tert-butyl 6-(tosyloxy)-2-azaspiro[3.3]heptane-2-carboxylate

To a stirred solution of tert-butyl 6-hydroxy-2-azaspiro[3.3]heptane-2-carboxylate (4.5 g, 21.1 mmol, 1 eq) in dry DCM (50 mL) was added TEA (4.3 g, 42.2 mmol, 2 eq) at 0° C., followed by TsCl (6.1 g, 31.6 mmol, 1.5 eq), and the mixture was stirred at room temperature for 16 hrs under N₂. The reaction mixture was quenched with water, extracted with DCM (3×30 mL). The combined organic layers were dried over anhydrous Na₂SO₄ and filtered. The filtrate was concentrated under reduced pressure. The residue was purified by silica gel column chromatography, eluting with hexanes/EtOAc (6:1) to afford the title compound (6.1 g, 78% yield) as a white solid.

### Scheme B, Step 2. Synthesis of tert-butyl 6-iodo-2-azaspiro[3.3]heptane-2-carboxylate

To a stirred solution of tert-butyl 6-(tosyloxy)-2-azaspiro[3.3]heptane-2-carboxylate. (9.5 g, 25.9 mmol, 1 eq) in methyl ethylketone (80 mL) was added NaI (15.6 g, 103.6 mmol, 4 eq). The reaction was stirred at 100° C. for 16 hrs under N₂. The reaction mixture was cooled to room temperature, filtered through a filter pad, and the filtrate was concentrated in vacuo. The residue was purified by silica gel column chromatography, eluting with hexanes/EtOAc (10:1) to afford the title compound (8.2 g, 98% yield) as a white solid.

### Scheme B, Step 3. Synthesis of tert-butyl 6-(4-amino-7-methyl-5-(4-(pyrimidin-2-yloxy)phenyl)-7H-pyrrolo[2,3-d]pyrimidin-6-yl)-2-azaspiro[3.3]heptane-2-carboxylate

A mixture of 6-iodo-7-methyl-5-(4-(pyrimidin-2-yloxy)phenyl)-7H-pyrrolo[2,3-d]-pyrimidin-4-amine (1 g, 2.25 mmol, 1 eq), tert-butyl 6-iodo-2-azaspiro[3.3]heptane-2-carboxylate (2.18 g, 6.75 mmol, 3 eq), NiBr-DME (79 mg, 0.225 mmol, 0.1 eq), picolinimidamide hydrochloride (36 mg, 0.225 mmol, 0.1 eq), manganese (619 mg, 11.25 mmol, 5 eq) and KI (112 mg, 6.75 mmol, 3 eq) in dry DMAc (20 mL) was stirred at 80° C. for 16 h under N₂. The resulting mixture was diluted with H₂O (20 mL) and extracted with EtOAc (3×20 mL). The combined organic layers were washed with brine (3×20 mL), dried over anhydrous Na₂SO₄ and filtered. The filtrate was concentrated under reduced pressure. The residue was purified by silica gel column chromatography, eluting with CH₂Cl₂/MeOH (30:1) to afford the title compound (250 mg, 22% yield) as a yellow solid.

LCMS: m/z 514.45 [M+1]⁺.

### Scheme B, Step 4. Synthesis of 6-(4-amino-7-methyl-5-(4-(pyrimidin-2-yloxy)phenyl)-7H-pyrrolo[2,3-d]pyrimidin-6-yl)-2-azaspiro[3.3]heptane

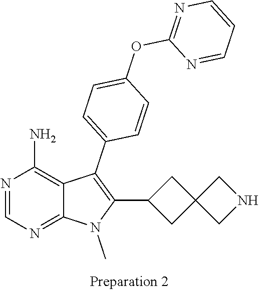 [SMILES: Cn1c(C2CC3(CNC3)C2)c(-c2ccc(Oc3ncccn3)cc2)c2c(N)ncnc21]

To a stirred solution of tert-butyl 6-(4-amino-7-methyl-5-(4-(pyrimidin-2-yloxy)phenyl)-7H-pyrrolo[2,3-d]pyrimidin-6-yl)-2-azaspiro[3.3]heptane-2-carboxylate (250 mg, 0.487 mmol, 1 eq) in DCM (5 mL) was added TFA (5 mL) under N₂, and the mixture was stirred at room temperature for 2 hrs. The solution was concentrated. The crude material was treated with NH₄OH aqueous solution and extracted with DCM (3×15 mL). The combined organic layer was washed by brine (15 mL), dried over anhydrous Na₂SO₄ and filtered. The filtrate was concentrated under reduced pressure and purified by Prep-TLC (CH₂Cl₂/MeOH=10/1) to afford the title compound (120 mg, 60% yield) as a yellow solid.

LCMS: m/z 414.40 [M+1]⁺.

### Example 3

### Synthesis of 1-(6-(4-amino-7-methyl-5-(4-(pyrimidin-2-yloxy)phenyl)-7H-pyrrolo[2,3-d]pyrimidin-6-yl)-2-azaspiro[3.3]heptan-2-yl)prop-2-en-1-one

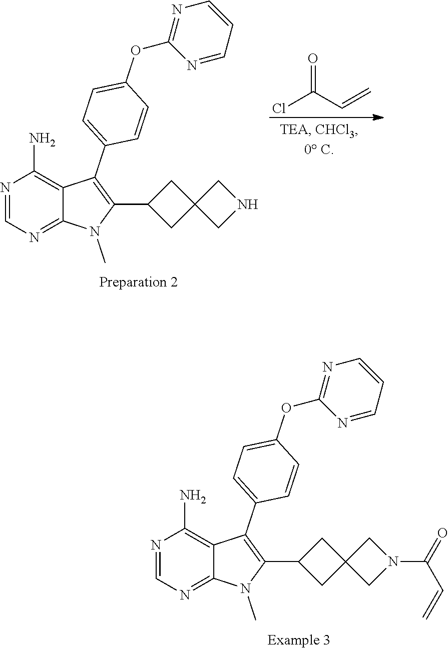

To a solution of 6-(4-amino-7-methyl-5-(4-(pyrimidin-2-yloxy)phenyl)-7H-pyrrolo-[2,3-d]pyrimidin-6-yl)-2-azaspiro[3.3]heptane (60 mg, 0.145 mmol, 1 eq) in dry CHCl₃ (5 mL) was added TEA (44 mg, 0.425 mmol, 3 eq). A solution of acryloyl chloride (16 mg, 0.174 mmol, 1.2 eq) in dry CHCl₃ (0.5 mL) was added dropwise at 0° C. and the mixture was stirred at 0° C. for 0.5 hr under N₂. The reaction mixture was quenched with MeOH. The solvent was removed under reduced pressure. The residue was purified by Prep-HPLC (0.1% TFA in ACN/H₂O) to afford the title compound (9.3 mg, 13.7% yield) as a white solid.

LCMS: m/z 468.45 [M+1]⁺.

¹H NMR (400 MHz, CD₃OD) δ 8.64 (d, J=4.0 Hz, 2H), 8.26 (s, 1H), 7.51 (d, J=8.0 Hz, 2H), 7.33 (d, J=8.0 Hz, 2H), 7.29-7.26 (m, 1H), 6.33-6.12 (m, 2H), 5.74-5.63 (m, 1H), 4.37 (s, 1H), 4.13 (s, 1H), 3.99 (s, 1H), 3.86-3.79 (m, 4H), 3.76 (s, 1H), 2.59-2.49 (m, 2H), 2.26-2.18 (m, 2H).

### Example 4

### Synthesis of 1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one

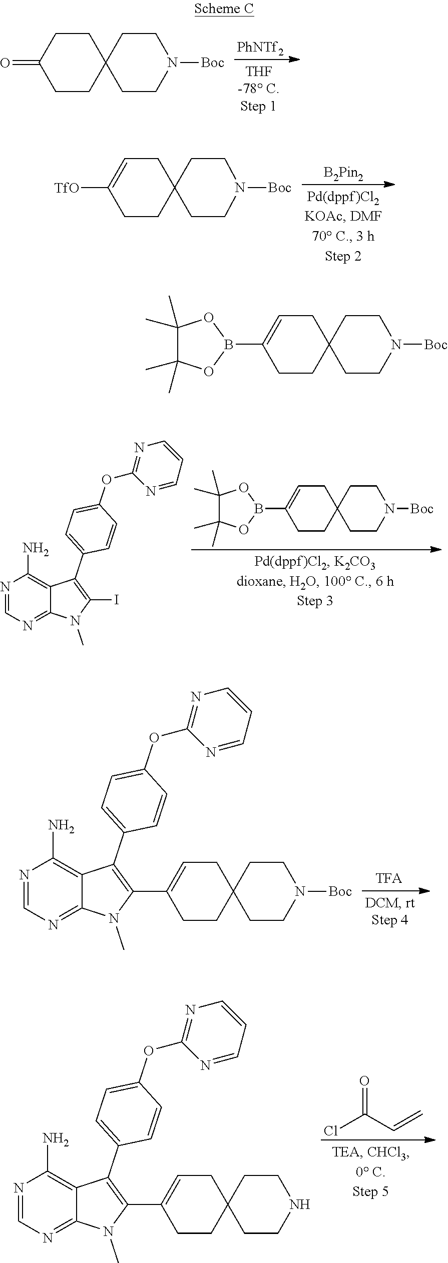

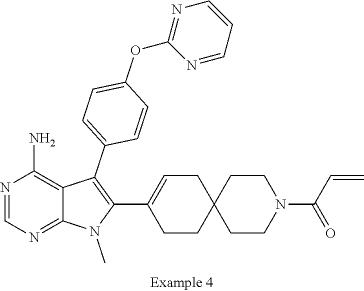 [SMILES: C=CC(=O)N1CCC2(CC=C(c3c(-c4ccc(Oc5ncccn5)cc4)c4c(N)ncnc4n3C)CC2)CC1]

### Scheme C, Step 1. Synthesis of tert-butyl 9-(((trifluoromethyl)sulfonyl)oxy)-3-azaspiro[5.5]undec-8-ene-3-carboxylate

To a stirred solution of tert-butyl 9-oxo-3-azaspiro[5.5]undecane-3-carboxylate (0.8 g, 3 mmol, 1 eq) in dry THF (20 mL) was added dropwise a solution of LiHMDS (3.6 mL, 3.6 mmol, 1.2 eq, 1M in THF) at −78° C. under N₂ and the mixture was stirred at −78° C. for 1 hr. Then PhNTf₂ (1.18 g, 3.3 mmol, 1.1 eq) was added, and the mixture was warmed to room temperature slowly and stirred for 16 hrs. The reaction mixture was quenched with sat. NH₄Cl aqueous solution, extracted with EtOAc (3×15 mL). The combined organic layers were dried over anhydrous Na₂SO₄ and filtered. The filtrate was concentrated under reduced pressure. The residue was purified by silica gel column chromatography, eluting with hexanes/EtOAc (5:1) to afford the title compound (247 mg, 21% yield) as a colorless oil.

### Scheme C, Step 2. Synthesis of tert-butyl 9-(4,4,5,5-tetramethyl-1,3,2-dioxaborolan-2-yl)-3-azaspiro[5.5]undec-8-ene-3-carboxylate

To a stirred solution of tert-butyl 9-(4,4,5,5-tetramethyl-1,3,2-dioxa-borolan-2-yl)-3-azaspiro[5.5]undec-8-ene-3-carboxylate (247 mg, 0.64 mmol, 1 eq) in DMF (5 mL) was added B₂Pin₂ (179 mg, 0.7 mmol, 1.1 eq), KOAc (188 mg, 1.92 mmol, 3 eq) and Pd(dppf)Cl₂ (52 mg, 0.064 mmol, 0.1 eq). The reaction mixture was stirred at 70° C. for 3 hrs under N₂, then concentrated in vacuo, diluted with EtOAc (20 mL), and filtered through a filter pad. The filtrate was concentrated in vacuo. The resulting residue was used in the next step.

### Scheme C, Step 3. Synthesis of tert-butyl 9-(4-amino-7-methyl-5-(4-(pyrimidin-2-yloxy)phenyl)-7H-pyrrolo[2,3-d]pyrimidin-6-yl)-3-azaspiro[5.5]undec-8-ene-3-carboxylate

A mixture of compound 6-iodo-7-methyl-5-(4-(pyrimidin-2-yloxy)phenyl)-7H-pyrrolo[2,3-d]pyrimidin-4-amine (177.6 mg, 0.4 mmol, 1 eq), (prepared essentially by the procedure described in WO2020231990), tert-butyl 9-(4,4,5,5-tetramethyl-1,3,2-dioxaborolan-2-yl)-3-azaspiro[5.5]undec-8-ene-3-carboxylate (241 mg, 0.64 mmol, 1.6 eq), Pd(dppf)Cl₂ (32 mg, 0.04 mmol, 0.1 eq) and K₂CO₃ (165 mg, 1.2 mmol, 3 eq) in dioxane (5 mL) and H₂O (0.5 mL) was stirred at 100° C. for 6 h under N₂. The resulting mixture was diluted with H₂O (20 mL) and extracted with EtOAc (3×15 mL). The combined organic layers were dried over anhydrous Na₂SO₄ and filtered. The filtrate was concentrated under reduced pressure. The residue was purified by silica gel column chromatography, eluting with DCM/MeOH (30:1) to afford the title compound (138 mg, 60% yield) as a yellow solid.

LCMS: m/z 568.25 [M+1]⁺.

### Scheme C, Step 4. Synthesis of 7-methyl-5-(4-(pyrimidin-2-yloxy)phenyl)-6-(3-azaspiro[5.5]undec-8-en-9-yl)-7H-pyrrolo[2,3-d]pyrimidin-4-amine

To a stirred solution of tert-butyl 9-(4-amino-7-methyl-5-(4-(pyrimidin-2-yloxy)phenyl)-7H-pyrrolo[2,3-d]pyrimidin-6-yl)-3-azaspiro[5.5]undec-8-ene-3-carboxylate (138 mg, 0.24 mmol, 1 eq) in DCM (5 mL) was added TFA (5 mL) under N₂ and the mixture was stirred at room temperature for 2 hrs. The solution was concentrated. The crude residue was treated with NH₄OH aqueous solution and extracted with DCM (3×15 mL). The combined organic layers were washed with brine (15 mL), dried over anhydrous Na₂SO₄ and filtered. The filtrate was concentrated in vacuo to afford the title compound (113.6 mg, 80% yield) as a yellow solid.

LCMS: m/z 468.24 [M+1]⁺.

### Scheme C, Step 5. Synthesis of 1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one

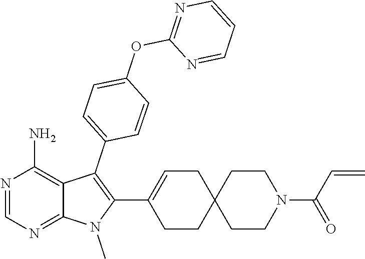 [SMILES: C=CC(=O)N1CCC2(CC=C(c3c(-c4ccc(Oc5ncccn5)cc4)c4c(N)ncnc4n3C)CC2)CC1]

### Example 4

To a stirred solution of compound 7-methyl-5-(4-(pyrimidin-2-yloxy)phenyl)-6-(3-azaspiro[5.5]undec-8-en-9-yl)-7H-pyrrolo[2,3-d]pyrimidin-4-amine (113.6 mg, 0.24 mmol, 1 eq) in dry CHCl₃ (5 mL) was added TEA (96.9 mg, 0.96 mmol, 4 eq). A solution of acryloyl chloride (21 mg, 0.24 mmol, 1 eq) in dry CHCl₃ (0.5 mL) was added dropwise at 0° C. and the mixture was stirred at 0° C. for 0.5 hr under N₂. The reaction mixture was quenched with MeOH. The solvent was removed under reduced pressure. The residue was purified by Prep-HPLC (0.1 TFA in ACN/H₂O) to afford the title compound 22 mg, 20% yield) as a white solid.

LCMS: m/z 522.20 [M+1]⁺.

¹H NMR (400 MHz, CD₃OD) δ 8.63 (d, J=4.0 Hz, 2H), 8.12 (s, 1H), 7.45 (d, J=8.0 Hz, 2H), 7.26-7.24 (m, 3H), 6.73 (dd, J=16.0, 8.0 Hz, 1H), 6.15 (d, J=16.0 Hz, 1H), 5.87 (brs, 1H), 5.70 (d, J=8.0 Hz, 1H), 3.69 (s, 3H), 3.68-3.46 (m, 4H), 2.18-2.02 (m, 4H), 1.62-1.58 (m, 2H), 1.47-1.39 (m, 4H).

### Example 5

### Synthesis of 1-(8-(4-amino-7-methyl-5-(4-(pyrimidin-2-yloxy)phenyl)-7H-pyrrolo[2,3-d]pyrimidin-6-yl)-2-azaspiro[4.5]dec-7-en-2-yl)prop-2-en-1-one

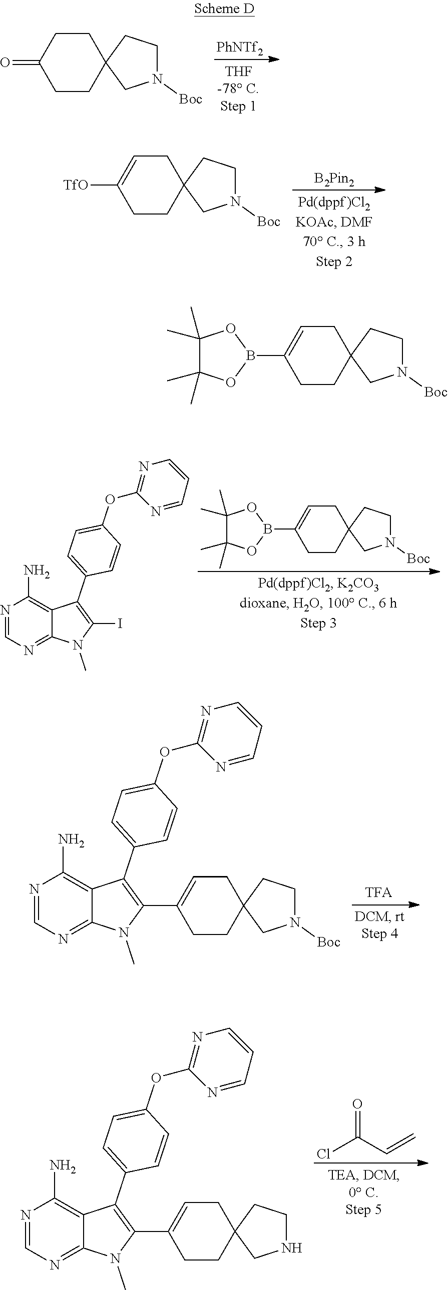

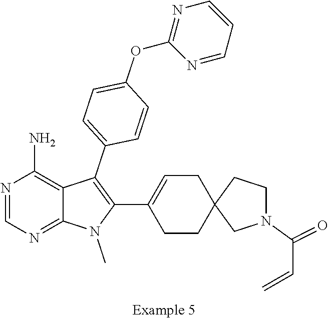 [SMILES: C=CC(=O)N1CCC2(CC=C(c3c(-c4ccc(Oc5ncccn5)cc4)c4c(N)ncnc4n3C)CC2)C1]

### Scheme D, Step 1. Synthesis of tert-butyl 8-(((trifluoromethyl)sulfonyl)oxy)-2-azaspiro[4.5]dec-7-ene-2-carboxylate

To a stirred solution of compound tert-butyl 8-oxo-2-azaspiro[4.5]decane-2-carboxylate (1 g, 3.91 mmol, 1 eq) in dry THF (20 mL) was added dropwise the solution of LiHMDS (4.7 mL, 4.69 mmol, 1.2 eq, 1M in THF) at −78° C. and the mixture was stirred at −78° C. for 1 hr under N2. Then PhNTf₂ (1.5 g, 4.31 mmol, 1.1 eq) was added, and the resulting mixture was warmed to room temperature slowly and stirred for 16 hrs. The reaction mixture was quenched with sat. NH₄Cl aqueous solution, extracted with EtOAc (3×15 mL). The combined organic layers were dried over anhydrous Na₂SO₄ and filtered. The filtrate was concentrated under reduced pressure. The residue was purified by silica gel column chromatography, eluting with hexanes/EtOAc (5:1) to afford the title compound (1.4 g, 94% yield) as a colorless oil.

### Scheme D, Step 2. Synthesis of tert-butyl 8-(4,4,5,5-tetramethyl-1,3,2-dioxaborolan-2-yl)-2-azaspiro[4.5]dec-7-ene-2-carboxylate

To a stirred solution of compound tert-butyl 8-(((trifluoromethyl)sulfonyl)oxy)-2-azaspiro[4.5]dec-7-ene-2-carboxylate (1.4 g, 3.7 mmol, 1 eq) in DMF (15 mL) was added B₂Pin₂ (0.99 g, 3.9 mmol, 1.1 eq), KOAc (1.15 g, 11.7 mmol, 3 eq) and Pd(dppf)Cl₂ (322 mg, 0.37 mmol, 0.1 eq). The reaction mixture was stirred at 70° C. for 3 hrs under N₂. The reaction mixture was concentrated in vacuo, diluted with EtOAc (20 mL) and filter through a filter pad, and concentrated in vacuo. The resulting residue was used in the next step.

### Scheme D, Step 3. Synthesis of tert-butyl 8-(4-amino-7-methyl-5-(4-(pyrimidin-2-yloxy)phenyl)-7H-pyrrolo[2,3-d]pyrimidin-6-yl)-2-azaspiro[4.5]dec-7-ene-2-carboxylate

A mixture of compound 6-iodo-7-methyl-5-(4-(pyrimidin-2-yloxy)phenyl)-7H-pyrrolo[2,3-d]pyrimidin-4-amine (821 mg, 1.85 mmol, 1 eq), tert-butyl 8-(4,4,5,5-tetramethyl-1,3,2-dioxaborolan-2-yl)-2-azaspiro[4.5]dec-7-ene-2-carboxylate (3.7 mmol, 2 eq), Pd(dppf)Cl₂ (150 mg, 0.18 mmol, 0.1 eq) and K₂CO₃ (765 mg, 5.5 mmol, 3 eq) in dioxane (15 mL)/H₂O (1.5 mL) was stirred at 100° C. for 6 h under nitrogen atmosphere. The resulting mixture was diluted H₂O (20 mL) and extracted with EtOAc (3×15 mL). The combined organic layers were dried over anhydrous Na₂SO₄ and filtered. The filtrate was concentrated under reduced pressure. The resulting residue was purified by silica gel column chromatography, eluting with CH₂Cl₂/MeOH (30:1) to afford the title compound (720 mg, 72% yield) as a yellow solid.

LCMS: m/z 554.28 [M+1]⁺.

### Scheme D, Step 4. Synthesis of 7-methyl-5-(4-(pyrimidin-2-yloxy)phenyl)-6-(2-azaspiro[4.5]dec-7-en-8-yl)-7H-pyrrolo[2,3-d]pyrimidin-4-amine

To a stirred solution of compound tert-butyl 8-(4-amino-7-methyl-5-(4-(pyrimidin-2-yloxy)phenyl)-7H-pyrrolo[2,3-d]pyrimidin-6-yl)-2-azaspiro[4.5]dec-7-ene-2-carboxylate (450 mg, 0.81 mmol, 1 eq) in CH₂Cl₂ (5 mL) was added TFA (5 mL) under N₂ at room temperature and stirred for 2 hrs. The solution was concentrated. The crude residue was treated with NH₄OH aqueous and extracted with CH₂Cl₂ (3×15 mL). The combined organic layers were washed with brine (15 mL), dried over anhydrous Na₂SO₄ and filtered. The filtrate was concentrated to afford the title compound (340 mg, 92% yield) as a yellow solid.

LCMS: m/z 454.23 [M+1]⁺.

### Scheme D, Step 5. Synthesis of 1-(8-(4-amino-7-methyl-5-(4-(pyrimidin-2-yloxy)phenyl)-7H-pyrrolo[2,3-d]pyrimidin-6-yl)-2-azaspiro[4.5]dec-7-en-2-yl)prop-2-en-1-one

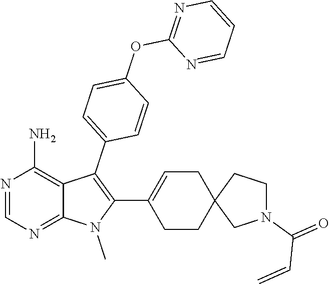 [SMILES: C=CC(=O)N1CCC2(CC=C(c3c(-c4ccc(Oc5ncccn5)cc4)c4c(N)ncnc4n3C)CC2)C1]

### Example 5

To a stirred solution of compound 7-methyl-5-(4-(pyrimidin-2-yloxy)phenyl)-6-(2-azaspiro[4.5]dec-7-en-8-yl)-7H-pyrrolo[2,3-d]pyrimidin-4-amine (130 mg, 0.28 mmol, 1 eq) in dry CHCl₃ (5 mL) was added TEA (133.3 mg, 1.32 mmol, 4 eq). A solution of acryloyl chloride (25.9 mg, 0.286 mmol, 1 eq) in dry CHCl₃ (0.5 mL) was added dropwise at 0° C. and stirred at 0° C. for 0.5 hrs under N₂. The reaction mixture was quenched with MeOH. The solvent was removed under reduced pressure. The resulting residue was purified by Prep-HPLC (0.1 TFA in ACN/H₂O) to afford the title compound (33.2 mg, 26% yield) as a white HCl salt solid.

LCMS: m/z 508.50 [M+1]⁺.

¹H NMR (400 MHz, CD₃OD) δ 8.63 (d, J=4.0 Hz, 2H), 8.30 (s, 1H), 7.49 (d, J=8.0 Hz, 2H), 7.33 (d, J=8.0 Hz, 2H), 7.26 (t, J=4.0 Hz, 1H), 6.63-6.40 (m, 1H), 6.25-6.20 (m, 1H), 6.00-5.95 (m, 1H), 5.78-5.70 (m, 1H), 3.80 (s, 3H), 3.75-3.70 (m, 1H), 3.60-3.50 (m, 1H), 3.42-3.37 (m, 1H), 3.29-3.24 (m, 1H), 2.20-2.16 (m, 4H), 1.88-1.83 (m, 1H), 1.77-1.65 (m, 3H).

### Example 6

### Synthesis of 1-(9-(4-amino-7-methyl-5-(4-(pyrimidin-2-yloxy)phenyl)-7H-pyrrolo[2,3-d]pyrimidin-6-yl)-3-azaspiro[5.5]undecan-3-yl)prop-2-en-1-one

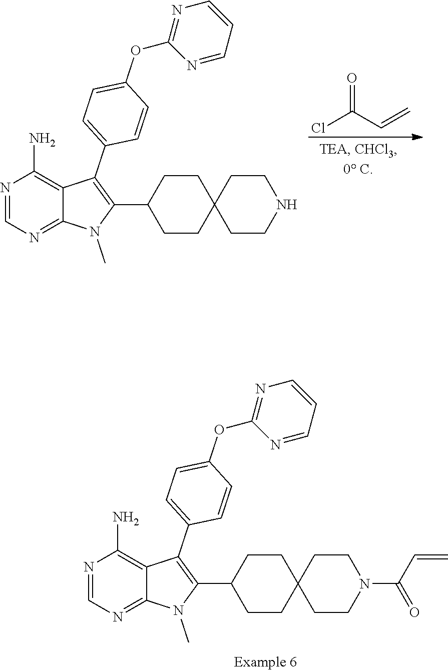

To a stirred solution of compound 7-methyl-5-(4-(pyrimidin-2-yloxy)phenyl)-6-(3-azaspiro[5.5]undecan-9-yl)-7H-pyrrolo[2,3-d]pyrimidin-4-amine (42 mg, 0.089 mmol, 1 eq) in dry CHCl₃ (5 mL) was added TEA (27 mg, 0.269 mmol, 3 eq). A solution of acryloyl chloride (7.6 mg, 0.106 mmol, 1.2 eq) in dry CHCl₃ (0.5 mL) was added dropwise at 0° C. and the mixture was stirred at 0° C. for 0.5 hr under N₂. The reaction mixture was quenched with MeOH. The solvent was removed under reduced pressure. The residue was purified by Prep-HPLC (0.1% TFA in ACN/H₂O) to afford the title compound (1.5 mg, 3.5% yield) as a white solid.

LCMS: m/z 524.40 [M+1]⁺.

¹H NMR (400 MHz, CD3OD) δ 8.62 (d, J=4.0 Hz, 2H), 7.57 (d, J=8.0 Hz, 2H), 7.42 (s, 1H), 7.32 (d, J=8.0 Hz, 2H), 7.26 (t, J=4.0 Hz, 1H), 6.77 (dd, J=16.0 Hz, 12.0 Hz, 1H), 6.18 (d, J=16.0 Hz, 1H), 5.73 (d, J=12.0 Hz, 1H), 3.91 (s, 3H), 3.69-3.62 (m, 4H), 2.89-2.81 (m, 1H), 2.03-1.92 (m, 6H), 1.70-1.65 (m, 2H), 1.51-1.36 (m, 4H).

### Example 7

### Synthesis of N-(7-(4-amino-5-(4-methoxyphenyl)-7-methyl-7H-pyrrolo[2,3-d]pyrimidin-6-yl)spiro[3.5]non-6-en-2-yl)acrylamide

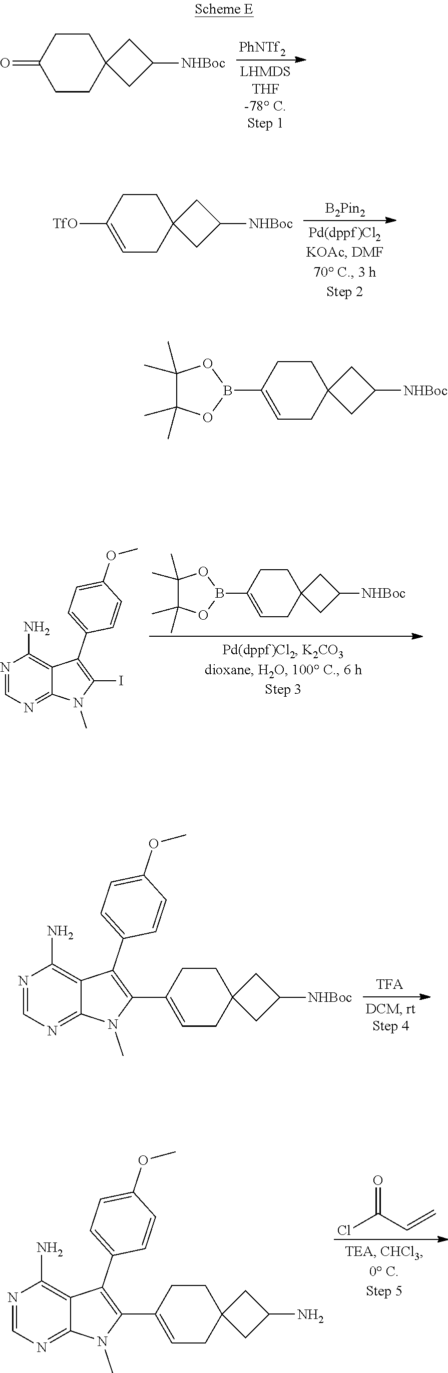

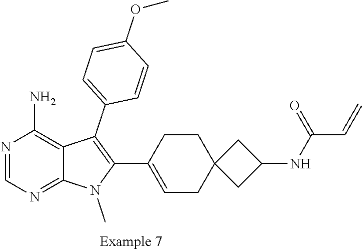 [SMILES: C=CC(=O)NC1CC2(CC=C(c3c(-c4ccc(OC)cc4)c4c(N)ncnc4n3C)CC2)C1]

### Scheme E, Step 1. Synthesis of 2-((tert-butoxycarbonyl)amino)spiro[3.5]non-6-en-7-yl trifluoromethanesulfonate

To a stirred solution of compound tert-butyl (7-oxospiro[3.5]nonan-2-yl)carbamate (253 mg, 1 mmol, 1 eq) in dry THF (5 mL) was added dropwise the solution of LiHMDS (2.2 mL, 2.2 mmol, 2.2 eq, 1M in THF) at −78° C. and the resulting mixture was stirred at −78° C. for 1 hr under N₂. Then PhNTf₂ (392 mg, 1.1 mmol, 1.1 eq) was added, and then mixture was warmed to room temperature slowly and stirred for 16 hrs. The reaction mixture was quenched with saturated NH₄Cl aqueous solution and extracted with EtOAc (3×10 mL). The combined organic layers were dried over anhydrous Na₂SO₄ and filtered. The filtrate was concentrated under reduced pressure. The residue was purified by silica gel column chromatography, eluting with hexanes/EtOAc (5:1) to afford the title compound (231 mg, 60% yield) as a colorless oil.

### Scheme E, Step 2. Synthesis of tert-butyl (7-(4,4,5,5-tetramethyl-1,3,2-dioxaborolan-2-yl)spiro[3.5]non-6-en-2-yl)carbamate

To a stirred solution of compound 2-((tert-butoxycarbonyl)amino)spiro[3.5]non-6-en-7-yl trifluoromethanesulfonate (231 mg, 0.6 mmol, 1 eq) in DMF (5 mL) was added B₂Pin₂ (167 mg, 0.66 mmol, 1.1 eq), KOAc (194 mg, 1.98 mmol, 3 eq) and Pd(dppf)Cl₂ (5.2 mg, 0.06 mmol, 0.1 eq). The resulting mixture was degassed three times with N₂ and then stirred at 70° C. for 3 hrs under N₂. The reaction mixture was concentrated in vacuo, diluted with EtOAc (10 mL), filtered through a filter pad, and concentrated in vacuo. The resulting residue was used in the next step.

### Scheme E, Step 3. Synthesis of tert-butyl (7-(4-amino-5-(4-methoxyphenyl)-7-methyl-7H-pyrrolo[2,3-d]pyrimidin-6-yl)spiro[3.5]non-6-en-2-yl)carbamate

A mixture of 6-iodo-5-(4-methoxyphenyl)-7-methyl-7H-pyrrolo[2,3-d]pyrimidin-4-amine (77 mg, 0.2 mmol, 1 eq), tert-butyl (7-(4,4,5,5-tetramethyl-1,3,2-dioxaborolan-2-yl)spiro[3.5]non-6-en-2-yl)carbamate (94 mg, 0.26 mmol, 1.3 eq), Pd(dppf)Cl₂ (16.5 mg, 0.02 mmol, 0.1 eq) and K₂CO₃ (82.8 mg, 0.6 mmol, 3 eq) in dioxane (10 mL) and H₂O (1 mL) was degassed three times with N₂ and then stirred at 100° C. for 6 h under N₂. The resulting mixture was diluted with H₂O (20 mL) and extracted with EtOAc (3×15 mL). The combined organic layers were dried over anhydrous Na₂SO₄ and filtered. The filtrate was concentrated under reduced pressure. The resulting residue was purified by silica gel column chromatography, eluting with DCM/MeOH (30:1) to afford the title compound (75 mg, 75% yield) as a yellow solid.

LCMS: m/z 490.30 [M+1]⁺.

### Scheme E, Step 4. Synthesis of 6-(2-aminospiro[3.5]non-6-en-7-yl)-5-(4-methoxyphenyl)-7-methyl-7H-pyrrolo[2,3-d]pyrimidin-4-amine

To a stirred solution of tert-butyl (7-(4-amino-5-(4-methoxyphenyl)-7-methyl-7H-pyrrolo[2,3-d]pyrimidin-6-yl)spiro[3.5]non-6-en-2-yl)carbamate (75 mg, 0.15 mmol, 1 eq) in DCM (2 mL) was added TFA (2 mL) under N₂, and the resulting mixture was stirred at room temperature for 2 hrs. The solution was concentrated. The crude residue was treated with NH₄OH aqueous solution and extracted with DCM (3×15 mL). The combined organic layers were washed with brine (15 mL), dried over anhydrous Na₂SO₄, and filtered. The filtrate was concentrated under reduced pressure. The residue was purified by silica gel column chromatography, eluting with DCM/MeOH (15:1) to afford the title compound (30 mg, 60% yield) as a yellow solid.

LCMS: m/z 390.33 [M+1]⁺.

### Scheme E, Step 5. Synthesis of N-(7-(4-amino-5-(4-methoxyphenyl)-7-methyl-7H-pyrrolo[2,3-d]pyrimidin-6-yl)spiro[3.5]non-6-en-2-yl)acrylamide

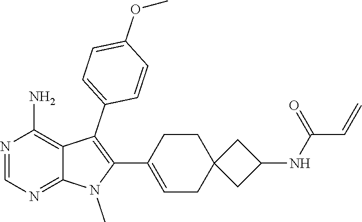 [SMILES: C=CC(=O)NC1CC2(CC=C(c3c(-c4ccc(OC)cc4)c4c(N)ncnc4n3C)CC2)C1]

### Example 7

To a stirred solution of 6-(2-aminospiro[3.5]non-6-en-7-yl)-5-(4-methoxyphenyl)-7-methyl-7H-pyrrolo[2,3-d]pyrimidin-4-amine (30 mg, 0.07 mmol, 1 eq) in dry CHCl₃ (5 mL) was added TEA (28 mg, 0.28 mmol, 4 eq). A solution of acryloyl chloride (6.9 mg, 0.07 mmol, 1 eq) in dry CHCl₃ (0.5 mL) was added dropwise at 0° C. and stirred at 0° C. for 0.5 hr under N₂. The reaction mixture was quenched with MeOH. The solvent was removed under reduced pressure. The resulting residue was purified by Prep-HPLC (0.1% TFA in ACN/H₂O) to afford the title compound (14.4 mg, 46% yield) as a white solid.

LCMS: m/z 444.35 [M+1]⁺.

¹H NMR (400 MHz, CD₃OD) δ 8.29 (s, 1H), 7.32 (d, J=8.0, 2H), 7.04 (d, J=8.0, 2H), 6.23-6.17 (m, 2H), 5.88-5.82 (m, 1H), 5.68-5.60 (m, 1H), 4.46-4.24 (m, 1H), 3.84 (s, 3H), 3.77 (d, J=4.0 Hz, 3H), 2.36-2.30 (m, 1H), 2.28-2.17 (m, 3H), 2.14-1.99 (m, 2H), 1.80-1.69 (m, 3H), 1.65 (t, J=6.0 Hz, 1H).

### Example 8

### Synthesis of 1-(9-(4-amino-5-(4-methoxyphenyl)-7-methyl-7H-pyrrolo[2,3-d]pyrimidin-6-yl)-3-azaspiro[5.5]undec-8-en-3-yl)prop-2-en-1-one

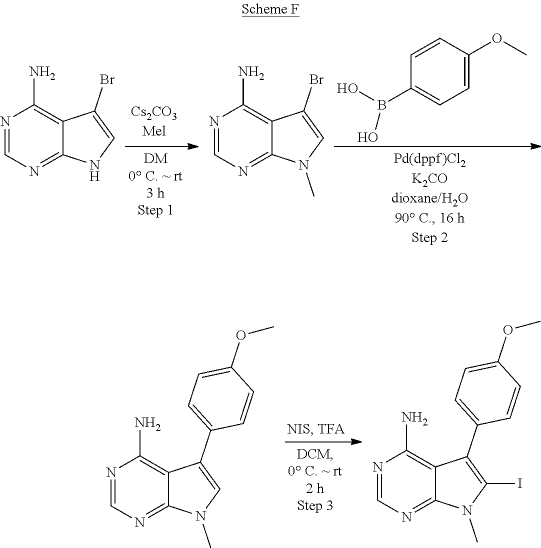

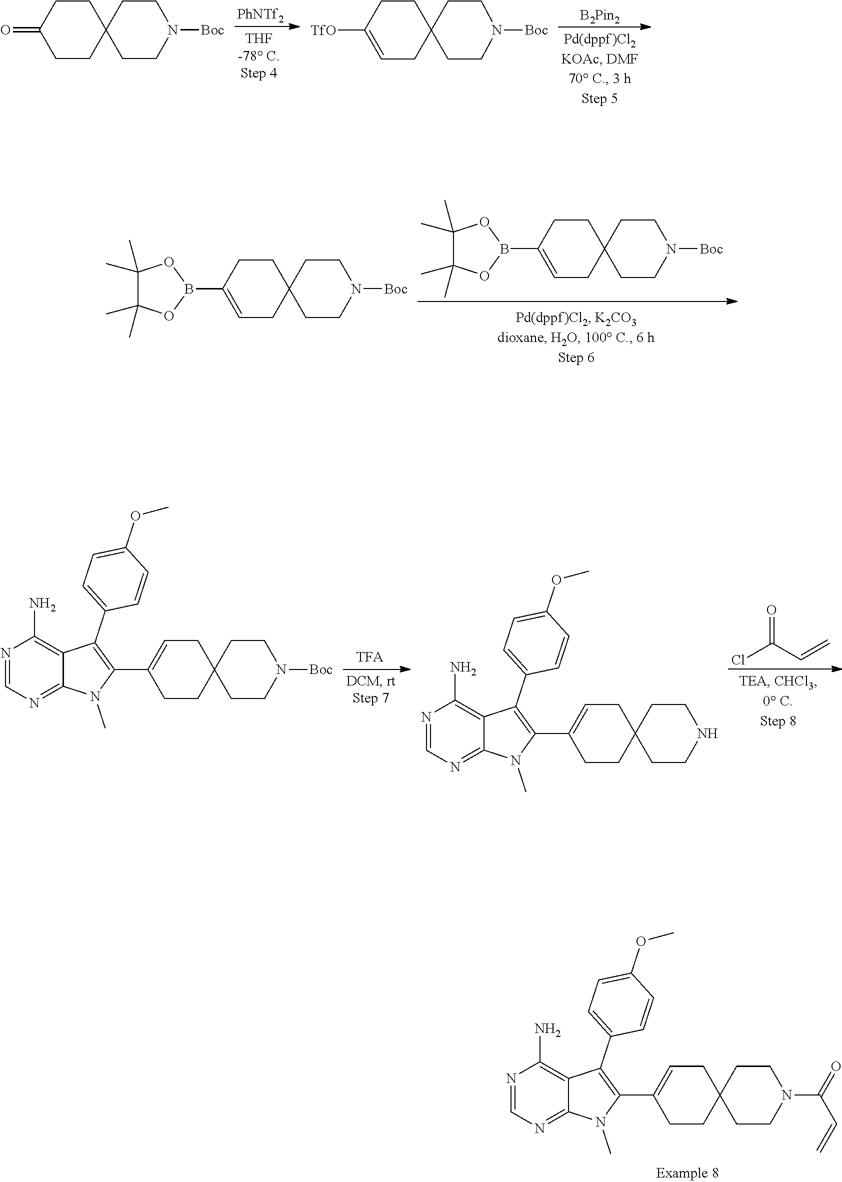

### Scheme F, Step 1. Synthesis of 5-bromo-7-methyl-7H-pyrrolo[2,3-d]pyrimidin-4-amine

To a stirred solution of compound 5-bromo-7H-pyrrolo[2,3-d]pyrimidin-4-amine (10 g, 46.9 mmol, 1 eq), Cs₂CO₃ (30.48 g, 93.8 mmol, 2 eq) in DMF (100 mL) was added Mel (7.98 g, 56.2 mmol, 1.2 eq) under N₂ at 0° C., and the resulting mixture was stirred at room temperature for 3 hrs. The solution was concentrated. The crude compound was partitioned between water (150 mL) and EtOAc (3×100 mL). The combined organic layers were washed with brine (3×100 mL), dried over anhydrous Na₂SO₄ and filtered. The filtrate was concentrated under reduced pressure and the resulting residue was purified by silica gel column chromatography, eluting with PE/EtOAc (1:1) to afford the title compound (4.2 g, 40%) as a yellow solid.

### Scheme F, Step 2. Synthesis of 5-(4-methoxyphenyl)-7-methyl-7H-pyrrolo[2,3-d]pyrimidin-4-amine

A mixture of 5-bromo-7-methyl-7H-pyrrolo[2,3-d]pyrimidin-4-amine (2.47 g, 10.8 mmol, 1 eq), compound 4-methoxyphenyl boronic acid (3.28 g, 21.6 mmol, 2 eq), K₃CO₃ (4.47 g, 32.4 mmol, 3 eq) and Pd(dppf)Cl₂ (830 mg, 1.08 mmol, 0.1 eq) in dioxane (25 mL) and H₂O (2.5 mL) was degassed three times with N₂ and then stirred at 90° C. for 16 h under N₂. The resulting mixture was diluted with H₂O (200 mL) and extracted with EtOAc (3×30 mL). The combined organic layers were dried over anhydrous Na₂SO₄ and filtered. The filtrate was concentrated under reduced pressure. The residue was purified by silica gel column chromatography, eluting with DCM/MeOH (14:1) to afford the title compound (1.7 g, 62%) as a white solid.

LCMS: m/z 255.10 [M+1]⁺.

### Scheme F, Step 3. Synthesis of 6-iodo-5-(4-methoxyphenyl)-7-methyl-7H-pyrrolo[2,3-d]pyrimidin-4-amine

To a solution of 5-(4-methoxyphenyl)-7-methyl-7H-pyrrolo[2,3-d]pyrimidin-4-amine (1.7 g, 6.69 mmol, 1 eq) in DCM (20 mL) was added NIS (1.8 g, 8 mmol, 1.2 eq) and CF₃COOH (1.52 g, 13.38 mmol, 2 eq) at 0° C. under N₂, and the resulting mixture was stirred at room temperature for 2 hrs. The resulting mixture was diluted under stirring with NaHCO₃ aqueous solution (10 mL), 10% Na₂SO₃ solution (10 mL) and H₂O (20 mL). The resulting precipitates were collected by filtration, washed with water and purified by silica gel column chromatography, eluting with DCM/MeOH (20:1) to afford the title compound (1.5 g, 59%) as a white solid.

LCMS: m/z 381.20 [M+1]⁺.

### Scheme F, Step 4. Synthesis of tert-butyl 9-(((trifluoromethyl)sulfonyl)oxy)-3-azaspiro[5.5]undec-8-ene-3-carboxylate

To a stirred solution of tert-butyl 9-oxo-3-azaspiro[5.5]undecane-3-carboxylate (400 mg, 1.5 mmol, 1 eq) in THF (5 mL) under N₂ was added dropwise the solution of LiHMDS (1.8 mL, 1.8 mmol, 1.2 eq, 1M in THF) at −78° C. and the resulting mixture was stirred at −78° C. for 1 hr under N₂. The solution of PhNTf₂ (586 mg, 1.65 mmol, 1.1 eq) in anhydrous THF (1 mL) was added dropwise at −78° C. and the mixture was stirred at room temperature for 16 hrs under N₂. The solution was concentrated. The crude residue was purified by silica gel column chromatography, eluting with PE/EtOAc (5:1) to afford the title compound (0.6 g, 100%) as a tan oil.

### Scheme F, Step 5. Synthesis of tert-butyl 9-(4,4,5,5-tetramethyl-1,3,2-dioxaborolan-2-yl)-3-azaspiro[5.5]undec-8-ene-3-carboxylate

A mixture of tert-butyl 9-(((trifluoromethyl)sulfonyl)oxy)-3-azaspiro[5.5]undec-8-ene-3-carboxylate (600 mg, 1.5 mmol, 1 eq), B₂Pin₂ (419 mg, 1.65 mmol, 1.1 eq), Pd(dppf)Cl₂ (123 mg, 0.15 mmol, 1 eq) and KOAc (441 mg, 4.5 mmol, 0.1 eq) in DMF (10 mL) was degassed with N₂ three times, and then stirred under N₂ at 80° C. for 3 hrs. The solution was concentrated. The crude compound was filtered over a filter pad. The filtrate was concentrated under reduced pressure to afford the title compound (566 mg, 99%) as an oil which was used in the next step directly.

### Scheme F, Step 6. Synthesis of tert-butyl 9-(4-amino-5-(4-methoxyphenyl)-7-methyl-7H-pyrrolo[2,3-d]pyrimidin-6-yl)-3-azaspiro[5.5]undec-8-ene-3-carboxylate

A mixture of 6-iodo-5-(4-methoxyphenyl)-7-methyl-7H-pyrrolo[2,3-d]pyrimidin-4-amine (ACE-2057-3), (380 mg, 1 mmol, 1 eq), tert-butyl 9-(4,4,5,5-tetramethyl-1,3,2-dioxa-borolan-2-yl)-3-azaspiro[5.5]undec-8-ene-3-carboxylate (566 mg, 1.5 mmol, 1.5 eq), K₂CO₃ (414 mg, 3 mmol, 3 eq) and Pd(dppf)Cl₂ (82.3 mg, 0.1 mmol, 0.1 eq) in dioxane (10 mL) and H₂O (1 mL) was degassed three times with N₂ and then stirred at 100° C. for 6 h under N₂. The resulting mixture was diluted with H₂O (20 mL) and extracted with EtOAc (3×10 mL). The combined organic layers were dried over anhydrous Na₂SO₄ and filtered. The residue was purified by silica gel column chromatography, eluting with DCM/MeOH (20:1) to afford the title compound (330 mg, 75%) as a white solid.

LCMS: m/z 504.3 [M+1]⁺.

### Scheme F, Step 7. Synthesis of 5-(4-methoxyphenyl)-7-methyl-6-(3-azaspiro[5.5]undec-8-en-9-yl)-7H-pyrrolo[2,3-d]pyrimidin-4-amine

A mixture of tert-butyl 9-(4-amino-5-(4-methoxyphenyl)-7-methyl-7H-pyrrolo[2,3-d]pyrimidin-6-yl)-3-azaspiro[5.5]undec-8-ene-3-carboxylate (130 mg, 0.258 mmol, 1 eq) in TFA (1 mL) and in DCM (2 mL) was stirred at room temperature for 2 hrs. The resulting mixture was filtered. The filtrate was concentrated under reduced pressure to afford the title compound (104 mg, 99%) as a yellow oil, which was used for the following step directly without further purification.

### Scheme F, Step 8. Synthesis of 1-(9-(4-amino-5-(4-methoxyphenyl)-7-methyl-7H-pyrrolo[2,3-d]pyrimidin-6-yl)-3-azaspiro[5.5]undec-8-en-3-yl)prop-2-en-1-one

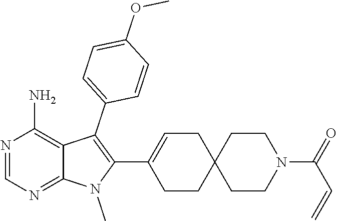 [SMILES: C=CC(=O)N1CCC2(CC=C(c3c(-c4ccc(OC)cc4)c4c(N)ncnc4n3C)CC2)CC1]

### Example 8

To a mixture of compound 5-(4-methoxyphenyl)-7-methyl-6-(3-azaspiro[5.5]undec-8-en-9-yl)-7H-pyrrolo[2,3-d]pyrimidin-4-amine (104 mg, 0.25 mmol, 1 eq) in dry CHCl₃ (3 mL) was added TEA (104 mg, 1.032 mmol, 4 eq). A solution of acryloyl chloride (20 mg, 0.258 mmol, 1 eq) in dry CHCl₃ (0.5 mL) was added dropwise at 0° C., and the mixture as stirred at 0° C. for 0.5 hr under N₂. The reaction mixture was quenched with MeOH. The solvent was removed under reduced pressure. The residue was purified by Prep-HPLC (0.1% TFA in ACN/H₂O) to afford the title compound (23.4 mg, 20% yield) as a white solid.

LCMS: m/z 458.2 [M+1]⁺.

¹H NMR (400 MHz, CD₃OD) δ 8.30 (s, 1H), 7.33 (d, J=8.0 Hz, 2H), 7.05 (d, J=8.0 Hz, 2H), 6.74 (dd, J=16.0, 12.0 Hz, 1H), 6.16 (d, J=16.0 Hz, 1H), 5.92 (brs, 1H), 5.72 (d, J=12.0 Hz, 1H), 3.85 (s, 3H), 3.80 (s, 3H), 3.70-3.62 (m, 2H), 3.60-3.50 (m, 2H), 2.16-2.13 (m, 2H), 2.05-2.01 (m, 2H), 1.61-1.57 (m, 2H), 1.45-1.39 (m, 4H).

### Example 9

### Synthesis of N-(7-(4-amino-7-methyl-5-(4-(pyrimidin-2-yloxy)phenyl)-7H-pyrrolo[2,3-d]pyrimidin-6-yl)spiro[3.5]non-6-en-2-yl)acrylamide

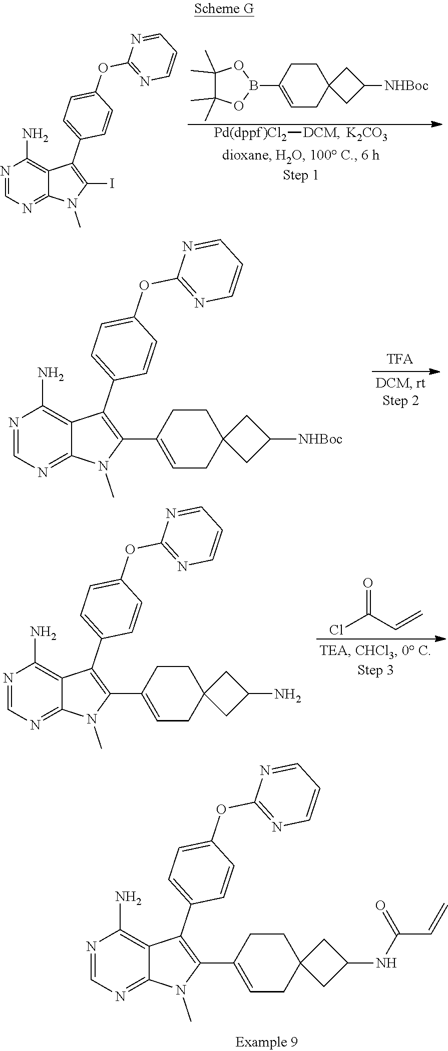

### Scheme G, Step 1. Synthesis of tert-butyl (7-(4-amino-7-methyl-5-(4-(pyrimidin-2-yloxy)phenyl)-7H-pyrrolo[2,3-d]pyrimidin-6-yl)spiro[3.5]non-6-en-2-yl)carbamate

A mixture of 6-iodo-7-methyl-5-(4-(pyrimidin-2-yloxy)phenyl)-7H-pyrrolo[2,3-d]pyrimidin-4-amine (209 mg, 0.47 mmol, 1 eq), tert-butyl (7-(4,4,5,5-tetramethyl-1,3,2-dioxaborolan-2-yl)spiro[3.5]non-6-en-2-yl)carbamate (341 mg, 0.94 mmol, 2 eq), Pd(dppf)Cl₂ (38 mg, 0.047 mmol, 0.1 eq) and K₂CO₃ (194 mg, 1.41 mmol, 3 eq) in dioxane (10 mL) and H₂O (1 mL) was degassed three times with N₂ and then stirred at 100° C. for 6 h under N₂. The resulting mixture was diluted with H₂O (10 mL) and extracted with EtOAc (3×5 mL). The combined organic layers were dried over anhydrous Na₂SO₄ and filtered. The filtrate was concentrated under reduced pressure. The residue was purified by silica gel column chromatography, eluting with DCM/MeOH (30:1) to afford the title compound (182 mg, 70% yield) as a yellow solid.

LCMS: m/z 445.56 [M+1]⁺.

### Scheme G, Step 2. Synthesis of 6-(2-aminospiro[3.5]non-6-en-7-yl)-7-methyl-5-(4-(pyrimidin-2-yloxy)phenyl)-7H-pyrrolo[2,3-d]pyrimidin-4-amine

To a stirred solution of tert-butyl (7-(4-amino-7-methyl-5-(4-(pyrimidin-2-yloxy)phenyl)-7H-pyrrolo[2,3-d]pyrimidin-6-yl)spiro[3.5]non-6-en-2-yl)carbamate (182 mg, 0.33 mmol, 1 eq) in DCM (5 mL) was added TFA (5 mL) under N₂, and the resulting mixture was stirred at room temperature for 2 hrs. The solution was concentrated. The crude residue was treated with NH₄OH aqueous solution and extracted with DCM (3×15 mL). The combined organic layer was washed by brine (15 mL), dried over anhydrous Na₂SO₄ and filtered. The filtrate was concentrated under reduced pressure. The residue was purified by silica gel column chromatography, eluting with DCM/MeOH (15:1) to afford the title compound (90 mg, 60% yield) as a yellow solid.

LCMS: m/z 454.33 [M+1]⁺.

### Scheme G, Step 3. Synthesis of N-(7-(4-amino-7-methyl-5-(4-(pyrimidin-2-yloxy)phenyl)-7H-pyrrolo[2,3-d]pyrimidin-6-yl)spiro[3.5]non-6-en-2-yl)acrylamide

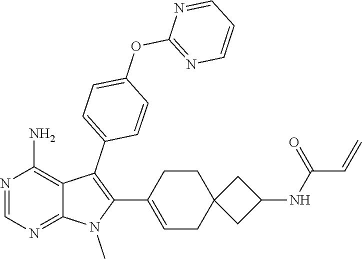 [SMILES: C=CC(=O)NC1CC2(CC=C(c3c(-c4ccc(Oc5ncccn5)cc4)c4c(N)ncnc4n3C)CC2)C1]

### Example 9

To a stirred solution of 6-(2-aminospiro[3.5]non-6-en-7-yl)-7-methyl-5-(4-(pyrimidin-2-yloxy)phenyl)-7H-pyrrolo[2,3-d]pyrimidin-4-amine (90 mg, 0.198 mmol, 1 eq) in dry CHCl₃ (4 mL) was added TEA (76 mg, 0.76 mmol, 4 eq). A solution of acryloyl chloride (17 mg, 0.198 mmol, 1 eq) in dry CHCl₃ (0.5 mL) was added dropwise at 0° C. The resulting mixture was stirred at 0° C. for 0.5 hr under N₂, and then quenched with MeOH. The solvent was removed under reduced pressure. The residue was purified by Prep-HPLC (0.1% HCOOH in ACN/H₂O) to afford the title compound (33.3 mg, 31% yield) as a white solid.

LCMS: m/z 508.40 [M+1]⁺.

¹H NMR (400 MHz, CD₃OD) δ 8.59 (d, J=5.0 Hz, 2H), 8.11 (s, 1H), 7.43 (d, J=8.0 Hz, 2H), 7.24-7.20 (m, 3H), 6.18 (d, J=8.0 Hz, 2H), 5.86-5.76 (m, 1H), 5.62 (t, J=4.0 Hz, 1H), 4.45-4.26 (m, 1H), 3.66 (s, 3H), 2.33-2.04 (m, 6H), 1.78-1.62 (m, 4H).

### Example 10

### Synthesis of 1-(9-(4-amino-5-(3-fluorophenyl)-7-methyl-7H-pyrrolo[2,3-d]pyrimidin-6-yl)-3-azaspiro[5.5]undec-8-en-3-yl)prop-2-en-1-one

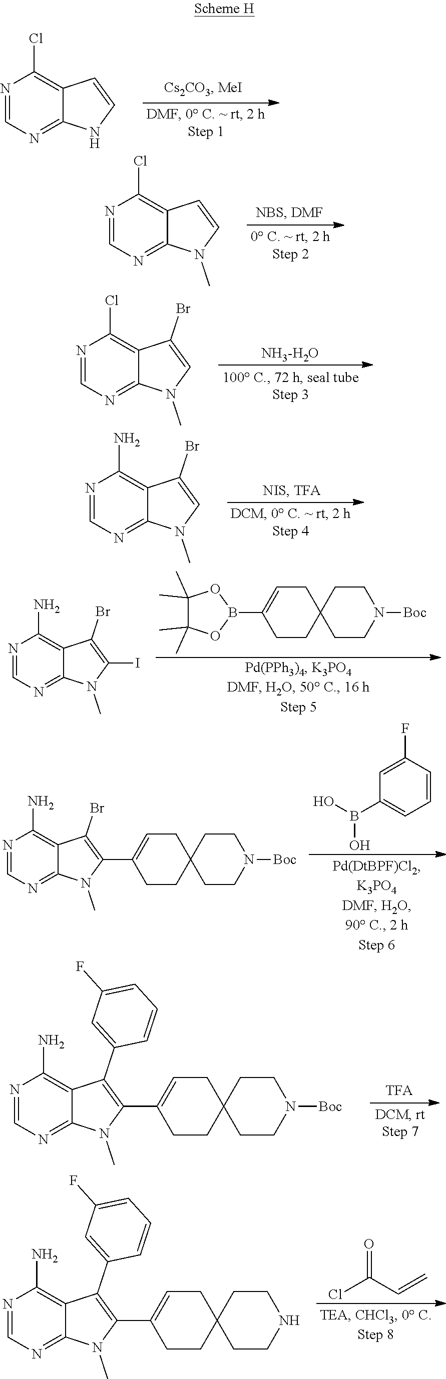

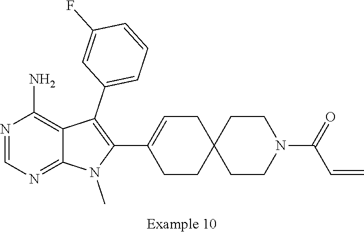 [SMILES: C=CC(=O)N1CCC2(CC=C(c3c(-c4cccc(F)c4)c4c(N)ncnc4n3C)CC2)CC1]

### Scheme H, Step 1. Synthesis of 4-Chloro-7-Methyl-7H-pyrrolo[2,3-d]pyrimidine

To a solution of 4-chloro-7H-pyrrolo[2,3-d]pyrimidine (50 g, 326 mmol, 1 eq) in DMF (400 mL) was added Cs₂CO₃ (160 g, 489 mmol, 1.5 eq) at 0° C. under N₂, followed by Mel (92 g, 652 mmol, 2 eq), and the resulting mixture was stirred at room temperature for 2 hrs. The mixture was poured into water (3000 mL). The precipitated solid was collected by filtration, washed with water, and dried under reduced pressure to afford the title compound (41 g, 75%) as a white solid.

LCMS: m/z 168.33 [M+1]⁺.

### Scheme H, Step 2. Synthesis of 5-bromo-4-chloro-7-methyl-7H-pyrrolo[2,3-d]pyrimidine

To a solution of compound 4-chloro-7-methyl-7H-pyrrolo[2,3-d]pyrimidine (41 g, 245 mmol, 1 eq) in DMF (350 mL) was added NBS (52.3 g, 294 mmol, 1.2 eq) in one portion under N₂ at 0° C., and the resulting mixture was stirred at room temperature for 2 hrs. The mixture was poured into water. The precipitated solid was collected by filtration, washed with water, and dried under reduced pressure to afford the title compound (53 g, 88%) as a white solid.

LCMS: m/z 247.80 [M+1]⁺.

### Scheme H, Step 3. Synthesis of 5-bromo-7-methyl-7H-pyrrolo[2,3-d]pyrimidin-4-amine

A mixture of 5-bromo-4-chloro-7-methyl-7H-pyrrolo[2,3-d]pyrimidine (15 g, 60.9 mmol, 1 eq) in NH₃—H₂O (150 mL) was stirred at 100° C. for 72 hrs in a sealed tube. The mixture was cooled to room temperature and diluted with water. The solid was filtered, washed with water and dried under reduced pressure to afford the title compound (12 g, 87%) as a white solid.

LCMS: m/z 227.03 [M+1]⁺.

### Scheme H, Step 4. Synthesis of 5-bromo-6-iodo-7-methyl-7H-pyrrolo[2,3-d]pyrimidin-4-amine

To a solution of 5-bromo-7-methyl-7H-pyrrolo[2,3-d]pyrimidin-4-amine (6 g, 26.4 mmol, 1 eq) in DCM (60 mL) was added TFA (15 g, 132 mmol, 5 eq) and NIS (5.6 g, 31.7 mmol, 1.2 eq) in one portion under N₂ at 0° C. The mixture was stirred at room temperature for 2 hrs. The mixture was poured into saturated aqueous Na₂SO₃ solution. The precipitated solid was collected by filtration, washed with water, and dried under reduced pressure to afford the title compound (8.2 g, 88%) as a gray solid.

LCMS: m/z 352.96 [M+1]⁺.

### Scheme H, Step 5. Synthesis of tert-butyl 9-(4-amino-5-bromo-7-methyl-7H-pyrrolo[2,3-d]pyrimidin-6-yl)-3-azaspiro[5.5]undec-8-ene-3-carboxylate

A mixture of 5-bromo-6-iodo-7-methyl-7H-pyrrolo[2,3-d]pyrimidin-4-amine (3 g, 8.5 mmol, 1 eq), tert-butyl 9-(4,4,5,5-tetramethyl-1,3,2-dioxaborolan-2-yl)-3-azaspiro[5.5]-undec-8-ene-3-carboxylate (6.4 g, 17 mmol, 2 eq), Pd(PPh₃)₄ (981 mg, 0.85 mmol, 0.1 eq) and K₃PO₄ (3.52 g, 25.5 mmol, 3 eq) in DMF (24 mL) and H₂O (3 mL) was degassed with N₂ three times and then stirred at 50° C. for 16 h under N₂. The resulting mixture was diluted with H₂O (50 mL) and extracted with EtOAc (3×40 mL). The combined organic layers were washed with brine (4×30 mL), dried over anhydrous Na₂SO₄, and filtered. The filtrate was concentrated under reduced pressure. The residue was purified by silica gel column chromatography, eluting with DCM/MeOH (70:1) to afford the title compound (892 mg, 22% yield) as a yellow solid.

LCMS: m/z 476.20 [M+1]⁺.

### Scheme H, Step 6. Synthesis of tert-butyl 9-(4-amino-5-(3-fluorophenyl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (100 mg, 0.442 mmol, 1 eq), 3-fluoro-phenylbronic acid (92 mg, 17 mmol, 1.5 eq), Pd(DtBPF)Cl₂ (28 mg, 0.044 mmol, 0.1 eq) and K₃PO₄ (182 mg, 1.3 mmol, 3 eq) in DMF (10 mL) and H₂O (1 mL) was degassed with N₂ three times and then stirred at 90° C. for 2 h under N₂. The resulting mixture was diluted with H₂O (20 mL) and extracted with EtOAc (3×15 mL). The combined organic layers were washed with brine (4×15 mL), dried over anhydrous Na₂SO₄, and filtered. The filtrate was concentrated under reduced pressure. The residue was purified by silica gel column chromatography, eluting with DCM/MeOH (20:1) to afford the title compound (40 mg, 18% yield) as a yellow solid.

LCMS: m/z 491.25 [M+1]⁺.

### Scheme H, Step 7. Synthesis of 5-(3-fluorophenyl)-7-methyl-6-(3-azaspiro[5.5]undec-8-en-9-yl)-7H-pyrrolo[2,3-d]pyrimidin-4-amine

To a stirred solution of tert-butyl 9-(4-amino-5-(3-fluorophenyl)-7-methyl-7H-pyrrolo[2,3-d]pyrimidin-6-yl)-3-azaspiro[5.5]undec-8-ene-3-carboxylate (40 mg, 0.08 mmol, 1 eq) in DCM (2 mL) was added TFA (0.5 mL) and the mixture was stirred at room temperature for 2 hrs. The solution was concentrated to afford the title compound (31 mg, 99% yield) as a yellow solid.

LCMS: m/z 492.30 [M+1]⁺.

### Scheme H, Step 8. Synthesis of 1-(9-(4-amino-5-(3-fluorophenyl)-7-methyl-7H-pyrrolo[2,3-d]pyrimidin-6-yl)-3-azaspiro[5.5]undec-8-en-3-yl)prop-2-en-1-one

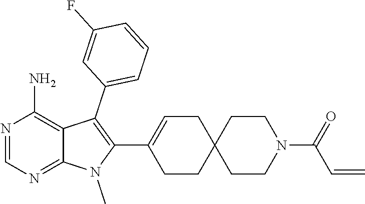 [SMILES: C=CC(=O)N1CCC2(CC=C(c3c(-c4cccc(F)c4)c4c(N)ncnc4n3C)CC2)CC1]

### Example 10

To a stirred solution of 5-(3-fluorophenyl)-7-methyl-6-(3-azaspiro[5.5]undec-8-en-9-yl)-7H-pyrrolo[2,3-d]pyrimidin-4-amine (31 mg, 0.081 mmol, 1 eq) in dry CHCl₃ (4 mL) was added TEA (32 mg, 0.32 mmol, 4 eq). A solution of acryloyl chloride (7.2 mg, 0.18 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (20.3 mg, 57% yield) as a white solid.

LCMS: m/z 446.30 [M+1]⁺.

¹HNMR (400 MHz, CD₃OD) δ 8.34 (s, 1H), 7.54-7.48 (m, 1H), 7.25-7.14 (m, 3H), 6.76 (dd, J=16.0, 12.0 Hz, 1H), 6.16 (d, J=16.0 Hz, 1H), 5.95 (brs, 1H), 5.72 (d, J=12.0 Hz, 1H), 3.81 (s, 3H), 3.75-3.61 (m, 2H), 3.58-3.46 (m, 2H), 2.17-2.14 (m, 2H), 2.06-2.01 (m, 2H), 1.60 (t, J=5.9 Hz, 2H), 1.48-1.38 (m, 4H).

### Example 11

### Synthesis of 1-(9-(4-amino-5-(4-fluorophenyl)-7-methyl-7H-pyrrolo[2,3-d]pyrimidin-6-yl)-3-azaspiro[5.5]undec-8-en-3-yl)prop-2-en-1-one

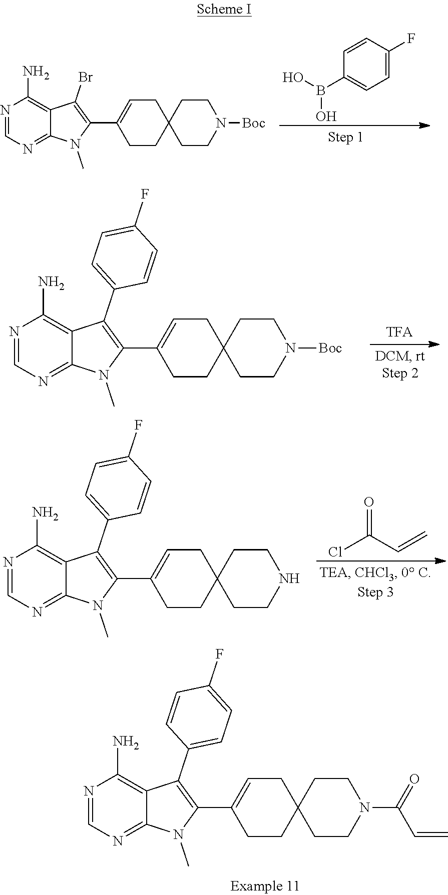

### Scheme I, Step 1. Synthesis of tert-butyl 9-(4-amino-5-(4-fluorophenyl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (100 mg, 0.21 mmol, 1 eq), 4-fluoro-phenyl-bronic acid (44 mg, 0.31 mmol, 1.5 eq), Pd(DtBPF)Cl₂ (14 mg, 0.02 mmol, 0.1 eq) and K₃PO₄ (87 mg, 0.63 mmol, 3 eq) in DMF (10 mL) and H₂O (1 mL) was degassed with N₂ three times, and then stirred at 90° C. for 2 hrs. The resulting mixture was diluted with H₂O (20 mL) and extracted with EtOAc (3×15 mL). The combined organic layers were washed with brine (4×15 mL), dried over anhydrous Na₂SO₄, filtered and concentrated under reduced pressure. The residue was purified by silica gel column chromatography, eluting with DCM/MeOH (20:1) to afford the title compound (91 mg, 88% yield) as a yellow solid.

LCMS: m/z 492.60 [M+1]⁺.

### Scheme I, Step 2. Synthesis of 5-(4-fluorophenyl)-7-methyl-6-(3-azaspiro[5.5]undec-8-en-9-yl)-7H-pyrrolo[2,3-d]pyrimidin-4-amine

To a stirred solution of tert-butyl 9-(4-amino-5-(4-fluorophenyl)-7-methyl-7H-pyrrolo[2,3-d]pyrimidin-6-yl)-3-azaspiro[5.5]undec-8-ene-3-carboxylate (90 mg, 0.18 mmol, 1 eq) in DCM (4 mL) was added TFA (2 mL) and the mixture was stirred at room temperature for 2 hrs. The solution was concentrated to afford the title compound (56 mg, 78% yield) as a yellow solid.

LCMS: m/z 392.60 [M+1]⁺.

### Scheme I, Step 3. Synthesis of 1-(9-(4-amino-5-(4-fluorophenyl)-7-methyl-7H-pyrrolo[2,3-d]pyrimidin-6-yl)-3-azaspiro[5.5]undec-8-en-3-yl)prop-2-en-1-one

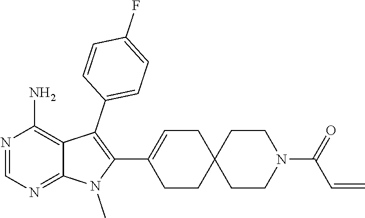 [SMILES: C=CC(=O)N1CCC2(CC=C(c3c(-c4ccc(F)cc4)c4c(N)ncnc4n3C)CC2)CC1]

### Example 11

To a stirred solution of 5-(4-fluorophenyl)-7-methyl-6-(3-azaspiro[5.5]undec-8-en-9-yl)-7H-pyrrolo[2,3-d]pyrimidin-4-amine (56 mg, 0.14 mmol, 1 eq) in dry CHCl₃ (4 mL) was added TEA (72 mg, 0.71 mmol, 5 eq). A solution of acryloyl chloride (13 mg, 0.14 mmol, 1 eq) in dry CHCl₃ (0.5 mL) was added dropwise at 0° C. and the resulting mixture was stirred at 0° C. for 0.5 hr under N₂. The reaction mixture was quenched with MeOH. The solvent was removed under reduced pressure. The residue was purified by Prep-HPLC (0.1% TFA in ACN/H₂O) to afford the title compound (21.2 mg, 33% yield) as a white solid.

LCMS: m/z 446.30 [M+1]⁺.

¹H NMR (400 MHz, CD₃OD) δ 8.30 (s, 1H), 7.44-7.40 (m, 2H), 7.24-7.20 (m, 2H), 6.75 (dd, J=16.0, 10.6 Hz, 1H), 6.15 (d, J=16.0 Hz, 1H), 5.91 (brs, 1H), 5.70 (d, J=10.6 Hz, 1H), 3.79 (s, 3H), 3.75-3.59 (m, 2H), 3.57-3.45 (m, 2H), 2.15-2.11 (m, 2H), 2.04-1.98 (m, 2H), 1.61-1.55 (m, 2H), 1.46-1.34 (m, 4H).

### Example 12

### Synthesis of 1-(9-(4-amino-7-methyl-5-(pyridin-3-yl)-7H-pyrrolo[2,3-d]pyrimidin-6-yl)-3-azaspiro[5.5]undec-8-en-3-yl)prop-2-en-1-one

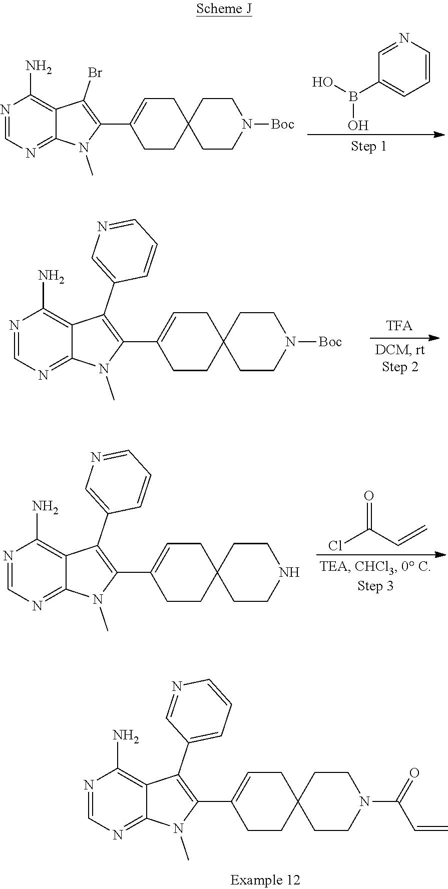

### Scheme J, Step 1. Synthesis of tert-butyl 9-(4-amino-7-methyl-5-(pyridin-3-yl)-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (200 mg, 0.42 mmol, 1 eq), pyridin-3-yl boronic acid (103 mg, 0.84 mmol, 2 eq), Pd(DtBPF)Cl (26 mg, 0.04 mmol, 0.1 eq) and K₃PO₄ (173 mg, 1.26 mmol, 3 eq) in DMF (10 mL) and H₂O (1 mL) was degassed with N₂ three times, and then stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄, filtered and concentrated under reduced pressure. The residue was purified by silica gel column chromatography, eluting with DCM/MeOH (15:1) to afford the title compound (160 mg, 80% yield) as a yellow solid.

LCMS: m/z 475.30 [M+1]⁺.

### Scheme J, Step 2. Synthesis of 7-methyl-5-(pyridin-3-yl)-6-(3-azaspiro[5.5]undec-8-en-9-yl)-7H-pyrrolo[2,3-d]pyrimidin-4-amine

To a stirred solution of tert-butyl 9-(4-amino-7-methyl-5-(pyridin-3-yl)-7H-pyrrolo-[2,3-d]pyrimidin-6-yl)-3-azaspiro[5.5]undec-8-ene-3-carboxylate (160 mg, 0.33 mmol, 1 eq) in DCM (4 mL) was added TFA (2 mL) and the mixture was stirred at room temperature for 2 hrs. The solution was concentrated to afford the title compound (100 mg, 80% yield) as a yellow solid.

LCMS: m/z 375.30 [M+1]⁺.

### Scheme J, Step 3. Synthesis of 1-(9-(4-amino-7-methyl-5-(pyridin-3-yl)-7H-pyrrolo[2,3-d]pyrimidin-6-yl)-3-azaspiro[5.5]undec-8-en-3-yl)prop-2-en-1-one

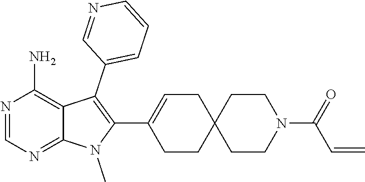 [SMILES: C=CC(=O)N1CCC2(CC=C(c3c(-c4cccnc4)c4c(N)ncnc4n3C)CC2)CC1]

### Example 12

To a stirred solution of 7-methyl-5-(pyridin-3-yl)-6-(3-azaspiro[5.5]undec-8-en-9-yl)-7H-pyrrolo[2,3-d]pyrimidin-4-amine (100 mg, 0.266 mmol, 1 eq) in dry CHCl₃ (4 mL) was added TEA (80 mg, 0.79 mmol, 3 eq). A solution of acryloyl chloride (24 mg, 0.246 mmol, 1 eq) in dry CHCl₃ (0.5 mL) was added dropwise at 0° C. and the resulting mixture was stirred at 0° C. for 0.5 hrs under N₂. The reaction was quenched with MeOH. The solvent was removed under reduced pressure. The residue was purified by Prep-HPLC (0.1% TFA in ACN/H₂O) to afford the title compound (22 mg, 19% yield) as a white solid.

LCMS: m/z 429.35 [M+1]⁺.

¹H NMR (400 MHz, CD₃OD) δ 9.00 (s, 1H), 8.92 (d, J=4.0 Hz, 1H), 8.67 (d, J=8.0 Hz, 1H), 8.42 (s, 1H), 8.21 (t, J=6.0 Hz, 1H), 6.77 (dd, J=18.0, 10.0 Hz, 1H), 6.17 (d, J=18.0 Hz, 1H), 6.00 (brs, 1H), 5.73 (d, J=10.0 Hz, 1H), 3.84 (s, 3H), 3.74-3.64 (m, 2H), 3.59-3.51 (m, 2H), 2.16 (brs, 4H), 1.66 (brs, 2H), 1.46-1.39 (m, 4H).

### Example 13

### Synthesis of 1-(9-(4-amino-7-methyl-5-(1-methyl-1H-pyrazol-5-yl)-7H-pyrrolo[2,3-d]pyrimidin-6-yl)-3-azaspiro[5.5]undec-8-en-3-yl)prop-2-en-1-one

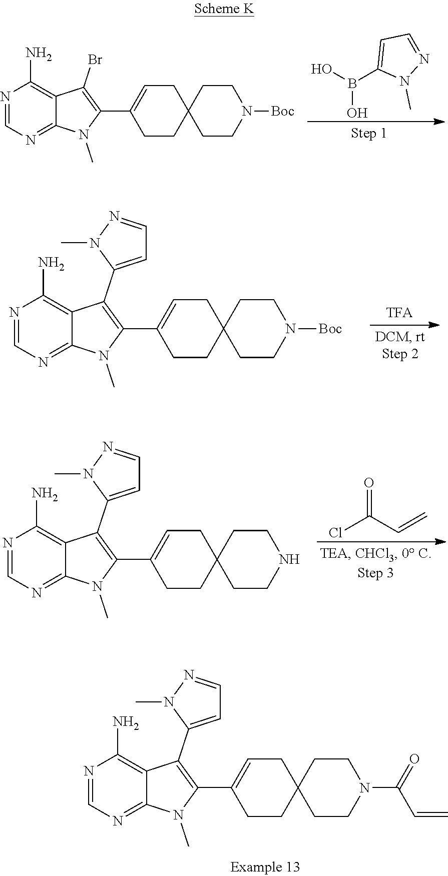

### Scheme K, Step 1. Synthesis of tert-butyl 9-(4-amino-7-methyl-5-(1-methyl-1H-pyrazol-5-yl)-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (200 mg, 0.42 mmol, 1 eq), (1-methyl-1H-pyrazol-5-yl)boronic acid (159 mg, 1.26 mmol, 3 eq), Pd(DtBPF)Cl₂ (27 mg, 0.04 mmol, 0.1 eq) and K₃PO₄ (267 mg, 0.1.26 mmol, 3 eq) in DMF (10 mL) and H₂O (1 mL) was degassed with N₂ three times, and then stirred at 90° C. for 2 h under N₂. The resulting mixture was diluted with H₂O (20 mL) and extracted with EtOAc (3×15 mL). The combined organic layers were washed with brine (4×15 mL), dried over anhydrous Na₂SO₄, filtered and concentrated under reduced pressure. The residue was purified by silica gel column chromatography, eluting with DCM/MeOH (20:1) to afford the title compound (70 mg, 35% yield) as a yellow solid.

LCMS: m/z 478.30 [M+1]⁺.

### Scheme K, Step 2. Synthesis of 7-methyl-5-(1-methyl-1H-pyrazol-5-yl)-6-(3-azaspiro[5.5]undec-8-en-9-yl)-7H-pyrrolo[2,3-d]pyrimidin-4-amine

To a stirred solution of tert-butyl 9-(4-amino-7-methyl-5-(1-methyl-1H-pyrazol-5-yl)-7H-pyrrolo[2,3-d]pyrimidin-6-yl)-3-azaspiro[5.5]undec-8-ene-3-carboxylate (70 mg, 0.15 mmol, 1 eq) in DCM (4 mL) was added TFA (2 mL), and the mixture was stirred at room temperature for 2 hrs. The solution was concentrated to afford the title compound (55 mg, 99% yield) as a yellow solid.

LCMS: m/z 377.0 [M+1]⁺.

### Scheme K, Step 3. Synthesis of 1-(9-(4-amino-7-methyl-5-(1-methyl-1H-pyrazol-5-yl)-7H-pyrrolo[2,3-d]pyrimidin-6-yl)-3-azaspiro[5.5]undec-8-en-3-yl)prop-2-en-1-one

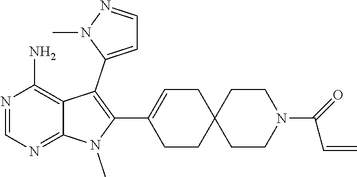 [SMILES: C=CC(=O)N1CCC2(CC=C(c3c(-c4ccnn4C)c4c(N)ncnc4n3C)CC2)CC1]

### Example 13

To a stirred solution of 7-methyl-5-(1-methyl-1H-pyrazol-5-yl)-6-(3-azaspiro[5.5]-undec-8-en-9-yl)-7H-pyrrolo[2,3-d]pyrimidin-4-amine (55 mg, 0.14 mmol, 1 eq) in dry CHCl₃ (4 mL) was added TEA (59 mg, 0.58 mmol, 4 eq). A solution of acryloyl chloride (13 mg, 0.146 mmol, 1 eq) in dry CHCl₃ (0.5 mL) was added dropwise at 0° C. and resulting mixture was stirred at 0° C. for 0.5 hrs under N₂. The reaction was quenched with MeOH. The solvent was removed under reduced pressure. The residue was purified by Prep-HPLC (0.1% TFA in ACN/H₂O) to afford the title compound (36.5 mg, 57% yield) as a white solid.

LCMS: m/z 432.35[M+1]⁺.

¹H NMR (400 MHz, CD₃OD) δ 8.40 (s, 1H), 8.16 (s, 1H), 6.81-6.74 (m, 2H), 6.17 (d, J=16.0, 1H), 5.99 (brs, 1H), 5.75 (d, J=12.0 Hz, 1H), 3.88 (s, 3H), 3.85 (s, 3H), 3.61-3.58 (m, 2H), 3.55-3.50 (m, 2H), 2.19-2.16 (m, 2H), 2.13-2.05 (m, 2H), 1.71-1.61 (m, 2H), 1.51-1.36 (m, 4H).

### Example 14

### Synthesis of 1-(9-(4-amino-7-methyl-5-(1-methyl-1H-pyrazol-4-yl)-7H-pyrrolo[2,3-d]pyrimidin-6-yl)-3-azaspiro[5.5]undec-8-en-3-yl)prop-2-en-1-one

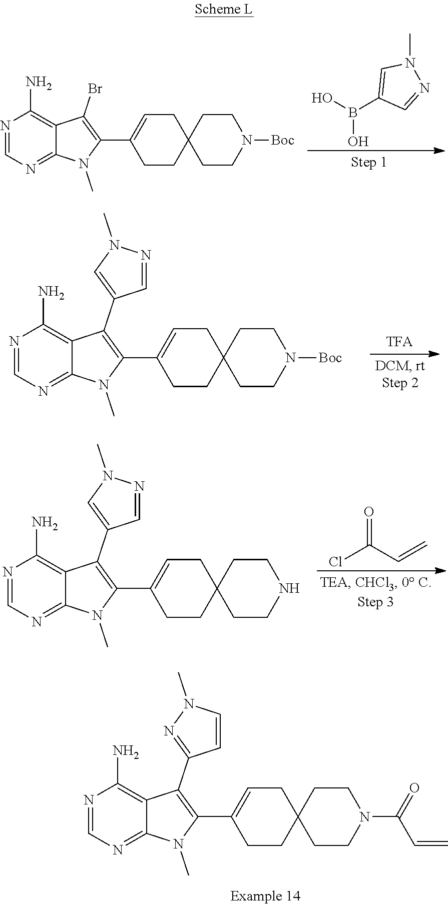

### Scheme L, Step 1. Synthesis of tert-butyl 9-(4-amino-7-methyl-5-(1-methyl-1H-pyrazol-4-yl)-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (200 mg, 0.42 mmol, 1 eq), (1-methyl-1H-pyrazol-4-yl)boronic acid (159 mg, 1.26 mmol, 3 eq), Pd(DtBPF)Cl₂ (27 mg, 0.042 mmol, 0.1 eq) and K₃PO₄ (267 mg, 1.26 mmol, 3 eq) in DMF (5 mL) and H₂O (0.5 mL) was stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄, filtered and concentrated under reduced pressure. The resulting residue was purified by silica gel column chromatography, eluting with DCM/MeOH (20:1) to afford the title compound (100 mg, 50% yield) as a yellow solid.

LCMS: m/z 478.6 [M+1]⁺.

### Scheme L, Step 2. Synthesis of 7-methyl-5-(1-methyl-1H-pyrazol-4-yl)-6-(3-azaspiro[5.5]undec-8-en-9-yl)-7H-pyrrolo[2,3-d]pyrimidin-4-amine

To a stirred solution of tert-butyl 9-(4-amino-7-methyl-5-(1-methyl-1H-pyrazol-4-yl)-7H-pyrrolo[2,3-d]pyrimidin-6-yl)-3-azaspiro[5.5]undec-8-ene-3-carboxylate (100 mg, 0.21 mmol, 1 eq) in DCM (5 mL) was added TFA (2 mL) and the mixture was stirred at room temperature for 2 hrs. The solution was concentrated to afford the title compound (70 mg, 88% yield) as a yellow solid.

LCMS: m/z 478.7 [M+1]⁺.

### Scheme L, Step 3. Synthesis of 1-(9-(4-amino-7-methyl-5-(1-methyl-1H-pyrazol-4-yl)-7H-pyrrolo[2,3-d]pyrimidin-6-yl)-3-azaspiro[5.5]undec-8-en-3-yl)prop-2-en-1-one

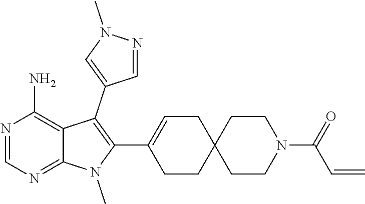 [SMILES: C=CC(=O)N1CCC2(CC=C(c3c(-c4cnn(C)c4)c4c(N)ncnc4n3C)CC2)CC1]

### Example 14

To a stirred solution of 7-methyl-5-(1-methyl-1H-pyrazol-4-yl)-6-(3-azaspiro[5.5]-undec-8-en-9-yl)-7H-pyrrolo[2,3-d]pyrimidin-4-amine (70 mg, 0.18 mmol, 1 eq) in dry CHCl₃ (4 mL) was added TEA (75 mg, 0.74 mmol, 4 eq). A solution of acryloyl chloride (17 mg, 0.18 mmol, 1 eq) in dry CHCl₃ (0.5 mL) was added dropwise at 0° C. and the resulting mixture was stirred at 0° C. for 0.5 hrs under N₂. The reaction was quenched with MeOH. The solvent was removed under reduced pressure. The residue was purified by Prep-HPLC (0.1% TFA in ACN/H₂O) to afford the title compound (7.9 mg, 10% yield) as a white solid.

LCMS: m/z 432.4 [M+1]⁺.

¹H NMR (400 MHz, CD₃OD) δ 8.31 (s, 1H), 7.95 (s, 1H), 7.80 (s, 1H), 6.77 (dd, J=16.0, 10.7 Hz, 1H), 6.17 (d, J=16.0 Hz, 1H), 5.95 (brs, 1H), 5.73 (d, J=12.0 Hz, 1H), 4.04 (s, 3H), 3.78 (s, 3H), 3.73-3.59 (m, 4H), 2.22-2.19 (m, 2H), 2.14-2.11 (m, 2H), 1.68-1.64 (m, 2H), 1.51-1.41 (m, 4H).

### Example 15

### Synthesis of 1-(9-(4-amino-5-isopropyl-7-methyl-7H-pyrrolo[2,3-d]pyrimidin-6-yl)-3-azaspiro[5.5]undec-8-en-3-yl)prop-2-en-1-one

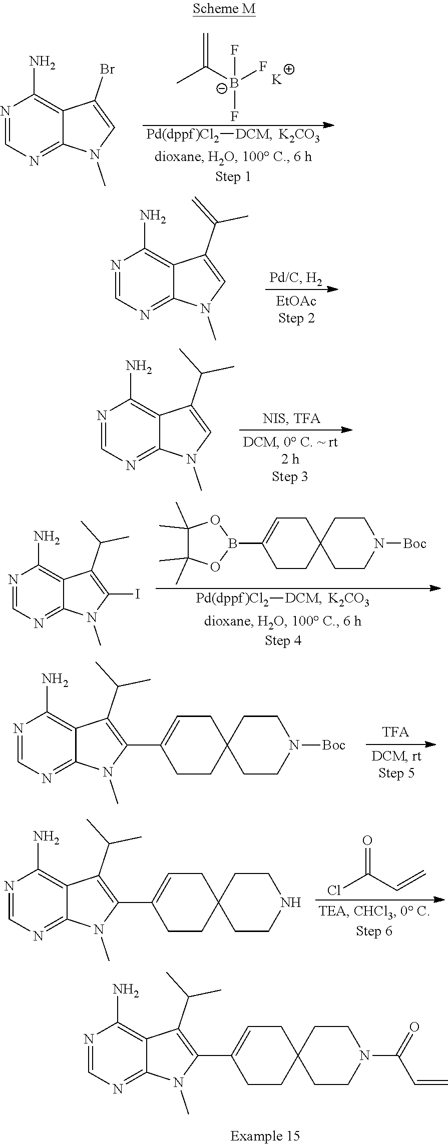

### Scheme M, Step 1. Synthesis of 7-methyl-5-(prop-1-en-2-yl)-7H-pyrrolo[2,3-d]pyrimidin-4-amine

A mixture of 5-bromo-7-methyl-7H-pyrrolo[2,3-d]pyrimidin-4-amine (1.8 g, 7.9 mmol, 1 eq), potassium trifluoro(prop-1-en-2-yl)borate (1.76 g, 11.9 mmol, 1.5 eq), Pd(dppf)Cl₂-DCM (650 mg, 0.79 mmol, 0.1 eq) and K₂CO₃ (2.74 g, 19.9 mmol, 3 eq) in dioxane (24 mL) and H₂O (3 mL) was degassed with N₂ three times, and then stirred at 100° C. for 16 h under N₂. The resulting mixture was diluted with H₂O (50 mL) and extracted with EtOAc (3×40 mL). The combined organic layers were washed with brine (30 mL), dried over anhydrous Na₂SO₄, filtered and then concentrated under reduced pressure. The residue was purified by silica gel column chromatography, eluting with DCM/MeOH (70:1) to afford the title compound (610 mg, 41% yield) as a yellow solid.

LCMS: m/z 189.23 [M+1]⁺.

### Scheme M, Step 2. Synthesis of 5-isopropyl-7-methyl-7H-pyrrolo[2,3-d]pyrimidin-4-amine

A suspension of 7-methyl-5-(prop-1-en-2-yl)-7H-pyrrolo[2,3-d]pyrimidin-4-amine (240 mg, 1.27 mmol, 1 eq) and Pd/C (50 mg) in MeOH (6 mL) was degassed with H₂ three times and then stirred at room temperature for 2 h under H₂. The resulting mixture is filtered. The filtrate was concentrated under reduced pressure to afford the title compound (220 mg, 91% yield) as a yellow solid, which is used for next step directly without further purification.

LCMS: m/z 191.38 [M+1]⁺.

### Scheme M, Step 3. Synthesis of 6-iodo-5-isopropyl-7-methyl-7H-pyrrolo[2,3-d]pyrimidin-4-amine

To a solution of compound 5-isopropyl-7-methyl-7H-pyrrolo[2,3-d]pyrimidin-4-amine (330 mg, 1.73 mmol, 1 eq) in DCM (6 mL) was added TFA (990 mg, 8.7 mmol, 5 eq) and NIS (508 mg, 2.26 mmol, 1.3 eq) in one portion under N₂ at 0° C. and the resulting mixture was stirred at room temperature for 2 hrs. The mixture was poured into saturated aqueous Na₂SO₃ solution. The precipitated solid was collected by filtration, washed with water, and dried under reduced pressure to afford the title compound (350 mg, 64%) as a gray solid.

LCMS: m/z 317.58 [M+1]⁺.

### Scheme M, Step 4. Synthesis of tert-butyl 9-(4-amino-5-isopropyl-7-methyl-7H-pyrrolo[2,3-d]pyrimidin-6-yl)-3-azaspiro[5.5]undec-8-ene-3-carboxylate

A mixture of 6-iodo-5-isopropyl-7-methyl-7H-pyrrolo[2,3-d]pyrimidin-4-amine (110 mg, 0.35 mmol, 1 eq), tert-butyl 9-(4,4,5,5-tetramethyl-1,3,2-dioxaborolan-2-yl)-3-azaspiro[5.5]undec-8-ene-3-carboxylate (196 mg, 0.52 mmol, 1.5 eq), Pd(dppf)Cl₂-DCM (29 mg, 0.035 mmol, 0.1 eq) and K₂CO₃ (120 mg, 120 mmol, 2.5 eq) in dioxane (6 mL) and H₂O (0.6 mL) was degassed with N₂ three times and then stirred at 100° C. for 16 h under N₂. The resulting mixture was diluted with H₂O (20 mL) and extracted with EtOAc (3×20 mL). The combined organic layers were washed with brine (30 mL), dried over anhydrous Na₂SO₄, filtered and concentrated under reduced pressure. The residue was purified by silica gel column chromatography, eluting with DCM/MeOH (20:1) to afford the title compound (110 mg, 72% yield) as a yellow solid.

LCMS: m/z 440.31 [M+1]⁺.

### Scheme M, Step 5. Synthesis of 5-isopropyl-7-methyl-6-(3-azaspiro[5.5]undec-8-en-9-yl)-7H-pyrrolo[2,3-d]pyrimidin-4-amine

To a stirred solution of tert-butyl 9-(4-amino-5-isopropyl-7-methyl-7H-pyrrolo[2,3-d]pyrimidin-6-yl)-3-azaspiro[5.5]undec-8-ene-3-carboxylate (110 mg, 0.25 mmol, 1 eq) in DCM (3 mL) was added TFA (1 mL) and the mixture was stirred at room temperature for 2 hrs. The solution was concentrated to afford the title compound (60 mg, 70% yield) as a yellow solid.

LCMS: m/z 340.29 [M+1]⁺.

### Scheme M, Step 6. Synthesis of 1-(9-(4-amino-5-isopropyl-7-methyl-7H-pyrrolo[2,3-d]pyrimidin-6-yl)-3-azaspiro[5.5]undec-8-en-3-yl)prop-2-en-1-one

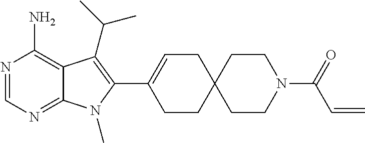 [SMILES: C=CC(=O)N1CCC2(CC=C(c3c(C(C)C)c4c(N)ncnc4n3C)CC2)CC1]

### Example 15

To a stirred solution of 5-isopropyl-7-methyl-6-(3-azaspiro[5.5]undec-8-en-9-yl)-7H-pyrrolo[2,3-d]pyrimidin-4-amine (60 mg, 0.17 mmol, 1 eq) in dry CHCl₃ (4 mL) was added TEA (58 mg, 0.54 mmol, 3 eq). A solution of acryloyl chloride (18.4 mg, 0.2 mmol, 1.2 eq) in dry CHCl₃ (0.5 mL) was added dropwise at 0° C., and the resulting mixture was stirred at 0° C. for 0.5 hr under N₂. The reaction was quenched with MeOH. The solvent was removed under reduced pressure. The residue was purified by Prep-HPLC (0.1% TFA in ACN/H₂O) to afford the title compound (29.8 mg, 39.5% yield) as a white solid.

LCMS: m/z 394.30 [M+1]⁺.

¹H NMR (400 MHz, CD₃OD) δ 8.24 (s, 1H), 6.80 (dd, J=16.0, 10.0 Hz, 1H), 6.18 (d, J=16.0 Hz, 1H), 5.90 (brs, 1H), 5.73 (d, J=10.0 Hz, 1H), 3.82-3.72 (m, 2H), 3.66 (s, 3H), 3.61-3.57 (m, 2H), 3.37-3.32 (m, 1H), 2.28-2.24 (m, 4H), 1.79-1.75 (m, 2H), 1.63-1.55 (m, 4H), 1.34 (d, J=8.0 Hz, 6H).

### Example 16

### Synthesis of 1-(9-(4-amino-5-(3-methoxyphenyl)-7-methyl-7H-pyrrolo[2,3-d]pyrimidin-6-yl)-3-azaspiro[5.5]undec-8-en-3-yl)prop-2-en-1-one

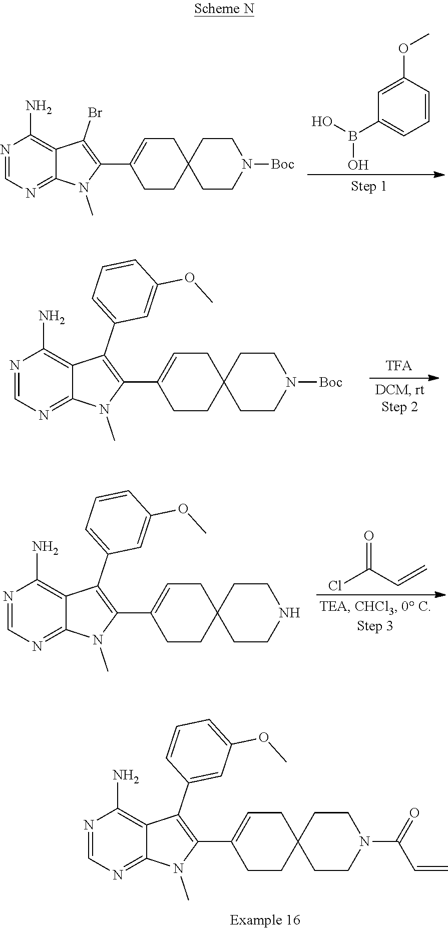

### Scheme N, Step 1. Synthesis of tert-butyl 9-(4-amino-5-(3-methoxyphenyl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (100 mg, 0.21 mmol, 1 eq), 3-methoxyphenyl boronic acid (64 mg, 0.42 mmol, 2 eq), Pd(DtBPF)Cl₂ (14 mg, 0.02 mmol, 0.1 eq) and K₃PO₄ (87 mg, 0.63 mmol, 3 eq) in DMF (10 mL) and H₂O (1 mL) was degassed with N₂ three times, and then stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄, filtered and concentrated under reduced pressure. The residue was purified by silica gel column chromatography, eluting with DCM/MeOH (20:1) to afford the title compound (74 mg, 70% yield) as a yellow solid.

LCMS: m/z 504.6 [M+1]⁺.

### Scheme N, Step 2. Synthesis of 5-(3-methoxyphenyl)-7-methyl-6-(3-azaspiro[5.5]undec-8-en-9-yl)-7H-pyrrolo[2,3-d]pyrimidin-4-amine

To a stirred solution of tert-butyl 9-(4-amino-5-(3-methoxyphenyl)-7-methyl-7H-pyrrolo[2,3-d]pyrimidin-6-yl)-3-azaspiro[5.5]undec-8-ene-3-carboxylate (74 mg, 0.15 mmol, 1 eq) in DCM (4 mL) was added TFA (2 mL) and the mixture was stirred at room temperature for 2 hrs. The solution was concentrated to afford the title compound (48 mg, 81% yield) as a yellow solid.

LCMS: m/z 404.50 [M+1]⁺.

### Scheme N, Step 3. Synthesis of 1-(9-(4-amino-5-(3-methoxyphenyl)-7-methyl-7H-pyrrolo[2,3-d]pyrimidin-6-yl)-3-azaspiro[5.5]undec-8-en-3-yl)prop-2-en-1-one

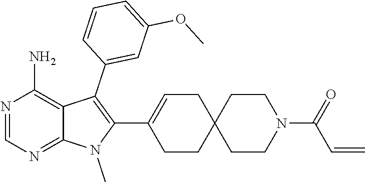 [SMILES: C=CC(=O)N1CCC2(CC=C(c3c(-c4cccc(OC)c4)c4c(N)ncnc4n3C)CC2)CC1]

### Example 16

To a stirred solution of 5-(3-methoxyphenyl)-7-methyl-6-(3-azaspiro[5.5]undec-8-en-9-yl)-7H-pyrrolo[2,3-d]pyrimidin-4-amine (48 mg, 0.12 mmol, 1 eq) in dry CHCl₃ (4 mL) was added TEA (61 mg, 0.6 mmol, 5 eq). A solution of acryloyl chloride (11 mg, 0.12 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (28.9 mg, 53% yield) as a white solid.

LCMS: m/z 458.40 [M+1]⁺.

¹H NMR (400 MHz, CD₃OD) δ 8.32 (s, 1H), 7.43-7.39 (m, 1H), 7.03-6.96 (m, 3H), 6.75 (dd, J=16.0, 12.0 Hz, 1H), 6.16 (dd, J=16.0 Hz, 1H), 5.94 (brs, 1H), 5.73-5.70 (d, J=12.0 Hz, 1H), 3.83 (s, 3H), 3.80 (s, 3H), 3.75-3.60 (m, 2H), 3.57-3.45 (m, 2H), 2.18-2.14 (m, 2H), 2.06-2.02 (m, 2H), 1.63-1.55 (m, 2H), 1.48-1.37 (m, 4H).

### Example 17

### Synthesis of 1-(9-(4-amino-5-(4-(difluoromethoxy)phenyl)-7-methyl-7H-pyrrolo[2,3-d]pyrimidin-6-yl)-3-azaspiro[5.5]undec-8-en-3-yl)prop-2-en-1-one

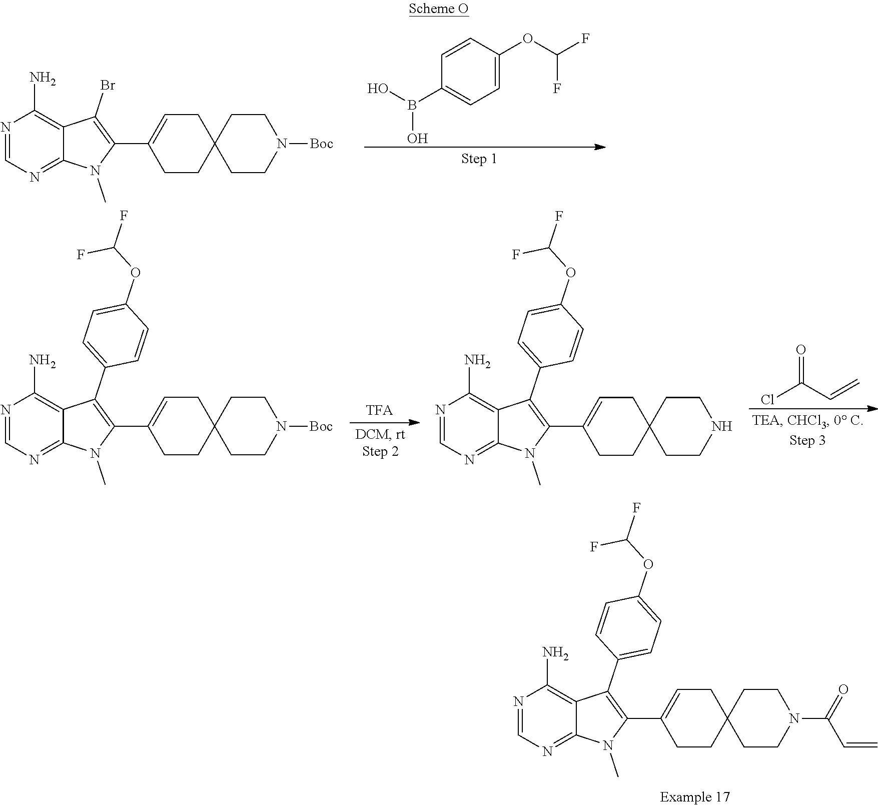

### Scheme O, Step 1. Synthesis of tert-butyl 9-(4-amino-5-(4-(difluoromethoxy)phenyl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (100 mg, 0.21 mmol, 1 eq), 4-difluoro-methoxyphenyl boronic acid (79 mg, 0.42 mmol, 2 eq), Pd(DtBPF)Cl₂ (14 mg, 0.02 mmol, 0.1 eq) and K₃PO₄ (87 mg, 0.63 mmol, 3 eq) in DMF (10 mL) and H₂O (1 mL) was degassed with N₂ three times and then stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄, filtered and then concentrated under reduced pressure. The residue was purified by silica gel column chromatography, eluting with DCM/MeOH (20:1) to afford the title compound (98 mg, 86% yield) as a yellow solid.

LCMS: m/z 540.27 [M+1]⁺.

### Scheme O, Step 2. Synthesis of 5-(4-(difluoromethoxy)phenyl)-7-methyl-6-(3-azaspiro[5.5]undec-8-en-9-yl)-7H-pyrrolo[2,3-d]pyrimidin-4-amine

To a stirred solution of tert-butyl 9-(4-amino-5-(4-(difluoromethoxy)phenyl)-7-methyl-7H-pyrrolo[2,3-d]pyrimidin-6-yl)-3-azaspiro[5.5]undec-8-ene-3-carboxylate (98 mg, 0.18 mmol, 1 eq) in DCM (4 mL) was added TFA (2 mL). The mixture was stirred at room temperature for 2 hrs. The solution was concentrated to afford the title compound (65 mg, 81% yield) as a yellow solid.

LCMS: m/z 404.50 [M+1]⁺.

### Scheme O, Step 3. Synthesis of 1-(9-(4-amino-5-(4-(difluoromethoxy)phenyl)-7-methyl-7H-pyrrolo[2,3-d]pyrimidin-6-yl)-3-azaspiro[5.5]undec-8-en-3-yl)prop-2-en-1-one

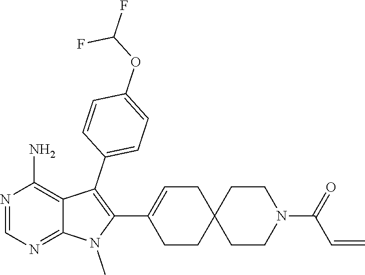 [SMILES: C=CC(=O)N1CCC2(CC=C(c3c(-c4ccc(OC(F)F)cc4)c4c(N)ncnc4n3C)CC2)CC1]

### Example 17

To a stirred solution of 5-(4-(difluoromethoxy)phenyl)-7-methyl-6-(3-azaspiro[5.5]-undec-8-en-9-yl)-7H-pyrrolo[2,3-d]pyrimidin-4-amine (65 mg, 0.15 mmol, 1 eq) in dry CHCl₃ (4 mL) was added TEA (76 mg, 0.75 mmol, 5 eq). A solution of acryloyl chloride (14 mg, 0.15 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (25.9 mg, 35% yield) as a white solid.

LCMS: m/z 494.35 [M+1]⁺.

¹H NMR (400 MHz, CD₃OD) δ 8.32 (s, 1H), 7.45 (d, J=8.0 Hz, 2H), 7.27 (d, J=8.0 Hz, 2H), 7.92 (t, J=76.0 Hz, 1H), 6.74 (dd, J=16.0, 12.0 Hz, 1H), 6.16 (d, J=16.0 Hz, 1H), 5.93 (brs, 1H), 5.72 (dd, J=12.0 Hz, 1H), 3.80 (s, 3H), 3.74-3.59 (m, 2H), 3.57-3.46 (m, 2H), 2.16-2.13 (m, 2H), 2.06-2.02 (m, 2H), 1.61-1.58 (m, 2H), 1.45-1.37 (m, 4H).

### Example 18

### Synthesis of 1-(9-(4-amino-5-(4-cyclopropoxyphenyl)-7-methyl-7H-pyrrolo[2,3-d]pyrimidin-6-yl)-3-azaspiro[5.5]undec-8-en-3-yl)prop-2-en-1-one

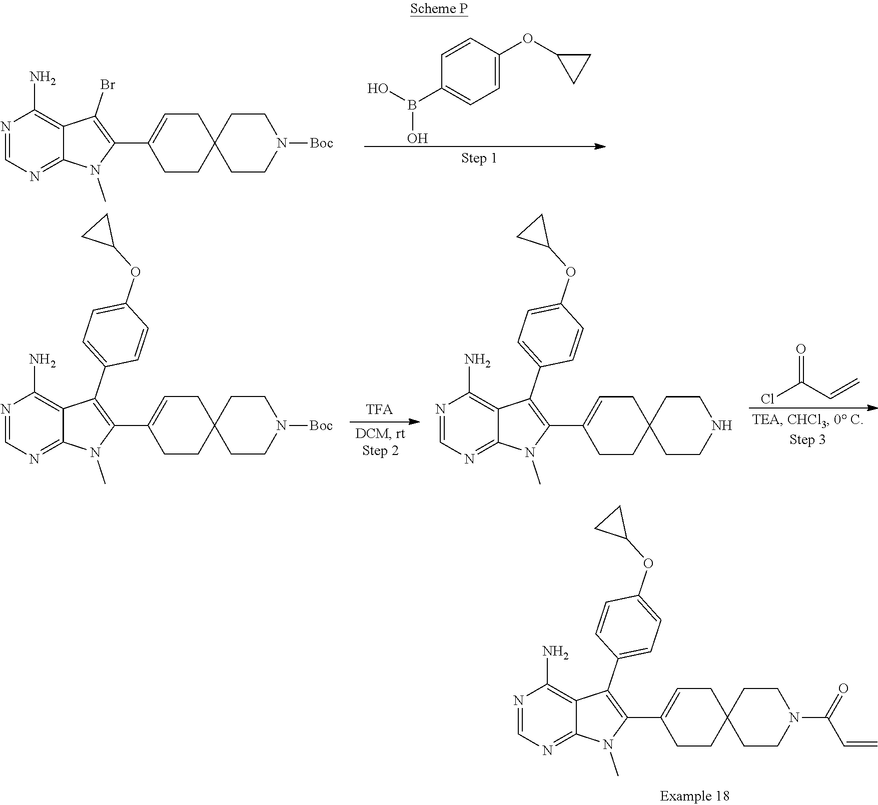

### Scheme P, Step 1. Synthesis of tert-butyl 9-(4-amino-5-(4-cyclopropoxyphenyl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (100 mg, 0.21 mmol, 1 eq), 4-cyclopropyloxy-phenyl boronic acid (75 mg, 0.42 mmol, 2 eq), Pd(DtBPF)Cl₂ (14 mg, 0.02 mmol, 0.1 eq) and K₃PO₄ (87 mg, 0.63 mmol, 3 eq) in DMF (10 mL) and H₂O (1 mL) was degassed with N₂ three times and stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄, filtered and then concentrated under reduced pressure. The residue was purified by silica gel column chromatography, eluting with DCM/MeOH (20:1) to afford the title compound (80 mg, 72% yield) as a yellow solid.

LCMS: m/z 530.70 [M+1]⁺.

### Scheme P, Step 2. Synthesis of 5-(4-cyclopropoxyphenyl)-7-methyl-6-(3-azaspiro[5.5]undec-8-en-9-yl)-7H-pyrrolo[2,3-d]pyrimidin-4-amine

To a stirred solution of tert-butyl 9-(4-amino-5-(4-cyclopropoxyphenyl)-7-methyl-7H-pyrrolo[2,3-d]pyrimidin-6-yl)-3-azaspiro[5.5]undec-8-ene-3-carboxylate (80 mg, 0.15 mmol, 1 eq) in DCM (4 mL) was added TFA (2 mL). The mixture _{was stirred} at room temperature for 2 hrs. The solution was concentrated to afford the title compound (41 mg, 61% yield) as a yellow solid.

LCMS: m/z 430.60 [M+1]⁺.

### Scheme P, Step 3. Synthesis of 1-(9-(4-amino-5-(4-cyclopropoxyphenyl)-7-methyl-7H-pyrrolo[2,3-d]pyrimidin-6-yl)-3-azaspiro[5.5]undec-8-en-3-yl)prop-2-en-1-one

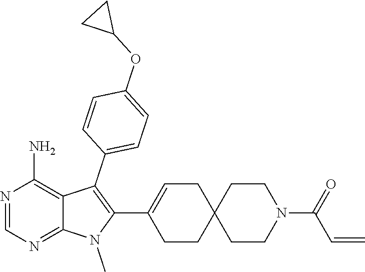 [SMILES: C=CC(=O)N1CCC2(CC=C(c3c(-c4ccc(OC5CC5)cc4)c4c(N)ncnc4n3C)CC2)CC1]

### Example 18

To a stirred solution of 5-(4-cyclopropoxyphenyl)-7-methyl-6-(3-azaspiro[5.5]undec-8-en-9-yl)-7H-pyrrolo[2,3-d]pyrimidin-4-amine (41 mg, 0.09 mmol, 1 eq) in dry CHCl₃ (4 mL) was added TEA (48 mg, 0.48 mmol, 5 eq). A solution of acryloyl chloride (9 mg, 0.09 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (15.5 mg, 33% yield) as a white solid.

LCMS: m/z 484.40 [M+1]⁺.

¹H NMR (400 MHz, CD₃OD) δ 8.29 (s, 1H), 7.32 (d, J=8.0 Hz, 2H), 7.16 (d, J=8.0 Hz, 2H), 6.74 (dd, J=16.0, 12.0 Hz, 1H), 6.15 (d, J=16.0 Hz, 1H), 5.91 (brs, 1H), 5.71 (dd, J=12.0 Hz, 1H), 3.86-3.80 (m, 1H), 3.79 (s, 3H), 3.73-3.59 (m, 2H), 3.57-3.44 (m, 2H), 2.16-2.12 (m, 2H), 2.05-2.00 (m, 2H), 1.62-1.52 (m, 2H), 1.47-1.31 (m, 4H), 0.87-0.78 (m, 2H), 0.77-0.66 (m, 2H).

### Example 19

### Synthesis of N-(7-(4-amino-5-(4-methoxyphenyl)-7-methyl-7H-pyrrolo[2,3-d]pyrimidin-6-yl)spiro[3.5]nonan-2-yl)acrylamide

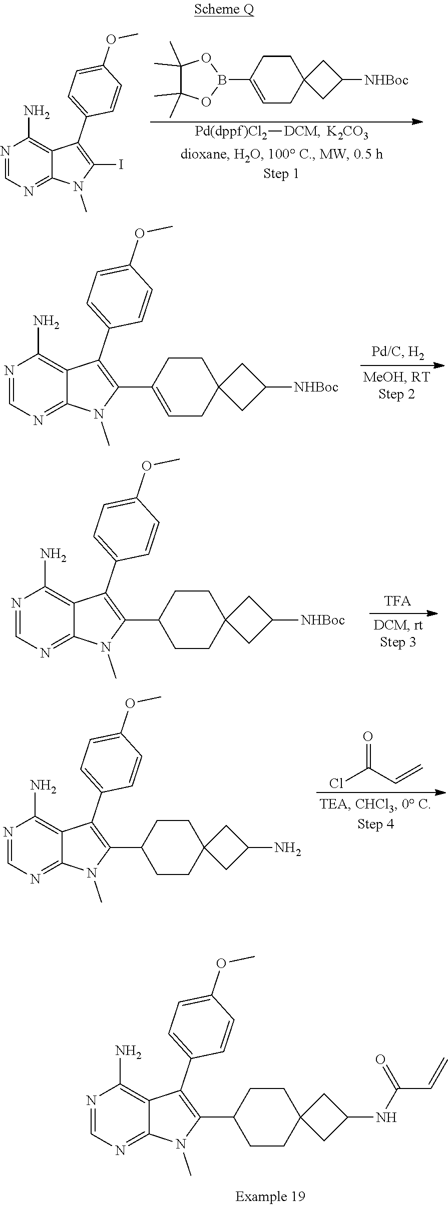

### Scheme Q, Step 1. Synthesis of tert-butyl (7-(4-amino-5-(4-methoxyphenyl)-7-methyl-7H-pyrrolo[2,3-d]pyrimidin-6-yl)spiro[3.5]non-6-en-2-yl)carbamate

A mixture of 6-iodo-5-(4-methoxyphenyl)-7-methyl-7H-pyrrolo[2,3-d]pyrimidin-4-amine (600 mg, 1.57 mmol, 1 eq), tert-butyl (7-(4,4,5,5-tetramethyl-1,3,2-dioxaborolan-2-yl)spiro[3.5]-non-6-en-2-yl)carbamate. (1.08 g, 3.14 mmol, 2 eq), Pd(dppf)Cl₂ (129 mg, 0.157 mmol, 0.1 eq) and K₂CO₃ (648 mg, 4.71 mmol, 3 eq) in dioxane (15 mL) and H₂O (1.5 mL) was degassed with N₂ three times, and then stirred at 100° C. under microwave irradiation for 30 min. The resulting mixture was diluted with H₂O (20 mL) and extracted with EtOAc (3×15 mL). The combined organic layers were dried over anhydrous Na₂SO₄ and filtered. The filtrate was concentrated under reduced pressure. The residue was purified by silica gel column chromatography, eluting with DCM/MeOH (30:1) to afford the crude product (390 mg, 50% purity). The crude product was purified by Prep-C18, eluting with H₂O/ACN (3:2) to afford the title compound (110 mg, 23% yield) as a yellow solid.

LCMS: m/z 490.30 [M+1]⁺.

### Scheme Q, Step 2. Synthesis of tert-butyl (7-(4-amino-5-(4-methoxyphenyl)-7-methyl-7H-pyrrolo[2,3-d]pyrimidin-6-yl)spiro[3.5]nonan-2-yl)carbamate

To a stirred solution of tert-butyl (7-(4-amino-5-(4-methoxyphenyl)-7-methyl-7H-pyrrolo[2,3-d]pyrimidin-6-yl)spiro[3.5]non-6-en-2-yl)carbamate (110 mg, 0.22 mmol, 1 eq) in MeOH (30 mL) was added Pd/C (100 mg). The resulting mixture was degassed with H₂ three times, stirred under H₂ at room temperature for 48 hrs, and then filtered. The filtrate was concentrated under reduced pressure. The resulting residue was purified by silica gel column chromatography, eluting with DCM/MeOH (15:1) to afford the title compound (23 mg, 12% yield) as a yellow solid.

LCMS: m/z 492.43 [M+1]⁺.

### Scheme Q, Step 3. Synthesis of 6-(2-aminospiro[3.5]nonan-7-yl)-5-(4-methoxyphenyl)-7-methyl-7H-pyrrolo[2,3-d]pyrimidin-4-amine

To a stirred solution of tert-butyl (7-(4-amino-5-(4-methoxyphenyl)-7-methyl-7H-pyrrolo[2,3-d]pyrimidin-6-yl)spiro[3.5]nonan-2-yl)carbamate. (23 mg, 0.046 mmol, 1 eq) in DCM (1 mL) was added TFA (0.5 mL). The mixture was stirred at room temperature for 2 hrs. The solution was concentrated to afford the title compound (16.7 mg, 92% yield) as a yellow solid.

LCMS: m/z 392.33 [M+1]⁺.

### Scheme Q, Step 4. Synthesis of N-(7-(4-amino-5-(4-methoxyphenyl)-7-methyl-7H-pyrrolo[2,3-d]pyrimidin-6-yl)spiro[3.5]nonan-2-yl)acrylamide

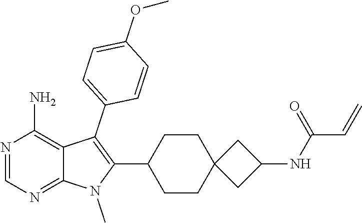 [SMILES: C=CC(=O)NC1CC2(CCC(c3c(-c4ccc(OC)cc4)c4c(N)ncnc4n3C)CC2)C1]

### Example 19

To a stirred solution of 6-(2-aminospiro[3.5]nonan-7-yl)-5-(4-methoxyphenyl)-7-methyl-7H-pyrrolo[2,3-d]pyrimidin-4-amine (16.7 mg, 0.042 mmol, 1 eq) in dry CHCl₃ (5 mL) was added TEA (12.7 mg, 0.126 mmol, 3 eq). A solution of acryloyl chloride (3.8 mg, 0.042 mmol, 1 eq) in dry CHCl₃ (0.5 mL) was added dropwise at 0° C., and the resulting mixture was stirred at 0° C. for 0.5 hrs under N₂.

The reaction mixture was quenched with MeOH. The solvent was removed under reduced pressure. The residue was purified by Prep-HPLC (0.1% TFA in ACN/H₂O) to afford the title compound (2.7 mg, 14% yield) as a white solid.

LCMS: m/z 446.40 [M+1]⁺.

¹H NMR (400 MHz, CD3OD) δ 8.25 (s, 1H), 7.33-7.30 (m, 2H), 7.08-7.05 (m, 2H), 6.18 (d, J=8.0 Hz, 2H), 5.62 (t, J=6.0 Hz, 1H), 4.27-4.18 (m, 1H), 3.90 (s, 3H), 3.86 (s, 3H), 2.89-2.82 (m, 1H), 2.11-2.05 (m, 2H), 1.82-1.79 (m, 1H), 1.71-1.66 (m, 2H), 1.62-1.56 (m, 3H), 1.49-1.40 (m, 4H).

### Example 20

### Synthesis of 1-(9-(4-amino-7-methyl-5-(4-(2-(pyrrolidin-1-yl)ethoxy)phenyl)-7H-pyrrolo[2,3-d]pyrimidin-6-yl)-3-azaspiro[5.5]undec-8-en-3-yl)prop-2-en-1-one

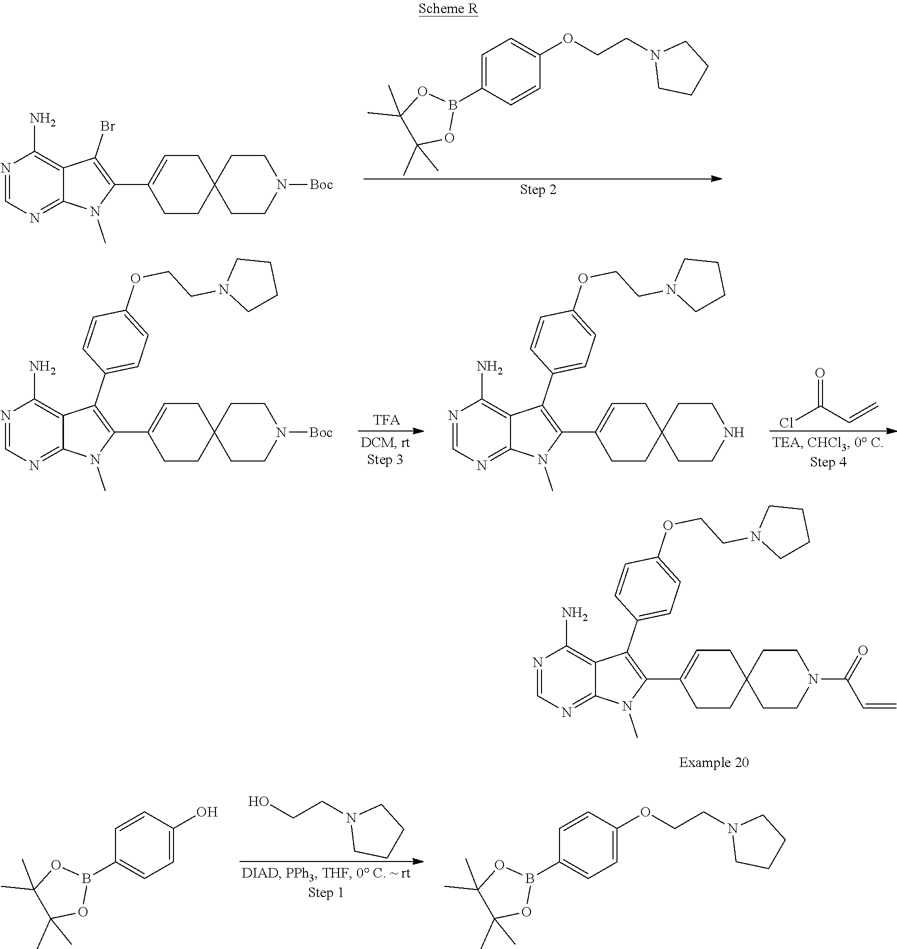

### Scheme R, Step 1. Synthesis of 1-(2-(4-(4,4,5,5-tetramethyl-1,3,2-dioxaborolan-2-yl)phenoxy)ethyl)pyrrolidine

To a mixture of 4-(4,4,5,5-tetramethyl-1,3,2-dioxaborolan-2-yl)phenol (2 g, 9.1 mmol, 1 eq), 2-(pyrrolidin-1-yl)ethan-1-ol (1.25 g, 10.9 mmol, 1.2 eq) and PPh₃ (3 g, 10.9 mmol, 1.2 eq) in dry THF (20 mL) was added DIAD (2.2 g, 10.9 mmol, 1.2 eq) dropwise at 0° C. under N₂ and the resulting mixture was stirred at room temperature for 16 hrs under N₂. The resulting mixture was diluted with H₂O (20 mL) and extracted with EtOAc (3×15 mL). The combined organic layers were washed with brine (30 mL), dried over anhydrous Na₂SO₄ and filtered. The filtrate was concentrated under reduced pressure. The residue was purified by silica gel column chromatography, eluting with PE/EA (5:1) to afford the title compound (1.8 g, 62% yield) as a white solid.

LCMS: m/z 318.5 [M+1]⁺.

### Scheme R, Step 2. Synthesis of tert-butyl 9-(4-amino-7-methyl-5-(4-(2-(pyrrolidin-1-yl)ethoxy)phenyl)-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (200 mg, 0.42 mmol, 1 eq), 1-(2-(4-(4,4,5,5-tetramethyl-1,3,2-dioxaborolan-2-yl)phenoxy)ethyl)pyrrolidine (318 mg, 0.84 mmol, 2 eq), Pd(DtBPF)Cl₂ (26 mg, 0.042 mmol, 0.1 eq) and K₃PO₄ (267 mg, 1.26 mmol, 3 eq) in DMF (5 mL) and H₂O (0.5 mL) is bubbled with N₂ for 2 minutes and then stirred at 120° C. under microwave irradiation for 30 min. The resulting mixture was diluted with H₂O (20 mL) and extracted with EtOAc (3×15 mL). The combined organic layers were washed with brine (4×15 mL), and the combined organic layers were dried over anhydrous Na₂SO₄ and filtered. The filtrate was concentrated under reduced pressure. The residue was purified by silica gel column chromatography, eluting with DCM/MeOH (20:1) to afford the title compound (115 mg, 47% yield) as a yellow solid.

LCMS: m/z 487.8 [M+1]⁺.

### Scheme R, Step 3. Synthesis of 7-methyl-5-(4-(2-(pyrrolidin-1-yl)ethoxy)phenyl)-6-(3-azaspiro[5.5]undec-8-en-9-yl)-7H-pyrrolo[2,3-d]pyrimidin-4-amine

To a stirred solution of tert-butyl 9-(4-amino-7-methyl-5-(4-(2-(pyrrolidin-1-yl)ethoxy)-phenyl)-7H-pyrrolo[2,3-d]pyrimidin-6-yl)-3-azaspiro[5.5]undec-8-ene-3-carboxylate. (115 mg, 0.19 mmol, 1 eq) in DCM (5 mL) was added TFA (2 mL) and the mixture was stirred at room temperature for 2 hrs. The solution was concentrated to afford the title compound (85 mg, 89% yield) as a yellow solid.

LCMS: m/z 487.7 [M+1]⁺.

### Scheme R, Step 4. Synthesis of 1-(9-(4-amino-7-methyl-5-(4-(2-(pyrrolidin-1-yl)ethoxy)phenyl)-7H-pyrrolo[2,3-d]pyrimidin-6-yl)-3-azaspiro[5.5]undec-8-en-3-yl)prop-2-en-1-one

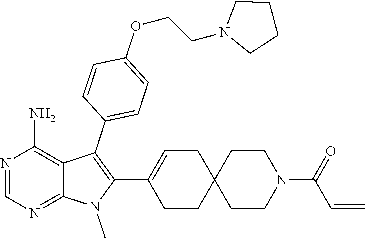 [SMILES: C=CC(=O)N1CCC2(CC=C(c3c(-c4ccc(OCCN5CCCC5)cc4)c4c(N)ncnc4n3C)CC2)CC1]

### Example 20

To a stirred solution of 7-methyl-5-(4-(2-(pyrrolidin-1-yl)ethoxy)phenyl)-6-(3-azaspiro[5.5]undec-8-en-9-yl)-7H-pyrrolo[2,3-d]pyrimidin-4-amine (85 mg, 0.17 mmol, 1 eq) in dry CHCl₃ (4 mL) was added TEA (71 mg, 0.70 mmol, 4 eq). A solution of acryloyl chloride (15 mg, 0.17 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (26.2 mg, 28% yield) as a white solid

LCMS: m/z 541.5 [M+1]⁺.

¹H NMR (400 MHz, CD₃OD) δ 8.32 (s, 1H), 7.39 (d, J=8.0 Hz, 2H), 7.17 (d, J=8.0 Hz, 2H), 6.78 (dd, J=16.0, 10.7 Hz, 1H), 6.17 (d, J=16.0 Hz, 1H), 5.93 (brs, 1H), 5.73 (d, J=10.7 Hz, 1H), 4.43-4.41 (m, 2H), 3.80 (s, 3H), 3.76-3.63 (m, 6H), 3.59-3.43 (m, 2H), 3.28-3.22 (m, 2H), 2.23-2.00 (m, 8H), 1.61-1.54 (m, 2H), 1.52-1.41 (m, 2H), 1.38-1.31 (m, 2H).

### Example 21

### Synthesis of 1-(9-(4-amino-7-methyl-5-(6-(methylamino)pyridin-3-yl)-7H-pyrrolo[2,3-d]pyrimidin-6-yl)-3-azaspiro[5.5]undec-8-en-3-yl)prop-2-en-1-one

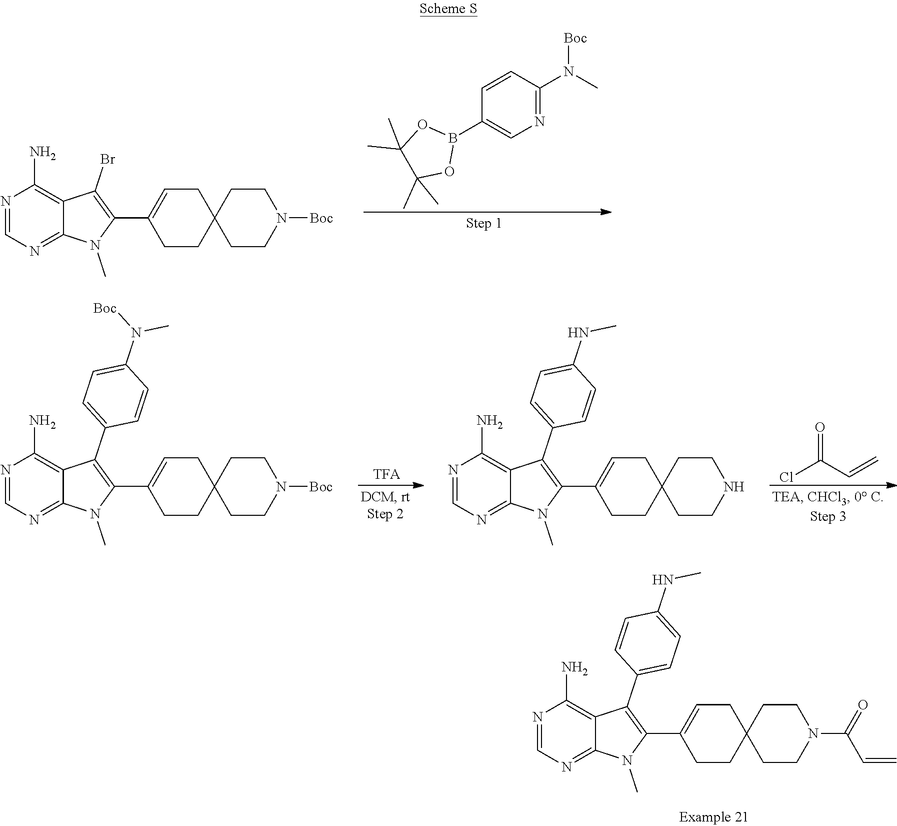

### Scheme S, Step 1. Synthesis of tert-butyl 9-(4-amino-5-(6-((tert-butoxycarbonyl)(methyl)amino)pyridin-3-yl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (260 mg, 0.54 mmol, 1 eq), tert-butyl methyl-(5-(4,4,5,5-tetramethyl-1,3,2-dioxaborolan-2-yl)pyridin-2-yl)carbamate (368 mg, 1.1 mmol, 2 eq), Pd(DtBPF)Cl₂ (36 mg, 0.05 mmol, 0.1 eq) and K₃PO₄ (226 mg, 1.63 mmol, 3 eq) in DMF (10 mL) and H₂O (1 mL) is bubbled with N₂ for 2 minutes, and then stirred at 120° C. under microwave irradiation for 30 min. The resulting mixture was diluted with H₂O (20 mL) and extracted with EtOAc (3×15 mL). The combined organic layers were washed with brine (4×15 mL), dried over anhydrous Na₂SO₄ and filtered. The filtrate was concentrated under reduced pressure. The residue was purified by silica gel column chromatography, eluting with DCM/MeOH (20:1) to afford the title compound (170 mg, 52% yield) as a red solid.

LCMS: m/z 604.70 [M+1]⁺.

### Scheme S, Step 2. Synthesis of 7-methyl-5-(6-(methylamino)pyridin-3-yl)-6-(3-azaspiro[5.5]undec-8-en-9-yl)-7H-pyrrolo[2,3-d]pyrimidin-4-amine

To a stirred solution of tert-butyl 9-(4-amino-5-(6-((tert-butoxycarbonyl)(methyl)amino)-pyridin-3-yl)-7-methyl-7H-pyrrolo[2,3-d]pyrimidin-6-yl)-3-azaspiro[5.5]undec-8-ene-3-carboxylate (120 mg, 0.2 mmol, 1 eq) in DCM (3 mL) was added TFA (3 mL) and the mixture was stirred at room temperature for 3 hrs. The solution was concentrated to afford the title compound crude (63 mg, 78% yield) as a yellow solid.

LCMS: m/z 404.55 [M+1]⁺.

### Scheme S, Step 3. Synthesis of 1-(9-(4-amino-7-methyl-5-(6-(methylamino)pyridin-3-yl)-7H-pyrrolo[2,3-d]pyrimidin-6-yl)-3-azaspiro[5.5]undec-8-en-3-yl)prop-2-en-1-one

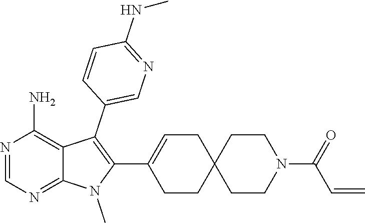 [SMILES: C=CC(=O)N1CCC2(CC=C(c3c(-c4ccc(NC)nc4)c4c(N)ncnc4n3C)CC2)CC1]

### Example 21

To a stirred solution of 7-methyl-5-(6-(methylamino)pyridin-3-yl)-6-(3-azaspiro[5.5]-undec-8-en-9-yl)-7H-pyrrolo[2,3-d]pyrimidin-4-amine (63 mg, 0.15 mmol, 1 eq) in dry CHCl₃ (4 mL) was added TEA (79 mg, 0.78 mmol, 5 eq). A solution of acryloyl chloride (14 mg, 0.15 mmol, 1 eq) in dry CHCl₃ (0.5 mL) was added dropwise at 0° C., and the resulting mixture was stirred at 0° C. for 0.5 hrs under N₂. The reaction mixture was quenched with MeOH. The solvent was removed under reduced pressure. The residue was purified by Prep-HPLC (0.1% TFA in ACN/H₂O) to afford the title compound (28 mg, 39% yield) as a white solid.

LCMS: m/z 458.40 [M+1]⁺.

¹H NMR (400 MHz, CD₃OD) δ 8.34 (s, 1H), 7.89-7.85 (m, 2H), 7.14 (d, J=8.0 Hz, 1H), 6.77 (dd, J=16.0, 12.0 Hz, 1H), 6.17 (d, J=16.0 Hz, 1H), 5.97 (brs, 1H), 5.72 (d, J=12.0 Hz, 1H), 3.80 (s, 3H), 3.72-3.55 (m, 2H), 3.63-3.48 (m, 2H), 3.10 (s, 3H), 2.22-2.11 (m, 4H), 1.71-1.62 (m, 2H), 1.49-1.37 (m, 4H).

### Example 22

### Synthesis of 1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one

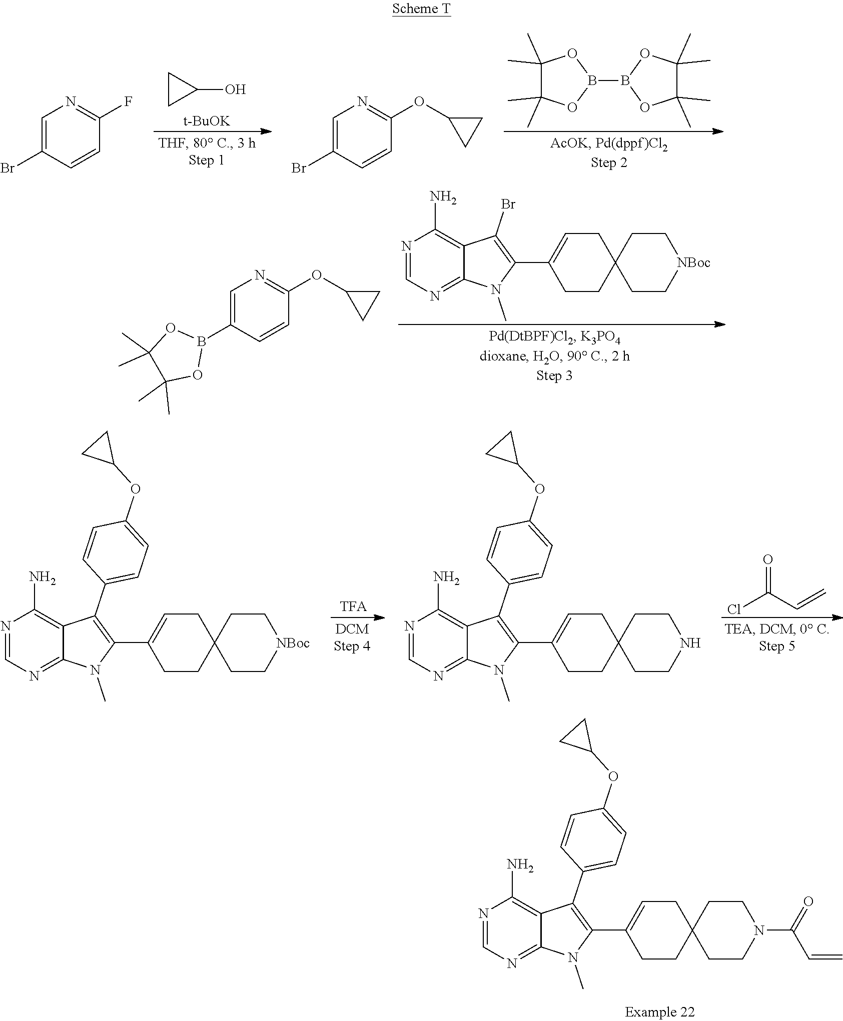

### Scheme T, Step 1. Synthesis of 5-bromo-2-cyclopropoxypyridine

To a solution of 5-bromo-2-fluoropyridine (7.5 g, 42.6 mmol) and cyclopropanol (2.97 g, 51.1 mmol) in THF (10 mL) was added t-BuOK (11.95 g, 106.5 mmol) in portions. The mixture was stirred at 80° C. for 4 h, then poured into water (200 mL) and extracted with EtOAc (100 mL×2). The combined organic layers were washed with brine (50 mL×2), dried over Na₂SO₄, filtered and concentrated under vacuum. The residue was purified by silica gel chromatography (EtOAc in PE=0-10%) to give 5-bromo-2-(cyclopropyloxy)-pyridine (4.8 g, 22.424 mmol, 52.63%) as a yellow oil.

LC-MS m/e: 214.1 (MH⁺).

### Scheme T, Step 2. Synthesis of 2-cyclopropoxy-5-(4,4,5,5-tetramethyl-1,3,2-dioxaborolan-2-yl)pyridine

To a mixture of 5-bromo-2-(cyclopropyloxy)pyridine (4.3 g, 20.088 mmol) and 4,4,5,5-tetramethyl-2-(tetramethyl-1,3,2-dioxaborolan-2-yl)-1,3,2-dioxaborolane (6.246 mL, 24.1 mmol) in dioxane (50 mL) were added Pd(dppf)Cl₂ (0.73 g, 1 mmol) and KOAc (5.91 g, 60.3 mmol) under nitrogen. The mixture was stirred at 100° C. for 3 h, then cooled to room temperature, poured into water (300 mL), and extracted with EtOAc (200 mL×2), the combined organic layers were washed with brine (100 mL×2), concentrated under vacuum. The residue was purified by chromatography on silica gel (eluted with EtOAc:PE from 10% to 30%) to give 2-(cyclopro-pyloxy)-5-(4,4,5,5-tetramethyl-1,3,2-dioxaborolan-2-yl)pyridine (4.6 g, 17.6 mmol, 87.6%) as a yellow oil.

### Scheme T, Step 3. Synthesis of tert-butyl 9-(4-amino-5-(6-cyclopropoxypyridin-3-yl)-7-methyl-7H-pyrrolo[2,3-d]pyrimidin-6-yl)-3-azaspiro[5.5]undec-8-ene-3-carboxylate

To a mixture of 2-methylpropan-2-yl 9-(4-amino-5-bromo-7-methylpyrrolo[2,3-d]pyrimidin-6-yl)-3-azaspiro[5.5]undec-8-ene-3-carboxylate (600 mg, 1.26 mmol) and 2-(cyclo-propyloxy)-5-(4,4,5,5-tetramethyl-1,3,2-dioxaborolan-2-yl)pyridine (493.3 mg, 1.889 mmol) in dioxane (40 mL) were added Pd(DtBPF)Cl₂ (81.3 mg, 0.126 mmol) and K₃PO₄ (802 mg, 3.78 mmol) in H₂O (5 mL). The mixture was stirred at 90° C. for 2 h, then poured into water, and extracted with EA (100 mL×3). The combined organic layers were concentrated under vacuum. The residue was purified by silica gel chromatography eluting with (EtOAc in PE=80-100% then DCM:MeOH=10:1) to give crude tert-butyl 9-(4-amino-5-(6-cyclopropoxypyridin-3-yl)-7-methyl-7H-pyrrolo[2,3-d]pyrimidin-6-yl)-3-azaspiro[5.5]undec-8-ene-3-carboxylate (1 g, 1.884 mmol, 149.63%) as a brown solid.

LC-MS m/e: 531.1 (MH⁺).

### Scheme T, Step 4. Synthesis of 5-(6-cyclopropoxypyridin-3-yl)-7-methyl-6-(3-azaspiro[5.5]undec-8-en-9-yl)-7H-pyrrolo[2,3-d]pyrimidin-4-amine

To a solution of tert-butyl 9-(4-amino-5-(6-cyclopropoxypyridin-3-yl)-7-methyl-7H-pyrrolo[2,3-d]pyrimidin-6-yl)-3-azaspiro[5.5]undec-8-ene-3-carboxylate (400 mg, 0.754 mmol) in DCM (3 mL) was added TFA (1 mL, 13.463 mmol) at 0° C., the mixture was stirred at 25° C. for 1 h. The mixture was concentrated under vacuum to give crude 5-(6-cyclopropoxypyridin-3-yl)-7-methyl-6-(3-azaspiro[5.5]undec-8-en-9-yl)-7H-pyrrolo[2,3-d]pyrimidin-4-amine (320 mg, 0.743 mmol, 98.60%) as a brown solid.

LC-MS m/e: 431.1 (MH⁺).

### Scheme T, Step 5. Synthesis of 1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one

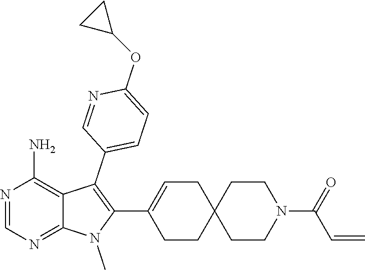 [SMILES: C=CC(=O)N1CCC2(CC=C(c3c(-c4ccc(OC5CC5)nc4)c4c(N)ncnc4n3C)CC2)CC1]

### Example 22

To a mixture of 5-(6-cyclopropoxypyridin-3-yl)-7-methyl-6-(3-azaspiro[5.5]undec-8-en-9-yl)-7H-pyrrolo[2,3-d]pyrimidin-4-amine (320 mg, 0.743 mmol) and TEA (0.207 mL, 1.49 mmol) in DCM (5 mL) was added prop-2-enoyl chloride (0.06 mL, 0.743 mmol) in DCM (2 mL) at 0° C., the mixture was stirred at 0° C. for 1 h. The reaction mixture was quenched by addition of MeOH then concentrated under vacuum. The residue was purified by silica gel chromatography (MeOH in DCM=5% to 10%) to give crude product. The crude product was purified by Prep-HPLC (0.1% FA in ACN/H₂O) to give 1-(9-(4-amino-5-(6-cyclopropoxypyridin-3-yl)-7-methyl-7H-pyrrolo[2,3-d]pyrimidin-6-yl)-3-azaspiro[5.5]-undec-8-en-3-yl)prop-2-en-1-one (73.7 mg, 0.152 mmol, 20.46%) as a yellow solid.

¹H NMR (400 MHz, DMSO-d₆) δ 8.15-8.11 (m, 2H), 7.65 (dd, J=8.5, 2.4 Hz, 1H), 6.91 (d, J=8.4 Hz, 1H), 6.78 (dd, J=16.7, 10.5 Hz, 1H), 6.06 (dd, J=16.7, 2.4 Hz, 1H), 5.88 (s, 2H), 5.78 (s, 1H), 5.64 (dd, J=10.5, 2.4 Hz, 1H), 4.22 (dt, J=9.2, 3.1 Hz, 1H), 3.61 (s, 3H), 3.58-3.40 (m, 4H), 2.08-1.92 (m, 4H), 1.50 (t, J=6.2 Hz, 2H), 1.37-1.11 (m, 4H), 0.82-0.74 (m, 2H), 0.74-0.64 (m, 2H).

LC-MS m/e: 485.3 (MH⁺).

Applying the same or a similar procedure described above, the compounds of Table 1 were prepared.

TABLE 1

| | | m/z | |
|---|---|---|---|
| Example | Structure | (MH⁺) | ¹H NMR |
| 23 | 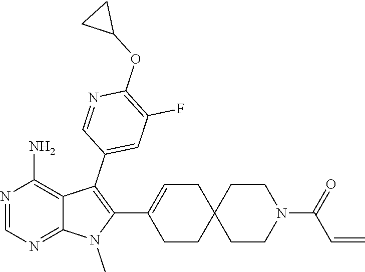 [SMILES: C=CC(=O)N1CCC2(CC=C(c3c(-c4cnc(OC5CC5)c(F)c4)c4c(N)ncnc4n3C)CC2)CC1] | 503.4 | ¹H NMR (400 MHz, DMSO-d₆) δ 8.13 (s, 1H), 7.93 (d, J = 1.8 Hz, 1H), 7.56 (dd, J = 11.4, 1.8 Hz, 1H), 6.77 (dd, J = 16.7, 10.5 Hz, 1H), 6.13-5.90 (m, 3H), 5.79 (s, 1H), 5.64 (dd, J = 10.5, 2.4 Hz, 1H), 4.38 (dd, J = 7.7, 4.5 Hz, 1H), 3.68-3.34 (m, 7H), 2.10-1.92 (m, 4H), 1.51 (t, J = 6.1 Hz, 2H), 1.40-1.20 (m, 4H), 0.87-0.70 (m, 4H). |
| 24 | 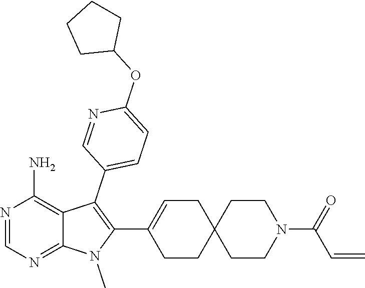 [SMILES: C=CC(=O)N1CCC2(CC=C(c3c(-c4ccc(OC5CCCC5)nc4)c4c(N)ncnc4n3C)CC2)CC1] | 513.3 | ¹HNMR (400 MHz, DMSO-d₆) δ 8.13 (s, 1H), 8.08 (d, J = 2.1 Hz, 1H), 7.60 (dd, J = 8.5, 2.5 Hz, 1H), 6.85-6.70 (m, 2H), 6.06 (dd, J = 16.7, 2.4 Hz, 1H), 5.88 (s, 1H), 5.79 (s, 2H), 5.64 (dd, J = 10.5, 2.4 Hz, 1H), 5.39 (ddd, J = 8.8, 6.1, 3.0 Hz, 1H), 3.61 (s, 3H), 3.58-3.33 (m, 4H), 2.08-1.90 (m, 6H), 1.79-1.68 (m, 4H), 1.67-1.56 (m, 2H), 1.50 (t, J = 6.2 Hz, 2H), 1.30-1.20 (m, 4H). |
| 25 | 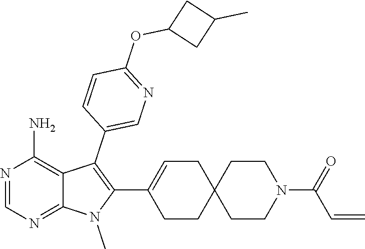 [SMILES: C=CC(=O)N1CCC2(CC=C(c3c(-c4ccc(OC5CC(C)C5)nc4)c4c(N)ncnc4n3C)CC2)CC1] | 513.3 | ¹H NMR (400 MHz, DMSO-d₆) δ 8.12 (s, 1H), 8.05 (d, J = 2.3 Hz, 1H), 7.62 (dd, J = 8.5, 2.4 Hz, 1H), 6.87-6.71 (m, 2H), 6.06 (dd, J = 16.7, 2.4 Hz, 1H), 5.87 (s, 2 H), 5.78 (s, 1H), 5.64 (dd, J = 10.5, 2.4 Hz, 1H), 5.04-4.97 (m, 1H), 3.60 (s, 3H), 3.59-3.35 (m, 4H), 2.69-2.57 (m, 1H), 2.10-1.93 (m, 4H), 1.72-1.60 (m, 2H), 1.50 (t, J = 6.1 Hz, 2H), 1.39-1.15 (m, 6H), 1.11 (d, J = 6.6 Hz, 3H). |
| 26 | 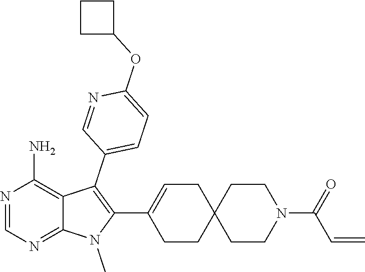 [SMILES: C=CC(=O)N1CCC2(CC=C(c3c(-c4ccc(OC5CCC5)nc4)c4c(N)ncnc4n3C)CC2)CC1] | 499.3 | ¹H NMR (400 MHz, DMSO-d₆) δ 8.13 (s, 1H), 8.06 (d, J = 2.0 Hz, 1H), 7.62 (dd, J = 8.5, 2.5 Hz, 1H), 6.83 (d, J = 8.4 Hz, 1H), 6.77 (dd, J = 16.7, 10.5 Hz, 1H), 6.06 (dd, J = 16.7, 2.4 Hz, 1H), 5.89 (s, 2H), 5.78 (s, 1H), 5.64 (dd, J = 10.5, 2.4 Hz, 1H), 5.17 (p, J = 7.1 Hz, 1H), 3.64-3.34 (m, 7H), 2.46- 2.36 (m, 2H), 2.14-2.02 (m, 4H), 2.00-1.92 (m, 2H), 1.85-1.74 (m, 1H), 1.71-1.60 (m, 1H), 1.50 (t, J = 6.2 Hz, 2H), 1.38-1.21 (m, 4H). |

### Example 27

### Synthesis of 1-(9-(4-amino-5-(5-fluoropyridin-3-yl)-7-methyl-7H-pyrrolo[2,3-d]pyrimidin-6-yl)-3-azaspiro[5.5]undec-8-en-3-yl)prop-2-en-1-one

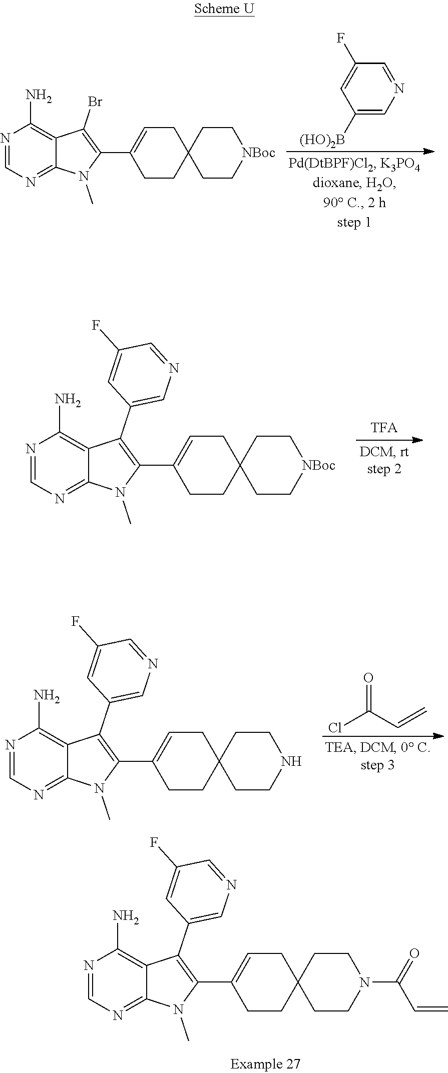

### Scheme U, Step 1. Synthesis of tert-butyl 9-(4-amino-5-(5-fluoropyridin-3-yl)-7-methyl-7H-pyrrolo[2,3-d]pyrimidin-6-yl)-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-3-azaspiro[5.5]undec-8-ene-3-carboxylate (200 mg, 0.42 mmol), (5-fluoropyridin-3-yl)boronic acid (88.73 mg, 0.63 mmol), Pd(DtBPF)Cl₂ (27.37 mg, 0.042 mmol) and K₃PO₄ (267.46 mg, 1.26 mmol) in dioxane (10 mL) and H₂O (1 mL) was degassed with N₂ three times and then stirred at 90° C. for 2 h under N₂. The resulting mixture was diluted with H₂O (20 mL) and extracted with EtOAc (3×15 mL). The combined organic layers were washed with brine dried over anhydrous Na₂SO₄ and filtered. The filtrate was concentrated under reduced pressure. The residue was purified by silica gel column chromatography, eluted with DCM/MeOH (20:1) to afford compound tert-butyl 9-(4-amino-5-(5-fluoropyridin-3-yl)-7-methyl-7H-pyrrolo[2,3-d]pyrimidin-6-yl)-3-azaspiro[5.5]undec-8-ene-3-carboxylate (100 mg, 0.203 mmol, 48.36%) as a yellow solid.

LC-MS m/e: 493.6 (MH⁺).

### Scheme U, Step 2. Synthesis of 5-(5-fluoropyridin-3-yl)-7-methyl-6-(3-azaspiro[5.5]undec-8-en-9-yl)-7H-pyrrolo[2,3-d]pyrimidin-4-amine

To a stirred solution of tert-butyl 9-[4-amino-5-(5-fluoropyridin-3-yl)-7-methyl-7H-pyrrolo[2,3-d]pyrimidin-6-yl]-3-azaspiro[5.5]undec-8-ene-3-carboxylate (45 mg, 0.091 mmol) in DCM (10 mL) was added TFA (0.068 mL, 0.914 mmol) and the mixture was stirred at room temperature for 2 hrs. The solution was concentrated to afford crude 5-(5-fluoropyridin-3-yl)-7-methyl-6-(3-azaspiro[5.5]undec-8-en-9-yl)-7H-pyrrolo[2,3-d]pyrimidin-4-amine (30 mg, 0.076 mmol, 83.67%) as a yellow solid.

LC-MS m/e: 393.4 (MH⁺).

### Scheme U, Step 3. Synthesis of 1-(9-(4-amino-5-(5-fluoropyridin-3-yl)-7-methyl-7H-pyrrolo[2,3-d]pyrimidin-6-yl)-3-azaspiro[5.5]undec-8-en-3-yl)prop-2-en-1-one

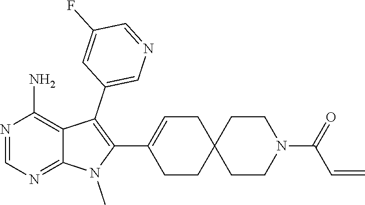 [SMILES: C=CC(=O)N1CCC2(CC=C(c3c(-c4cncc(F)c4)c4c(N)ncnc4n3C)CC2)CC1]

### Example 27

To a stirred solution of 5-(5-fluoropyridin-3-yl)-7-methyl-6-(3-azaspiro[5.5]undec-8-en-9-yl)-7H-pyrrolo[2,3-d]pyrimidin-4-amine (30 mg, 0.076 mmol) in DCM (10 mL) was added TEA (0.011 mL, 0.076 mmol). Then a solution of prop-2-enoyl chloride (0.007 mL, 0.084 mmol) in dry DCM (1 mL) was added dropwise at 0° C. After addition, the resulting mixture was stirred at 0° C. for 0.5 hrs under N₂. The reaction mixture was quenched by addition of MeOH. The solvent was removed under reduced pressure. The residue was purified by Prep-HPLC (0.1% FA in ACN/H₂O) to afford compound 1-(9-(4-amino-5-(5-fluoropyridin-3-yl)-7-methyl-7H-pyrrolo[2,3-d]pyrimidin-6-yl)-3-azaspiro[5.5]undec-8-en-3-yl)prop-2-en-1-one (4.1 mg, 0.009 mmol, 12%) as a white solid.

LC-MS m/e: 447.3 (MH⁺).

¹H NMR (400 MHz, DMSO-d₆) δ 8.51 (s, 1H), 8.38 (s, 1H), 8.16 (s, 1H), 7.60 (d, J=9.4 Hz, 1H), 6.79 (dd, J=16.0, 10.3 Hz, 1H), 6.15-6.00 (m, 3H), 5.81 (s, 1H), 5.64 (d, J=10.3 Hz, 1H), 3.65-3.37 (m, 7H), 2.10-1.96 (m, 4H), 1.55-1.47 (m, 2H), 1.37-1.26 (m, 4H).

Applying the same or a similar procedure described above, the examples of Table 2 were prepared.

TABLE 2

| | | m/z | |
|---|---|---|---|
| Example | Structure | (MH⁺) | ¹H NMR |
| 28 | 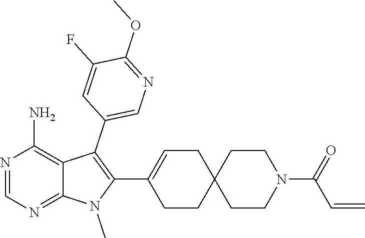 [SMILES: C=CC(=O)N1CCC2(CC=C(c3c(-c4cnc(OC)c(F)c4)c4c(N)ncnc4n3C)CC2)CC1] | 477.3 | ¹H NMR (400 MHz, DMSO-d₆) δ 8.13 (s, 1H), 7.89 (d, J = 1.7 Hz, 1H), 7.56 (dd, J = 11.4, 1.8 Hz, 1H), 6.78 (dd, J = 16.7, 10.5 Hz, 1H), 6.15-6.00 (m, 3H), 5.79 (s, 1H), 5.64 (dd, J = 10.5, 2.4 Hz, 1H), 3.98 (s, 3H), 3.58 (d, J = 24.3 Hz, 7H), 2.08-1.96 (m, 4H), 1.51 (t, J = 6.1 Hz, 2H), 1.40-1.20 (m, 4H). |
| 29 | 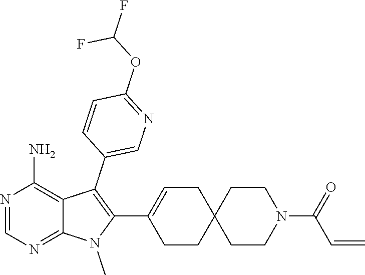 [SMILES: C=CC(=O)N1CCC2(CC=C(c3c(-c4ccc(OC(F)F)nc4)c4c(N)ncnc4n3C)CC2)CC1] | 495.3 | ¹H NMR (400 MHz, DMSO-d₆) δ 8.17 (d, J = 2.3 Hz, 1H), 8.14 (s, 1H), 7.82 (dd, J = 8.4, 2.3 Hz, 1H), 7.74 (t, J = 72.9 Hz, 1H), 7.13 (d, J = 8.4 Hz, 1H), 6.77 (dd, J = 16.7, 10.5 Hz, 1H), 6.07 (dd, J= 16.7, 2.3 Hz, 1H), 5.99 (s, 2H), 5.78 (s, 1H), 5.64 (dd, J = 10.5, 2.3 Hz, 1H), 3.67-3.40 (m, 7H), 2.10-1.90 (m, 4H), 1.50 (t, J = 6.0 Hz, 2H), 1.38-1.18 (m, 4H). |
| 30 | 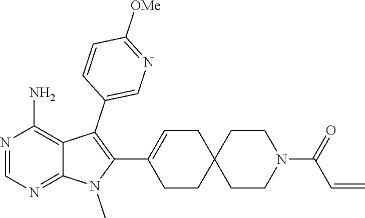 [SMILES: C=CC(=O)N1CCC2(CC=C(c3c(-c4ccc(OC)nc4)c4c(N)ncnc4n3C)CC2)CC1] | 459.2 | ¹H NMR (400 MHz, DMSO-d₆) δ 8.15- 8.07 (m, 2H), 7.64 (dd, J = 8.5, 2.2 Hz, 1H), 6.88 (d, J = 8.5 Hz, 1H), 6.78 (dd, J = 16.7, 10.5 Hz, 1H), 6.06 (dd, J = 16.7, 2.2 Hz, 1H), 5.88 (s, 2H), 5.78 (s, 1H), 5.64 (dd, J = 10.4, 2.2 Hz, 1H), 3.89 (s, 3H), 3.65-3.47 (m, 7H), 2.07- 1.95 (m, 4H), 1.49 (t, J = 6.0 Hz, 2H), 1.38-1.20 (m, 4H). |
| 31 | 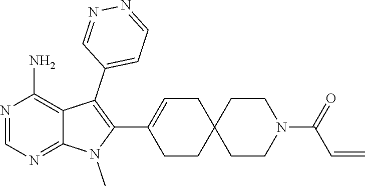 [SMILES: C=CC(=O)N1CCC2(CC=C(c3c(-c4ccnnc4)c4c(N)ncnc4n3C)CC2)CC1] | 430.2 | ¹H NMR (400 MHz, DMSO-d₆) δ 9.20- 9.04 (m, 2H), 8.19 (s, 1H), 7.53 (s, 1H), 6.81 (dd, J = 16.9, 10.2 Hz, 1H), 6.30- 6.22 (m, 2H), 6.08 (d, J = 16.6 Hz, 1H), 5.81 (s, 1H), 5.65 (d, J = 10.2 Hz, 1H), 3.63 (s, 3H), 3.61-3.47(m, 4H), 2.10- 1.98 (m, 4H), 1.60-1.50 (m, 2H), 1.42- 1.24 (m, 4H). |
| 32 | 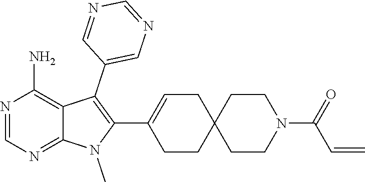 [SMILES: C=CC(=O)N1CCC2(CC=C(c3c(-c4cncnc4)c4c(N)ncnc4n3C)CC2)CC1] | 430.2 | ¹H NMR (400 MHz, DMSO-d₆) δ 9.10 (s, 1H), 8.70 (s, 2H), 8.16 (s, 1H), 6.79 (dd, J= 16.7, 10.5 Hz, 1H), 6.25-6.15 (m, 2H), 6.07 (dd, J = 16.7, 2.4 Hz, 1H), 5.80 (s, 1H), 5.64 (dd, J = 10.5, 2.4 Hz, 1H), 3.63 (s, 3H), 3.60-3.53 (m, 2H), 3.48-3.42 (m, 2H), 2.06-2.00 (m, 4H), 1.57-1.48 (m, 2H), 1.36-1.25 (m, 4H). |

### Example 33

### Synthesis of 1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one

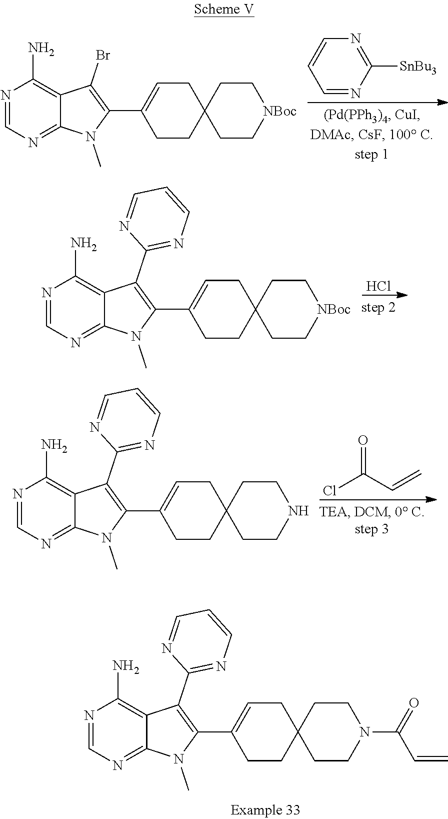

### Scheme V, Step 1. Synthesis of tert-butyl 9-(4-amino-7-methyl-5-(pyrimidin-2-yl)-7H-pyrrolo[2,3-d]pyrimidin-6-yl)-3-azaspiro[5.5]undec-8-ene-3-carboxylate

To a solution of tert-butyl 9-(4-amino-5-bromo-7-methyl-7H-pyrrolo[2,3-d]pyrimidin-6-yl)-3-azaspiro[5.5]undec-8-ene-3-carboxylate (300 mg, 0.63 mmol) in DMAc (10 mL) were added copper iodide (12 mg, 0.063 mmol), CsF (191 mg, 1.26 mmol), Pd(PPh₃)₄ (73 mg, 0.063 mmol) and 2-(tributylstannyl)pyrimidine (350 mg, 0.95 mmol). The suspension was degassed under vacuum and purged with N₂ several times. The resulting mixture was stirred at 90° C. for 16 hrs. under N₂. After completion, the reaction mixture was cooled to room temperature, diluted with water and extracted with EtOAc. The combined organic layers were washed with brine, dried over anhydrous Na₂SO₄, and concentrated under reduced pressure. The residue was purified by flash column chromatography on silica gel to afford tert-butyl 9-(4-amino-7-methyl-5-(pyrimidin-2-yl)-7H-pyrrolo[2,3-d]pyrimidin-6-yl)-3-azaspiro[5.5]undec-8-ene-3-carboxylate (100 mg, 0.21 mmol, 33%) as a brown solid.

LC-MS m/e: 476 (MH⁺).

### Scheme V, Step 2. Synthesis of 7-methyl-5-(pyrimidin-2-yl)-6-(3-azaspiro[5.5]undec-8-en-9-yl)-7H-pyrrolo[2,3-d]pyrimidin-4-amine

To a solution of tert-butyl 9-(4-amino-7-methyl-5-(pyrimidin-2-yl)-7H-pyrrolo[2,3-d]pyrimidin-6-yl)-3-azaspiro[5.5]undec-8-ene-3-carboxylate (100 mg, 0.21 mmol) in MeOH (2 mL) was added 4M HCl in dioxane (3 mL). The resulting mixture was stirred at room temperature for 1 hr and then concentrated under reduced pressure to afford 7-methyl-5-(pyrimidin-2-yl)-6-(3-azaspiro[5.5]undec-8-en-9-yl)-7H-pyrrolo[2,3-d]pyrimidin-4-amine (78 mg, 0.21 mmol, quant.) as a solid, which is used directly in next step without further purification.

LC-MS m/e: 376 (MH⁺).

### Scheme V, Step 3. Synthesis of 1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one

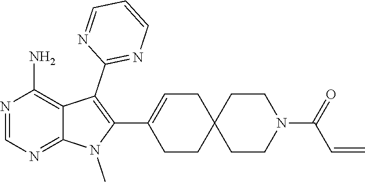 [SMILES: C=CC(=O)N1CCC2(CC=C(c3c(-c4ncccn4)c4c(N)ncnc4n3C)CC2)CC1]

### Example 33

To a suspension of 7-methyl-5-(pyrimidin-2-yl)-6-(3-azaspiro[5.5]undec-8-en-9-yl)-7H-pyrrolo[2,3-d]pyrimidin-4-amine (78 mg, 0.21 mmol) and triethylamine (0.22 mL, 1.6 mmol) in DCM (10 mL) was added prop-2-enoyl chloride (22 mg, 0.25 mmol) in DCM (2 mL) at 0° C. The reaction mixture was stirred at 0° C. for 30 min and then concentrated under reduced pressure. The residue was purified by prep-HPLC to afford 1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one (20 mg, 46 μmol, 22%) as a white solid.

LC-MS m/e: 430.2 (MH⁺).

¹H NMR (400 MHz, DMSO-d₆) δ 8.77 (d, J=5.0 Hz, 2H), 8.10 (s, 1H), 7.24 (t, J=5.0 Hz, 1H), 6.89-6.82 (m, 3H), 6.11 (dd, J=16.8, 2.4 Hz, 1H), 5.69-5.64 (m, 2H), 3.69-3.66 (m, 2H), 3.59 (s, 3H), 3.55-3.52 (m, 2H), 2.35-2.25 (m, 2H), 2.15-2.05 (m, 2H), 1.71-1.68 (m, 2H), 1.59-1.55 (m, 4H).

Applying the same or a similar procedure described above, the examples of Table 3 were prepared.

TABLE 3

| | | m/z | |
|---|---|---|---|
| Example | Structure | (MH⁺) | ¹H NMR |
| 34 | 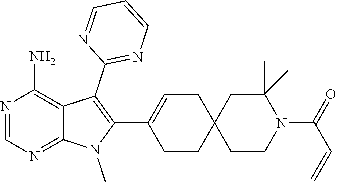 [SMILES: C=CC(=O)N1CCC2(CC=C(c3c(-c4ncccn4)c4c(N)ncnc4n3C)CC2)CC1(C)C] | 458.2 | ¹H NMR (400 MHz, DMSO-d₆) δ 9.33 (s, 1H), 8.78 (d, J = 4.9 Hz, 2H), 8.10 (s, 1H), 7.25 (t, J = 4.9 Hz, 1H), 7.15 (s, 1H), 6.70 (dd, J = 16.8, 10.5 Hz, 1H), 5.98 (dd, J = 16.8, 2.3 Hz, 1H), 5.63- 5.60 (m, 1H), 5.57 (dd, J = 10.5, 2.3 Hz, 1H), 3.60 (s, 3H), 3.58-3.45 (m, 2H), 2.45-2.05 (m, 4H), 1.90-1.64 (m, 6H), 1.55 (s, 3H), 1.51 (s, 3H). |
| 35 | 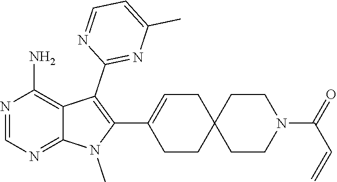 [SMILES: C=CC(=O)N1CCC2(CC=C(c3c(-c4nccc(C)n4)c4c(N)ncnc4n3C)CC2)CC1] | 444.5 | ¹H NMR (400 MHz, DMSO-d₆) δ 8.61 (d, J =4 Hz, 1H), 8.09 (s, 1H), 7.13 (d, J = 4 Hz, 1H), 6.85 (dd, J = 16.7, 10.5 Hz, 1H), 6.10 (dd, J = 16.7, 2.4 Hz, 1H), 5.67 (dd, J = 10.5, 2.4 Hz, 1H), 5.64-5.59 (m, 1H), 3.67-3.66 (m, 2H), 3.59 (s, 3H), 3.56-3.48 (m, 2H), 2.47(s, 3H), 2.35-2.25 (m, 2H), 2.15-2.07 (m, 2H), 1.71 (t, J = 6.1 Hz, 2H), 1.62-1.48 (m, 4H). |
| 59 | 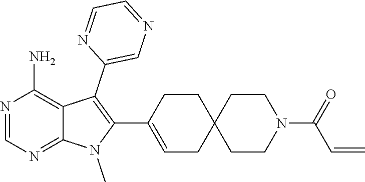 [SMILES: C=CC(=O)N1CCC2(CC=C(c3c(-c4cnccn4)c4c(N)ncnc4n3C)CC2)CC1] | 430.5 | 1H NMR (400 MHz, DMSO-d₆) δ 8.84 (d, J = <4 Hz, 1H), 8.66 (dd, 1 H), 8.45 (d, J = 4 Hz, 1H), 8.14 (S, 1H), 7.59 (hump, 2H), 6.82 (dd, J = 16, 4 Hz, 1H), 6.09 (dd, J = 16, <4 Hz, 1H), 5.98 (br. S, 1H), 5.66 (dd, J = 12, 4 Hz, 1H), 3.63 (S, 3H), 3.66-3.49 (m, 4H), 2.18 (m, 4H), 1.66 (m, 2H), 1.47 (m, 4H). |
| 60 | 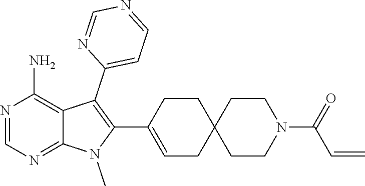 [SMILES: C=CC(=O)N1CCC2(CC=C(c3c(-c4ccncn4)c4c(N)ncnc4n3C)CC2)CC1] | 430.5 | 1H NMR (400 MHz, DMSO-d₆) δ 9.16 (d, J = 4 Hz, 1H), 8 8.76 (d, J = 4 Hz, 1H), δ 8.14 (S, 1H), 8 7.66 (dd, J = 8, 4 Hz, 1H), δ 6.83 (dd, J = 12, 8 Hz, 1H), 6.19 (d, J =16, 4 Hz, 1H), δ 6.01 (s, 1H), δ 5.67 (d, J = 12, 4 Hz, 1H), 3.67-3.54 (m, 4H), 3.61 (S, 3H), 2.21 (m, 2H), 2.13 (m, 2H), 1.70 (m, 2H), 1.48 (m, 4H) |

### Preparation 3

### Synthesis of tert-butyl 9-(4-amino-5-bromo-7-(difluoromethyl)-7H-pyrrolo[2,3-d]pyrimidin-6-yl)-3-azaspiro[5.5]undec-8-ene-3-carboxylate

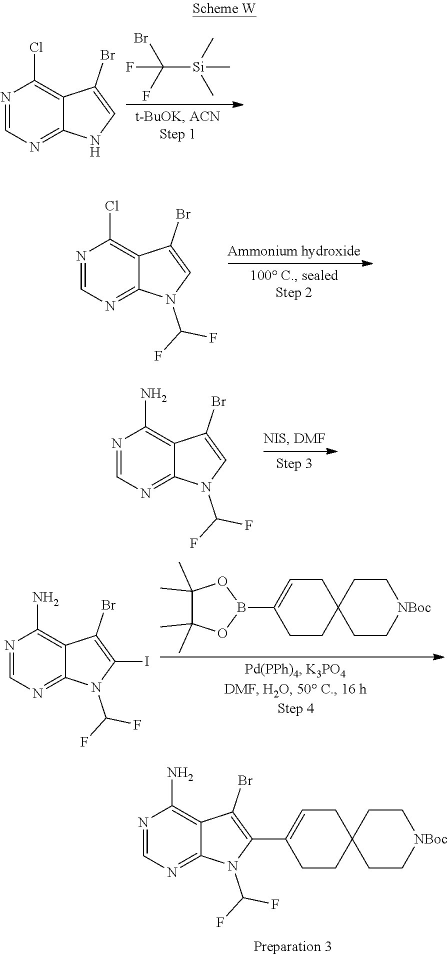

### Scheme W, Step 1. Synthesis of 5-bromo-4-chloro-7-(difluoromethyl)-7H-pyrrolo[2,3-d]pyrimidine

To a stirred mixture of 5-bromo-4-chloro-7H-pyrrolo[2,3-d]pyrimidine (1.8 g, 7.74 mmol, 1 eq) and t-BuOK (1.7 g, 15.48 mmol, 2 eq) in ACN (30 mL) was added (bromodifluoromethyl)trimethylsilane (1.7 g, 8.51 mmol, 1.1 eq) under N₂ at 0° C. The resulting mixture was stirred at 35° C. overnight and then poured into water (100 mL). The precipitated solid was collected by filtration, washed with water, and dried under reduced pressure to afford the title compound (1.1 g, 45.3%) as a yellow solid.

LC-MS m/e: 282 (MH⁺).

### Scheme W, Step 2. Synthesis of 5-bromo-7-(difluoromethyl)-7H-pyrrolo[2,3-d]pyrimidin-4-amine

A mixture of 5-bromo-4-chloro-7-(difluoromethyl)-7H-pyrrolo[2,3-d]pyrimidine (1.1 g, 3.89 mmol, 1 eq) in NH₃—H₂O (20 mL) was stirred at 100° C. for 24 hrs. in a sealed tube. The mixture was cooled to room temperature and diluted with water. The solid is filtered, washed with water and dried under reduced pressure to afford the title compound (1 g, 98%) as a white solid.

LC-MS m/e: 263 (MH⁺).

### Scheme W, Step 3. Synthesis of 5-bromo-7-(difluoromethyl)-6-iodo-7H-pyrrolo[2,3-d]pyrimidin-4-amine

To a solution of 5-bromo-7-(difluoromethyl)-7H-pyrrolo[2,3-d]pyrimidin-4-amine (1 g, 3.8 mmol, 1 eq) in DMF (10 mL) was added NIS (1.28 g, 5.7 mmol, 1.5 eq) and CF₃COOH (0.5 mL) at 0° C. under N₂, and the resulting mixture was stirred at room temperature for 2 hrs. A saturated Na₂SO₃ solution (10 mL) was added and the mixture was poured into water (30 mL). The precipitated solid was collected by filtration, washed with water, and dried under reduced pressure to afford the title compound (0.9 g, 60.8%) as a yellow solid.

LC-MS m/e: 389 (MH⁺).

### Scheme W, Step 4. Synthesis of tert-butyl 9-(4-amino-5-bromo-7-(difluoromethyl)-7H-pyrrolo[2,3-d]pyrimidin-6-yl)-3-azaspiro[5.5]undec-8-ene-3-carboxylate

A mixture of 5-bromo-7-(difluoromethyl)-6-iodo-7H-pyrrolo[2,3-d]pyrimidin-4-amine (0.4 g, 1 mmol, 1 eq), tert-butyl 9-(4,4,5,5-tetramethyl-1,3,2-dioxaborolan-2-yl)-3-azaspiro[5.5]-undec-8-ene-3-carboxylate (0.5 g, 1.34 mmol, 1.3 eq), Pd(PPh₃)₄ (115.6 mg, 0.1 mmol, 0.1 eq) and K₃PO₄ (658.2 mg, 3.09 mmol, 3 eq) in DMF (10 mL) and H₂O (2 mL) was degassed with N₂ three times and then stirred at 55° C. for 24 h under N₂. The resulting mixture was diluted with H₂O (50 mL) and extracted with EtOAc (3×40 mL). The combined organic layers were washed with brine (4×30 mL), dried over anhydrous Na₂SO₄ and filtered. The filtrate was concentrated under reduced pressure. The residue was purified by silica gel column chromatography, eluting with DCM/MeOH (97:3) to afford the title compound (400 mg, 75.9% yield) as a yellow solid.

LC-MS m/e: 512 (MH⁺).

### Example 36

### Synthesis of 1-(9-(4-amino-5-(4-cyclopropoxyphenyl)-7-(difluoromethyl)-7H-pyrrolo[2,3-d]pyrimidin-6-yl)-3-azaspiro[5.5]undec-8-en-3-yl)prop-2-en-1-one

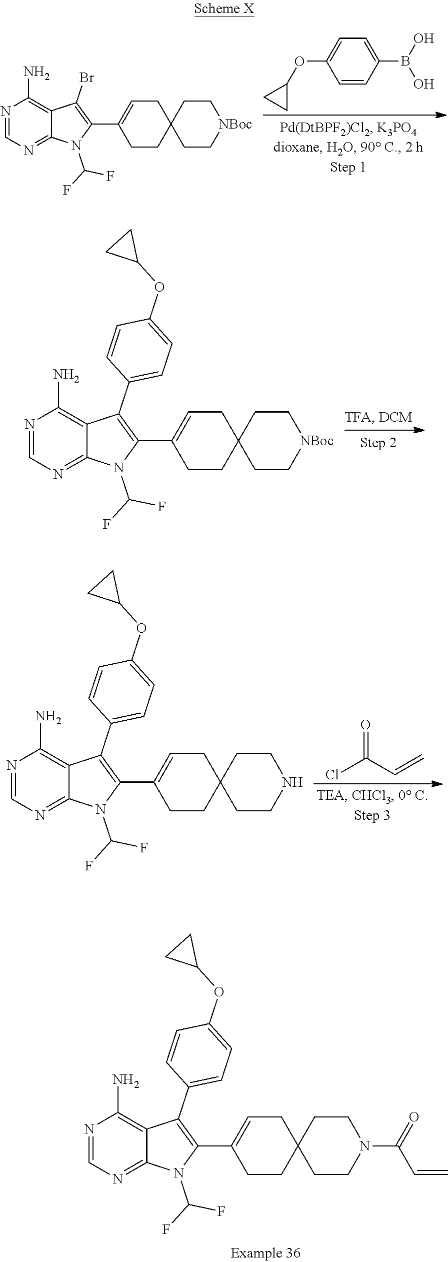

### Scheme X, Step 1. Synthesis of tert-butyl 9-(4-amino-5-(4-cyclopropoxyphenyl)-7-(difluoro-methyl)-7H-pyrrolo[2,3-d]pyrimidin-6-yl)-3-azaspiro[5.5]undec-8-ene-3-carboxylate

A mixture of tert-butyl 9-(4-amino-5-bromo-7-(difluoromethyl)-7H-pyrrolo[2,3-d]-pyrimidin-6-yl)-3-azaspiro[5.5]undec-8-ene-3-carboxylate (400 mg, 0.78 mmol, 1 eq), 2-(4-cyclopropoxyphenyl)-4,4,5,5-tetramethyl-1,3,2-dioxaborolane (304.6 mg, 1.17 mmol, 1.5 eq), Pd(DtBPF)Cl₂ (50.4 mg, 0.078 mmol, 0.1 eq) and K₃PO₄ (498.4 mg, 2.34 mmol, 3 eq) in dioxane (10 mL) and H₂O (1 mL) was degassed with N₂ three times and then stirred at 90° C. for 3 h under N₂. The resulting mixture was diluted with H₂O (20 mL) and extracted with EtOAc (3×15 mL). The combined organic layers were washed with brine (4×15 mL), dried over anhydrous Na₂SO₄ and filtered. The filtrate was concentrated under reduced pressure. The residue was purified by silica gel column chromatography, eluting with DCM/MeOH (97:3) to afford the title compound (300 mg, 67.9% yield) as a yellow solid.

LC-MS m/e: 566 (MH⁺).

### Scheme X, Step 2. Synthesis of 5-(4-cyclopropoxyphenyl)-7-(difluoromethyl)-6-(3-azaspiro[5.5]undec-8-en-9-yl)-7H-pyrrolo[2,3-d]pyrimidin-4-amine

To a stirred solution of tert-butyl 9-(4-amino-5-(4-cyclopropoxyphenyl)-7-(difluoromethyl)-7H-pyrrolo[2,3-d]pyrimidin-6-yl)-3-azaspiro[5.5]undec-8-ene-3-carboxylate (300 mg, 0.53 mmol, 1 eq) in DCM (3 mL) was added TFA (1 mL) and the mixture was stirred at room temperature for 1 hr. The solution was concentrated to afford the title compound (150 mg, 60.8% yield) as a yellow solid.

LC-MS m/e: 466 (MH⁺).

### Scheme X, Step 3. Synthesis of 1-(9-(4-amino-5-(4-cyclopropoxyphenyl)-7-(difluoromethyl)-7H-pyrrolo[2,3-d]pyrimidin-6-yl)-3-azaspiro[5.5]undec-8-en-3-yl)prop-2-en-1-one (Example 36)

To a stirred solution of 5-(4-cyclopropoxyphenyl)-7-(difluoromethyl)-6-(3-azaspiro-[5.5]undec-8-en-9-yl)-7H-pyrrolo[2,3-d]pyrimidin-4-amine (150 mg, 0.289 mmol) in DCM (20 mL) was added TEA (0.5 mL). Then a solution of acryloyl chloride (26.1 mg, 0.289 mmol) in dry DCM (10 mL) was added dropwise at 0° C. After addition, the resulting mixture was stirred at 0° C. for 0.5 hrs. under N₂. The reaction mixture was quenched by addition of NH₄Cl and extracted with DCM (3×15 mL). The combined organic layers were dried over anhydrous Na₂SO₄ and filtered. The filtrate was concentrated under reduced pressure. The residue was purified by Prep-HPLC (0.1% FA in ACN/H₂O) to afford the title compound (20 mg, 11.9% yield) as a white solid.

¹H NMR (400 MHz, DMSO-d₆) δ 8.21 (s, 1H), 7.92 (t, J=58.3 Hz, 1H), 7.31 (d, J=8.6 Hz, 2H), 7.14 (d, J=8.6 Hz, 2H), 6.77 (dd, J=16.7, 10.5 Hz, 1H), 6.06 (dd, J=16.7, 2.4 Hz, 1H), 5.85 (s, 1H), 5.63 (dd, J=10.5, 2.4 Hz, 1H), 3.89 (dt, J=9.0, 3.0 Hz, 1H), 3.62-3.49 (m, 2H), 3.45-3.32 (m, 2H), 2.04-1.90 (m, J=18.4 Hz, 4H), 1.45 (t, J=6.0 Hz, 2H), 1.33-1.10 (m, 4H), 0.88-0.78 (m, 2H), 0.72-0.64 (m, 2H).

LC-MS m/e: 520.3 (MH⁺).

Applying the same or a similar procedure described above, the example of Table 4 was prepared.

TABLE 4

| | | m/z | |
|---|---|---|---|
| Example | Structure | (MH⁺) | ¹H NMR |
| 37 | 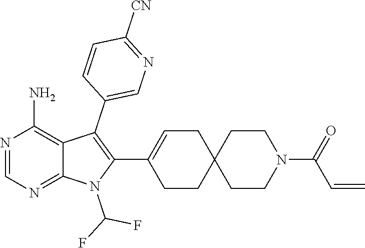 [SMILES: C=CC(=O)N1CCC2(CC=C(c3c(-c4ccc(C#N)nc4)c4c(N)ncnc4n3C(F)F)CC2)CC1] | 490.3 | ¹H NMR (400 MHz, DMSO-d₆) δ 8.69 (d, J = 1.5 Hz, 1H), 8.25 (s, 1H), 8.14-7.80 (m, 3H), 6.79 (dd, J = 16.7, 10.5 Hz, 1H), 6.50 (s, 2H), 6.13-6.02 (m, 1H), 5.86 (s, 1H), 5.64 (dd, J = 10.5, 2.4 Hz, 1H), 3.65-3.38 (m, 4H), 2.05- 1.92 (m, 4H), 1.52-1.42 (m, 2H), 1.30-1.18 (m, 4H). |

### Example 38

### Synthesis of 1-(9-(4-amino-5-(4-(methoxy-d3)phenyl)-7-methyl-7H-pyrrolo[2,3-d]pyrimidin-6-yl)-8-fluoro-3-azaspiro[5.5]undec-8-en-3-yl)prop-2-en-1-one

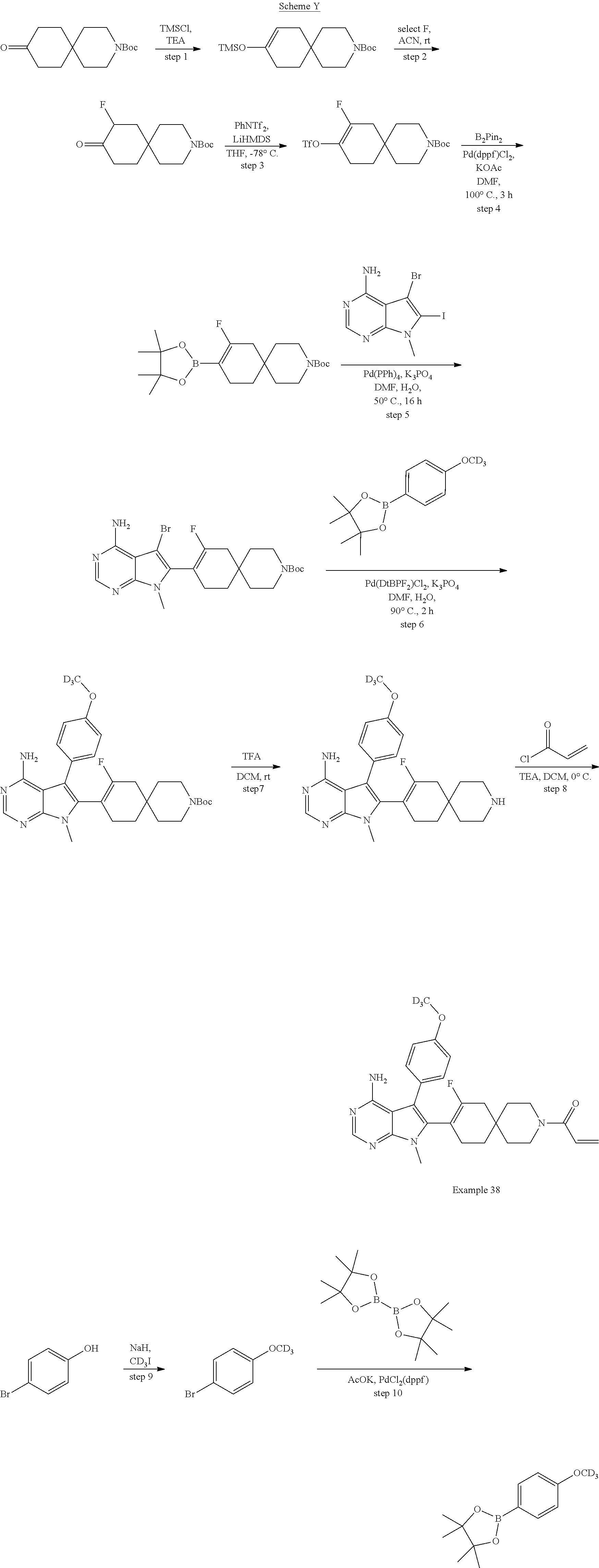

### Scheme Y, Step 1 Synthesis of tert-butyl 9-((trimethylsilyl)oxy)-3-azaspiro[5.5]undec-8-ene-3-carboxylate

Tert-butyl 9-oxo-3-azaspiro[5.5]undecane-3-carboxylate (2 g, 7.48 mmol) was dissolved in DMF (30 mL) and placed under an inert atmosphere. Chlorotrimethylsilane (1.91 mL, 15 mmol) was then added dropwise, followed by TEA (3.12 mL, 22.4 mmol). The resulting solution was heated to 80° C. and stirred overnight, after which time the reaction mixture was cooled to room temperature and diluted with DCM and sat. NaHCO₃ solution. The aqueous layer was extracted with DCM (20 mL×3). The combined organic extracts were washed with H₂O and filtered through a phase separator. Solvents were removed. The crude residue was purified by column chromatography (1-10% EtOAc in hexanes) to give the title compound tert-butyl 9-[(trimethylsilyl)oxy]-3-azaspiro[5.5]undec-8-ene-3-carboxylate (2.5 g, 7.363 mmol, 98.43%).

### Scheme Y, Step 2. Synthesis of tert-butyl 8-fluoro-9-oxo-3-azaspiro[5.5]undecane-3-carboxylate

Tert-butyl 9-[(trimethylsilyl)oxy]-3-azaspiro[5.5]undec-8-ene-3-carboxylate (2.5 g, 7.4 mmol) was dissolved in CH₃CN (30 mL) and cooled to 0° C. 1-Chloromethyl-4-fluoro-1,4-diazoniabicyclo[2.2.2]octane bis(tetrafluoroborate) (Select-F, 3.91 g, 11 mmol, 1.5 eq.) was then added. The resulting solution was warmed to r.t. and stirred for 3 h. The reaction mixture was diluted with EtOAc and brine, and the aqueous layer was extracted with EtOAc. The combined organic extracts were dried with MgSO₄ and filtered. Removal of the solvent gave the title compound tert-butyl 8-fluoro-9-oxo-3-azaspiro[5.5]undecane-3-carboxylate (850 mg, 2.98 mmol, 40.5%) as a colorless oil.

### Scheme Y, Step 3. Synthesis of tert-butyl 8-fluoro-9-(((trifluoromethyl)sulfonyl)oxy)-3-azaspiro[5.5]undec-8-ene-3-carboxylate

To a stirred solution of tert-butyl 8-fluoro-9-oxo-3-azaspiro[5.5]undecane-3-carboxylate (700 mg, 2.45 mmol) in dry THF (15 mL) was added dropwise a solution of LiHMDS (2.94 mL, 2.94 mmol) at −78° C. under N₂ and the mixture was stirred at −78° C. for 1 hr. Then a solution of 1,1,1-trifluoro-N-phenyl-N-trifluoromethanesulfonylmethane-sulfonamide (1314 mg, 3.68 mmol) in dry THF (15 mL) was added dropwise and the mixture is warmed slowly to room temperature and stirred for 16 hrs. The reaction mixture was quenched with sat. NH₄Cl aqueous solution, extracted with EtOAc (3×100 mL). The combined organic layers were dried over anhydrous Na₂SO₄ and filtered. The filtrate was concentrated under reduced pressure. The residue was purified by silica gel column chromatography, eluted with hexanes/EtOAc (10:1-8:1) to afford tert-butyl 8-fluoro-9-(trifluoro-methanesulfonyloxy)-3-azaspiro[5.5]undec-8-ene-3-carboxylate (595 mg, 1.425 mmol, 58.11%) as a colorless oil.

### Scheme Y, Step 4. Synthesis of tert-butyl 8-fluoro-9-(4,4,5,5-tetramethyl-1,3,2-dioxaborolan-2-yl)-3-azaspiro[5.5]undec-8-ene-3-carboxylate

To a stirred solution of tert-butyl 8-fluoro-9-(((trifluoromethyl)sulfonyl)oxy)-3-azaspiro[5.5]undec-8-ene-3-carboxylate (595 mg, 1.425 mmol) in DMF (10 mL) were added 4,4,5,5-tetra-methyl-2-(tetramethyl-1,3,2-dioxaborolan-2-yl)-1,3,2-dioxaborolane (0.739 mL, 2.85 mmol), potassium acetate (419.7 mg, 4.28 mmol) and Pd(dppf)Cl₂ (31.3 mg, 0.043 mmol). The reaction was stirred at 100° C. for 3 hrs. under N₂, diluted with EtOAc (200 mL) and H₂O (200 mL), extracted with EtOAc (3×100 mL). The combined organic layers were washed with brine (5×100 mL), dried over anhydrous Na₂SO₄ and filtered. The filtrate was concentrated under reduced pressure. The residue was purified by silica gel column chromatography, eluted with hexanes/EtOAc (10:1-8:1) to afford tert-butyl 8-fluoro-9-(tetra-methyl-1,3,2-dioxaborolan-2-yl)-3-azaspiro-[5.5]undec-8-ene-3-carboxylate (400 mg, 1 mmol, 71%) as a colorless oil.

### Scheme Y, Step 5. Synthesis of tert-butyl 9-(4-amino-5-bromo-7-methyl-7H-pyrrolo[2,3-d]pyrimidin-6-yl)-8-fluoro-3-azaspiro[5.5]undec-8-ene-3-carboxylate

A mixture of compound 5-bromo-6-iodo-7-methyl-7H-pyrrolo[2,3-d]pyrimidin-4-amine (600 mg, 1.7 mmol), tert-butyl 8-fluoro-9-(tetramethyl-1,3,2-dioxaborolan-2-yl)-3-azaspiro[5.5]undec-8-ene-3-carboxylate (1008 mg, 2.55 mmol), Pd(PPh₃)₄ (196.43 mg, 0.17 mmol) and K₃PO₄ (1082.52 mg, 5.1 mmol) in DMF (16 mL) and H₂O (2 mL) was degassed with N₂ three times and then stirred at 50° C. for 16 h under N₂. The resulting mixture was diluted with H₂O (50 mL) and extracted with EtOAc (3×40 mL). The combined organic layers were washed with brine (4×30 mL), dried over anhydrous Na₂SO₄ and filtered. The filtrate was concentrated under reduced pressure. The residue was purified by silica gel column chromatography, eluted with DCM/MeOH (70:1) to afford tert-butyl 9-(4-amino-5-bromo-7-methyl-7H-pyrrolo[2,3-d]pyrimidin-6-yl)-8-fluoro-3-azaspiro[5.5]undec-8-ene-3-carboxylate (600 mg, 1.214 mmol, 71.39%) as a yellow solid.

LC-MS m/e: 496.4 (M+1)+.

### Scheme Y, Step 6. Synthesis of tert-butyl 9-(4-amino-5-(4-(methoxy-d3)phenyl)-7-methyl-7H-pyrrolo[2,3-d]pyrimidin-6-yl)-8-fluoro-3-azaspiro[5.5]undec-8-ene-3-carboxylate

A mixture of tert-butyl 9-(4-amino-5-bromo-7-methyl-7H-pyrrolo[2,3-d]pyrimidin-6-yl)-8-fluoro-3-azaspiro[5.5]undec-8-ene-3-carboxylate (800 mg, 1.679 mmol), 2-(4-(methoxy-d3)-phenyl)-4,4,5,5-tetramethyl-1,3,2-dioxaborolane (95.92 mg, 0.405 mmol), 1,1′-Bis (di-t-butyl-phosphino)ferrocene palladium dichloride, (41.04 mg, 0.063 mmol) and K₃PO₄ (128.8 mg, 0.607 mmol) in DMF (5 mL) and H₂O (0.5 mL) was degassed with N₂ three times and then stirred at 90° C. for 3 h under N₂. The resulting mixture was diluted with H₂O (20 mL) and extracted with EtOAc (3×15 mL). The combined organic layers were washed with brine (4×15 mL), dried over anhydrous Na₂SO₄ and filtered. The filtrate was concentrated under reduced pressure. The residue was purified by silica gel column chromatography, eluted with PE:EA 0-100% to afford tert-butyl 9-(4-amino-5-(4-(methoxy-d3)phenyl)-7-methyl-7H-pyrrolo[2,3-d]pyrimidin-6-yl)-8-fluoro-3-azaspiro[5.5]undec-8-ene-3-carboxylate (236 mg, 0.466 mmol, 27.7%) as a yellow solid.

LC-MS m/e: 525.6 (M+1)*.

### Scheme Y, Step 7. Synthesis of 6-(8-fluoro-3-azaspiro[5.5]undec-8-en-9-yl)-5-(4-(methoxy-d3)phenyl)-7-methyl-7H-pyrrolo[2,3-d]pyrimidin-4-amine

To a stirred solution of tert-butyl 9-(4-amino-5-(4-(methoxy-d3)phenyl)-7-methyl-7H-pyrrolo[2,3-d]pyrimidin-6-yl)-8-fluoro-3-azaspiro[5.5]undec-8-ene-3-carboxylate (80 mg, 0.152 mmol) in DCM (10 mL) was added TFA (0.034 mL, 0.456 mmol) and the mixture was stirred at room temperature for 2 hrs. The solution was concentrated to afford crude 6-(8-fluoro-3-azaspiro[5.5]undec-8-en-9-yl)-5-(4-(methoxy-d3)phenyl)-7-methyl-7H-pyrrolo[2,3-d]pyrimidin-4-amine (50 mg, 0.118 mmol, 77.24%) as a yellow solid.

LC-MS m/e: 425.6 (M+1)+.

### Scheme Y, Step 8. Synthesis of 1-(9-(4-amino-5-(4-(methoxy-d3)phenyl)-7-methyl-7H-pyrrolo[2,3-d]pyrimidin-6-yl)-8-fluoro-3-azaspiro[5.5]undec-8-en-3-yl)prop-2-en-1-one

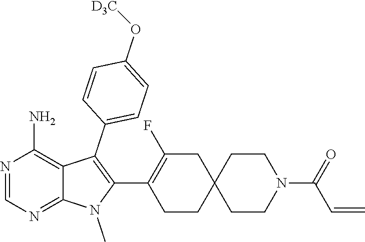 [SMILES: [2H]C([2H])([2H])Oc1ccc(-c2c(C3=C(F)CC4(CC3)CCN(C(=O)C=C)CC4)n(C)c3ncnc(N)c23)cc1]

### Example 38

To a stirred solution of 6-(8-fluoro-3-azaspiro[5.5]undec-8-en-9-yl)-5-(4-(methoxy-d3)phenyl)-7-methyl-7H-pyrrolo[2,3-d]pyrimidin-4-amine (60 mg, 0.141 mmol) in DCM (10 mL) was added TEA (0.059 mL, 0.424 mmol). A solution of prop-2-enoyl chloride (0.014 mL, 0.17 mmol) in dry DCM (1 mL) was added dropwise at 0° C. The resulting mixture was stirred at 0° C. for 0.5 hrs under N₂ and then quenched with MeOH. The solvent was removed under reduced pressure. The residue was purified by Prep-HPLC (0.1% TFA in ACN/H₂O) to afford compound 1-(9-(4-amino-5-(4-(methoxy-d3)phenyl)-7-methyl-7H-pyrrolo[2,3-d]pyrimidin-6-yl)-8-fluoro-3-azaspiro[5.5]undec-8-en-3-yl)prop-2-en-1-one (40 mg, 0.084 mmol, 59.14%) as a white solid.

LC-MS m/e: 479.8 (MH⁺).

¹H NMR (400 MHz, DMSO-d₆) δ 8.20 (s, 1H), 7.25 (d, J=8.5 Hz, 2H), 7.03 (d, J=8.5 Hz, 2H), 6.79 (dd, J=16.7, 10.5 Hz, 1H), 6.23 (s, 2H), 6.07 (dd, J=16.7, 2.3 Hz, 1H), 5.65 (dd, J=10.5, 2.3 Hz, 1H), 3.68-3.42 (m, 7H), 2.29 (s, 2H), 2.15-1.70 (m, 2H), 1.54-1.22 (m, 6H).

### Scheme Y, Step 9. Synthesis of 1-bromo-4-(methoxy-d₃)benzene

A solution of 4-bromophenol (2 g, 11.6 mmol) in THF (30 mL) was cooled to 0° C., sodium hydride 60% dispersion in mineral oil (0.924 g, 23.1 mmol) was added portion wise. After addition, the mixture was stirred at rt for 30 min, then CD₃I (1.08 mL, 17.3 mmol) was added. The mixture was stirred at rt for 2 hr. The reaction mixture was quenched by addition of sodium sulfite solution, then extracted with EtOAc (3×20 mL). The combined organic layers were dried, filtered, and concentrated in vacuo to afford 1-bromo-4-(methoxy-d3)benzene (2 g, 10.5 mmol, 91%) as a colorless oil.

### Scheme Y, Step 10. Synthesis of 2-(4-(methoxy-d₃)phenyl)-4,4,5,5-tetramethyl-1,3,2-dioxaborolane

A mixture of 1-bromo-4-(methoxy-d₃)benzene (2.21 g, 11.6 mmol), bis(pinacolato)diboron (3.54 g, 14 mmol), 1,1′-bis(diphenylphosphino)ferrocene-palladium(II)dichloride dichloromethane complex (0.47 g, 0.58 mmol), and potassium acetate (2.85 g, 29 mmol) in dry dioxane (50 mL) is purged three times with argon and placed under vacuum three times. The mixture was stirred at 90° C. for 21 h, the reaction mixture was cooled to rt then filtered through a filter pad. The organic solvent was removed under reduced pressure, and the black residue 2-(4-(methoxy-d3)phenyl)-4,4,5,5-tetramethyl-1,3,2-dioxaborolane (2 g, 8.435 mmol, 72.46%) is obtained and used directly in the next step without purification.

### Example 39

### Synthesis of 1-(9-(4-amino-5-(6-methoxypyridin-3-yl)-7-methyl-7H-pyrrolo[2,3-d]pyrimidin-6-yl)-2-methyl-3-azaspiro[5.5]undec-8-en-3-yl)prop-2-en-1-one

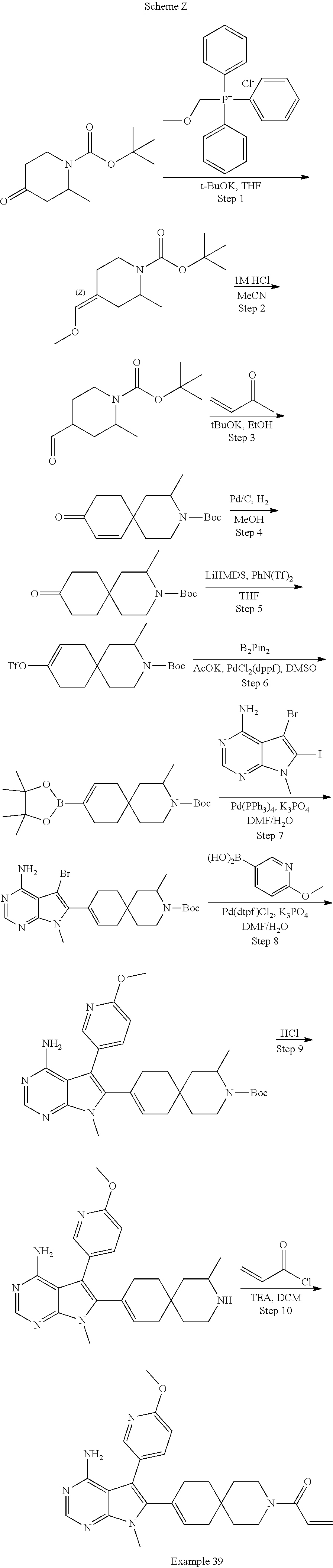

### Scheme Z, Step 1. Synthesis of tert-butyl (Z)-4-(methoxymethylene)-2-methylpiperidine-1-carboxylate

To a stirred solution of chloro(methoxymethyl)triphenyl-λ5-phosphane (12 g, 35 mmol) in anhydrous THF (150 mL) was added tert-BuOK (3.94 g, 35.17 mmol) in portions under atmosphere of N₂ at 4° C. A vivid orange color was noted and the reaction was left as such for 1 h. 2-Methylpropan-2-yl 2-methyl-4-oxohexahydropyridine-1-carboxylate (5 g, 23.4 mmol) was added slowly at a temperature below 10° C. The resultant mixture was then allowed to warm to room temperature and agitated overnight. It was quenched with water and extracted with EtOAc. The combined extracts were dried with anhydrous Na₂SO₄, filtered and concentrated. The residue was purified by silica gel column chromatography to afford tert-butyl (Z)-4-(methoxymethylene)-2-methylpiperidine-1-carboxylate (5 g, 20.7 mmol, 88.3%) as colorless oil.

LC-MS m/e: 242.1 (MH⁺).

### Scheme Z, Step 2. Synthesis of tert-butyl 4-formyl-2-methylpiperidine-1-carboxylate

To a solution of tert-butyl (Z)-4-(methoxymethylene)-2-methylpiperidine-1-carboxylate (5.4 g, 22.38 mmol) in MeCN (550 mL) was added 1M HCl (22.4 mL) at 0° C. The reaction was then allowed to stir at RT for 2 hours. The mixture was quenched with NaHCO₃ and extracted with EtOAc. The extracts were dried with anhydrous Na₂SO₄, filtered, and concentrated to afford tert-butyl 4-formyl-2-methylpiperidine-1-carboxylate (4 g, 19.4 mmol, 86.5%) as a colorless oil.

LC-MS m/e: 228.1 (MH⁺).

### Scheme Z, Step 3. Synthesis of tert-butyl 2-methyl-9-oxo-3-azaspiro[5.5]undec-7-ene-3-carboxylate

To a solution of tert-butyl 4-formyl-2-methylpiperidine-1-carboxylate (6.3 g, 28 mmol) in EtOH (100 mL) was added but-3-en-2-one (4.5 mL, 55.4 mmol), followed by K tert-BuOK (9.3 g, 83 mmol) in portions at R.T. The mixture was stirred at R.T for 1 h. The reaction mixture was concentrated, acidified with 1 N HCl to pH=7 and extracted with EA. The combined organic layers were dried, concentrated and purified by chromatography to afford tert-butyl 2-methyl-9-oxo-3-azaspiro[5.5]undec-7-ene-3-carboxylate (3 g, 10.7 mmol, 38.7%) as yellow oil.

LC-MS m/e: 224 [M+H−56]⁺.

### Scheme Z, Step 4. Synthesis of tert-butyl 2-methyl-9-oxo-3-azaspiro[5.5]undecane-3 carboxylate

To a solution of tert-butyl 2-methyl-9-oxo-3-azaspiro[5.5]undec-7-ene-3-carboxylate (4 g, 14.32 mmol) in EtOH (50 mL) was added Pd/C 10% (3 g). The mixture was stirred at 50 psi of H₂ for 1 h. The mixture was filtered through a filter pad. The filtrate was concentrated to afford tert-butyl 2-methyl-9-oxo-3-azaspiro[5.5]undecane-3-carboxylate (3.5 g, 12.4 mmol, 86.9%) as colorless oil.

LC-MS m/e: 226.1 [M−55]⁺.

### Scheme Z, Step 5. Synthesis of tert-butyl 2-methyl-9-(((trifluoromethyl)sulfonyl)oxy)-3-azaspiro[5.5]undec-8-ene-3-carboxylate

To a solution of tert-butyl 2-methyl-9-oxo-3-azaspiro[5.5]undecane-3-carboxylate (3 g, 10.7 mmol) in THF (50 mL) was added LiHMDS (21.3 mL) drop wise under N₂ atmosphere at −60° C. and stirred for 1 h. N-[Dioxo(trifluoromethyl)-λ6-sulfanyl]-1,1,1-trifluoro-N-phenylmethanesulfonamide (7.62 g, 21.3 mmol) in THF was added drop wise at −60° C. and the mixture is then allowed to warm slowly to R.T and stirred overnight. The mixture was quenched with NH₄Cl solution and extracted with EtOAc. The combined organic layers were dried, concentrated and purified by chromatography to afford tert-butyl 2-methyl-9-(((trifluoromethyl)sulfonyl)oxy)-3-azaspiro[5.5]undec-8-ene-3-carboxylate (1.8 g, 4.35 mmol, 40.84%) as colorless oil.

### Scheme Z, Step 6. Synthesis of 2-methylpropan-2-yl 2-methyl-9-(4,4,5,5-tetramethyl-1,3,2-dioxaborolan-2-yl)-3-azaspiro[5.5]undec-8-ene-3-carboxylate

To a solution of tert-butyl 2-methyl-9-(((trifluoromethyl)sulfonyl)oxy)-3-azaspiro-[5.5]undec-8-ene-3-carboxylate (1 g, 2.42 mmol) in DMSO (10 mL) were added potassium acetate (0.47 g, 4.84 mmol), 4,4,5,5-tetramethyl-2-(4,4,5,5-tetramethyl-1,3,2-dioxaborolan-2-yl)-1,3,2-dioxaborolane (0.92 g, 3.63 mmol), followed by Pd(dppf)Cl₂ (0.18 g, 0.242 mmol). The mixture is purged with N₂ for three times and stirred at 90° C. for 3 h. The mixture was cooled to rt, diluted with water, and extracted with EtOAc. The combined organic layers were dried, concentrated and purified by chromatography to afford 2-methylpropan-2-yl 2-methyl-9-(4,4,5,5-tetramethyl-1,3,2-dioxaborolan-2-yl)-3-azaspiro[5.5]undec-8-ene-3-carboxylate (600 mg, 1.53 mmol, 63.4%) as colorless oil.

LC-MS m/e: 336.1 [M−55]⁺.

### Scheme Z, Step 7. Synthesis of 2-methylpropan-2-yl 9-(4-amino-5-bromo-7-methylpyrrolo[2,3-d]pyrimidin-6-yl)-2-methyl-3-azaspiro[5.5]undec-8-ene-3-carboxylate

To a solution of 5-bromo-6-iodo-7-methylpyrrolo[2,3-d]pyrimidin-4-amine (400 mg, 1.13 mmol) in DMF (20 mL) were added 2-methylpropan-2-yl 2-methyl-9-(4,4,5,5-tetramethyl-1,3,2-dioxaborolan-2-yl)-3-azaspiro[5.5]undec-8-ene-3-carboxylate (720 mg, 1.47 mmol), tripotassium phosphate (481 mg, 2.27 mmol) in water (2 mL), followed by Pd(PPh₃)₄ (131 mg, 0.113 mmol). The mixture was purged with N₂ for three times and stirred at 50° C. for 16 h. It was cooled to rt, diluted with water, and extracted with EtOAc. The combined organic layers were dried, concentrated, and purified by chromatography to afford 2-methylpropan-2-yl 9-(4-amino-5-bromo-7-methylpyrrolo[2,3-d]pyrimidin-6-yl)-2-methyl-3-azaspiro[5.5]undec-8-ene-3-carboxylate (350 mg, 0.714 mmol, 62.5%) as colorless oil.

LCMS: m/z 490, 492 (MH⁺).

### Scheme Z, Step 8. Synthesis of tert-butyl 9-(4-amino-5-(6-methoxypyridin-3-yl)-7-methyl-7H-pyrrolo[2,3-d]pyrimidin-6-yl)-2-methyl-3-azaspiro[5.5]undec-8-ene-3-carboxylate

To a solution of 2-methylpropan-2-yl 9-(4-amino-5-bromo-7-methylpyrrolo[2,3-d]pyrimidin-6-yl)-2-methyl-3-azaspiro[5.5]undec-8-ene-3-carboxylate (350 mg, 0.714 mmol) in DMF (20 mL) was added (6-methoxypyridin-3-yl)boranediol (218 mg, 1.43 mmol), K₃PO₄ (303 mg, 1.43 mmol), followed by Pd(dtpf)Cl₂ (46 mg, 0.07 mmol). The mixture was purged with N₂ three times and stirred at 90° C. for 2 hrs. The mixture was cooled, diluted with water, and extracted with EtOAc. The combined organic layers were dried, concentrated and purified by chromatography to afford tert-butyl 9-(4-amino-5-(6-methoxypyridin-3-yl)-7-methyl-7H-pyrrolo[2,3-d]pyrimidin-6-yl)-2-methyl-3-azaspiro[5.5]undec-8-ene-3-carboxylate (230 mg, 0.443 mmol, 62.2%) as a brown solid.

LC-MS m/e: 519 (MH⁺).

### Scheme Z, Step 9. Synthesis of 5-(6-methoxypyridin-3-yl)-7-methyl-6-(2-methyl-3-azaspiro[5.5]undec-8-en-9-yl)-7H-pyrrolo[2,3-d]pyrimidin-4-amine

To a solution of tert-butyl 9-(4-amino-5-(6-methoxypyridin-3-yl)-7-methyl-7H-pyrrolo[2,3-d]pyrimidin-6-yl)-2-methyl-3-azaspiro[5.5]undec-8-ene-3-carboxylate (200 mg, 0.386 mmol) in MeOH (1 mL) was added HCl/dioxane (5 mL). The mixture was stirred at R.T. for 1 h and then concentrated to afford 5-(6-methoxypyridin-3-yl)-7-methyl-6-(2-methyl-3-azaspiro[5.5]undec-8-en-9-yl)-7H-pyrrolo[2,3-d]pyrimidin-4-amine (180 mg, 0.43 mmol) as a brown solid.

LC-MS m/e: 419 (MH⁺).

### Scheme Z, Step 10. Synthesis of 1-(9-(4-amino-5-(6-methoxypyridin-3-yl)-7-methyl-7H-pyrrolo[2,3-d]pyrimidin-6-yl)-2-methyl-3-azaspiro[5.5]undec-8-en-3-yl)prop-2-en-1-one

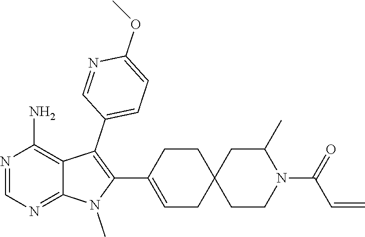 [SMILES: C=CC(=O)N1CCC2(CC=C(c3c(-c4ccc(OC)nc4)c4c(N)ncnc4n3C)CC2)CC1C]

### Example 39

To a suspension of tert-butyl 9-(4-amino-5-(6-methoxypyridin-3-yl)-7-methyl-7H-pyrrolo[2,3-d]pyrimidin-6-yl)-2-methyl-3-azaspiro[5.5]undec-8-ene-3-carboxylate (160 mg, 0.31 mmol) in DCM (10 mL) was added Et₃N (0.2 mL, 1.53 mmol), followed by addition of prop-2-enoyl chloride (36 mg, 0.4 mmol) in DCM (2 mL) at 0° C. After stirring at 0° C. for 30 min, the mixture was quenched with MeOH, concentrated, and purified by Prep-HPLC to afford 1-(9-(4-amino-5-(6-methoxypyridin-3-yl)-7-methyl-7H-pyrrolo[2,3-d]pyri-midin-6-yl)-2-methyl-3-azaspiro[5.5]undec-8-en-3-yl)prop-2-en-1-one (30 mg, 0.063 mmol, 20.8%) as a white solid.

LC-MS m/e: 473.5 (MH⁺).

¹H NMR (400 MHz, DMSO-d₆) δ 8.16 (s, 1H), 8.08 (d, J=2.2 Hz, 1H), 7.64-7.61 (m, 1H), 6.88 (d, J=8.5 Hz, 1H), 6.69-6.65 (m, 1H), 6.07 (s, 2H), 6.03 (m, 1H), 5.74 (d, J=4.0 Hz, 1H), 5.66-5.62 (m, 1H), 4.36-4.07 (m, 3H), 3.87 (s, 3H), 3.61 (s, 3H), 2.09-2.03 (m, 1H), 1.98-1.84 (m, 3H), 1.56-1.54 (m, 1H), 1.43-1.16 (m, 5H), 1.14-1.07 (m, 3H).

Example 39 can be separated to further give the following peak (optically isomeric compound): Instrument: Waters Thar 80™ preparative SFC, Column: ChiralPak™ AD, 250×21.2 mm I.D., 5 μm, Mobile phase: A for CO₂ and B for 0.1% 7 mol/L NH3 in MeOH, Gradient: B 40%, Flow rate: 40 mL/min, Back pressure: 100 bar, Column temperature: 35° C., Wavelength: 220 nm, Cycle-time: 30 min, Eluted time: 3 H) to afford peak 1 (retention time: 2.473 min), peak 2 (retention time: 2.911 min) and peak 3 (retention time: 4.377 min).

Applying the same or a similar procedure described above, the example of Table 5 was prepared.

TABLE 5

| | | m/z | |
|---|---|---|---|
| Example | Structure | (MH⁺) | ¹H NMR |
| 40 | 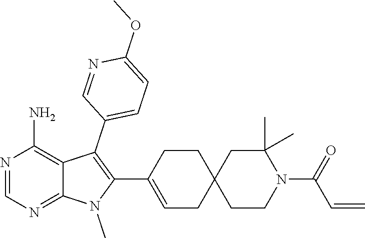 [SMILES: C=CC(=O)N1CCC2(CC=C(c3c(-c4ccc(OC)nc4)c4c(N)ncnc4n3C)CC2)CC1(C)C] | 487.8 | ¹H NMR (400 MHz, CDCl₃) δ 8.28 (s, 1H), 8.19 (d, J = 2.0 Hz, 1H), 7.59 (dd, J = 8.5, 2.4 Hz, 1H), 6.82 (d, J = 8.3 Hz, 1H), 6.48 (dd, J = 16.9, 10.5 Hz, 1H), 6.14 (dd, J = 16.9, 1.8 Hz, 1H), 5.78 (s, 1H), 5.57 (dd, J = 10.5, 1.8 Hz, 1H), 5.45 (s, 2H), 3.97 (s, 3H), 3.72 (s, 3H), 3.46 (t, J = 5.7 Hz, 2H), 2.18- 1.97 (m, 4H), 1.65-1.54 (m, 4H), 1.52 (s, 6H), 1.47 (s, 2H). |

### Example 41

### Synthesis of 1-(3-(4-amino-5-(4-fluorophenyl)-7-methyl-7H-pyrrolo[2,3-d]pyrimidin-6-yl)-1-oxa-9-azaspiro[5.5]undec-3-en-9-yl)prop-2-en-1-one

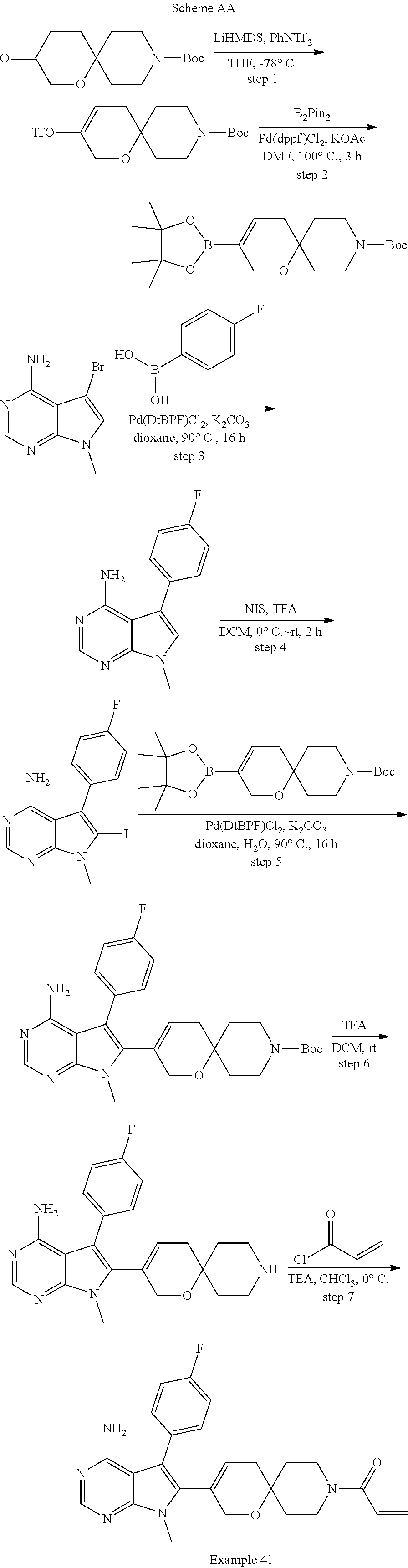

### Scheme AA, Step 1. Synthesis of tert-butyl 3-(((trifluoromethyl)sulfonyl)oxy)-1-oxa-9-azaspiro[5.5]undec-3-ene-9-carboxylate

To a stirred solution of compound tert-butyl 3-oxo-1-oxa-9-azaspiro[5.5]undecane-9-carboxylate (300 mg, 1.11 mmol, 1 eq) in dry THF (8 mL) was added dropwise the solution of LiHMDS (1.4 mL, 1.34 mmol, 1.2 eq, 1M in THF) at −78° C. under N₂ and the mixture was stirred at −78° C. for 1 hr. Then 1,1,1-trifluoro-N-phenyl-N-((trifluoromethyl)sulfonyl)-methanesulfonamide (436 mg, 3.3 mmol, 1.1 eq) was added, and the mixture was warmed to room temperature slowly and stirred for 16 hrs. The reaction mixture was quenched with sat. NH₄Cl aqueous solution, extracted with EtOAc (3×10 mL). The combined organic layers were dried over anhydrous Na₂SO₄ and filtered. The filtrate was concentrated under reduced pressure. The residue was purified by silica gel column chromatography, eluted with hexanes/EtOAc (10:1) to afford the title compound (380 mg, 85% yield) as a colorless oil.

### Scheme AA, Step 2. Synthesis of tert-butyl 3-(4,4,5,5-tetramethyl-1,3,2-dioxaborolan-2-yl)-1-oxa-9-azaspiro[5.5]undec-3-ene-9-carboxylate

To a stirred solution of compound tert-butyl 3-(((trifluoromethyl)sulfonyl)oxy)-1-oxa-9-azaspiro[5.5]undec-3-ene-9-carboxylate (380 mg, 0.95 mmol, 1 eq) in DMF (5 mL) was added B₂Pin₂ (265 mg, 1.05 mmol, 1.1 eq), KOAc (280 mg, 2.85 mmol, 3 eq) and Pd(dppf)Cl₂ (77.5 mg, 0.095 mmol, 0.1 eq). The reaction was stirred at 100° C. for 3 hrs. under N₂. The reaction mixture was concentrated in vacuo, diluted with EtOAc (10 mL) and filtered through a filter pad. The residue was purified by silica gel column chromatography, eluted with hexanes/EtOAc (10:1) to afford the title compound (280 mg, 77% yield) as a colorless oil.

### Scheme AA, Step 3. Synthesis of 5-(4-fluorophenyl)-7-methyl-7H-pyrrolo[2,3-d]pyrimidin-4-amine

A mixture of 5-bromo-7-methyl-7H-pyrrolo[2,3-d]pyrimidin-4-amine (10 g, 44 mmol, 1 eq), compound (4-fluorophenyl)boronic acid (9.2 g, 66 mmol, 1.5 eq), K₂CO₃ (18.2 g, 132 mmol, 3 eq) and Pd(DtBPF)Cl₂ (1.43 g, 2.2 mmol, 0.05 eq) in dioxane (150 mL) and H₂O (15 mL) was stirred at 90° C. for 16 h under N₂. The resulting mixture was diluted with H₂O (200 mL) and extracted with EtOAc (3×100 mL). The combined organic layers were dried over anhydrous Na₂SO₄ and filtered. The filtrate was concentrated under reduced pressure. The residue was purified by silica gel column chromatography, eluting with DCM/MeOH (70:1) to afford the title compound (9.5 g, 89%) as a brown solid.

LCMS: m/z 243.15 [M+1]⁺.

### Scheme AA, Step 4. Synthesis of 5-(4-fluorophenyl)-6-iodo-7-methyl-7H-pyrrolo[2,3-d]pyrimidin-4-amine

To a solution of 5-(4-fluorophenyl)-7-methyl-7H-pyrrolo[2,3-d]pyrimidin-4-amine (9.5 g, 39.2 mmol, 1 eq) in DCM (120 mL) were added TFA (17.9 g, 157 mmol, 4 eq) and NIS (10.6 g, 47.1 mmol, 1.2 eq) in one portion under N₂ at 0° C. and stirred at room temperature for 2 hrs. The mixture was poured into a sat NaHCO₃ aqueous solution (30 mL) and a 10% Na₂SO₃ aqueous solution (20 mL). The resulting mixture was extracted with DCM (3×30 mL), dried over anhydrous Na₂SO₄ and filtered. The filtrate was concentrated under reduced pressure. The residue was purified by silica gel column chromatography, eluting with DCM/MeOH (70:1-50:1) to afford the title compound (11.2 g, 78%) as a white solid.

LCMS: m/z 369.40 [M+1]⁺.

### Scheme AA, Step 5. Synthesis of tert-butyl 3-(4-amino-5-(4-fluorophenyl)-7-methyl-7H-pyrrolo[2,3-d]pyrimidin-6-yl)-1-oxa-9-azaspiro[5.5]undec-3-ene-9-carboxylate

A mixture of 5-(4-fluorophenyl)-6-iodo-7-methyl-7H-pyrrolo[2,3-d]pyrimidin-4-amine (141 mg, 0.38 mmol, 1.1 eq), compound tert-butyl 3-(4,4,5,5-tetramethyl-1,3,2-dioxa-borolan-2-yl)-1-oxa-9-azaspiro[5.5]undec-3-ene-9-carboxylate (280 mg, 0.35 mmol, 1 eq), Pd(DtBPF)Cl₂ (26 mg, 0.04 mmol, 0.1 eq) and K₂CO₃ (145 mg, 1.05 mmol, 3 eq) in dioxane (5 mL)/H₂O (0.5 mL) was stirred at 90° C. for 16 h under nitrogen atmosphere. The resulting mixture was dried over anhydrous Na₂SO₄ and filtered. The filtrate was concentrated under reduced pressure. The residue was purified by silica gel column chromatography, eluted with CH₂Cl₂/MeOH (60:1) to afford the title compound (90 mg, 48% yield) as a yellow solid.

LCMS: m/z 494.15 [M+1]⁺.

### Scheme AA, Step 6. Synthesis of 5-(4-fluorophenyl)-7-methyl-6-(1-oxa-9-azaspiro[5.5]undec-3-en-3-yl)-7H-pyrrolo[2,3-d]pyrimidin-4-amine

To a mixture of tert-butyl 3-(4-amino-5-(4-fluorophenyl)-7-methyl-7H-pyrrolo[2,3-d]pyrimidin-6-yl)-1-oxa-9-azaspiro[5.5]undec-3-ene-9-carboxylate (90 mg, 0.18 mmol, 1 eq) in CH₂Cl₂ (4 mL) was added TFA (2 mL) under N₂. The reaction mixture was stirred at 0° C. for 1 hr and then concentrated. The resulting residue was purified by TLC with DCM/MeOH(10:1) to afford the title compound (36 mg, 51% yield) as a yellow solid.

LCMS: m/z 394.20 [M+1]⁺.

### Scheme AA, Step 7. Synthesis of 1-(3-(4-amino-5-(4-fluorophenyl)-7-methyl-7H-pyrrolo[2,3-d]pyrimidin-6-yl)-1-oxa-9-azaspiro[5.5]undec-3-en-9-yl)prop-2-en-1-one

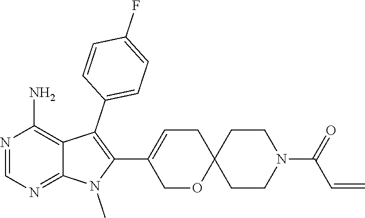 [SMILES: C=CC(=O)N1CCC2(CC=C(c3c(-c4ccc(F)cc4)c4c(N)ncnc4n3C)CO2)CC1]

### Example 41

To a mixture of 5-(4-fluorophenyl)-7-methyl-6-(1-oxa-9-azaspiro[5.5]undec-3-en-3-yl)-7H-pyrrolo[2,3-d]pyrimidin-4-amine (36 mg, 0.091 mmol, 1 eq) in dry CHCl₃ (10 mL) was added TEA (27.6 mg, 0.273 mmol, 3 eq). A solution of acryloyl chloride (8.5 mg, 0.093 mmol, 1 eq) in dry CHCl₃ (1 mL) was added dropwise at 0° C. and stirred for 1 h under N₂. The reaction mixture was quenched with H₂O, extracted with DCM (3×10 mL), dried over anhydrous Na₂SO₄ and filtered. The filtrate was concentrated under reduced pressure. The resulting residue was purified by TLC with DCM/MeOH(12:1) to afford the title compound (18 mg, 44.3% yield).

LCMS: m/z 448.25 [M+1]⁺.

¹H NMR (400 MHz, CD₃OD) δ 8.12 (s, 1H), 7.46-7.38 (m, 2H), 7.20 (t, J=8.0 Hz, 2H), 6.74 (dd, J=16.0, 10.0 Hz, 1H), 6.15 (dd, J=16.0, 2.0 Hz, 1H), 6.04 (s, 1H), 5.70 (dd, J=10.0, 2.0 Hz, 1H), 4.19 (d, J=12.0 Hz, 1H), 3.97-3.88 (m, 2H), 3.79 (d, J=12.0 Hz, 1H), 3.71 (s, 3H), 3.35 (t, J=12.0 Hz, 1H), 2.99 (t, J=12.0 Hz, 1H), 2.18-2.11 (m, 2H), 1.77 (d, J=12.0 Hz, 2H), 1.55-1.38 (m, 2H).

### Example 42

### Synthesis of 1-(9-(4-amino-7-methyl-5-(5-(trifluoromethyl)pyrimidin-2-yl)-7H-pyrrolo[2,3-d]pyrimidin-6-yl)-3-azaspiro[5.5]undec-8-en-3-yl)prop-2-en-1-one

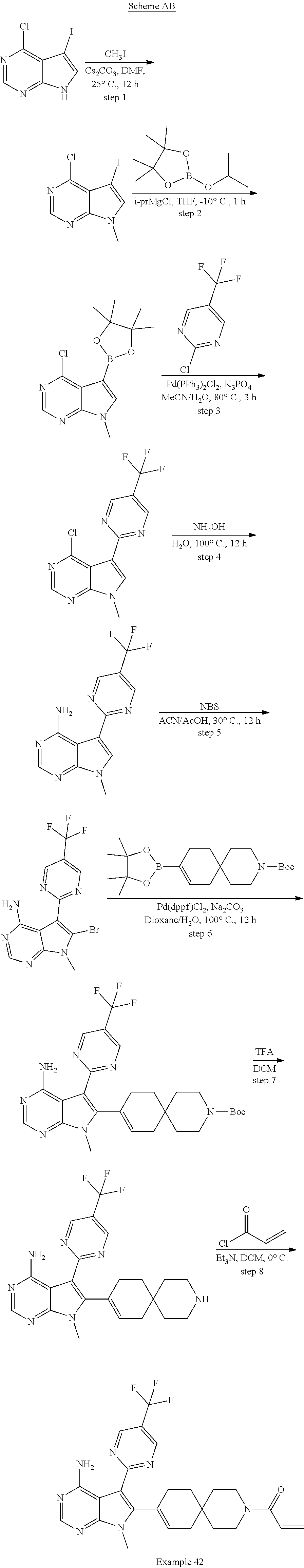

### Scheme AB, Step 1. Synthesis of 4-chloro-5-iodo-7-methyl-7H-pyrrolo[2,3-d]pyrimidine

To a solution of 4-chloro-5-iodo-7H-pyrrolo[2,3-d]pyrimidine (20 g, 71.6 mmol) and CH₃I (6.68 mL, 107 mmol) in DMF (500 mL) was added Cs₂CO₃ (46.6 g, 143 mmol). The reaction mixture was stirred at 25° C. for 12 h. The reaction mixture was poured into iced water. The precipitate was collected by filtration, washed with water and ethanol successively. The filter cake was dried under vacuum to give 4-chloro-5-iodo-7-methyl-7H-pyrrolo[2,3-d]pyrimidine (20.5 g, 69.84 mmol, 97% yield) without further purification.

¹H NMR (400 MHz, DMSO-d6): 8.65 (s, 1H), 7.98 (s, 1H), 3.83 (s, 3H).

### Scheme AB, Step 2. 4-chloro-7-methyl-5-(4,4,5,5-tetramethyl-1,3,2-dioxaborolan-2-yl)-7H-pyrrolo[2,3-d]pyrimidine

To a solution of 4-chloro-5-iodo-7-methyl-7H-pyrrolo[2,3-d]pyrimidine (1.16 g, 3.96 mmol) in dry THF (50 mL) at −10° C. was slowly added i-PrMgCl in THF (2 N, 2.4 mL, 4.8 mmol). After 30 min, 1-isopropoxy-3,3,4,4-tetramethyl-borolane (1.2 mL, 5.94 mmol) was added and stirred for 2 hours. The reaction mixture was diluted with saturated aqueous NH₄CI and extracted with EtOAc. The combined organic phases were washed with brine, dried over Na₂SO₄ and evaporated. The crude was triturated with petroleum ether to afford 4-Chloro-7-methyl-5-(4,4,5,5-tetramethyl-[1,3,2]dioxaborolan-2-yl)-7H-pyrrolo[2,3-d]pyrimidine (850 mg, 2.89 mol, 73% yield) as a white solid.

¹H NMR (400 MHz, DMSO-d6): 8.65 (s, 1H), 8.04 (s, 1H), 3.84 (s, 3H), 1.30 (s, 12H).

### Scheme AB Step 3. Synthesis of 4-chloro-7-methyl-5-(5-(trifluoromethyl)pyrimidin-2-yl)-7H-pyrrolo[2,3-d]pyrimidine

To a solution of 4-chloro-7-methyl-5-(4,4,5,5-tetramethyl-1,3,2-dioxaborolan-2-yl)-7H-pyrrolo[2,3-d]pyrimidine (500 mg, 1.71 mmol) in MeCN (6 mL) and H₂O (2 mL) were added Pd(PPh₃)₂Cl₂ (240 mg, 0.342 mmol), K₃PO₄ (1.1 g, 5.18 mmol) and 2-chloro-5-(trifluoro-methyl)pyrimidine (310 mg, 1.7 mmol) at 25° C. The mixture was stirred at 80° C. for 16 hrs under N₂. The reaction mixture was cooled to room temperature and partitioned between water (10 mL) and EtOAc (15 mL). The organic layer was washed with brine (20 mL), dried over anhydrous Na₂SO₄, filtered and concentrated to dryness. The residue was purified by flash column chromatography on silica gel (gradient, PE:EtOAc=5:1) to give the title compound (450 mg, 1.44 mmol, 84.3% yield) as a yellow solid.

LCMS m/e: 314 (MH⁺).

### Scheme AB, Step 4. Synthesis of 7-methyl-5-(5-(trifluoromethyl)pyrimidin-2-yl)-7H-pyrrolo[2,3-d]pyrimidin-4-amine

A solution of 4-chloro-7-methyl-5-(5-(trifluoromethyl)pyrimidin-2-yl)-7H-pyrrolo[2,3-d]pyrimidine (450 mg, 1.44 mmol) in NH₄OH (30 ml, 28% in H₂O) was stirred at 100° C. overnight, cooled to room temperature, diluted with water (30 mL) and extracted with DCM (40 mL×2). The combined organic layers were washed with brine (30 mL), dried over anhydrous Na₂SO₄, filtered and concentrated to give the title compound (200 mg, 0.68 mmol, 47.3% yield) as a pale solid.

LCMS m/e: 295 (MH⁺).

### Scheme AB, Step 5. Synthesis of 6-bromo-7-methyl-5-[5-(trifluoromethyl)pyrimidin-2-yl]pyrrolo[2,3-d]pyrimidin-4-amine

To a solution of 7-methyl-5-(5-(trifluoromethyl)pyrimidin-2-yl)-7H-pyrrolo[2,3-d]pyrimidin-4-amine (100 mg, 0.34 mmol) in MeCN (0.5 mL) and AcOH (0.5 mL) were added NBS (72 mg, 0.408 mmol). The resulting mixture was stirred at 30° C. for 18 hrs, diluted with sat. NaHCO₃ (5 mL) at RT, and extracted with EtOAc (20 mL×2). The combined organic layers were washed with brine, dried over anhydrous Na₂SO₄, filtered and concentrated to dryness. The residue was purified by flash chromatography on silica gel (gradient, PE:EtOAc=1:1) to give the title compound (60 mg, 0.161 mmol, 47.3%) as a yellow solid.

LCMS m/e: 373/375 (MH⁺).

### Scheme AB Step 6. Synthesis of tert-butyl 9-(4-amino-7-methyl-5-(5-(trifluoromethyl)-pyrimidin-2-yl)-7H-pyrrolo[2,3-d]pyrimidin-6-yl)-3-azaspiro[5.5]undec-8-ene-3-carboxylate

To a solution of 6-bromo-7-methyl-5-(5-(trifluoromethyl)pyrimidin-2-yl)-7H-pyrrolo-[2,3-d]pyrimidin-4-amine (60 mg, 0.161 mmol) and tert-butyl 9-(4,4,5,5-tetramethyl-1,3,2-dioxaborolan-2-yl)-3-azaspiro[5.5]undec-8-ene-3-carboxylate (79 mg, 0.209 mmol) in dioxane (2 mL) and water (0.2 mL) were added Na₂CO₃ (51 mg, 0.482 mmol) and Pd(dppf)Cl₂ (12 mg, 0.016 mmol). The resulting mixture was stirred under N₂ at 100° C. for 18 hrs. It was then cooled and purified by flash chromatography on silica gel (gradient, DCM:EtOAc=1:1) to give the title compound (60 mg, 0.11 mmol, 68.6%) as a pale solid.

LCMS m/e: 544 (MH⁺).

### Scheme AB Step 7. Synthesis of 7-methyl-6-(3-azaspiro[5.5]undec-8-en-9-yl)-5-(5-(trifluoromethyl)pyrimidin-2-yl)-7H-pyrrolo[2,3-d]pyrimidin-4-amine

To a solution of tert-butyl 9-(4-amino-7-methyl-5-(5-(trifluoromethyl)pyrimidin-2-yl)-7H-pyrrolo[2,3-d]pyrimidin-6-yl)-3-azaspiro[5.5]undec-8-ene-3-carboxylate (60 mg, 0.11 mmol) in DCM (10 mL) was added dropwise TFA (0.5 mL) at RT, the mixture was stirred for 1 hr at RT and concentrated to give the title compound (60 mg, 0.108 mmol, 97.5%) as a yellow solid.

LCMS m/e: 444 (MH⁺).

### Scheme AB, Step 8. Synthesis of 1-(9-(4-amino-7-methyl-5-(5-(trifluoromethyl)pyrimidin-2-yl)-7H-pyrrolo[2,3-d]pyrimidin-6-yl)-3-azaspiro[5.5]undec-8-en-3-yl)prop-2-en-1-one

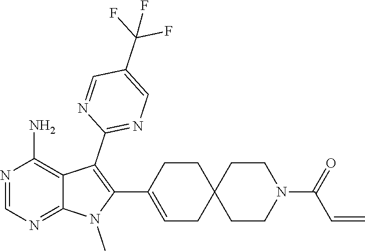 [SMILES: C=CC(=O)N1CCC2(CC=C(c3c(-c4ncc(C(F)(F)F)cn4)c4c(N)ncnc4n3C)CC2)CC1]

### Example 42

To a solution of 7-methyl-6-(3-azaspiro[5.5]undec-8-en-9-yl)-5-(5-(trifluoromethyl)-pyrimidin-2-yl)-7H-pyrrolo[2,3-d]pyrimidin-4-amine (50 mg, 0.113 mmol) in DCM (1 mL) and TEA (45 mg, 0.45 mmol) was added dropwise acryloyl chloride (10 mg, 0.113 mmol) at 0° C. The mixture was stirred for 0.5 hrs, diluted with water (3 mL), and extracted with DCM (10 mL×2). The combined organic layers were washed with brine, dried over anhydrous Na2SO4, filtered and concentrated to dryness. The residue was purified by reverse phase chromatography (SilaSep™ C18 silica flash cartridge, 0%-40% MeCN in H₂ with 0.100 formic acid) to give the title compound (8.2 mg, 0.016 mmol, 14.6%) as a white solid.

¹H NMR (400 MHz, DMSO) δ 9.16 (s, 2H), 9.04 (s, 1H), 8.13 (s, 1H), 7.32 (s, 1H), 6.86 (dd, 1=16.7, 10.5 Hz, 1H), 6.11 (d, 1=18.6 Hz, 1H), 5.68 (d, J=7.7 Hz, 2H), 3.76-3.64 (m, 2H), 3.62 (s, 3H), 3.57-3.48 (m, 2H), 2.31-2.22 (m, 2H), 2.12 (s, 2H), 1.73 (t, =4.9 Hz, 2H), 1.62-1.46 (3, 4H).

LCMS m/e: 498 (MH⁺).

Applying the same or a similar procedure described above, the examples of Table 6 were prepared.

TABLE 6

| | | m/z | |
|---|---|---|---|
| Example | structure | (MH⁺) | ¹H NMR |
| 43 | 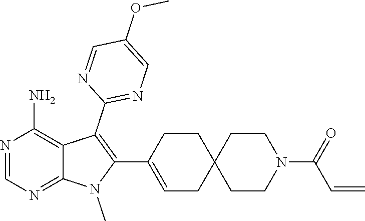 [SMILES: C=CC(=O)N1CCC2(CC=C(c3c(-c4ncc(OC)cn4)c4c(N)ncnc4n3C)CC2)CC1] | 460.5 | ¹HNMR(400 MHz, DMSO) (with HCOOH) δ 9.08 (hump, 0.7H), 8.55 (s, 2H), 8.13 (s, 1H), 8.08 (s, 1H), 7.15 (hump, 0.9H), 6.87 (dd, J = 16.7, 10.5 Hz, 1H), 6.11 (dd, J = 16.7, 2.5 Hz, 1H), 5.67 (dd, J = 10.4, 2.5 Hz, 1H), 5.63 (s, 1H), 3.94 (s, 3H), 3.73-3.63 (m, 2H), 3.59 (s, 3H), 3.57-3.46 (m, 2H), 2.27 (br. S, 2H), 2.10 (br. S, 2H), 1.69 (t, J = 6.6 Hz, 2H), 1.63-1.46 (m, 4H) |
| 44 | 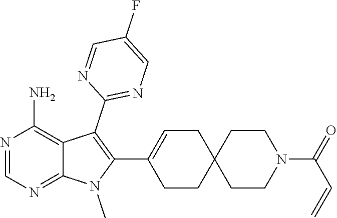 [SMILES: C=CC(=O)N1CCC2(CC=C(c3c(-c4ncc(F)cn4)c4c(N)ncnc4n3C)CC2)CC1] | 448.5 | ¹H NMR (400 MHz, DMSO) δ 10.51 (hump, 0.7H), 8.98 (s, 2H), 8.60 (hump, .7H), 8.46 (s, 1H), 6.88 (dd, J = 16.7, 10.5 Hz, 1H), 6.13 (dd, J = 16.7, 2.4 Hz, 1H), 5.81-5.60 (m, 2H), 3.70 (s, 3H), 3.7-3.59 (m, 4H), 2.29 (m, 2H), 2.14 (br. s, 2H), 1.72 (m, 2H), 1.60 (m, 2H), 1.54 (m, 2H). |
| 45 | 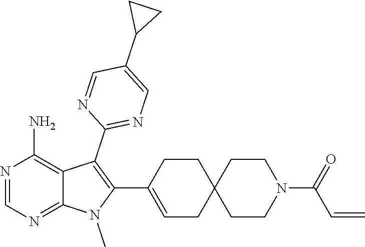 [SMILES: C=CC(=O)N1CCC2(CC=C(c3c(-c4ncc(C5CC5)cn4)c4c(N)ncnc4n3C)CC2)CC1] | 470.6 | ¹H NMR (400 MHz, CDCl₃) δ 8.36 (s, 2H), 8.23 (s, 1H), 6.63 (dd, J = 16.7, 10.6 Hz, 1H), 6.30 (d, J = 16.9 Hz, 1H), 5.70 (d, J = 8.2 Hz, 2H), 3.84 (m, 1H), 3.69 (s, 3H), 3.69-3.56 (m, 4H), 2.42 (m, 2H), 2.27-2.18 (m, 4H), 1.86- 1.65 (m, 4H), 1.10 (d, J = 7.6 Hz, 2H), 0.78 (d, J = 4.4 Hz, 2H). |
| 46 | 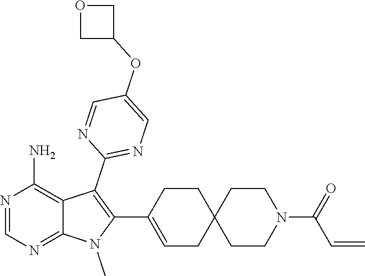 [SMILES: C=CC(=O)N1CCC2(CC=C(c3c(-c4ncc(OC5COC5)cn4)c4c(N)ncnc4n3C)CC2)CC1] | 502.6 | ¹H NMR (400 MHz, DMSO) δ 9.38 (hump, 0.7H), 8.46 (s, 2H), 8.16 (s, 1H), 7.43 (hump, 0.8H), 6.90 (dd, J = 16.7, 10.4 Hz, 1H), 6.14 (d, J = 16.4 Hz, 1H), 5.78- 5.60 (m, 2H), 5.57-5.43 (m, 1H), 4.99 (t, J = 6.6 Hz, 2H), 4.75-4.53 (m, 2H), 3.71 (m, 2H), 3.62 (s, 3H), 3.57 (m, 2H), 2.29 (m, 2H), 2.12 (br.s, 2H), 1.72 (br.s, 2H), 1.58 (m, 4H). |
| 47 | 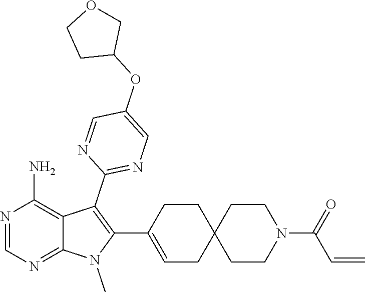 [SMILES: C=CC(=O)N1CCC2(CC=C(c3c(-c4ncc(OC5CCOC5)cn4)c4c(N)ncnc4n3C)CC2)CC1] | 516.6 | ¹H NMR (400 MHz, DMSO) (with HCOOH) δ 9.13 (hump, .7H), 8.54 (s, 2H), 8.13 (s, 1H), 8.10 (s, 1H), 7.19 (hump, 0.85H), 6.87 (dd, J = 16.6, 10.5 Hz, 1H), 6.11 (d, J = 17.1 Hz, 1H), 5.72-5.53 (m, 2H), 5.24 (br s, 1H), 3.94-3.83 (m, 3H), 3.83-3.74 (m, 1H), 3.68 (m, 2H), 3.59 (s, 3H), 3.59-3.54 (m, 2H), 2.33-2.26 (m, 2H), 2.15-1.93 (m, 4H), 1.69 (m, 2H), 1.56 (m, 4H). |
| 48 | 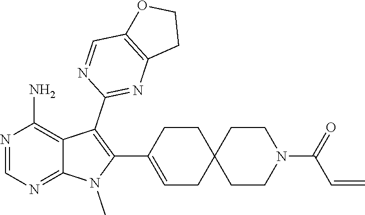 [SMILES: C=CC(=O)N1CCC2(CC=C(c3c(-c4ncc5c(n4)CCO5)c4c(N)ncnc4n3C)CC2)CC1] | 472.5 | ¹H NMR (400 MHz, DMSO) δ 9.23 (s, 1H), 8.74 (s, 1H), 8.13 (s, 1H), 8.10 (s, 1H), 7.13 (s, 1H), 6.85 (dd, J = 16.7, 10.5 Hz, 1H), 6.10 (dd, J = 16.7, 2.4 Hz, 1H), 5.71-5.62 (m, 2H), 5.11 (s, 2H), 4.91 (s, 2H), 3.67 (s, 2H), 3.59 (s, 3H), 3.51 (d, J = 14.8 Hz, 2H), 2.28 (s, 2H), 2.09 (s, 2H), 1.67 (s, 2H), 1.53 (s, 4H). |
| 49 | 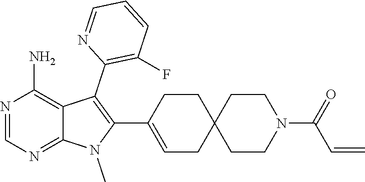 [SMILES: C=CC(=O)N1CCC2(CC=C(c3c(-c4ncccc4F)c4c(N)ncnc4n3C)CC2)CC1] | 447.2 | ¹H NMR (400 MHz, DMSO) δ 8.54 (d, J = 4.2 Hz, 1H), 8.14 (d, J = 5.5 Hz, 1H), 7.78 (t, J = 9.4 Hz, 1H), 7.55-7.32 (m, 1H), 6.85- 6.79 (dd, J = 16, 8.4 Hz 1H), 6.76 (d, J = 8.4 Hz, 2H), 6.08 (d, J = 16.0 Hz, 1H), 5.65 (d, J = 10.7 Hz, 1H), 5.53 (br.s, 1H), 3.66 (s, 3H), 3.67 (m, 2H), 3.40 (m, 2H), 2.24 (br.s, 2H), 1.96 (br.s, 2H), 1.61 (br.s, 2H), 1.40 (m, 4H). |
| 61 | 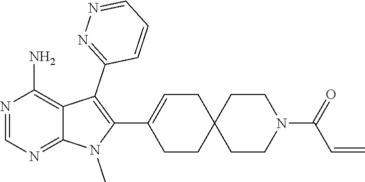 [SMILES: C=CC(=O)N1CCC2(CC=C(c3c(-c4cccnn4)c4c(N)ncnc4n3C)CC2)CC1] | 430.5 | ¹H NMR (400 MHz, DMSO) δ 9.06 (d, J = 4.2 Hz, 1H), 8.13 (s, 1H), 7.86 (d, J = 8 Hz, 1H), 7.76- 7.72 (m, 1H), 8.82 (d, J = 16, 8 Hz, 1H), 6.09 (d, J = 16 Hz, 1H), 5.97 (br S, 1H), 5.66 (d, J = 12 Hz, 1H), 3.62 (S, 3H), 3.7-3.5 (m, 4H), 2.17-2.15 ( m, 4H), 1.67 (m, 2H), 1.45 (m, 4H) |

### Example 50

### Synthesis of 1-(9-(4-amino-5-(5-cyclopropoxypyrimidin-2-yl)-7-methyl-7H-pyrrolo[2,3-d]pyrimidin-6-yl)-3-azaspiro[5.5]undec-8-en-3-yl)prop-2-en-1-one

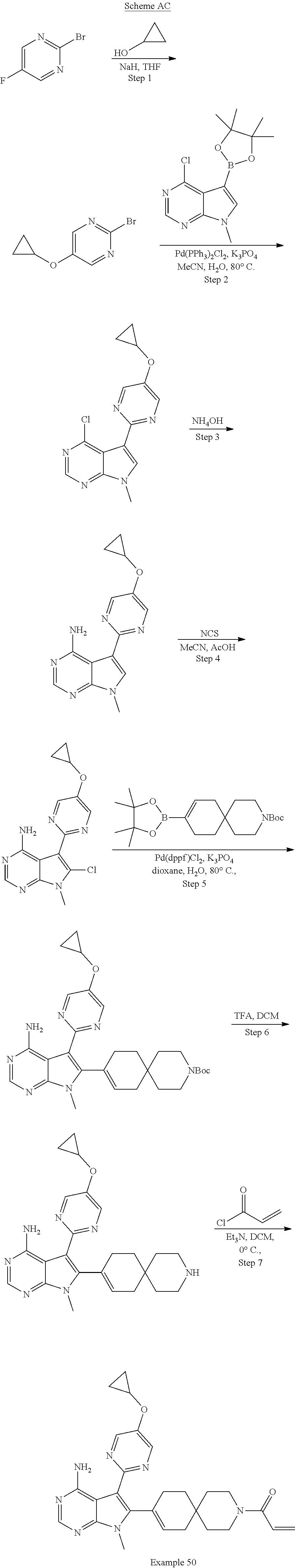

### Scheme AC, Step 1. Synthesis of 2-bromo-5-cyclopropoxypyrimidine

To a solution of cyclopropanol (5.91 g, 102 mmol) in THF (200 mL) was added NaH (4.88 g, 204 mmol, 60% suspension in mineral oil) portion wise at 0° C. under N₂. After stirring at 0° C. for 0.5 hrs, 2-bromo-5-fluoropyrimidine (12 g, 67.8 mmol) was added under 0° C. and stirred at 25° C. for 2 hrs. The resulting solution was poured into water (500 mL) and extracted with EtOAc (500 mL×2). The combined organic layers were washed with brine (500 mL), dried over anhydrous Na₂SO₄ and filtered. The filtrate was concentrated under reduced pressure and purified by silica gel chromatography (gradient, PE to PE:DCM=1:4) to give the title compound (320 mg, 1.49 mmol, 2.19% yield) as a light-yellow solid.

LC-MS m/e: 215.1, 217.1 (MH⁺).

### Scheme AC, Step 2. Synthesis of 4-chloro-5-(5-cyclopropoxypyrimidin-2-yl)-7-methyl-7H-pyrrolo[2,3-d]pyrimidine

To a solution of 2-bromo-5-(cyclopropyloxy)pyrimidine (320 mg, 1.49 mmol) and 4-chloro-7-methyl-5-(4,4,5,5-tetramethyl-1,3,2-dioxaborolan-2-yl)pyrrolo[2,3-d]pyrimidine (437 mg, 1.49 mmol) in MeCN (20 mL) and water (10 mL) were added Pd(PPh₃)₂Cl₂ (105 mg, 0.149 mmol) and K₃PO₄ (948 mg, 4.47 mmol). The resultant mixture was stirred at 80° C. under N₂ for 2 hrs, cooled to room temperature, poured into water (10 mL) and extracted with EtOAc (20 mL×3). The combined organic layers were washed with brine (20 mL), dried over anhydrous Na₂SO₄ and filtered. The filtrate was concentrated under vacuum and purified by an aluminum oxide column (gradient, PE to PE:EtOAc=2:1) to give the title compound (250 mg, 0.829 mmol, 55.7% yield) as a light-yellow solid.

LC-MS m/e: 302 (MH⁺).

### Scheme AC, Step 3. Synthesis of 5-(5-cyclopropoxypyrimidin-2-yl)-7-methyl-7H-pyrrolo[2,3-d]pyrimidin-4-amine

A solution of 4-chloro-5-[5-(cyclopropyloxy)pyrimidin-2-yl]-7-methylpyrrolo[2,3-d]pyrimidine (250 mg, 0.829 mmol) in NH₃ solution (5 mL, 7 M in MeOH) was stirred at 60° C. overnight, cooled to room temperature, concentrated under vacuum and purified by flash chromatography on silica gel (gradient, DCM to DCM:EtOAc=1:1) to give the title compound (140 mg, 0.496 mmol, 59.9% yield) as a light-yellow solid.

LC-MS m/e: 235 (MH⁺).

### Scheme AC, Step 4. Synthesis of 6-chloro-5-(5-cyclopropoxypyrimidin-2-yl)-7-methyl-7H-pyrrolo[2,3-d]pyrimidin-4-amine

To a solution of 5-(5-cyclopropoxypyrimidin-2-yl)-7-methyl-7H-pyrrolo[2,3-d]-pyrimidin-4-amine (130 mg, 0.46 mmol) in MeCN (15 mL) was added NCS (92 mg, 0.691 mmol) and stirred at 40° C. for 2 hrs. The resulting solution was cooled to room temperature, pour into water (50 mL) and extracted with EtOAc (50 mL×2). The combined organic layers were washed with brine (50 mL), dried over anhydrous Na₂SO₄ and filtered. The filtrate was concentrated under reduced pressure and purified by silica gel chromatography (gradient, DCM to DCM:MeOH=20:1) to give the title compound (110 mg, 0.347 mmol, 75.5% yield) as a light-yellow solid.

LC-MS m/e: 317.1, 319.1 (MH⁺).

### Scheme AC, Step 5. Synthesis of tert-butyl 9-(4-amino-5-(5-cyclopropoxypyrimidin-2-yl)-7-methyl-7H-pyrrolo[2,3-d]pyrimidin-6-yl)-3-azaspiro[5.5]undec-8-ene-3-carboxylate

To a solution of 6-chloro-5-(5-cyclopropoxypyrimidin-2-yl)-7-methyl-7H-pyrrolo[2,3-d]pyrimidin-4-amine (90 mg, 0.284 mmol) and tert-butyl 9-(4,4,5,5-tetramethyl-1,3,2-dioxa-borolan-2-yl)-3-azaspiro[5.5]undec-8-ene-3-carboxylate (214 mg, 0.568 mmol) in dioxane/water (40 mL, 4/1 v/v) were added K₃PO₄ (181 mg, 0.852 mmol) and Pd(dppf)Cl₂ (21 mg, 0.29 mmol). The mixture was stirred at 80° C. under N₂ for 2 hrs, cooled to room temperature, poured into water (10 mL) and extracted with EtOAc (20 mL×3). The combined organic layers were washed with brine (20 mL), dried over anhydrous Na₂SO₄ and filtered. The filtrate was concentrated under vacuum and purified by silica gel chromatography (gradient, DCM to DCM:MeOH=20:1) to give the title compound (130 mg, 0.147 mmol, 51.6% yield) as a light-yellow solid.

LC-MS m/e: 532 (MH⁺).

### Scheme AC, Step 6. Synthesis of 5-(5-cyclopropoxypyrimidin-2-yl)-7-methyl-6-(3-azaspiro[5.5]undec-8-en-9-yl)-7H-pyrrolo[2,3-d]pyrimidin-4-amine

To a solution of 9-(4-amino-5-(5-cyclopropoxypyrimidin-2-yl)-7-methyl-7H-pyrrolo-[2,3-d]pyrimidin-6-yl)-3-azaspiro[5.5]undec-8-ene-3-carboxylate (130 mg, 0.147 mmol) in DCM (10 mL) was added TFA (2 mL) at 0° C. and stirred at room temperature for 1 hr. The resulting solution was concentrated under reduced pressure and purified by an aluminum oxide column (gradient, DCM to DCM:MeOH=33:1) to give the title compound (60 mg, 0.139 mmol, 94.8% yield) as a yellow solid.

LC-MS m/e: 432 (MH⁺).

### Scheme AC, Step 7. Synthesis of 1-(9-(4-amino-5-(5-cyclopropoxypyrimidin-2-yl)-7-methyl-7H-pyrrolo[2,3-d]pyrimidin-6-yl)-3-azaspiro[5.5]undec-8-en-3-yl)prop-2-en-1-one

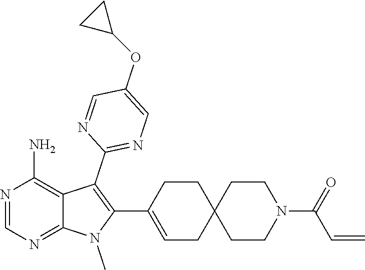 [SMILES: C=CC(=O)N1CCC2(CC=C(c3c(-c4ncc(OC5CC5)cn4)c4c(N)ncnc4n3C)CC2)CC1]

### Example 50

To a solution of 5-(5-cyclopropoxypyrimidin-2-yl)-7-methyl-6-(3-azaspiro[5.5]undec-8-en-9-yl)-7H-pyrrolo[2,3-d]pyrimidin-4-amine (60 mg, 0.139 mmol) and TEA (0.058 mL, 0.417 mmol) in DCM (10 mL) was added acryloyl chloride (0.011 mL, 0.139 mmol) dropwise at 0° C. and stirred at 0° C. for 5 min. The resulting solution was poured into water (10 mL) and extracted with EtOAc (30 mL×2). The combined organic layers were washed with brine (30 mL), dried over anhydrous Na₂SO₄ and filtered. The filtrate was concentrated under reduced pressure and purified by prep-HPLC to give the title compound (14.6 mg, 0.03 mmol, 21.6% yield) as a white solid.

¹H NMR (400 MHz, DMSO-d6) δ 9.06 (hump, 0.6H), 8.62 (s, 2H), 8.08 (br s, 0.84H), 7.12 (hump s, 0.7H), 6.86 (t, J=15.4 Hz, 1H), 6.11 (d, J=16.0 Hz, 1H), 5.73-5.48 (m, 2H), 4.11 (br s, 1H), 3.76-3.63 (m, 2H), 3.58 (s, 3H), 3.53-3.44 (m, 2H), 2.28 (m, 2H), 2.10 (m, 2H), 1.74-1.64 (m, 2H), 1.56 (d, J=19.6 Hz, 4H), 0.82 (d, J=30.2 Hz, 4H).

LC-MS m/e: 486 (MH⁺).

### Example 51

### Synthesis of 2-(6-(3-acryloyl-3-azaspiro[5.5]undec-8-en-9-yl)-4-amino-7-methyl-7H-pyrrolo[2,3-d]pyrimidin-5-yl)pyrimidine-5-carbonitrile

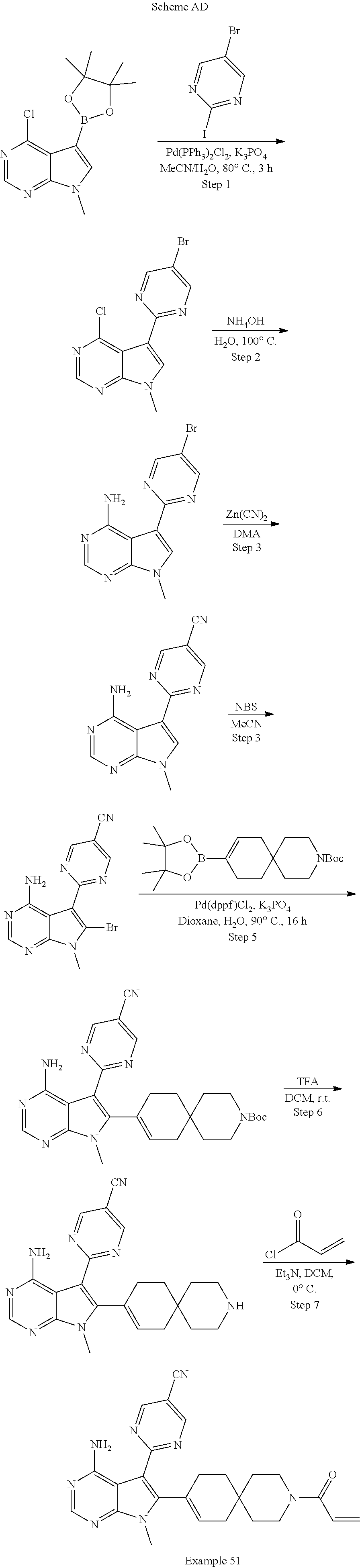

### Scheme AD, Step 1. Synthesis of 5-(5-bromopyrimidin-2-yl)-4-chloro-7-methyl-7H-pyrrolo[2,3-d]

To a solution of 4-chloro-7-methyl-5-(4,4,5,5-tetramethyl-1,3,2-dioxaborolan-2-yl)-pyrrolo[2,3-d]pyrimidine (2.5 g, 8.51 mmol) and 5-bromo-2-iodopyrimidine (2.91 g, 10.2 mmol) in MeCN (40 mL) and water (5 mL) were added Pd(PPh₃)₂Cl₂ (0.99 g, 1.28 mmol) and K₃PO₄ (5.42 g, 25.5 mmol). The reaction mixture was stirred at 80° C. for 3 hrs under N₂, then cooled to room temperature, poured into water (200 mL) and extracted with EtOAc (100 mL×2). The combined organic layers were washed with brine (50 mL), dried over anhydrous Na₂SO₄ and filtered. The filtrate was concentrated under vacuum and purified by silica gel column chromatography (gradient, DCM to DCM:MeOH=20:1) to give the title compound (1.3 g, 4 mmol, 47% yield) as a yellow solid.

LC-MS m/e: 324.6, 326.6 (MH⁺).

### Scheme AD, Step 2. Synthesis of 5-(5-bromopyrimidin-2-yl)-7-methyl-7H-pyrrolo[2,3-d]pyrimidin-4-amine

A solution of 5-(5-bromopyrimidin-2-yl)-4-chloro-7-methyl-7H-pyrrolo[2,3-d]pyri-midine (1.3 g, 4 mmol) in NH₃·H₂O (20 mL) was stirred at 80° C. in a sealed tube for 12 hrs, cooled to room temperature and poured into water (100 mL). The resulting suspension was filtered. The cake was washed with water (100 mL) and dried under vacuum to give the title compound (1.1 g, 3.6 mmol, 89.8% yield) as a gray solid.

LC-MS m/e: 305.3, 307.3 (MH⁺).

### Scheme AD, Step 3. Synthesis of 2-(4-amino-7-methyl-7H-pyrrolo[2,3-d]pyrimidin-5-yl)pyrimidine-5-carbonitrile

To a solution of 5-(5-bromopyrimidin-2-yl)-7-methyl-7H-pyrrolo[2,3-d]pyrimidin-4-amine (500 mg, 1.65 mmol) in DMA (10 mL) were added Zn(CN)₂ (962 mg, 0.328 mmol), X-Phos (313 mg, 0.65 mmol) and Pd₂(dba)₃ (300 mg, 0.325 mmol). The reaction mixture was stirred at 120° C. for 12 hrs under N₂. The reaction mixture was cooled to room temperature and partitioned between water (50 mL) and EtOAc (150 mL). The organic layer was washed with brine (50 mL), dried over anhydrous Na₂SO₄, filtered and concentrated to dryness. The residue was purified by flash column chromatography on silica gel (gradient, DCM to DCM:MeOH=10:1) to give the title compound (400 mg, 1.59 mmol, 96.3% yield) as a white solid.

LC-MS m/e: 252.2 (MH⁺)

### Scheme AD, Step 4. Synthesis of 2-(4-amino-6-bromo-7-methyl-7H-pyrrolo[2,3-d]pyrimidin-5-yl)pyrimidine-5-carbonitrile

To a solution of 2-(4-amino-7-methyl-7H-pyrrolo[2,3-d]pyrimidin-5-yl)pyrimidine-5-carbonitrile (300 mg, 1.19 mmol) in MeCN (10 mL) and AcOH (10 mL) was added NBS (255 mg, 1.46 mmol) and stirred at 20° C. for 12 hrs. The reaction mixture was diluted with EtOAc (50 mL), washed with brine (20 mL), dried over anhydrous Na₂SO₄, filtered and concentrated under vacuum. The crude product was subjected to column chromatography (gradient:PE to PE:EtOAc=1:1) to give the title compound (300 mg, 0.909 mmol, 76.3% yield) as a yellow solid.

LC-MS m/e: 332.1, 332.1 (MH⁺).

### Scheme AD, Step 5. Synthesis of tert-butyl 9-(4-amino-5-(5-cyanopyrimidin-2-yl)-7-methyl-7H-pyrrolo[2,3-d]pyrimidin-6-yl)-3-azaspiro[5.5]undec-8-ene-3-carboxylate

To a mixture of 2-(4-amino-6-bromo-7-methyl-7H-pyrrolo[2,3-d]pyrimidin-5-yl)pyrimidine-5-carbonitrile (300 mg, 0.909 mmol) and tert-butyl 9-(4,4,5,5-tetramethyl-1,3,2-dioxaborolan-2-yl)-3-azaspiro[5.5]undec-8-ene-3-carboxylate (356 mg, 0.909 mmol) in dioxane (4 mL) and water (1 mL) were added K₂CO₃ (376 mg, 2.73 mmol) and Pd(dppf)Cl₂ (133 mg, 0.182 mmol). The reaction mixture was stirred at 100° C. under N₂ for 12 hrs, then cooled to room temperature, diluted with H₂O (20 mL) and extracted with EtOAc (50 mL×3). The organic phases were washed with brine (30 mL), dried over anhydrous Na₂SO₄, filtered and concentrated under vacuum. The crude product was subjected to column chromatography (gradient, DCM to DCM:MeOH=20:1) to give the title compound (170 mg, 0.34 mmol, 37.4% yield) as a yellow solid.

LC-MS m/e: 501.3 (MH⁺).

### Scheme AD, Step 6. Synthesis of 2-(4-amino-7-methyl-6-(3-azaspiro[5.5]undec-8-en-9-yl)-7H-pyrrolo[2,3-d]pyrimidin-5-yl)pyrimidine-5-carbonitrile

To a solution of tert-butyl 9-(4-amino-5-(5-cyanopyrimidin-2-yl)-7-methyl-7H-pyrrolo[2,3-d]pyrimidin-6-yl)-3-azaspiro[5.5]undec-8-ene-3-carboxylate (170 mg, 0.34 mmol) in DCM (4 mL) was added TFA (1 mL) at 0° C. and stirred at room temperature for 1 hr. The reaction solution was concentrated under vacuum to give the title compound (130 mg, 0.325 mmol, 95.6% yield, crude) as a yellow solid.

LC-MS m/e: 401 (MH⁺).

### Scheme AD, Step 7. Synthesis of 2-(6-(3-acryloyl-3-azaspiro[5.5]undec-8-en-9-yl)-4-amino-7-methyl-7H-pyrrolo[2,3-d]pyrimidin-5-yl)pyrimidine-5-carbonitrile

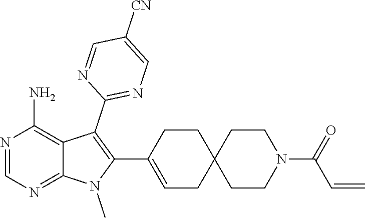 [SMILES: C=CC(=O)N1CCC2(CC=C(c3c(-c4ncc(C#N)cn4)c4c(N)ncnc4n3C)CC2)CC1]

### Example 51

To a solution of 2-(4-amino-7-methyl-6-(3-azaspiro[5.5]undec-8-en-9-yl)-7H-pyrrolo-[2,3-d]pyrimidin-5-yl)pyrimidine-5-carbonitrile (130 mg, 0.325 mmol) and TEA (0.135 mL, 0.974 mmol) in DCM (5 mL) was added acryloyl chloride (0.029 mL, 0.357 mmol) at 0° C. and stirred at 0° C. under N₂ for 0.5 hr. The resulting solution was poured into water (20 mL) and extracted with EtOAc (20 mL×2). The combined organic layers were washed with brine (20 mL), dried over anhydrous Na₂SO₄ and filtered. The filtrate was concentrated under vacuum and purified by silica gel column chromatography (gradient, DCM to DCM:MeOH=15:1) followed by prep-HPLC to give the title compound (5 mg, 0.011 mmol, 3.39% yield) as a light-yellow solid.

¹H NMR (400 MHz, DMSO-d6) δ 9.19 (s, 2H), 9.03 (s, 1H), 8.13 (hump, 0.7H), 7.32 (hump, 1H), 6.87 (dd, J=16.9, 11.3 Hz, 1H), 6.12 (d, J=17.0 Hz, 1H), 5.68 (d, J=5.0 Hz, 2H), 3.76-3.65 (m, 2H), 3.61 (s, 3H), 3.58-3.49 (m, 1H), 2.33-2.19 (m, 2H), 2.11 (br s, 2H), 2.02-1.95 (m, 1H), 1.74-1.64 (m, 2H), 1.56 (d, J=23.4 Hz, 4H).

LC-MS m/e: 455.3 (MH⁺).

### Example 52

### Synthesis of 1-(9-(4-amino-7-methyl-5-(5-(tetrahydrofuran-3-yl)pyrimidin-2-yl)-7H-pyrrolo[2,3-d]pyrimidin-6-yl)-3-azaspiro[5.5]undec-8-en-3-yl)prop-2-en-1-one

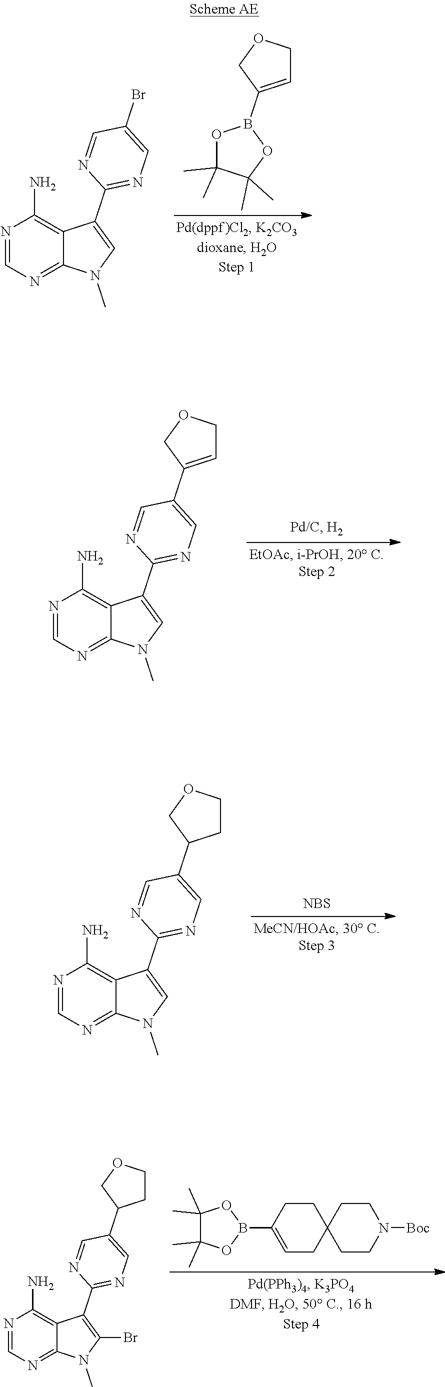

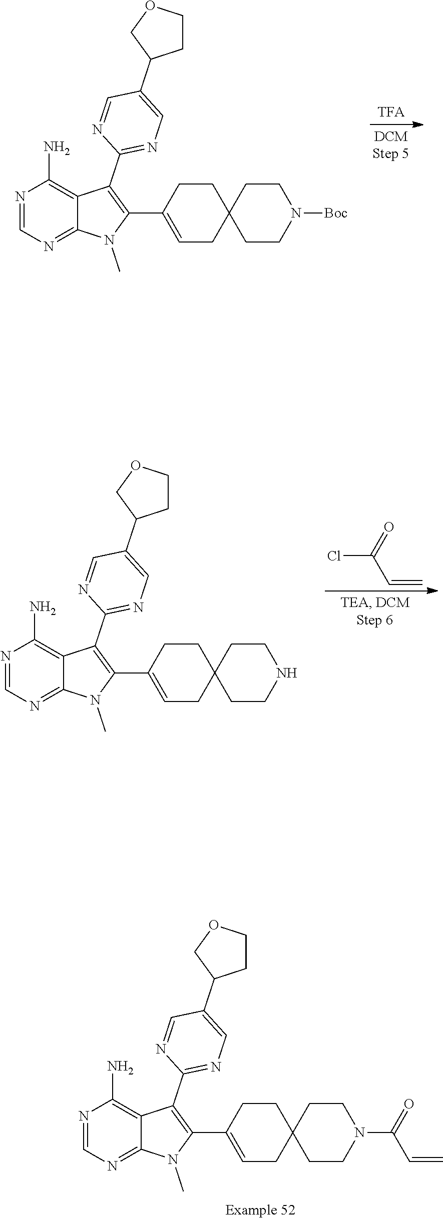

### Scheme AE, Step 1. Synthesis of 5-(5-(2,5-dihydrofuran-3-yl)pyrimidin-2-yl)-7-methyl-7H-pyrrolo[2,3-d]pyrimidin-4-amine

To a solution of 5-(5-bromopyrimidin-2-yl)-7-methyl-7H-pyrrolo[2,3-d]pyrimidin-4-amine (170 mg, 0.453 mmol) and 2-(2,5-dihydrofuran-3-yl)-4,4,5,5-tetramethyl-1,3,2-dioxaborolane (178 mg, 0.453 mmol) in dioxane (4 mL) and water (1 mL) were added Pd(dppf)Cl₂ (66.3 mg, 0.091 mmol) and K₂CO₃ (188 mg, 1.36 mmol) and stirred at 100° C. under N₂ for 12 hrs. The resulting solution was cooled to room temperature, poured into water (10 mL) and extracted with EtOAc (50 mL×2). The combined organic layers were washed with brine (10 mL), dried over anhydrous Na₂SO₄ and filtered. The filtrate was concentrated under vacuum and purified by silica gel column chromatography (gradient, DCM to DCM:MeOH=20:1) to give the title compound (130 mg, 0.442 mmol, 98.2% yield) as a yellow solid.

LC-MS m/e: 295.2 (MH⁺)

### Scheme AE, Step 2. Synthesis of 7-methyl-5-(5-(tetrahydrofuran-3-yl)pyrimidin-2-yl)-7H-pyrrolo[2,3-d]pyrimidin-4-amine

To a solution of 5-(5-(2,5-dihydrofuran-3-yl)pyrimidin-2-yl)-7-methyl-7H-pyrrolo-[2,3-d]pyrimidin-4-amine (150 mg, 0.51 mmol) in EtOAc (6 mL) and i-PrOH (2 mL) was added 10% Pd/C (150 mg, 55% wet). The reaction mixture was stirred at 20° C. for 12 hrs under H₂. The resulting suspension is filtered through a pad of Celite® and the cake was washed with MeOH (20 mL). The filtrate was concentrated under vacuum to give the title compound (150 mg, 0.5 mmol, 99.1% yield) as a yellow solid.

LC-MS m/e: 297.2 (MH⁺)

### Scheme AE, Step 3. Synthesis of 6-bromo-7-methyl-5-(5-(tetrahydrofuran-3-yl)pyrimidin-2-yl)-7H-pyrrolo[2,3-d]pyrimidin-4-amine

To a solution of 7-methyl-5-(5-(tetrahydrofuran-3-yl)pyrimidin-2-yl)-7H-pyrrolo[2,3-d]pyrimidin-4-amine (150 mg, 0.506 mmol) in AcOH (2 mL) and MeCN (2 mL) was added NBS (108 mg, 0.607 mmol) and stirred at 20° C. for 12 hrs. The reaction mixture was diluted with EtOAc (20 mL) and basified with saturated NaHCO₃ solution (20 mL). The organic layer was washed with brine (10 mL), dried over anhydrous Na₂SO₄, filtered and concentrated under vacuum. The residue was purified by silica gel column chromatography (gradient, DCM to DCM:MeOH=30:1) to give the title compound (170 mg, 0.453 mmol, 89.5% yield) as a yellow solid.

LC-MS m/e: 375.2/377.2 (MH⁺).

### Scheme AE, Step 4. Synthesis of tert-butyl 9-(4-amino-7-methyl-5-(5-(tetrahydrofuran-3-yl)-pyrimidin-2-yl)-7H-pyrrolo[2,3-d]pyrimidin-6-yl)-3-azaspiro[5.5]undec-8-ene-3-carboxylate

To a solution of 6-bromo-7-methyl-5-(5-(tetrahydrofuran-3-yl)pyrimidin-2-yl)-7H-pyrrolo[2,3-d]pyrimidin-4-amine (170 mg, 0.453 mmol) and tert-butyl 9-(4,4,5,5-tetramethyl-1,3,2-dioxaborolan-2-yl)-3-azaspiro[5.5]undec-8-ene-3-carboxylate (178 mg, 0.453 mmol) in dioxane (4 mL) and water (1 mL) were added Pd(dppf)Cl₂ (66 mg, 0.091 mmol) and K₂CO₃ (187 mg, 1.36 mmol) and stirred at 100° C. under N₂ for 12 hrs. The resulting solution was cooled to room temperature, poured into water (10 mL) and extracted with EtOAc (30 mL×2). The combined organic layers were washed with brine (30 mL), dried over anhydrous Na₂SO₄ and filtered. The filtrate was concentrated under vacuum and purified by silica gel column chromatography (gradient, DCM to DCM:MeOH=30:1) to give the title compound (130 mg, 0.238 mmol, 52.6% yield) as a yellow solid.

LC-MS m/e: 546.8 (MH⁺)

### Scheme AE, Step 5. Synthesis of 7-methyl-6-(3-azaspiro[5.5]undec-8-en-9-yl)-5-(5-(tetrahydrofuran-3-yl)pyrimidin-2-yl)-7H-pyrrolo[2,3-d]pyrimidin-4-amine

To a solution of tert-butyl 9-(4-amino-7-methyl-5-(5-(tetrahydrofuran-3-yl)pyrimidin-2-yl)-7H-pyrrolo[2,3-d]pyrimidin-6-yl)-3-azaspiro[5.5]undec-8-ene-3-carboxylate (130 mg, 0.238 mmol) in DCM (4 mL) was added TFA (1 mL) at 0° C. and stirred at room temperature for 1 hr. The reaction solution was concentrated under vacuum to give the title compound (80 mg, 0.18 mmol, 75.6% yield) as a brown solid.

LC-MS m/e: 446.3 (MH⁺).

### Scheme AE, Step 6. Synthesis of 1-(9-(4-amino-7-methyl-5-(5-(tetrahydrofuran-3-yl)pyrimidin-2-yl)-7H-pyrrolo[2,3-d]pyrimidin-6-yl)-3-azaspiro[5.5]undec-8-en-3-yl)prop-2-en-1-one

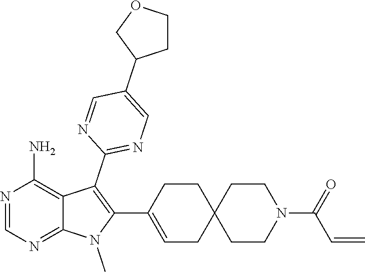 [SMILES: C=CC(=O)N1CCC2(CC=C(c3c(-c4ncc(C5CCOC5)cn4)c4c(N)ncnc4n3C)CC2)CC1]

### Example 52

To a solution of 7-methyl-6-(3-azaspiro[5.5]undec-8-en-9-yl)-5-(5-(tetrahydrofuran-3-yl)pyrimidin-2-yl)-7H-pyrrolo[2,3-d]pyrimidin-4-amine (80 mg, 0.180 mmol) and TEA (0.1 mL, 0.718 mmol) in DCM (5 mL) was added acryloyl chloride (90.5 mg, 0.197 mmol) at 0° C. and stirred under N₂ for 0.5 hrs. The resulting solution was poured into water (20 mL) and extracted with EtOAc (20 mL×2). The combined organic layers were washed with brine (20 mL), dried over anhydrous Na₂SO₄ and filtered. The filtrate was concentrated under vacuum and purified by silica gel column chromatography (gradient, DCM:MeOH=15:1) followed by prep-HPLC to give the title compound (22 mg, 0.044 mmol, 24.5% yield) as a light-yellow solid.

¹H NMR (400 MHz, DMSO-d6) δ 9.30 (br s, 0.75H), 8.69 (s, 2H), 8.09 (s, 1H), 7.27 (br, 2H), 6.86 (dd, J=15.9, 10.8 Hz, 1H), 6.67 (br, 1H), 6.11 (d, J=16.9 Hz, 1H), 5.68 (d, J=10.4 Hz, 1H), 5.63 (br. s, 1H), 4.04 (t, J=7.7 Hz, 1H), 3.98 (m, 1H), 3.86-3.79 (m, 1H), 3.69 (m, 1H), 3.67-3.62 (m, 2H), 3.59 (s, 3H), 2.39-2.26 (m, 3H), 2.03 (m, 1H), 1.75 (s, 4H), 1.70 (m, 2H), 1.56 (m, 4H).

LC-MS m/e: 500.5 (MH⁺).

### Example 53

### Synthesis of 1-(9-(4-amino-7-methyl-5-(oxazol-2-yl)-7H-pyrrolo[2,3-d]pyrimidin-6-yl)-3-azaspiro[5.5]undec-8-en-3-yl)prop-2-en-1-one

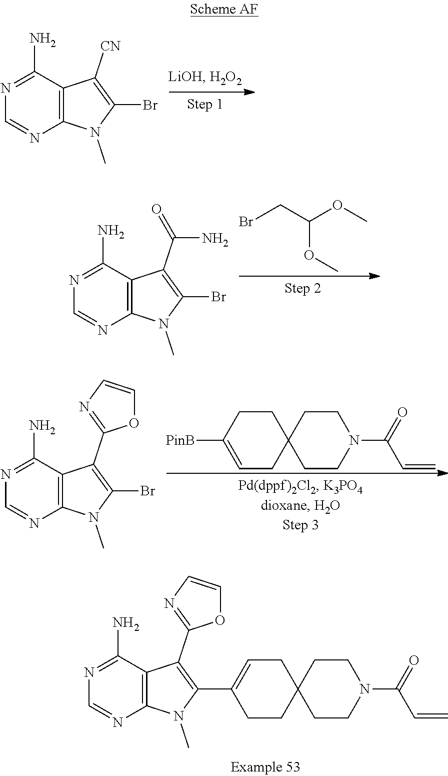

### Scheme AF, Step 1. Synthesis of 4-amino-6-bromo-7-methyl-7H-pyrrolo[2,3-d]pyrimidine-5-carboxamide

To a solution of 4-amino-6-bromo-7-methyl-7H-pyrrolo[2,3-d]pyrimidine-5-carbonitrile (120 mg, 0.476 mmol) in DMSO (8 mL) was added lithium hydroxide (22.8 mg, 0.952 mmol) and H₂O₂ (270 mg, 2.38 mmol, 30%) at 0° C. and stirred at 25° C. for 6 hrs. The mixture was quenched with water (50 mL) and extracted with EtOAc (50 mL×3). The combined organic layers were dried over anhydrous Na₂SO₄, filtered and concentrated to give the title compound (110 mg, 0.407 mmol, 85.6% yield) as a colorless oil.

LC-MS m/e: 270 (MH⁺).

### Scheme AF, Step 2. 6-bromo-7-methyl-5-(oxazol-2-yl)-7H-pyrrolo[2,3-d]pyrimidin-4-amine

A mixture of 4-amino-6-bromo-7-methyl-7H-pyrrolo[2,3-d]pyrimidine-5-carboxamide (110 mg, 0.407 mmol) and 2-bromo-1,1-dimethoxyethane (4.8 mL, 40.7 mmol) was irradiated in the microwave at 100° C. for 2 hrs. The mixture was cooled to room temperature, diluted with water (50 mL) and extracted with DCM (70 mL×3). The combined organic layers were dried over anhydrous Na₂SO₄, filtered and concentrated to dryness. The residue was purified by prep-TLC to give the title compound (15 mg, 0.051 mmol, 12.5% yield) as a yellow solid.

LC-MS m/e: 294 (MH⁺)/296.

### Scheme AF, Step 3. Synthesis of 1-(9-(4-amino-7-methyl-5-(oxazol-2-yl)-7H-pyrrolo[2,3-d]pyrimidin-6-yl)-3-azaspiro[5.5]undec-8-en-3-yl)prop-2-en-1-one

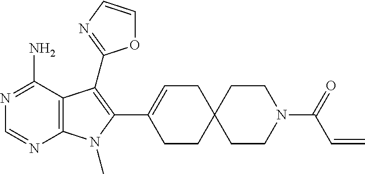 [SMILES: C=CC(=O)N1CCC2(CC=C(c3c(-c4ncco4)c4c(N)ncnc4n3C)CC2)CC1]

### Example 53

To a solution of 6-bromo-7-methyl-5-(oxazol-2-yl)-7H-pyrrolo[2,3-d]pyrimidin-4-amine (15 mg, 0.051 mmol) in dioxane (6 mL) and water (1.5 mL) were added Pd(PPh₃)₂Cl₂ (7 mg, 0.01 mmol), K₃PO₄ (33 mg, 0.153 mmol) and 1-(9-(4,4,5,5-tetramethyl-1,3,2-dioxaborolan-2-yl)-3-azaspiro[5.5]undec-8-en-3-yl)prop-2-en-1-one (25 mg, 0.077 mmol). The mixture was stirred at 80° C. for 3 hrs under N₂. The resulting mixture was cooled to room temperature, diluted with water (10 mL) and extracted with EtOAc (20 mL×3). The combined organic layers were dried over anhydrous Na₂SO₄, filtered and concentrated to dryness. The residue was purified by prep-TLC to give the title compound (2.3 mg, 0.005 mmol, 10.8% yield) as a white solid.

¹H NMR (400 MHz, DMSO-d6) δ 10.05 (s, 1H), 9.23 (s, 1H), 8.08 (d, J=1.1 Hz, 1H), 7.58 (d, J=1.1 Hz, 1H), 7.13 (s, 1H), 6.84 (dd, J=16.7, 10.3 Hz, 1H), 6.09 (dd, J=16.7, 2.5 Hz, 1H), 5.68 (m, 1H), 5.64 (d, J=4.0. 1H), 3.74 (s, 3H), 3.67-3.49 (m, 4H), 2.33 (br. s, 2H), 2.14 (s, 2H), 1.67 (t, J=6.1 Hz, 2H), 1.61-1.48 (m, 4H).

LC-MS m/e: 419 (MH⁺).

### Example 54

### Synthesis of 1-(9-(4-amino-7-(2-hydroxyethyl)-5-(pyrimidin-2-yl)-7H-pyrrolo[2,3-d]pyrimidin-6-yl)-3-azaspiro[5.5]undec-8-en-3-yl)prop-2-en-1-one

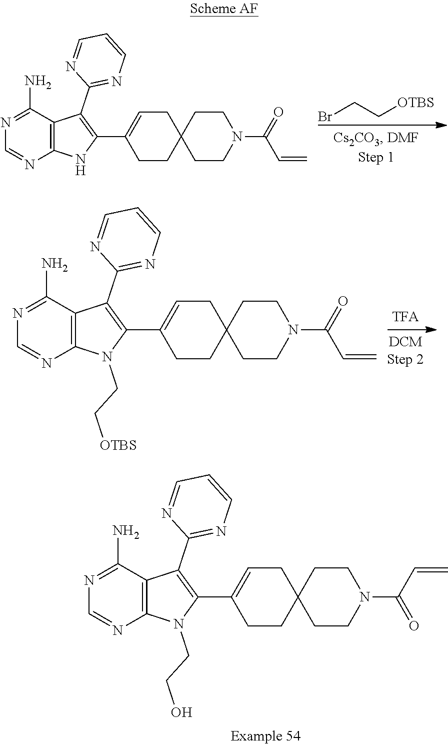 [SMILES: C=CC(=O)N1CCC2(CC=C(c3[nH]c4ncnc(N)c4c3-c3ncccn3)CC2)CC1 ; CC(C)(C)[Si](C)(C)OCCBr ; C=CC(=O)N1CCC2(CC=C(c3c(-c4ncccn4)c4c(N)ncnc4n3CCO[Si](C)(C)C(C)(C)C)CC2)CC1 ; C=CC(=O)N1CCC2(CC=C(c3c(-c4ncccn4)c4c(N)ncnc4n3CCO)CC2)CC1]

### Scheme AF, Step 1. Synthesis of 1-(9-(4-amino-7-(2-((tert-butyldimethylsilyl)oxy)ethyl)-5-(pyrimidin-2-yl)-7H-pyrrolo[2,3-d]pyrimidin-6-yl)-3-azaspiro[5.5]undec-8-en-3-yl)prop-2-en-1-one

A mixture of 1-(9-(4-amino-5-(pyrimidin-2-yl)-7H-pyrrolo[2,3-d]pyrimidin-6-yl)-3-azaspiro[5.5]undec-8-en-3-yl)prop-2-en-1-one (46 mg, 0.11 mmol), Cs₂CO₃ (36.1 mg, 0.11 mmol) and 1-bromo-4,4,5,5-tetramethyl-3-oxa-4-silahexane (32 mg, 0.13 mmol) in DMF (10 mL) was stirred at 80° C. for 6 hrs. The reaction mixture was cooled to room temperature, diluted with water (10 mL) and extracted with EtOAc (20 mL×2). The combined extracts were dried over anhydrous Na₂SO₄, filtered and concentrated to afford crude tittle compound (40 mg, 0.07 mmol, 62.7% yield) as a yellow solid.

LC-MS m/e:574 (MH⁺).

### Scheme AF, Step 2. Synthesis of 1-(9-(4-amino-7-(2-hydroxyethyl)-5-(pyrimidin-2-yl)-7H-pyrrolo[2,3-d]pyrimidin-6-yl)-3-azaspiro[5.5]undec-8-en-3-yl)prop-2-en-1-one

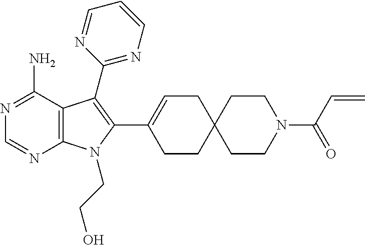 [SMILES: C=CC(=O)N1CCC2(CC=C(c3c(-c4ncccn4)c4c(N)ncnc4n3CCO)CC2)CC1]

### Example 54

A solution of 1-(9-(4-amino-7-(2-((tert-butyldimethylsilyl)oxy)ethyl)-5-(pyrimidin-2-yl)-7H-pyrrolo[2,3-d]pyrimidin-6-yl)-3-azaspiro[5.5]undec-8-en-3-yl)prop-2-en-1-one (40 mg, 0.07 mmol) in a mixture of TFA (0.5 mL) and DCM (1.5 mL) was stirred at 0° C. for 1 hr. The mixture was treated with saturated NaHCO₃ solution (40 mL) and extracted with DCM (40 mL×2). The combined organic layers were dried over anhydrous Na₂SO₄, filtered and concentrated to dryness. The residue was purified by prep-HPLC to afford the tittle compound (15 mg, 0.03 mmol, 46.8% yield) as a yellow solid.

¹H NMR (400 MHz, DMSO-d6) δ 9.34 (hump, 0.70H), 8.78 (d, J=4.8 Hz, 2H), 8.09 (s, 1H), 7.27-7.06 (m, 2H), 6.90-6.80 (m, 1H), 6.11 (dd, J=16.4, 2.0 Hz, 1H), 5.71-5.59 (m, 2H), 4.95 (t, J=5.4 Hz, 1H), 4.19-4.06 m, 2H), 3.71-3.66 (m, 4H), 3.58-3.51 (m, 2H), 2.34-2.29 (m, 2H), 2.11 (br.s, 2H), 1.69-1.67 (m, 2H), 1.63-1.49 (m, 4H).

LC-MS m/e:460 (MH⁺).

### Example 55

### Synthesis of 1-(9-(4-amino-7-methyl-5-(oxetan-3-yl)-7H-pyrrolo[2,3-d]pyrimidin-6-yl)-3-azaspiro[5.5]undec-8-en-3-yl)prop-2-en-1-one

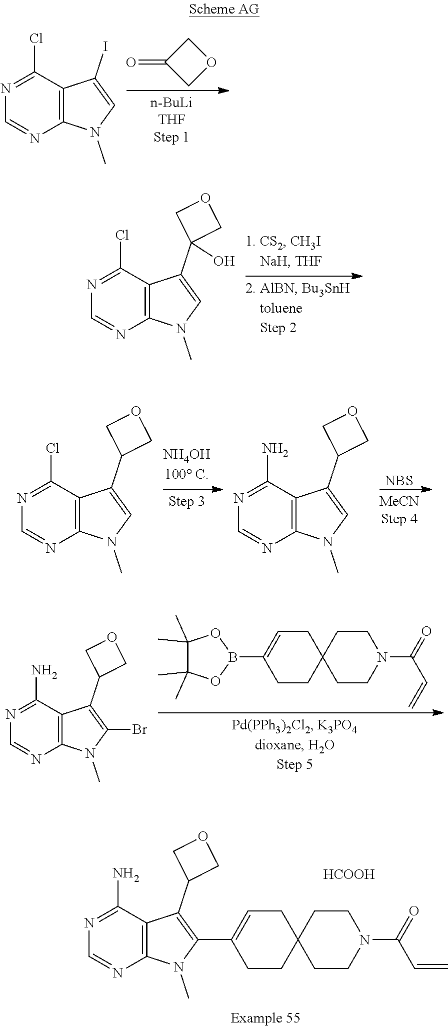

### Scheme AG, Step 1. Synthesis of 3-(4-chloro-7-methyl-7H-pyrrolo[2,3-d]pyrimidin-5-yl)oxetan-3-ol

To a solution of 4-chloro-5-iodo-7-methylpyrrolo[2,3-d]pyrimidine (2 g, 6.8 mmol) in THF (40 mL) was added n-BuLi (3.4 mL, 6.8 mmol, 2 M in THF) at −15° C. The mixture was stirred at −15° C. for 30 mins, then oxetan-3-one (740 mg, 10.2 mmol) was added to the mixture at −15° C. The reaction was stirred at −5° C. for 30 mins, quenched with saturated NH₄Cl solution (50 mL) and extracted with EtOAc (150 mL×2). The combined extracts were dried over anhydrous Na₂SO₄, filtered and concentrated to dryness. The residue was purified by silica gel column chromatography (gradient, DCM to DCM:MeOH=20:1) to afford the tittle compound (1.2 g, 5.04 mmol, 73.5% yield) as a yellow solid.

LC-MS m/e:240 (MH⁺)

### Scheme AG, Step 2. Synthesis of 4-chloro-7-methyl-5-(oxetan-3-yl)-7H-pyrrolo[2,3-d]pyrimidine

To a solution of 3-(4-chloro-7-methylpyrrolo[2,3-d]pyrimidin-5-yl)oxetan-3-ol (1.2 g, 5 mmol) in THF (40 mL) was added NaH (1.42 g, 35.5 mmol, 60% suspended in mineral oil) at 0° C. and stirred at room temperature for 2 hrs. Then carbon disulfide (2.4 mL, 40 mmol) was added to the mixture and stirred for another 30 mins, followed by the addition of Mel (3.1 mL, 50 mmol). The resulting mixture was stirred at room temperature for 1 hr, quenched with saturated NH₄Cl solution (100 mL) and extracted with EtOAc (200 mL×2). The combined organic layers were dried over anhydrous Na₂SO₄, filtered and concentrated to dryness. The residue was dissolved in toluene (20 mL), AIBN (0.16 g, 1 mmol) and Bu₃SnH (5.83 g, 20 mmol) were added to the solution and stirred at 100° C. for 3 hrs. The reaction mixture was cooled to room temperature and concentrated. The residue was purified by silica gel column chromatography (gradient, DCM to DCM:MeOH=50:1) to afford the tittle compound (600 mg, 2.68 mmol, 53.6% yield) as a yellow solid.

LC-MS m/e:224 (MH⁺).

### Scheme AG, Step 3. Synthesis of 7-methyl-5-(oxetan-3-yl)-7H-pyrrolo[2,3-d]pyrimidin-4-amine

A solution of 4-chloro-7-methyl-5-(oxetan-3-yl)-7H-pyrrolo[2,3-d]pyrimidine (500 mg, 2.236 mmol) in NH₃ solution (10 mL, 25% in water) was stirred at 100° C. in a sealed tube for 16 hrs. The reaction was cooled to room temperature and concentrated to dryness. The residue was purified by silica gel column chromatography (gradient, DCM to DCM:MeOH=10:1) to afford the tittle compound (360 mg, 1.76 mmol, 78.9% yield) as a yellow solid.

LC-MS m/e:205 (MH⁺).

### Scheme AG, Step 4. Synthesis of 6-bromo-7-methyl-5-(oxetan-3-yl)-7H-pyrrolo[2,3-d]pyrimidin-4-amine

A solution of 7-methyl-5-(oxetan-3-yl)pyrrolo[2,3-d]pyrimidin-4-amine (300 mg, 1.47 mmol) and NBS (392 mg, 2.2 mmol) in MeCN (10 mL) was stirred at room temperature for 4 hrs. The reaction mixture was treated with water (50 mL) and extracted with EtOAc (100 mL). The organic layer was dried over anhydrous Na₂SO₄, filtered and concentrated to dryness. The residue was purified by silica gel column chromatography (gradient, DCM to DCM:MeOH=10:1) to afford the tittle compound (100 mg, 0.35 mmol, 24% yield) as a yellow solid.

LC-MS m/e:283, 285 (MH⁺).

### Scheme AG, Step 5. Synthesis of 1-(9-(4-amino-7-methyl-5-(oxetan-3-yl)-7H-pyrrolo[2,3-d]pyrimidin-6-yl)-3-azaspiro[5.5]undec-8-en-3-yl)prop-2-en-1-one

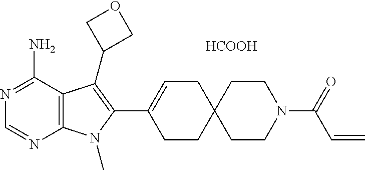 [SMILES: C=CC(=O)N1CCC2(CC=C(c3c(C4COC4)c4c(N)ncnc4n3C)CC2)CC1 ; "HCOOH"]

### Example 55

A solution of -bromo-7-methyl-5-(oxetan-3-yl)-7H-pyrrolo[2,3-d]pyrimidin-4-amine (80 mg, 0.28 mmol), 1-[9-(4,4,5,5-tetramethyl-1,3,2-dioxaborolan-2-yl)-3-azaspiro[5.5]-undec-8-en-3-yl]prop-2-en-1-one (112 mg, 0.338 mmol), Pd(dppf)Cl₂ (21 mg, 0.0289 mmol) and K₃PO₄ (180 mg, 0.848 mmol) in dioxane (8 mL) and water (20 mL) was stirred at 100° C. for 2 hrs under N₂. Then the mixture was cooled to room temperature, diluted with water (10 mL) and extracted with EtOAc (20 mL×2). The combined layers were dried over anhydrous Na₂SO₄, filtered and concentrated to dryness. The residue was purified by prep-HPLC to afford the tittle compound (8 mg, 0.02 mmol, 6.95% yield) as a yellow solid.

¹H NMR (400 MHz, DMSO-d6) with HCOOH, δ 8.14 (s, 1H, HCOOH), 8.09 (s, 1H), 7.09 (s, 2H), 6.82 (dd, J=16.6, 10.6 Hz, 1H), 6.09 (dd, J=16.8, 2.6 Hz, 1H), 5.70-5.62 (m, 2H), 5.21-5.12 (m, 2H), 4.48 (t, J=6.0 Hz, 2H), 4.29-4.16 (m, 1H), 3.69-3.59 (m, 2H), 3.59-3.48 (m, 5H), 2.17-2.04 (m, 4H), 1.65 (t, J=6.0 Hz, 2H), 1.52-1.38 (m, 4H).

LC-MS m/e:408 (MH⁺).

### Example 56

### Synthesis of 1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one

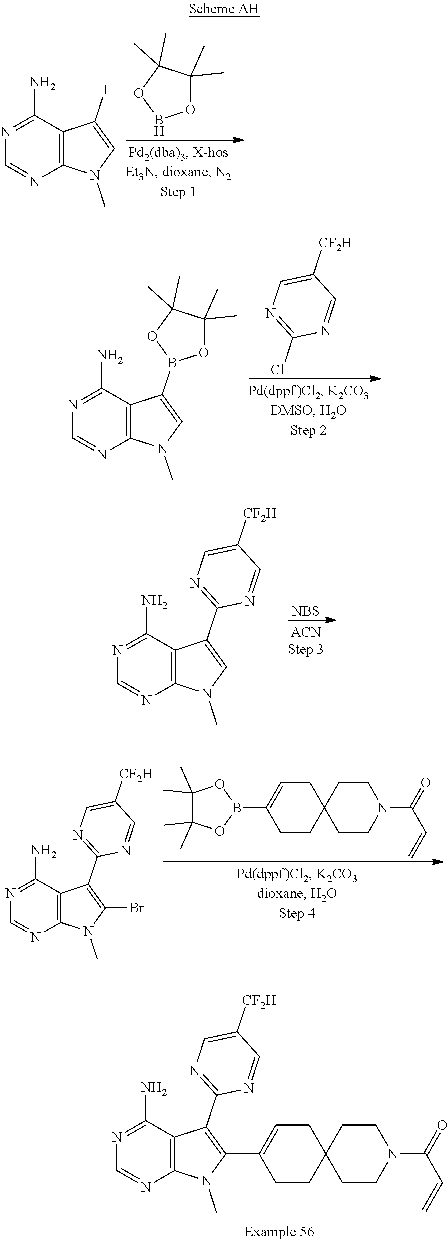

### Scheme AH, Step 1. Synthesis of 7-methyl-5-(4,4,5,5-tetramethyl-1,3,2-dioxaborolan-2-yl)-7H-pyrrolo[2,3-d]pyrimidin-4-amine

To a solution of 5-iodo-7-methyl-7H-pyrrolo[2,3-d]pyrimidin-4-amine (1 g, 3.65 mmol) in dioxane (25 mL) were added 4,4,5,5-tetramethyl-1,3,2-dioxaborolane (2.8 g, 21.9 mmol), Pd₂(dba)₃ (2.95 g, 3.65 mmol), X-Phos (1.74 g, 3.65 mmol) and Et₃N (0.507 mL, 3.65 mmol) at 25° C. under N₂. The mixture was stirred at 75° C. for 3 hrs under N₂. The reaction mixture was cooled to room temperature, diluted with water (20 mL) and extracted with EtOAc (40 mL×2). The combined extracts were dried over anhydrous Na₂SO₄, filtered, concentrated and purified by silica gel column chromatography (gradient, DCM to DCM:MeOH=9:1) to afford the title compound (270 mg, 0.985 mmol, 27% yield) as a yellow solid.

LC-MS m/e: 275 (MH⁺).

### Scheme AH, Step 2. Synthesis of 5-(5-(difluoromethyl)pyrimidin-2-yl)-7-methyl-7H-pyrrolo[2,3-d]pyrimidin-4-amine

To a mixture of 7-methyl-5-(4,4,5,5-tetramethyl-1,3,2-dioxaborolan-2-yl)-7H-pyrrolo[2,3-d]pyrimidin-4-amine (300 mg, 1.09 mmol) and 2-chloro-5-(difluoromethyl)-pyrimidine (120 mg, 0.729 mmol) in DMSO (12 mL) and H₂O (2 mL) was added Pd(PPh₃)₂Cl₂ (102 mg, 0.146 mmol) and K₃PO₄ (464 mg, 2.19 mmol). The reaction mixture was stirred at 75° C. under N₂ for 3 hrs. After allowing the reaction mixture to cool to room temperature, the reaction mixture was diluted with H₂O (20 mL) and extracted with EtOAc (20 mL×3). The organic phase was washed with brine (30 mL×2), dried over anhydrous Na₂SO₄, filtered and concentrated to dryness. The residue was purified by silica gel column chromatography (gradient, DCM to DCM:MeOH=9:1) to afford the title compound (190 mg, 0.688 mmol, 94.3% yield) as a white solid.

LC-MS m/e:275 (MH⁺).

### Scheme AH, Step 3. Synthesis of 6-bromo-5-(5-(difluoromethyl)pyrimidin-2-yl)-7-methyl-7H-pyrrolo[2,3-d]pyrimidin-4-amine

To a solution of 5-(5-(difluoromethyl)pyrimidin-2-yl)-7-methyl-7H-pyrrolo[2,3-d]pyrimidin-4-amine (200 mg, 0.724 mmol) in CH₃CN (1.5 mL) was added NBS (193 mg, 1.09 mmol) at 25° C. and stirred at 25° C. for 3 hrs. The reaction mixture was diluted with water (10 mL) and extracted with EtOAc (20 mL×2). The combined extracts were dried over anhydrous Na₂SO₄, filtered, concentrated and purified by silica gel column chromatography (gradient, DCM to DCM:MeOH=9:1) to afford the title compound (50 mg, 0.141 mmol, 19.45%) as a yellow solid.

LC-MS m/e: 355, 357 (MH⁺).

### Scheme AH, Step 4. Synthesis of 1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one

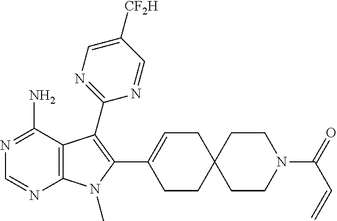 [SMILES: C=CC(=O)N1CCC2(CC=C(c3c(-c4ncc(C(F)F)cn4)c4c(N)ncnc4n3C)CC2)CC1]

### Example 56

To a mixture of 6-bromo-5-(5-(difluoromethyl)pyrimidin-2-yl)-7-methyl-7H-pyrrolo[2,3-d]pyrimidin-4-amine (40 mg, 0.113 mmol) and 1-(9-(4,4,5,5-tetramethyl-1,3,2-dioxaborolan-2-yl)-3-azaspiro[5.5]undec-8-en-3-yl)prop-2-en-1-one (56 mg, 0.169 mmol) in dioxane (5 mL) and H₂O (1 mL) were added Pd(PPh₃)₂Cl₂ (79 mg, 0.113 mmol) and K₃PO₄ (24 mg, 0.113 mmol). The reaction mixture was stirred at 100° C. for 3 hrs under N₂. After allowing the reaction mixture to cool to room temperature, the reaction mixture was diluted with H₂O (20 mL) and extracted with EtOAc (20 mL×2). The organic phase was washed with brine (20 mL), dried over anhydrous Na₂SO₄, filtered and concentrated to dryness. The residue was purified by prep-HPLC to afford the title compound (40 mg, 0.083 mmol, 74.1% yield) as a white solid.

¹H NMR (400 MHz, DMSO-d6) δ 9.14 (hump, 0.65H), 8.98 (s, 2H), 8.12 (s, 1H), 7.37-7.00 (m, 2H), 6.86 (dd, J=16, 12 Hz, 1H), 6.12 (dd, J=16, 1 Hz, 1H), 5.71-5.64 (m, 2H), 3.75-3.64 (m, 2H), 3.61 (s, 3H), 3.58-3.49 (m, 2H), 2.34-2.24 (m, 2H), 2.12 (s, 2H), 1.72 (t, J=5.8 Hz, 2H), 1.63-1.50 (m, 4H).

LC-MS m/e:480 (MH⁺).

### Example 57

### Synthesis of 6-(3-acryloyl-3-azaspiro[5.5]undec-8-en-9-yl)-4-amino-7-methyl-7H-pyrrolo[2,3-d]pyrimidine-5-carbonitrile

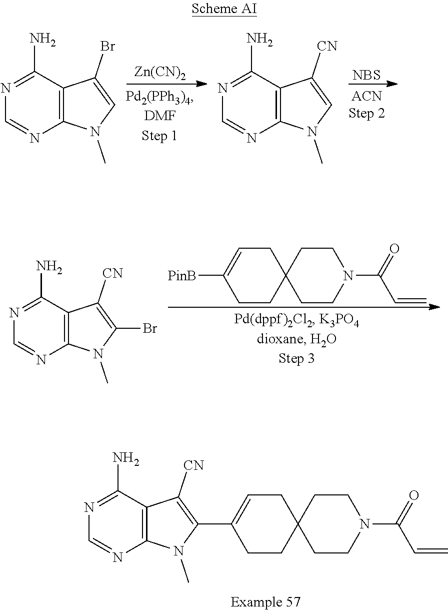

### Scheme AI, Step 1. Synthesis of 4-amino-7-methyl-7H-pyrrolo[2,3-d]pyrimidine-5-carbonitrile

To a solution of 5-bromo-7-methylpyrrolo[2,3-d]pyrimidin-4-amine (2 g, 8.8 mmol) and Zn(CN)₂ (4.93 g, 42 mmol) in DMF (25 mL) were added Pd(PPh₃)₄ (1.94 g, 1.68 mmol), the mixture was stirred at 100° C. for 3 hrs under N₂. The resulting mixture was cooled to room temperature and filtered. The filtrate was quenched with saturated NaHCO₃ solution (50 mL) and extracted with EtOAc (50 mL×3). The combined organic layers were washed with brine (100 mL), dried over anhydrous Na₂SO₄, filtered and concentrated to dryness. The residue was purified by flash chromatography on alumina (gradient, DCM to DCM:MeOH=20:1) to afford the title compound (630 mg, 3.64 mmol, 43.3% yield) as a yellow solid.

LC-MS m/e: 174 (MH⁺).

### Scheme AI, Step 2. Synthesis of 4-amino-6-bromo-7-methyl-7H-pyrrolo[2,3-d]pyrimidine-5-carbonitrile

To a solution of 4-amino-7-methyl-7H-pyrrolo[2,3-d]pyrimidine-5-carbonitrile (250 mg, 1.44 mmol) in MeCN (6 mL) was added NBS (511 mg, 2.87 mmol) at 0° C. and stirred for 1 hr under N₂. The reaction mixture was poured into water (10 mL) and extracted with EtOAc (20 mL×2). The combined organic layers were washed with brine (20 mL), dried over anhydrous Na₂SO₄, filtered and concentrated under reduced pressure to dryness. The residue was purified by flash chromatography on silica gel (gradient, DCM to DCM:MeOH=20:1) to afford the title compound (237 mg, 0.94 mmol, 65.1% yield) as a yellow solid.

LC-MS m/e: 252, 254 (MH⁺).

### Scheme AI, Step 3. Synthesis of 6-(3-acryloyl-3-azaspiro[5.5]undec-8-en-9-yl)-4-amino-7-methyl-7H-pyrrolo[2,3-d]pyrimidine-5-carbonitrile

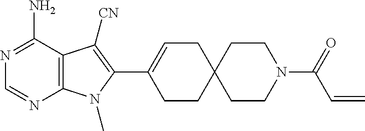 [SMILES: C=CC(=O)N1CCC2(CC=C(c3c(C#N)c4c(N)ncnc4n3C)CC2)CC1]

### Example 57

To a solution of 4-amino-6-bromo-7-methyl-7H-pyrrolo[2,3-d]pyrimidine-5-carbonitrile (50 mg, 0.2 mmol), 1-(9-(4,4,5,5-tetramethyl-1,3,2-dioxaborolan-2-yl)-3-azaspiro[5.5]undec-8-en-3-yl)prop-2-en-1-one (100 mg, 0.3 mmol) and K₃PO₄ (126 mg, 0.594 mmol) in dioxane (2 mL) and H₂O (0.4 mL) was added Pd(PPh₃)₂Cl₂ (28 mg, 0.04 mmol). The mixture was stirred at 90° C. for 12 hrs under N₂. The resulting mixture was cooled to room temperature and concentrated to dryness. The residue is partitioned between water (10 mL) and EtOAc (20 mL). The organic layer was washed with brine (10 mL), dried over anhydrous Na₂SO₄, filtered, concentrated under reduced pressure, and purified by prep-HPLC to afford the title compound (15 mg, 0.04 mmol, 20% yield) as a yellow solid.

¹H NMR (400 MHz, DMSO-d6) δ 8.22 (s, 1H), 6.8.3 (dd, K=16, 8 Hz, 1H), 6.72 (hump, 2H), 6.15-6.04 (m, 2H), 5.66 (dd, J=10.4, 2.4 Hz, 1H), 3.64 (s, 3H), 3.61-3.43 (m, 4H), 2.40-2.33 (m, 2H), 2.27-2.15 (m, 2H), 1.69 (t, J=6.0 Hz, 2H), 1.53-1.37 (m, 4H).

LC-MS m/e: 377 (MH⁺).

### Example 58

### Synthesis of 1-(9-(4-amino-5-(pyrimidin-2-yl)-7H-pyrrolo[2,3-d]pyrimidin-6-yl)-3-azaspiro[5.5]undec-8-en-3-yl)prop-2-en-1-one

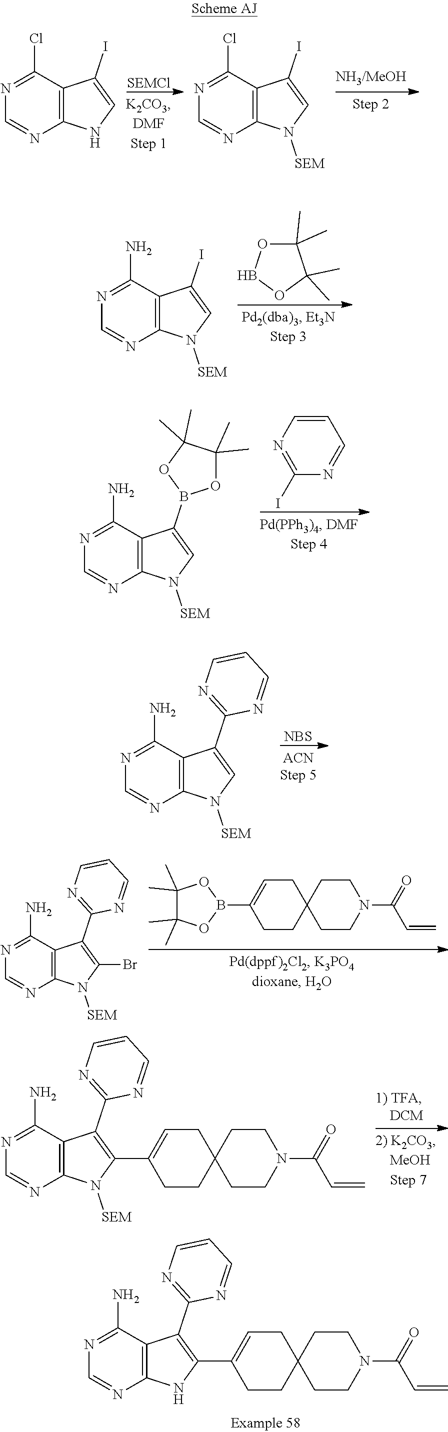

### Scheme AJ, Step 1. Synthesis of 4-chloro-5-iodo-7-((2-(trimethylsilyl)ethoxy)methyl)-7H-pyrrolo[2,3-d]pyrimidine

To a solution of 4-chloro-5-iodo-7H-pyrrolo[2,3-d]pyrimidine (2 g, 7.156 mmol) in DMF (30 mL) was added K₂CO₃ (2.97 g, 21.4 mmol) at room temperature. After stirring for 20 mins, SEMCl (1.9 mL, 10.7 mmol) was added dropwise. The reaction mixture was stirred at room temperature for 2 hrs, poured into water and extracted with EtOAc (50 mL×3). The combined organic layers were washed with brine (50 mL), dried over anhydrous Na₂SO₄, filtered and concentrated to dryness. The residue was purified by flash column chromatography on silica gel (gradient, PE to PE:EtOAc=5:1) to afford the title compound (2.8 g, 6.83 mmol, 95.5% yield) as a brown solid.

LC-MS m/e:410.2 (MH⁺).

### Scheme AJ, Step 2. Synthesis of 5-iodo-7-((2-(trimethylsilyl)ethoxy)methyl)-7H-pyrrolo[2,3-d]pyrimidin-4-amine

A solution of 4-chloro-5-iodo-7-((2-(trimethylsilyl)ethoxy)methyl)-7H-pyrrolo[2,3-d]pyrimidine (2.8 g, 6.83 mmol) and NH₄OH (30 mL, 25% in water) in dioxane (10 mL) was stirred at 120° C. in a sealed tube for 16 hrs. The reaction mixture was cooled to room temperature, filtered and the filter cake was washed with PE (50 mL). The cake was dried under vacuum to afford the title compound (2.4 g, 6.15 mmol, 90% yield) as a white solid.

LC-MS m/e: 391.1 (MH⁺).

### Scheme AJ, Step 3. Synthesis of 5-(4,4,5,5-tetramethyl-1,3,2-dioxaborolan-2-yl)-7-((2-(trimethylsilyl)ethoxy)methyl)-7H-pyrrolo[2,3-d]pyrimidin-4-amine

To a solution of 5-iodo-7-((2-(trimethylsilyl)ethoxy)methyl)-7H-pyrrolo[2,3-d]pyrimidin-4-amine (2.3 g, 5.89 mmol) in dioxane (50 mL) were added Pd₂(dba)₃ (1.08 g, 1.18 mmol), X-phos (1.12 g, 2.36 mmol), 4,4,5,5-tetramethyl-1,3,2-dioxaborolane (4.52 g, 35.3 mmol) and Et₃N (4.9 mL, 35.4 mmol) at 25° C. under N₂. The mixture was stirred at 80° C. for 4 hrs under N₂. The reaction mixture was cooled to room temperature and partitioned between water (50 mL) and EtOAc (150 mL). The organic layer was washed with brine (50 mL), dried over anhydrous Na₂SO₄, filtered and concentrated to dryness. The residue was purified by flash column chromatography on silica gel (gradient, DCM to DCM:MeOH=10:1) to afford the title compound (1 g, 2.56 mmol, 43.4% yield) as a white solid.

LC-MS m/e:391.3 (MH⁺).

### Scheme AJ, Step 4. Synthesis of 5-(pyrimidin-2-yl)-7-((2-(trimethylsilyl)ethoxy)methyl)-7H-pyrrolo[2,3-d]pyrimidin-4-amine

To a solution of 2-iodopyrimidine (350 mg, 1.7 mmol) and 5-(4,4,5,5-tetramethyl-1,3,2-dioxaborolan-2-yl)-7-((2-(trimethylsilyl)ethoxy)methyl)-7H-pyrrolo[2,3-d]pyrimidin-4-amine (700 mg, 1.79 mmol) in DMSO (10 mL) and water (2 mL) were added Pd(PPh₃)₄ (310 mg, 0.269 mmol) and K₃PO₄ (1.14 g, 5.38 mmol) at 20° C. under N₂. The mixture was stirred at 70° C. for 3 hrs under N₂. The reaction mixture was cooled to room temperature and partitioned between water (20 mL) and EtOAc (80 mL). The organic layer was washed with brine (50 mL), dried over anhydrous Na₂SO₄, filtered and concentrated to dryness. The residue was purified by flash column chromatography on silica gel (gradient, DCM to DCM:MeOH=10:1) to afford the title compound (420 mg, 1.23 mmol, 68.4% yield) as a white solid.

LC-MS m/e:343.3 (MH⁺).

### Scheme AJ, Step 5. Synthesis of 6-bromo-5-(pyrimidin-2-yl)-7-((2-(trimethylsilyl)ethoxy)-methyl)-7H-pyrrolo[2,3-d]pyrimidin-4-amine

To a solution of 5-(pyrimidin-2-yl)-7-((2-(trimethylsilyl)ethoxy)methyl)-7H-pyrrolo-[2,3-d]pyrimidin-4-amine (320 mg, 0.934 mmol) in MeCN (5 mL) was added NBS (262 mg, 1.47 mmol) at 15° C. and stirred for 2 hrs. The reaction mixture was partitioned between water (10 mL) and EtOAc (30 mL). The organic layer was washed with brine (20 mL), dried over anhydrous Na₂SO₄, filtered and concentrated to dryness. The residue was purified by flash column chromatography on silica gel (gradient, DCM to DCM:MeOH=10:1) to afford the title compound (250 mg, 0.593 mmol, 63.5% yield) as a white solid.

LC-MS m/e:421.2, 423.2 (MH⁺).

### Scheme AJ, Step 6. Synthesis of 1-(9-(4-amino-5-(pyrimidin-2-yl)-7-((2-(trimethylsilyl)ethoxy)methyl)-7H-pyrrolo[2,3-d]pyrimidin-6-yl)-3-azaspiro[5.5]undec-8-en-3-yl)prop-2-en-1-one

To a solution of 6-bromo-5-(pyrimidin-2-yl)-7-((2-(trimethylsilyl)ethoxy)methyl)-7H-pyrrolo[2,3-d]pyrimidin-4-amine (250 mg, 0.593 mmol) and 1-(9-(4,4,5,5-tetramethyl-1,3,2-dioxaborolan-2-yl)-3-azaspiro[5.5]undec-8-en-3-yl)prop-2-en-1-one (294 mg, 0.89 mmol) in dioxane (5 mL) and water (1 mL) were added Pd(dppf)Cl₂ (87 mg, 0.119 mmol) and K₃PO₄ (378 mg, 1.78 mmol) at 25° C. under N₂. The reaction mixture was stirred at 80° C. for 4 hrs under N₂. It was then cooled to room temperature and partitioned between water (10 mL) and EtOAc (50 mL). The organic layer was washed with brine (30 mL), dried over anhydrous Na₂SO₄, filtered and concentrated to dryness. The residue was purified by flash column chromatography on silica gel (gradient, DCM to DCM:MeOH=10:1) to afford the title compound (320 mg, 0.528 mmol, 88.9% yield) as a yellow oil.

LC-MS m/e:546.5 (MH⁺).

### Scheme AJ, Step 7. Synthesis of 1-(9-(4-amino-5-(pyrimidin-2-yl)-7H-pyrrolo[2,3-d]pyrimidin-6-yl)-3-azaspiro[5.5]undec-8-en-3-yl)prop-2-en-1-one

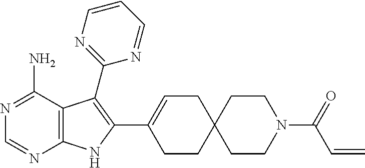 [SMILES: C=CC(=O)N1CCC2(CC=C(c3[nH]c4ncnc(N)c4c3-c3ncccn3)CC2)CC1]

### Example 58

A solution of 1-(9-(4-amino-5-(pyrimidin-2-yl)-7-((2-(trimethylsilyl)ethoxy)methyl)-7H-pyrrolo[2,3-d]pyrimidin-6-yl)-3-azaspiro[5.5]undec-8-en-3-yl)prop-2-en-1-one (250 mg, 0.458 mmol) in TFA (1 mL) and DCM (1 mL) was stirred at 25° C. for 1 hr. The mixture was concentrated and dissolved in MeOH (5 mL). Then K₂CO₃ (315 mg, 2.29 mmol) was added. The reaction mixture was stirred at 25° C. for 0.5 hrs. It was then partitioned between water (10 mL) and DCM (20 mL). The organic layer was washed with brine (10 mL), dried over anhydrous Na₂SO₄, filtered and concentrated to dryness. The residue was purified by reverse phase chromatography (SilaSep™ C18 silica flash cartridge, 0%-40% MeCN in H₂O with 0.1% formic acid) to afford the title compound (60 mg, 0.144 mmol, 31.5% yield) as a yellow solid.

¹H NMR (400 MHz, DMSO-d6) with HCOOH, δ 11.96 (s, 1H), 8.77 (d, J=4.8 Hz, 2H), 8.14 (s, 1H), 8.03 (s, 1H), 7.25 (t, J=4.8 Hz, 1H), 6.86 (dd, J=16.8, 10.8 Hz, 1H), 6.10 (dd, J=16.8, 2.4 Hz, 1H), 5.75-5.60 (m, 2H), 3.75-3.62 (m, 2H), 3.34-3.56 (m, 6.2 Hz, 2H), 2.32-2.22 (m, 2H), 2.12-2.02 (m, 2H), 1.67-1.44 (m, 6H).

LC-MS m/e:416.4 (MH⁺).

In a like manner, Example 63, 1-(9-(4-amino-5-(5-(difluoromethyl)pyrimidin-2-yl)-7H-pyrrolo[2,3-d]pyrimidin-6-yl)-3-azaspiro[5.5]undec-8-en-3-yl)prop-2-en-1-one, was prepared.

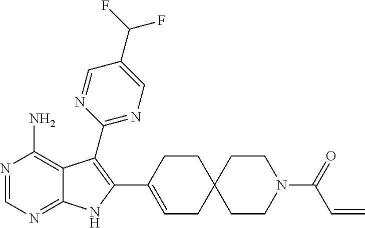 [SMILES: C=CC(=O)N1CCC2(CC=C(c3[nH]c4ncnc(N)c4c3-c3ncc(C(F)F)cn3)CC2)CC1]

### Example 6

LC-MS m/e:466.3 (MH⁺).

1H NMR (399 MHz, CD3OD) δ 8.90 (s, 2H), 8.18 (s, 1H), 6.92 (t, J=55.1 Hz, 1H), 6.74 (dd, J=16.8, 10.7 Hz, 1H), 6.11 (dd, J=16.8, 1.8 Hz, 1H), 5.79 (s, 1H), 5.66 (dd, J=10.7, 1.9 Hz, 1H), 3.80-3.63 (m, 2H), 3.62-3.49 (m, 2H), 2.39-2.31 (m, 2H), 2.16-2.09 (m, 2H), 1.68 (t, J=6.3 Hz, 2H), 1.64-1.53 (m, 4H).

### Preparation 4

### Synthesis of 5-(3-fluoro-4-((4-methylpyrimidin-2-yl)oxy)phenyl)-7-methyl-6-(3-azaspiro[5.5]undec-8-en-9-yl)-7H-pyrrolo[2,3-d]pyrimidin-4-amine

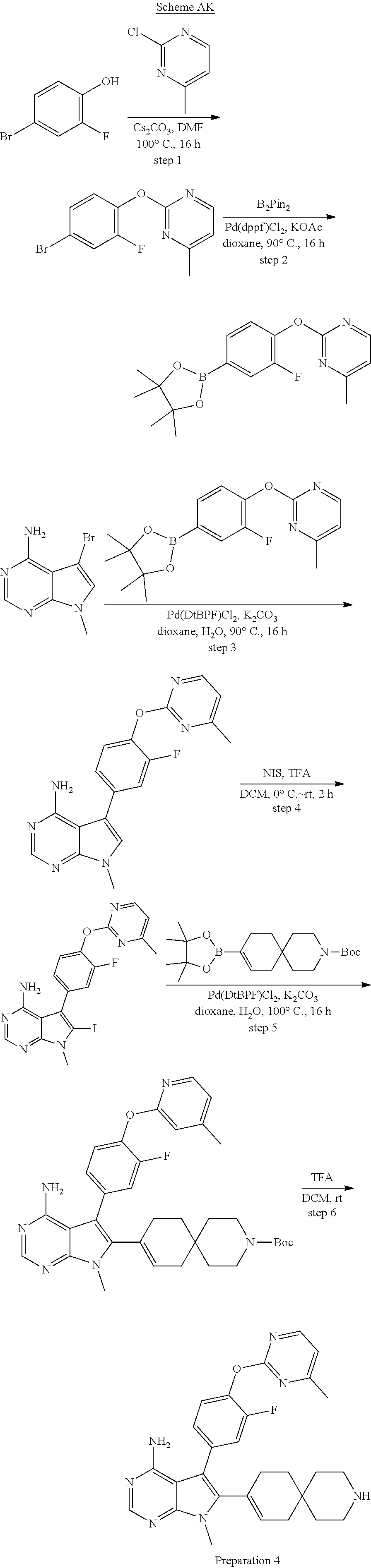

### Scheme AK, Step 1. Synthesis of 2-(4-bromo-2-fluorophenoxy)-4-methylpyrimidine

A mixture of 4-bromo-2-fluorophenol (25 g, 131 mmol, 1 eq.), 2-chloro-4-methylpyrimidine (17.67 g, 137 mmol, 1.05 eq.) and Cs₂CO₃ (85.3 g, 262 mmol, 2 eq.) in NMP (300 mL) was stirred at 100° C. for 16 hrs. The reaction mixture was partitioned between water (400 mL) and EtOAc (3×200 mL). The combined organic layers were washed with brine (3×200 mL), dried over anhydrous Na₂SO₄ and filtered. The filtrate was concentrated under reduced pressure. The resulting residue was purified by silica gel column chromatography, eluting with PE/EtOAc (8:1) to afford the title compound (27.3 g, 74%) as a white solid.

LCMS: m/z 283.15 [M+1]⁺.

### Scheme AK, Step 2. Synthesis of 2-(2-fluoro-4-(4,4,5,5-tetramethyl-1,3,2-dioxaborolan-2-yl)phenoxy)-4-methylpyrimidine

A mixture of 2-(4-bromo-2-fluorophenoxy)-4-methylpyrimidine (3 g, 10.6 mmol, 1 eq.), B₂Pin₂ (2.97 g, 11.7 mmol, 1.1 eq.), Pd(dppf)Cl₂ (865 mg, 1.06 mmol, 0.1 eq.) and KOAc (3.12 g, 31.8 mmol, 3 eq.) in dioxane (40 mL) was degassed with N₂ three times, and then stirred under N₂ at 90° C. for 16 hrs. The solution was filtered over a filter pad. The filtrate was concentrated under reduced pressure to afford the title compound (3.3 g, 99%) as a brown solid which was used in the next step directly.

### Scheme AK, Step 3. Synthesis of 5-(3-fluoro-4-((4-methylpyrimidin-2-yl)oxy)phenyl)-7-methyl-7H-pyrrolo[2,3-d]pyrimidin-4-amine

The mixture of 5-bromo-7-methyl-7H-pyrrolo[2,3-d]pyrimidin-4-amine (1.14 g, 5 mmol, 1 eq.), 2-(2-fluoro-4-(4,4,5,5-tetramethyl-1,3,2-dioxaborolan-2-yl)phenoxy)-4-methylpyrimidine (3.3 g, 10 mmol, 2 eq.), K₃CO₃ (2.07 g, 15 mmol, 3 eq.) and Pd(DtBPF)Cl₂ (163 mg, 0.25 mmol, 0.05 eq.) in dioxane (25 mL) and H₂O (2.5 mL) was degassed three times with N₂ and then stirred at 90° C. for 16 h under N₂. The resulting mixture was diluted with H₂O (100 mL) and extracted with EtOAc (3×50 mL).

The combined organic layers were dried over anhydrous Na₂SO₄ and filtered. The filtrate was concentrated under reduced pressure. The residue was purified by silica gel column chromatography, eluting with DCM/MeOH (20:1) to afford the title compound (1.25 g, 71%) as a yellow solid.

LCMS: m/z 351.30 [M+1]⁺.

### Scheme AK, Step 4. Synthesis of 5-(3-fluoro-4-((4-methylpyrimidin-2-yl)oxy)phenyl)-6-iodo-7-methyl-7H-pyrrolo[2,3-d]pyrimidin-4-amine

To a solution of 5-(3-fluoro-4-((4-methylpyrimidin-2-yl)oxy)phenyl)-7-methyl-7H-pyrrolo[2,3-d]pyrimidin-4-amine (1.25 g, 3.57 mmol, 1 eq.) in DCM (20 mL) were added NIS (1.2 g, 5.36 mmol, 1.5 eq.) and CF₃COOH (2.03 g, 17.9 mmol, 5 eq.) at 0° C. under N₂. The resulting mixture was stirred at room temperature for 2 hrs, diluted with NaHCO₃ aqueous solution (20 mL), 10% Na₂SO₃ solution (15 mL) and H₂O (20 mL). The resulting precipitates were collected by filtration, washed with water and purified by silica gel column chromatography, eluting with DCM/MeOH (60:1) to afford the title compound (1.34 g, 79%) as a white solid.

LCMS: m/z 477.25 [M+1]⁺.

### Scheme AK, Step 5. Synthesis of tert-butyl 9-(4-amino-5-(3-fluoro-4-((4-methylpyridin-2-yl)oxy)phenyl)-7-methyl-7H-pyrrolo[2,3-d]pyrimidin-6-yl)-3-azaspiro[5.5]undec-8-ene-3-carboxylate

A mixture of 5-(3-fluoro-4-((4-methylpyrimidin-2-yl)oxy)phenyl)-6-iodo-7-methyl-7H-pyrrolo[2,3-d]pyrimidin-4-amine (1.34 g, 2.82 mmol, 1 eq.), tert-butyl 9-(4,4,5,5-tetramethyl-1,3,2-dioxaborolan-2-yl)-3-azaspiro[5.5]undec-8-ene-3-carboxylate (1.59 g, 4.22 mmol, 1.5 eq.), K₂CO₃ (1.17 g, 8.46 mmol, 3 eq.) and Pd(DtBPF)Cl₂ (92 mg, 0.14 mmol, 0.05 eq.) in dioxane (20 mL) and H₂O (2 mL) was degassed three times with N₂ and then stirred at 90° C. for 16 h under N₂. The resulting mixture was diluted with H₂O (40 mL) and extracted with EtOAc (3×30 mL). The combined organic layers were dried over anhydrous Na₂SO₄ and filtered. The residue was purified by silica gel column chromatography, eluting with DCM/MeOH (60:1) to afford the title compound (1.03 g, 61%) as a brown solid.

LCMS: m/z 599.35 [M+1]⁺.

### Scheme AK, Step 6. Synthesis of 5-(3-fluoro-4-((4-methylpyrimidin-2-yl)oxy)phenyl)-7-methyl-6-(3-azaspiro[5.5]undec-8-en-9-yl)-7H-pyrrolo[2,3-d]pyrimidin-4-amine

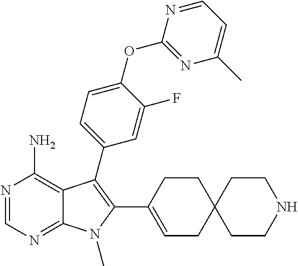 [SMILES: Cc1ccnc(Oc2ccc(-c3c(C4=CCC5(CCNCC5)CC4)n(C)c4ncnc(N)c34)cc2F)n1]

### Preparation 4

A mixture of tert-butyl 9-(4-amino-5-(3-fluoro-4-((4-methylpyridin-2-yl)oxy)phenyl)-7-methyl-7H-pyrrolo[2,3-d]pyrimidin-6-yl)-3-azaspiro[5.5]undec-8-ene-3-carboxylate (1.03 g, 1.72 mmol, 1 eq.) in TFA (5 mL) and in DCM (10 mL) was stirred at room temperature for 2 hrs. The resulting mixture was concentrated under reduced pressure. The residue was dissolved in DCM (10 mL), adjusted the pH to 9 with NH₄OH. The resulting precipitates were collected by filtration, washed with water and DCM to afford the title compound (515 mg, 60%) as a white solid.

LCMS: m/z 499.15 [M+1]⁺.

### Example 62

### Synthesis of 1-(9-(4-amino-5-(3-fluoro-4-((4-methylpyrimidin-2-yl)oxy)phenyl)-7-methyl-7H-pyrrolo[2,3-d]pyrimidin-6-yl)-3-azaspiro[5.5]undec-8-en-3-yl)prop-2-en-1-one

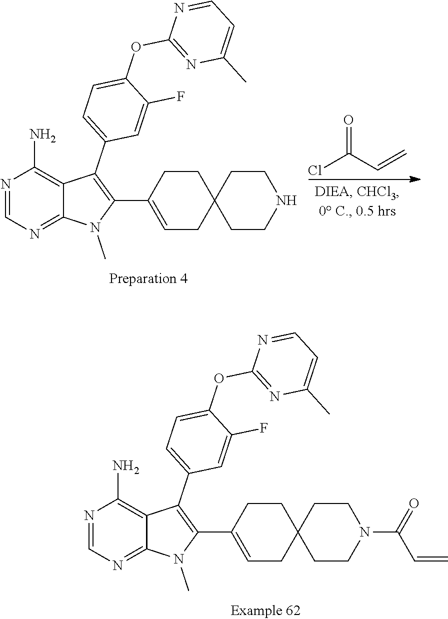

To a stirred solution of 5-(3-fluoro-4-((4-methylpyrimidin-2-yl)oxy)phenyl)-7-methyl-6-(3-azaspiro[5.5]undec-8-en-9-yl)-7H-pyrrolo[2,3-d]pyrimidin-4-amine (50 mg, 0.1 mmol, 1 eq.) in dry CHCl₃ (5 mL) was added DIEA (38.7 mg, 0.3 mmol, 3 eq.). A solution of acryloyl chloride (9.5 mg, 0.105 mmol, 1.05 eq) in dry CHCl₃ (0.5 mL) was added dropwise at 0° C. and the mixture was stirred at 0° C. for 0.5 hrs under N₂. The reaction mixture was quenched with water. The resulting mixture was extracted with DCM (3×10 mL). The combined organic layers were dried over anhydrous Na₂SO₄ and filtered. The solvent was removed under reduced pressure. The residue was purified by Prep-TLC (DCM/MeOH=20/1) to afford the title compound (25 mg, 45% yield) as a white solid.

LCMS: m/z 554.35 [M+1]⁺.

¹H NMR (400 MHz, CD₃OD) δ 8.42 (d, J=5.1 Hz, 1H), 8.13 (s, 1H), 7.35 (t, J=8.4 Hz, 1H), 7.25 (t, J=9.0 Hz, 2H), 7.15 (d, J=5.0 Hz, 1H), 6.72 (dd, J=16.8, 10.7 Hz, 1H), 6.14 (dd, J=16.8, 1.9 Hz, 1H), 5.88 (s, 1H), 5.69 (dd, J=10.7, 1.7 Hz, 1H), 3.74-3.62 (m, 5H), 3.52-3.44 (m, 2H), 2.48 (s, 3H), 2.15 (s, 2H), 2.15-2.06 (m, 2H), 1.61 (t, J=6.3 Hz, 2H), 1.48-1.40 (m, 4H).

1. A compound of formula I 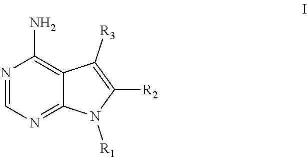
wherein
R₁ is C1-C6 alkyl or substituted C1-C6 alkyl;
R₂ is substituted C7-C19 spiro bicycloalkanyl, substituted C7-C19 spiro bicycloalkenyl, substituted C7-C19 spiro heterobicycloalkanyl or substituted C7-C19 spiro heterobicycloalkenyl; and
R₃ is C1-C6 alkyl, substituted C1-C6 alkyl, C3-C10 cycloalkyl, substituted aryl, heteroaryl or substituted heteroaryl;
or a pharmaceutically acceptable salt thereof.

2. The compound of claim 1, wherein
R₁ is C1-C6 alkyl or substituted C1-C6 alkyl;
R₂ is 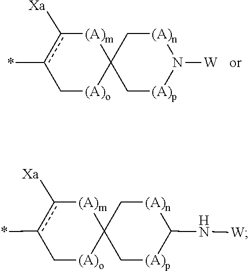
(A)ₘ is CRₐR_{b}
(A)ₙ is CRₐR_{b};
(A)ₒ is CRₐR_{b} or O;
(A)ₚ is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
provided when (A)ₒ is O, o is 1;
Xa is H or halogen;
W is COR₅;
R₅ is 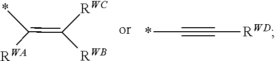
R^{WA}, R^{WB}, R^{WC} are on each occurrence independently selected from the group consisting of H, C1-C6 alkyl, substituted C1-C6 alkyl, halogen and CN;
R^{WD} is H or C1-C6 alkyl; and
R₃ is C1-C6 alkyl, substituted C1-C6 alkyl, substituted aryl, heteroaryl or substituted heteroaryl;
or a pharmaceutically acceptable salt thereof.

3. The compound of claim 2 wherein m, n, o and p are on each occurrence independently selected from the group consisting of 0, 1 or 2; or a pharmaceutically acceptable salt thereof.

4. The compound of claim 2 wherein
R₅ is 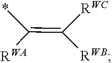
and
R^{WA}, R^{WB}, R^{WC} are on each occurrence independently selected from the group consisting of H and C1-C6 alkyl;
or a pharmaceutically acceptable salt thereof.

5. The compound of claim 2, wherein
R₂ is 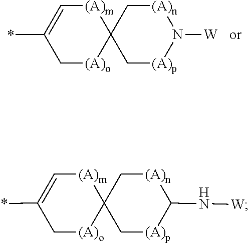
or a pharmaceutically acceptable salt thereof.

6. The compound of claim 2, wherein
R₂ is 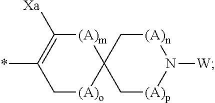
(A)ₘ is CRₐR_{b}
(A)ₙ is CRₐR_{b};
(A)ₒ is CRₐR_{b} or O;
(A)ₚ is CRₐR_{b};
Rₐ is on each occurrence independently selected from the group consisting of H and C1-C3 alkyl;
R_{b} is on each occurrence independently selected from the group consisting of H and C1-C3 alkyl;
m, n, o and p are on each occurrence independently selected from the group consisting of 0, 1, 2 or 3;
provided when (A)ₒ is O, o is 1;
Xa is H or halogen;
W is COR₅;
R₅ is 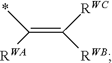
R^{WA}, R^{WB}, R^{WC} are on each occurrence independently selected from the group consisting of H, C1-C6 alkyl, substituted C1-C6 alkyl, halogen and CN;
or a pharmaceutically acceptable salt thereof.

7. The compound of claim 2, wherein
R₁ is CH₃;
R₂ is
R₃ is 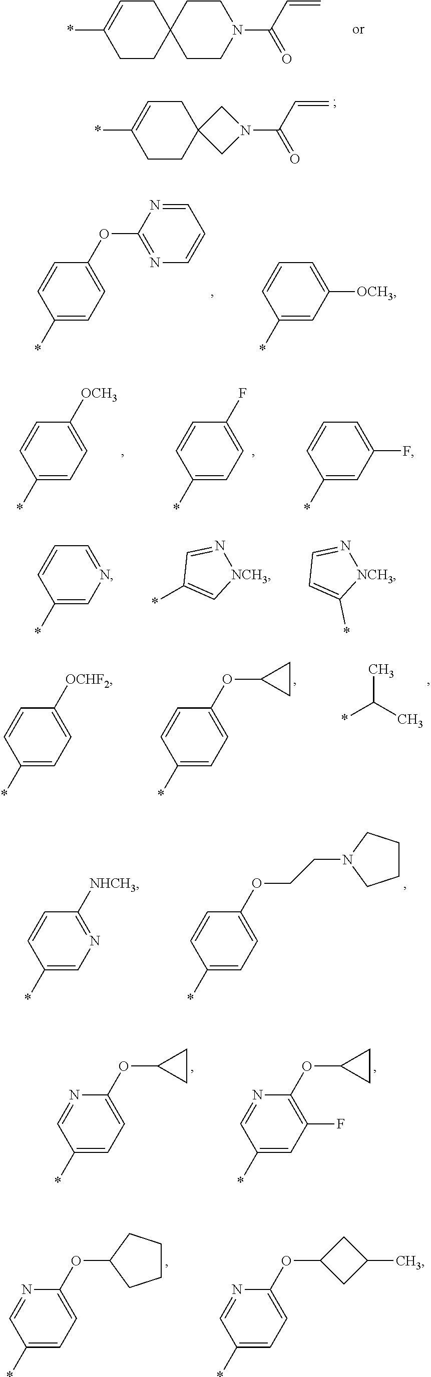 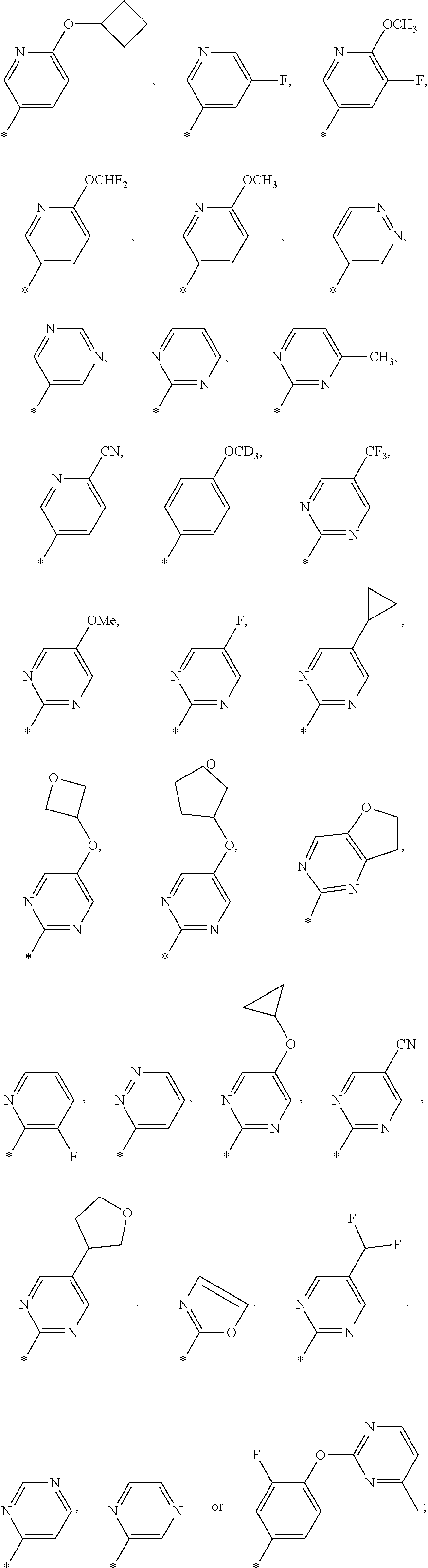
or a pharmaceutically acceptable salt thereof.

8. The compound of claim 1, wherein
R₁ is CH₃ or CHF₂;
R₂ is 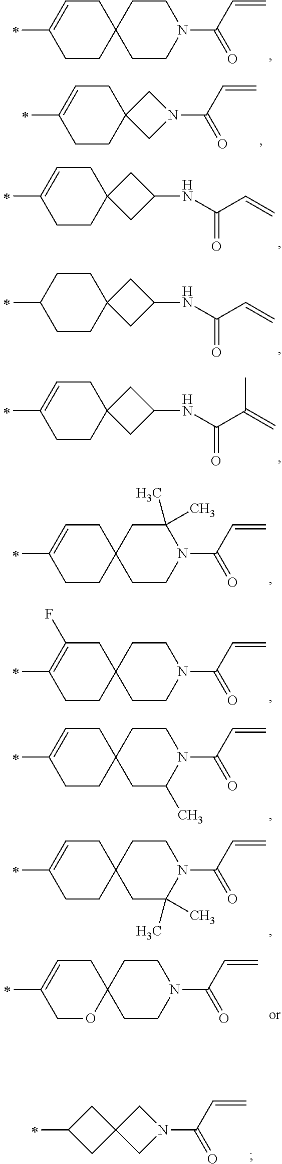
R₃ is 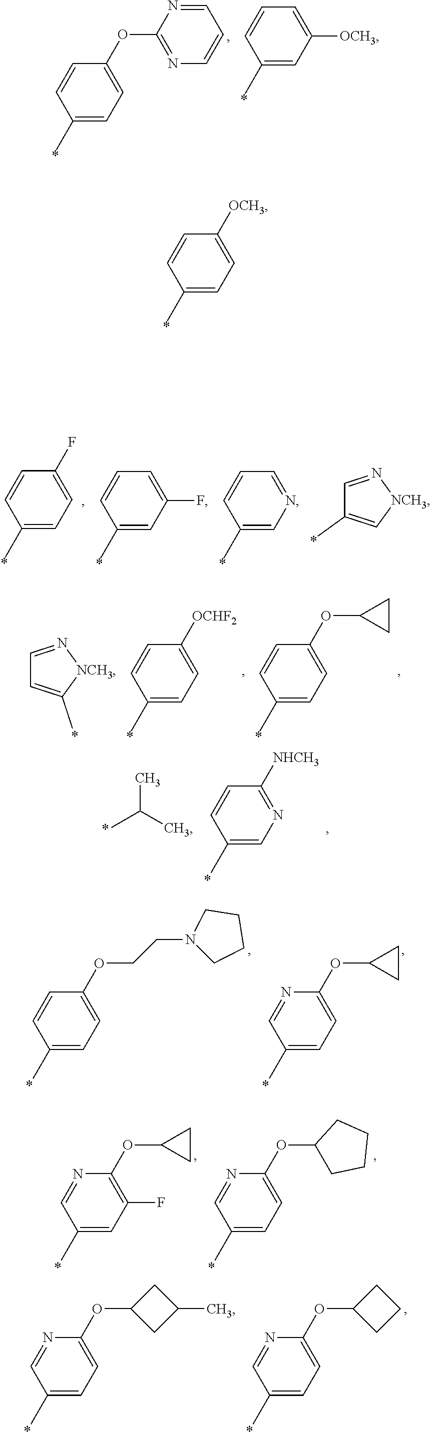 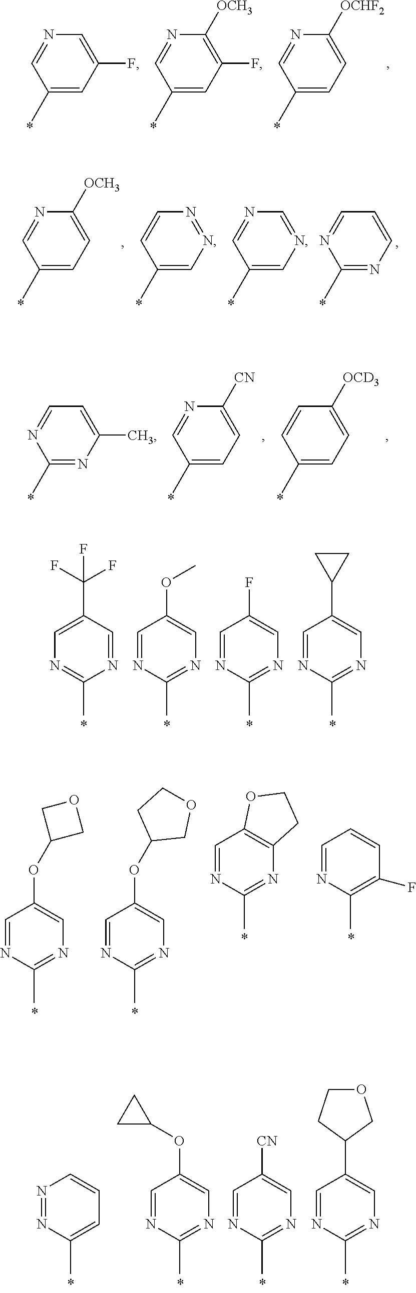 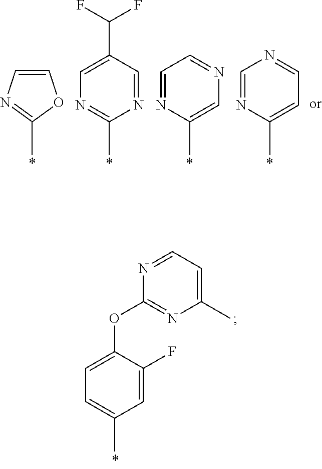
or a pharmaceutically acceptable salt thereof.

9. A compound or a pharmaceutically acceptable salt thereof selected from the group consisting of
1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)prop-2-en-1-one;
1-(7-(4-amino-7-methyl-5-(4-(pyrimidin-2-yloxy)phenyl)-7H-pyrrolo[2,3-d]pyrimidin-6-yl)-2-azaspiro[3.5]non-6-en-2-yl)-2-methylprop-2-en-1-one;
1-(6-(4-amino-7-methyl-5-(4-(pyrimidin-2-yloxy)phenyl)-7H-pyrrolo[2,3-d]pyrimidin-6-yl)-2-azaspiro[3.3]heptan-2-yl)prop-2-en-1-one;
1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one;
1-(8-(4-amino-7-methyl-5-(4-(pyrimidin-2-yloxy)phenyl)-7H-pyrrolo[2,3-d]pyrimidin-6-yl)-2-azaspiro[4.5]dec-7-en-2-yl)prop-2-en-1-one;
N-(7-(4-amino-5-(4-methoxyphenyl)-7-methyl-7H-pyrrolo[2,3-d]pyrimidin-6-yl)spiro[3.5]non-6-en-2-yl)acrylamide;
1-(9-(4-amino-5-(4-methoxyphenyl)-7-methyl-7H-pyrrolo[2,3-d]pyrimidin-6-yl)-3-azaspiro[5.5]undec-8-en-3-yl)prop-2-en-1-one;
N-(7-(4-amino-7-methyl-5-(4-(pyrimidin-2-yloxy)phenyl)-7H-pyrrolo[2,3-d]pyrimidin-6-yl)spiro[3.5]non-6-en-2-yl)acrylamide;
1-(9-(4-amino-5-(3-fluorophenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-fluorophenyl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyridin-3-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(1-methyl-1H-pyrazol-5-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(1-methyl-1H-pyrazol-4-yl)-7H-pyrrolo[2,3-d]pyrimidin-6-yl)-3-azaspiro[5.5]undec-8-en-3-yl)prop-2-en-1-one;
1-(9-(4-amino-5-isopropyl-7-methyl-7H-pyrrolo[2,3-d]pyrimidin-6-yl)-3-azaspiro[5.5]undec-8-en-3-yl)prop-2-en-1-one;
1-(9-(4-amino-5-(3-methoxyphenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-(difluoromethoxy)phenyl)-7-methyl-7H-pyrrolo[2,3-d]pyrimidin-6-yl)-3-azaspiro[5.5]undec-8-en-3-yl)prop-2-en-1-one;
1-(9-(4-amino-5-(4-cyclopropoxyphenyl)-7-methyl-7H-pyrrolo[2,3-d]pyrimidin-6-yl)-3-azaspiro[5.5]undec-8-en-3-yl)prop-2-en-1-one;
N-(7-(4-amino-5-(4-methoxyphenyl)-7-methyl-7H-pyrrolo[2,3-d]pyrimidin-6-yl)spiro[3.5]nonan-2-yl)acrylamide;
1-(9-(4-amino-7-methyl-5-(4-(2-(pyrrolidin-1-yl)ethoxy)phenyl)-7H-pyrrolo[2,3-d]pyrimidin-6-yl)-3-azaspiro[5.5]undec-8-en-3-yl)prop-2-en-1-one;
1-(9-(4-amino-7-methyl-5-(6-(methylamino)pyridin-3-yl)-7H-pyrrolo[2,3-d]pyrimidin-6-yl)-3-azaspiro[5.5]undec-8-en-3-yl)prop-2-en-1-one;
1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-cyclopropoxy-5-fluoropyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-(cyclopentyloxy)pyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(6-(3-methylcyclobutoxy)pyridin-3-yl)-7H-pyrrolo[2,3-d]pyrimidin-6-yl)-3-azaspiro[5.5]undec-8-en-3-yl)prop-2-en-1-one;
1-(9-(4-amino-5-(6-cyclobutoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoropyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoro-6-methoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-(difluoromethoxy)pyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-3-azaspiro[5.5]undec-8-en-3-yl)prop-2-en-1-one
1-(9-(4-amino-7-methyl-5-(pyridazin-4-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-5-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-2,2-dimethyl-3-azaspiro[5.5]undec-8-en-3-yl)prop-2-en-1-one;
1-(9-(4-amino-7-methyl-5-(4-methylpyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(4-cyclopropoxyphenyl)-7-(difluoromethyl)-7H-pyrrolo[2,3-d]pyrimidin-6-yl)-3-azaspiro[5.5]undec-8-en-3-yl)prop-2-en-1-one;
5-(6-(3-acryloyl-3-azaspiro[5.5]undec-8-en-9-yl)-4-amino-7-(difluoromethyl)-7H-pyrrolo[2,3-d]pyrimidin-5-yl)picolinonitrile;
1-(9-(4-amino-5-(4-(methoxy-d3)phenyl)-7-methyl-7H-pyrrolo[2,3-d]pyrimidin-6-yl)-8-fluoro-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-2-methyl-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-2,2-dimethyl-3-azaspiro[5.5]undec-8-en-3-yl)prop-2-en-1-one;
1-(3-(4-amino-5-(4-fluorophenyl)-7-methyl-7H-pyrrolo[2,3-d]pyrimidin-6-yl)-1-oxa-9-azaspiro[5.5]undec-3-en-9-yl)prop-2-en-1-one;
1-(9-(4-amino-7-methyl-5-(5-(trifluoromethyl)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(5-methoxypyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-fluoropyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(5-cyclopropylpyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(5-(oxetan-3-yloxy)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(5-((tetrahydrofuran-3-yl)oxy)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-5-(6,7-dihydrofuro[3,2-d]pyrimidin-2-yl)-7-methyl-7H-pyrrolo[2,3-d]pyrimidin-6-yl)-3-azaspiro[5.5]undec-8-en-3-yl)prop-2-en-1-one
1-(9-(4-amino-5-(3-fluoropyridin-2-yl)-7-methyl-7H-pyrrolo[2,3-d]pyrimidin-6-yl)-3-azaspiro[5.5]undec-8-en-3-yl)prop-2-en-1-one;
2-(6-(3-acryloyl-3-azaspiro[5.5]undec-8-en-9-yl)-4-amino-7-methyl-7H-pyrrolo[2,3-d]pyrimidin-5-yl)pyrimidine-5-carbonitrile;
1-(9-(4-amino-7-methyl-5-(5-(tetrahydrofuran-3-yl)pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(oxazol-2-yl)-7H-pyrrolo[2,3-d]pyrimidin-6-yl)-3-azaspiro[5.5]undec-8-en-3-yl)prop-2-en-1-one;
1-(9-(4-amino-7-(2-hydroxyethyl)-5-(pyrimidin-2-yl)-7H-pyrrolo[2,3-d]pyrimidin-6-yl)-3-azaspiro[5.5]undec-8-en-3-yl)prop-2-en-1-one;
1-(9-(4-amino-7-methyl-5-(pyridazin-3-yl)-7H-pyrrolo[2,3-d]pyrimidin-6-yl)-3-azaspiro[5.5]undec-8-en-3-yl)prop-2-en-1-one;
1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyrazin-2-yl)-7H-pyrrolo[2,3-d]pyrimidin-6-yl)-3-azaspiro[5.5]undec-8-en-3-yl)prop-2-en-1-one;
1-(9-(4-amino-5-(5-cyclopropoxypyrimidin-2-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-4-yl)-7H-pyrrolo[2,3-d]pyrimidin-6-yl)-3-azaspiro[5.5]undec-8-en-3-yl)prop-2-en-1-one; and
1-(9-(4-amino-5-(3-fluoro-4-((4-methylpyrimidin-2-yl)oxy)phenyl)-7-methyl-7H-pyrrolo[2,3-d]pyrimidin-6-yl)-3-azaspiro[5.5]undec-8-en-3-yl)prop-2-en-1-one.

10. The compound of claim 9 or a pharmaceutically acceptable salt thereof selected from the group consisting of
1-(9-(4-amino-7-methyl-5-(4-(pyrimidin-2-yloxy)phenyl)-7H-pyrrolo[2,3-d]pyrimidin-6-yl)-3-azaspiro[5.5]undec-8-en-3-yl)prop-2-en-1-one;
1-(9-(4-amino-5-(6-cyclopropoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-5-(6-methoxypyridin-3-yl)-7-methyl-7H-pyrrolo[2,3-d]pyrimidin-6-yl)-3-azaspiro[5.5]undec-8-en-3-yl)prop-2-en-1-one;
1-(9-(4-amino-7-methyl-5-(pyrimidin-2-yl)-7H-pyrrolo[2,3-d]pyrimidin-6-yl)-3-azaspiro[5.5]undec-8-en-3-yl)prop-2-en-1-one; and
1-(9-(4-amino-5-(5-(difluoromethyl)pyrimidin-2-yl)-7-methyl-7H-pyrrolo[2,3-d]pyrimidin-6-yl)-3-azaspiro[5.5]undec-8-en-3-yl)prop-2-en-1-one.

11. A method of treating cancer comprising administrating to a patient in need thereof an effective amount of a compound according to claim 1.

12. The method according to claim 11 wherein the cancer is selected from the group consisting of endometrial carcinoma, gastric cancer, and intrahepatic cholangiocarcinoma.

13. A pharmaceutical composition comprising a compound according to claim 1, or a pharmaceutically acceptable salt thereof, and a pharmaceutically acceptable carrier, diluent or excipient.

14-16. (canceled)
